(19) United States
(12) Patent Application Publication
(10) Pub. No.: US 2008/0090834 A1
(43) Pub. Date: Apr. 17, 2008

(54) SELECTIVE AZOLE PDE10A INHIBITOR COMPOUNDS

(75) Inventors: Dennis J. Hoover, Mystic, CT (US); Kevin G. Witter, Norwich, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 11/770,793

(22) Filed: Jun. 29, 2007

Related U.S. Application Data
(60) Provisional application No. 60/819,554, filed on Jul. 6, 2006.

(51) Int. Cl. A61K 31/437; A61K 31/4184; A61K 31/444; A61K 31/496; A61K 31/497; A61K 31/506; A61K 31/52; A61P 25/00; A61P 3/04; C07D 401/14; C07D 487/00; C07D 487/04; C07D 495/14

(52) U.S. Cl. 514/253.06; 514/255.05; 514/256; 514/263.22; 514/293; 514/300; 514/303; 514/333; 514/394; 544/277; 544/328; 544/363; 544/405; 546/113; 546/118; 546/256; 546/83; 548/306.1

(57) ABSTRACT

The invention pertains to heteroaromatic compounds of the formula I,

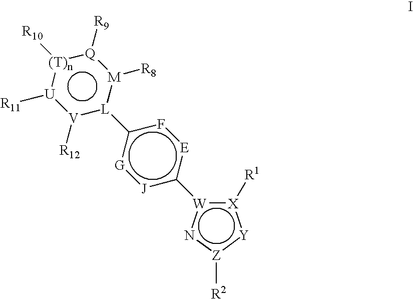

as defined herein, that serve as effective phosphodiesterase (PDE) inhibitors. In particular, the invention relates to said compounds which are selective inhibitors of PDE10. The invention also relates to pharmaceutical compositions comprising said compounds; and the use of said compounds in a method for treating certain central nervous system (CNS) or other disorders.

### CROSS REFERENCE TO RELATED APPLICATION

The present invention claims benefit of U.S. Ser. No. 60/819,554 filed on Jul. 6, 2008, which is hereby incorporated by reference in its entirety.

### FIELD OF THE INVENTION

The invention pertains to heteroaromatic compounds. This invention also relates to compounds that serve as effective phosphodiesterase (PDE) inhibitors. The invention also relates to compounds which are selective inhibitors of PDE10. The invention further relates to pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### BACKGROUND OF INVENTION

Phosphodiesterases (POEs) are a class of intracellular enzymes involved in the hydrolysis of the nucleotides cyclic adenosine monophosphate (cAMP) and cyclic guanosine monophosphates (cGMP) into their respective nucleotide monophosphates. The cyclic nucleotides cAMP and cGMP are synthesized by adenylyl and guanylyl cyclases, respectively, and serve as secondary messengers in several cellular pathways.

The cAMP and cGMP function as intracellular second messengers regulating a vast array of intracellular processes particularly in neurons of the central nervous system. In neurons, this includes the activation of cAMP and cGMP-dependent kinases and subsequent phosphorylation of proteins involved in acute regulation of synaptic transmission as well as in neuronal differentiation and survival. The complexity of cyclic nucleotide signaling is indicated by the molecular diversity of the enzymes involved in the synthesis and degradation of cAMP and cGMP. There are at least ten families of adenylyl cyclases, two of guanylyl cyclases, and eleven of phosphodiesterases. Furthermore, different types of neurons are known to express multiple isozymes of each of these classes, and there is good evidence for compartmentalization and specificity of function for different isozymes within a given neuron.

A principal mechanism for regulating cyclic nucleotide signaling is by phosphodiesterase-catalyzed cyclic nucleotide catabolism. There are 11 known families of PDEs encoded by 21 different genes. Each gene typically yields multiple splice variants that further contribute to the isozyme diversity. The PDE families are distinguished functionally based on cyclic nucleotide substrate specificity, mechanism(s) of regulation, and sensitivity to inhibitors. Furthermore, PDEs are differentially expressed throughout the organism, including in the central nervous system. As a result of these distinct enzymatic activities and localization, different PDEs isozymes can serve distinct physiological functions. Furthermore, compounds that can selectively inhibit distinct PDE families or isozymes may offer particular therapeutic effects, fewer side effects, or both.

PDE10 is identified as a unique family based on primary amino acid sequence and distinct enzymatic activity. Homology screening of EST databases revealed mouse PDE10A as the first member of the PDE10 family of PDEs (Fujishige et al., J. Bioi. Chem. 274: 18438-18445, 1999; Loughney, K. et al., Gene 234: 109-117, 1999). The murine homologue has also been cloned (Soderling, S. et al., Proc. Natl. Acad. Sci, USA 96: 7071-7076, 1999) and N-terminal splice variants of both the rat and human genes have been identified (Kotera, J. et al., Biochem. Biophys. Res. Comm. 261: 551-557, 1999; Fujishige, K. et al., Eur. J. Biochem. 266: 1118-1127, 1999). There is a high degree of homology across species. The mouse PDE10A1 is a 779 amino acid protein that hydrolyzes both cAMP and cGMP to AMP and GMP, respectively. The affinity of PDE10 for cAMP (Km=0.05 μM) is higher than for cGMP (Km=3 μM). However, the approximately 5-fold greater Vmax for cGMP over cAMP has lead to the suggestion that PDE10 is a unique cAMP-inhibited cGMPase (Fujishige et al., J. Bioi. Chem. 274: 18438-18445, 1999).

The PDE10 family of polypeptides shows a lower degree of sequence homology as compared to previously identified PDE families and has been shown to be insensitive to certain inhibitors that are known to be specific for other PDE families. U.S. Pat. No. 6,350,603, incorporated herein by reference.

PDE10 also is uniquelylocalized in mammals relative to other PDE families. mRNA for PDE10 is highly expressed only in testis and brain (Fujishige, K. et al., Eur J. Biochem. 266: 1118-1127, 1999; Soderling, S, et al. Proc. Natl. Acad. Sci. 96: 7071-7076, 1999; Loughney, K. et at., Gene 234: 109-117, 1999). These initial studies indicated that within the brain PDE10 expression is highest in the striatum (caudate and putamen), n, accumbens, and olfactory tubercle. More recently, a detailed analysis has been made of the expression pattern in rodent brain of PDE10 mRNA (Seeger, T. F. et al., Abst. Soc. Neurosci. 26: 345.10, 2000) and PDE10 protein (Menniti, F. S, Stick, C. A., Seeger, T. F, and Ryan, A. M., Immunohistochemical localization of PDE10 in the rat brain. William Harvey Research Conference Phosphodiesterase in Health and Disease, Porto, Portugal, Dec. 5-7, 2001).

A variety of therapeutic uses for PDE inhibitors has been reported including obtrusive lung disease, allergies, hypertension, angina, congestive heart failure, depression and erectile dysfunction (WO 01/41807 A2, incorporated herein by reference).

The use of selected benzimidazole and related heterocyclic compounds in the treatment of ischemic heart conditions has been disclosed based upon inhibition of PDE associated cGMP activity. U.S. Pat. No. 5,693,652, incorporated herein by reference.

United States Patent Application Publication No. 2003/0032579 discloses a method for treating certain neurologic and psychiatric disorders with the selective PDE10 inhibitor papaverine. In particular, the method relates to psychotic disorders such as schizophrenia, delusional disorders and drug-induced psychosis; to anxiety disorders such as panic and obsessive-compulsive disorder; and to movement disorders including Parkinson's disease and Huntington's disease. Other indications which may be treated using a PDE10 inhibitor are described in WO 2005/5120514.

The entire teachings of the aforementioned patents and patent applications are incorporated herein by reference.

### SUMMARY OF THE INVENTION

The present invention provides for compounds of formula I.

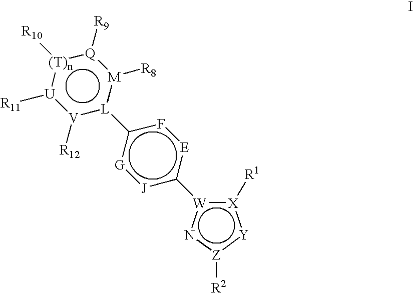

and pharmaceutical acceptable salts thereof;

wherein N, W, X, Y, and Z together form a 5-membered heteroaromatic ring;

W, X, and Z are independently selected from the group consisting of carbon and nitrogen;

Y is selected from the group consisting of CR²⁰, N, N(O), NR²¹, S, and O;

with the proviso that at least two of W, X, and Z are carbon or at least one of W, X, and Z is carbon and Y is CR²⁰;

R¹ is selected from the group consisting of phenyl, a 5 to 6-membered heteroaryl, naphthyl, a 5 to 6-membered heteroaryl fused to a 5 to 6-membered heteroaromatic ring, phenyl fused to a 5 to 6-membered heteroaromatic ring, a 5 to 6-membered heteroaryl fused to benzene, a phenyl fused to a 5 to 7-membered cycloalkane, a 5 to 6-membered heteroaryl fused to a 5 to 7-membered cycloalkane, phenyl fused to a 5 to 7-membered heterocycloalkane, and a 5 to 6-membered heteroaryl fused to a 5 to 7-membered heterocycloalkane, wherein said heteroaromatic rings, heteroaryls, and heterocycloalkanes independently contain 1 to 4 heteroatoms independently selected from the group consisting of O, N, and S; and wherein said phenyl and heteroaryl groups of said fused groups are directly bonded to X; and wherein R¹ is optionally substituted with 1 to 3 substituents, independently selected from the group consisting of hydroxy, nitro, oxo, and R³; wherein one of said substituents is optionally further selected from the group consisting of R^{3a};

wherein R³ is independently selected from the group consisting of halo, cyano, formyl, carbamoyl, carboxy, amino, (C₁-C₆)alkyl, cyclopropyl, (C₃-C₇)cycloalkyl-(C₁-C₃)alkyl-, cyano-(C₁-C₄)alkyl-, —OR¹³, hydroxy(C₁-C₆)alkyl-, R¹³O—(C₁-C₆)alkyl-, R¹³S—(C₁-C₆)alkyl-hydroxy-(C₁-C₈)alkoxy-, R¹³O—(C₁-C₈)alkoxy-, amino-(C₂-C₆)alkoxy-, R¹³R¹⁴N—(C₂-C₆)alkoxy-, hydroxy-(C₂-C₆)alkyl-N(R¹⁴)—, R¹³O—(C₂-C₆)alkyl-N(R¹⁴)—, hydroxy-(C₁-C₈)alkyl-S—, R¹³O—(C₃-C₆)alkyl-S—, —SR¹³, —S(O)R¹⁸, —S(O)₂R¹³, —S(O)₂NH₂, —S(O)₂NR¹³R¹⁴, —C(═O)R¹³, —OC(═O)H, —OC(═O)R¹³, —OC(═O)OR¹³, —C(═O)OR¹³, carboxy-(C₁-C₄)alkyl-, R¹³OC(═O)—(C₁-C₄)alkyl-, carbamoyl-(C₁-C₄)alkyl-, R¹³R¹⁴NC(═O)—(C₁-C₄)alkyl-, carboxy-(C₁₋C₄)alkoxy-, R¹³OC(═O)—(C₁-C₄)alkoxy-, carbamoyl-(C₁-C₄)alkoxy-, R¹³R¹⁴NC(═O)—(C₁₋C₄)alkoxy-, amino-(C₁-C₆)alkyl-, R¹³R¹⁴N—(C₁-C₆)alkyl-, R¹³R¹⁴N—(C₂-C₈)alkoxy-, —C(═O)NR¹³R¹⁴, —OC(═O)NH₂, —OC(═O)NR¹³R¹⁴, —N(R¹⁴)C(═O)H, —N(R¹⁴)C(═O)R¹³, phenyl-A-, 5 to 6-membered heteroaryl-A-, phenyl-(A)ₘ-(C₁-C₄ alkyl), and 5 to 6-membered heteroaryl-(A)ₘ-(C₁-C₄ alkyl); wherein said phenyls and heteroaryls are optionally substituted with 1 to 3 substituents independently selected from halo, trifluoromethyl, hydroxy, cyano, cyano-(C₁-C₄)alkyl, R¹³, —OR¹³, hydroxy-(C₁-C₆)alkyl, and R¹³O—(C₁-C₆)alkyl; and wherein said alkyl, cycloalkyl, cycloalkyl-alkyl, and alkoxy groups are optionally independently substituted with 1 to 5 fluorine atoms; wherein A is independently O or S; and wherein m is independently 0 or 1;

wherein R^{3a} is (C₄-C₇)cycloalkyl, (C₂-C₅)alkenyl, (C₂-C₆)alkynyl, NR¹³R¹⁴, phenyl, 5 to 6-membered heteroaryl, or 4 to 6-membered heterocycyl containing 1 to 3 heteroatoms selected from N, O, and S; wherein said cycloalkyl, alkenyl, and alkynyl groups are optionally independently substituted with 1 to 3 fluorine atoms; and wherein said phenyl, heteroaryl, and heterocyclic groups are optionally substituted with 1 to 3 substituents independently selected from halo, trifluoromethyl, hydroxy, cyano, cyano-(C₁-C₄)alkyl, R¹³, —OR¹³, hydroxy-(C₁-C₅)alkyl, and R¹³O—(C₁-C₆)alkyl;

wherein R¹³ is independently selected from the group consisting of (C₁-C₆)alkyl, (C₃-C₇)cycloalkyl, and (C₃-C₇)cycloalkane-(C₁-C₃)alkyl-; wherein said alkyl, cycloalkyl, and cycloalkyl-alkyl- groups are optionally independently substituted with 1 to 5 fluorine atoms;

wherein R¹⁴ is independently selected from the group consisting of H, (C₁-C₆)alkyl, (C₃-C₅)alkoxy, (C₃-C₅)cycloalkyl, and (C₃-C₅)cycloalkane-(C₁-C₃)alkyl-; wherein said alkyl, alkoxy, and cycloalkyl groups are optionally independently substituted with 1 to 3 fluorine atoms;

or optionally R¹³ and R¹⁴ together with the nitrogen to which they are attached form a 4 to 6-membered heterocyclic ring containing 1 to 3 heteroatoms selected from N, O, and S; wherein said heterocyclic ring may be optionally substituted with 1 to 4 substituents independently selected from fluoro, (C₁-C₄)alkyl, and (C₁-C₄)alkoxy; and wherein 1 to 2 of said substituents may be further selected from hydroxy, oxo, and trifluoromethyl;

R² is selected from the group consisting of phenyl, a 5 to 6-membered heteroaryl, naphthyl, a 5 to 6-membered heteroaryl fused to a 5 to 6-membered heteroaromatic ring, phenyl fused to a 5 to 6-membered heteroaromatic ring, and a 5 to 6-membered heteroaryl fused to benzene; wherein said heteroaryls and heteroaromatic rings each independently contain 1 to 3 heteroatoms independently selected from the group consisting of O, N, and S; and wherein said phenyl and heteroaryl groups of said fused groups are directly bonded to Z:

and wherein R² is optionally substituted with 1 to 3 substituents, wherein one substituent may be selected from the group consisting of halo, OH, CN, amino, R¹⁵, hydroxy-(C₁₋C₄)alkyl, R¹⁵O—(C₁-C₂)alkyl, cyano-(C₃-C₄)alkyl, OR¹⁵, SR¹⁵, SO₂R¹⁵, and NR¹⁵R¹⁶; and wherein 1 to 2 substituents may be independently selected from halo, methyl, ethyl, n-propyl, methoxy, ethoxy, difluoromethyl, and trifluoromethyl;

wherein R¹⁵ is selected from the group consisting of (C₁-C₄)-alkyl, (C₂-C₄)alkenyl, cyclopropyl, and cyclopropylmethyl, optionally independently substituted with 1 to 3 fluorine atoms.

R¹⁶ is H, (C₁-C₃)alkyl, or (C₁-C₃)alkoxy;

R²⁰ is selected from the group consisting of H, NHR¹³, (C₂-C₆)alkynyl, and R³;

R²¹ is selected from the group consisting of H, (C₁-C₆)alkyl, (C₃-C₅)cycloalkyl-(C₃-C₃)alkyl, (C₂-C₆)alkenyl, (C₂-C₆)alkynyl, cyano-(C₁-C₄)alkyl, hydroxy, —OR¹³, hydroxy-(C₁-C₆)alkyl-, R¹³O—(C₁-C₈)alkyl-, R¹³S—(C₁-C₆)alkyl, hydroxy-(C₁-C₆)alkoxy-, R¹³O—(C₁-C₈)alkoxy-, amino-(C₂-C₆)alkoxy, R¹³R¹⁴N—(C₂-C₆)alkoxy, —S(O)₂R¹³, —S(O)₂NR¹³R¹⁴, —S(O)₂NH₂, carboxy-(C₁-C₄)alkyl, R¹³OC(═O)—(C₁-C₄)alkyl, R¹³R¹⁴NC(═O)(C₃-C₄)alkyl, carbamoyl-(C₁-C₄)alkyl, carboxy-(C₁-C₄)alkoxy, R¹³OC—(═O)—(C₁-C₄)alkoxy, carbamoyl-(C₁-C₄)alkoxy, R¹³R¹⁴NC(═O)—(C₁-C₄)alkoxy, amino-(C₂-C₅)alkyl-, R¹³R¹⁴N—(C₂-C₆)alkyl-, amino-(C₂-C₆)alkoxy, R¹³R¹⁴N—(C₂-C₆)alkoxy, —OC(═O)NR¹³R¹⁴, phenyl-A-, 5 to 6-membered heteroaryl-A-, phenyl-(A)ₘ-(C₁-C₄ alkyl), and heteroaryl-(A)ₘ-C₁-C₄ alkyl); wherein said phenyls or heteroaryl of R²¹ is optionally substituted with 1 to 3 substituents independently selected from halo, cyano, cyano-(C₁-C₄)alkyl, R¹³, OR¹³, and R¹³O—(C₁-C₆)alkyl; and wherein said alkenyl, alkynyl, alkyl, or alkoxy group of R²¹ is optionally substituted with 1 to 3 fluorine atoms;

E, F, G, J, and the two carbons to which they are attached, together form a 6-membered aromatic or heteroaromatic ring;

wherein E is selected from N, N(O), and CR⁴; wherein R⁴ is selected from the group consisting of H, halogen, methyl, —OH, and —NH₂;

F is selected from N, N(O), and CR⁵;

G is selected from N, N(O), and CR⁶;

J is selected from N, N(O), and CR⁷;

wherein R⁵, R⁶, and R⁷ are independently selected from the group consisting of H, halogen, cyano, hydroxy, amino, (C₁-C₄)alkyl, cyclopropyl, cyclopropylmethyl, hydroxy(C₁-C₃)alkyl, (C₁-C₃)alkoxy, (C₁-C₃)alkylamino, and di(C₁-C₃)alkylamino; wherein said alkyl and alkoxy groups are independently optionally substituted with 1 to 3 fluorine atoms;

L, M, Q, T, U, and V together form an aromatic or a heteroaromatic ring;

L is carbon or nitrogen;

n is zero or 1;

wherein when n is zero, then M, Q, U, and V are independently selected from the group consisting of C, N, O, and S; and

when n is 1, then M, Q, T, U, and V are independently selected from the group consisting of carbon and nitrogen;

R⁸, R⁹, R¹¹, and R¹², when present, are independently selected from the group consisting of H, hydroxy, nitro, R³, and R^{3a};

R¹⁰, when present, is selected from the group consisting of H, hydroxy, nitro, NHR¹³, and R³;

or optionally R⁸-M-Q-R⁹ are taken together to form a ring, or R⁸-M-Q-R⁹ are taken together to form a ring and R¹¹-U—V—R¹² are taken together to form another ring;

or optionally when n is zero, R⁹-Q-U—R¹¹ are taken together to form a ring;

or optionally when n is 1, R⁹-Q-T-R¹⁰ are taken together to form a ring

or R⁸-M-Q-R⁹ are taken together to form a ring and R¹⁰-T-U—R¹¹ are taken together to form another ring;

wherein said rings formed from R⁸-M-Q-R⁹, R¹¹-U—V—R¹²-R⁹-Q-U—R¹¹, R⁹-Q-T-R¹⁰, and/or R¹⁰-T-U—R¹¹ are 5 to 7 membered carbocyclic or heterocyclic rings, wherein said heterocyclic rings independently contain 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₁)alkyl); wherein said alkyl and alkoxy groups are optionally independently substituted with 1 to 5 fluorine atoms;

and wherein when the ring formed by W, X, Y, Z, and the nitrogen to which W and Z are attached, is selected from the group consisting of b, c, f, and i;

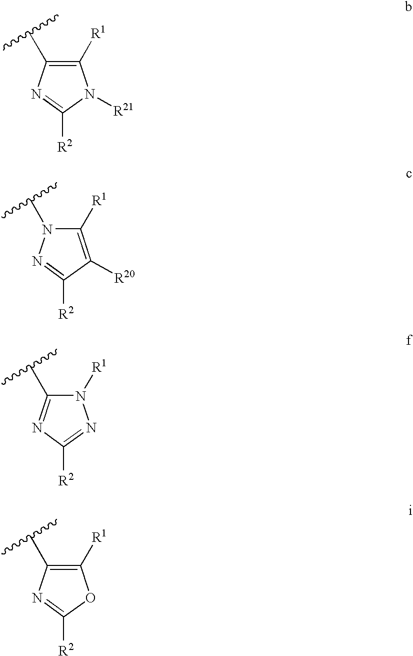

then 2 or more of the group consisting of R¹; R²; and the ring formed by L, M, Q: (T)ₙ, U, and V; must be heteroaryls.

One embodiment of the present invention includes a compound of formula I, or a pharmaceutically acceptable salt thereof, wherein the ring formed by W, X, Y, Z, and the nitrogen to which W and Z are attached (hereafter “WXYZ ring”);

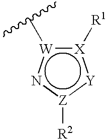

is selected from the group consisting of a, b, c, d, e, f, g, h, and i;

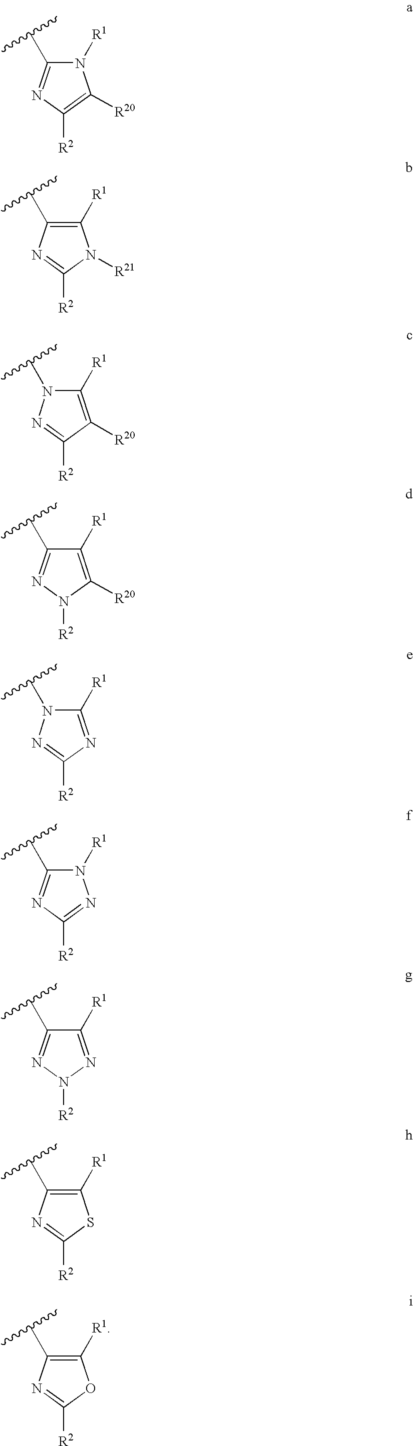

The WXYZ ring of formula I, may also be selected from a, c, d, e, f, and g;

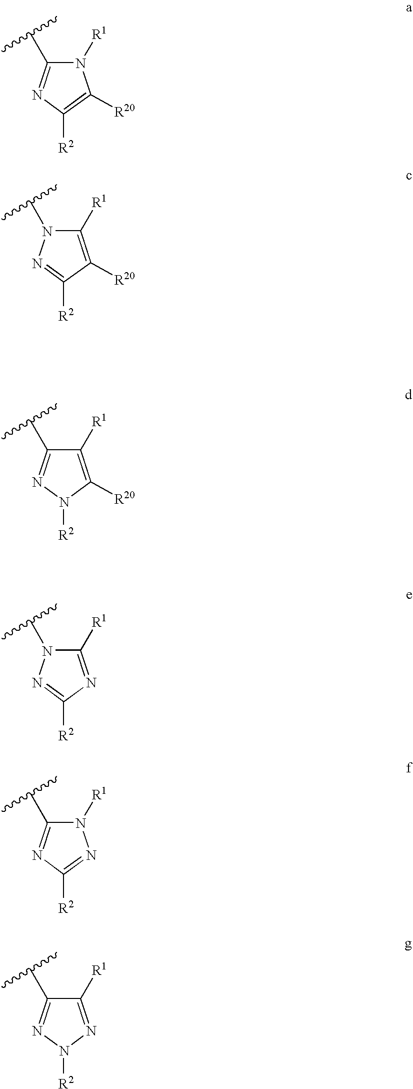

The WXYZ ring may also be defined such that W, X, and Z are carbon and Y is NR²¹. The WXYZ ring may also be defined such that W and Z are carbon, X Is nitrogen, and Y is CR²⁰.

The present invention also includes a compounds of formula I, wherein the group formed by L, M, Q, (T)ₙ, U, and V, and attached substituents, (hereafter “LMQ(T)ₙUV ring”);

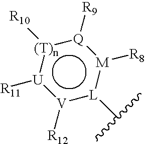

may be a monocyclic, bicyclic, or tricyclic ring or ring system.

The LMQ(T)ₙUV ring may be a monocyclic ring wherein M, Q, U, and V are independently selected from the group consisting of carbon and nitrogen; R⁸, R⁹, R¹¹, and R¹², when present, are independently selected from the group consisting of H, hydroxy, nitro, R³, and R^{3a}; and R¹⁰, when present, is selected from the group consisting of H, hydroxy, nitro, NHR¹³ and R³.

The LMQ(T)ₙUV ring may be a bicyclic ring wherein R⁸-M-G-R⁹ are taken together to form a ring; R¹¹ and R¹², when present, are independently selected from the group consisting of H, hydroxy, nitro, R³, and R^{3a}; and wherein R¹⁰, when present, is selected from the group consisting of H, hydroxy, nitro, NHR¹³, and R³; or optionally when n is zero, R⁹-Q-U—R¹¹ are taken together to form a ring; and R⁸, R¹¹ and R¹², when present, are independently selected from the group consisting of H, hydroxy, nitro, R³, and R^{3a}; or optionally when n is 1, R⁹-Q-T-R¹⁰ are taken together to form a ring; R⁸, R¹¹, and R¹² when present, are independently selected from the group consisting of H, hydroxy, nitro, R³, and R^{3a}; wherein said rings are 5 to 7 membered carbocyclic or heterocyclic rings; wherein said heterocyclic ring contains 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₅)alkoxy, (C₃-C₂)alkylthio, hydroxy(C₁-C₃)alkyl, (C₁-C₃)alkylthio-(C₁-C₂)alkyl, and (C₁-C₃)alkylthio(C₁-C₂)alkyl); and wherein said alkyl and alkoxy groups are optionally independently substituted with 1 to 5 fluorine atoms. The LMQ(T)ₙUV moiety may also be as defined in this paragraph, but wherein R⁸-M-Q-R⁹ are taken together to form a 6-membered aromatic or heteroaromatic ring; wherein said heteroaromatic ring contains 1 to 4 heteroatoms selected independently from the group consisting of N, O, and S; and wherein said ring is optionally substituted with 1 to 3 substituents each independently selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally independently substituted with 1 to 5 fluorine atoms; R¹¹ and R¹² when present, are independently selected from the group consisting of H, hydroxy, nitro, R³, and R^{3a}; and R¹⁶, when present, is selected from the group consisting of H, hydroxy, nitro, NHR¹³, and R³.

The LMQ(T)ₙUV ring may be tricyclic wherein R⁸-M-G-R⁹ are taken together to form a ring and R¹¹-U—V—R¹² are taken together to form another ring; or optionally when n is 1, R⁸-M-Q-R⁹ are taken together to form a ring and R¹⁰-T-U—R¹¹ are taken together to form another ring.

In another aspect of the invention, R² of formula I may be selected from the following substituents: In one embodiment, R² is selected from a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, ON, amino, R¹⁵, hydroxy-(C₁-C₄)alkyl, R¹⁵O—(C₁-C₂)alkyl, cyano-(C₁-C₄)alkyl, OR¹⁵, SR¹⁵, SO₂R¹⁵, and NR¹⁵R¹⁶; and wherein 1 to 2 substituents may be independently selected from halo, methyl, ethyl, n-propyl, methoxy, ethoxy, difluoromethyl, and trifluoromethyl. In another embodiment, R² is be selected from the group consisting of pyridyl and a 5-membered heteroaryl containing 1 to 2 heteroatoms independently selected from N, O, and S; and wherein said pyridyl or 5-membered heteroaryl group is optionally substituted with 1 to 2 substituents independently selected form chloro, fluoro, or methyl. In another embodiment, R² is be selected from the group consisting of thienyl, thiazoyl, oxazolyl, 2-pyridyl, and 3-pyridyl; wherein said group is optionally substituted with 1 to 2 substituents independently selected from chloro, fluoro, or methyl.

The present invention includes embodiments of formula I, as defined above; wherein any of the moieties of formula I, defined herein (i.e. WXYZ ring, LMQ(T)ₙUV ring, R², etc.), may be combined in any number and in any manner, without restriction, to arrive at further embodiments of the invention. For example, one embodiment may include a compound of formula I, wherein the LMQ(T)ₙUV ring is bicyclic, and wherein the WXYZ ring is selected from one of the options defined above. As another example, one embodiment may include a compound of formula I, wherein one of the WXYZ rings defined herein may be combined with one of the definitions of R² defined herein. Yet another example of an embodiment may include a compound of formula I, wherein one of the WXYZ rings, defined herein, may be combined with a LMQ(T)ₙUV tricyclic ring, and one of the definitions of R², as defined herein.

Another embodiment of the present invention relates to a compound of formula I, wherein the WXYZ ring is selected from the group consisting of a, c, d, e, f, and g;

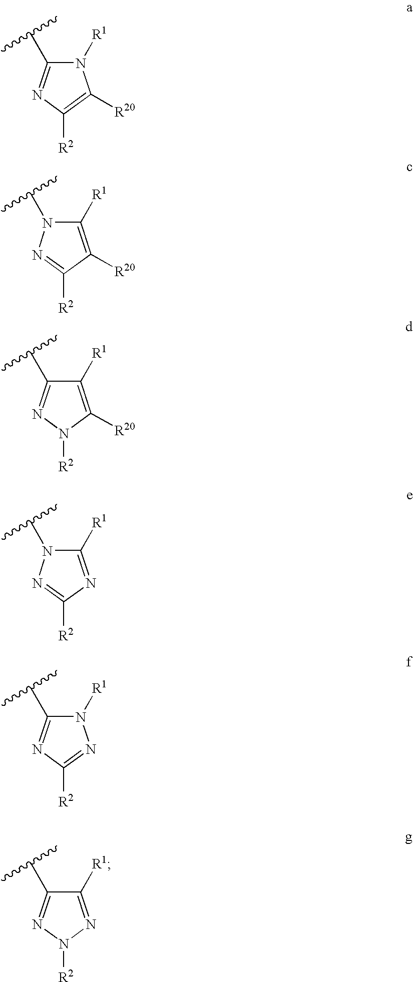

or a pharmaceutically acceptable salt thereof.

Another embodiment of the present invention relates to a compound of formula I, wherein W, X, and Z are carbon and Y is NR²¹; or a pharmaceutically acceptable salt thereof.

Another embodiment of the present invention relates to a compound of formula I, wherein W and Z are carbon, X is nitrogen, and Y is CR²⁰; or a pharmaceutically acceptable salt thereof.

Another embodiment of the present invention relates to a compound of formula I, wherein R⁸-M-Q-R⁹ are taken together to form a ring; R¹¹ and R¹², when present, are independently selected from the group consisting of H, hydroxy, nitro, R³, and R^{3a}, and wherein R¹⁰, when present, is selected from the group consisting of H, hydroxy, nitro, NHR¹³, and R³; or optionally when n is zero, R⁹-Q-U—R¹¹ are taken together to form a ring, and R⁸ and R¹², when present, are independently selected from the group consisting of H, hydroxy, nitro, R³, and R^{3a}; or optionally when n is 1, R⁹-Q-T-R¹⁰ are taken together to form a ring; R⁸, R¹¹, and R¹², when present, are independently selected from the group consisting of H, hydroxy, nitro, R³, and R^{3a}; wherein said rings are 5 to 7 membered carbocyclic or heterocyclic rings; wherein said heterocyclic ring contains 1 to 4 heteroatoms selected independently from the group consisting of N, O, and S; and wherein said rings are optionally substituted with 1 to 3 substituents independently selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms; or a pharmaceutically acceptable salt thereof.

Another embodiment of the present invention relates to a compound of formula I, wherein R⁸-M-Q-R⁸ are taken together to form a 6-membered aromatic or heteroaromatic ring; wherein said heteroaromatic ring contains 1 to 4 heteroatoms selected independently from the group consisting of N, O, and S; and wherein said ring is optionally substituted with 1 to 3 substituents selected independently from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl, and (C₁-C₃)alkylthio(C₁-C₂)alkyl; wherein said alkyl and alkoxy groups are optionally independently substituted with 1 to 5 fluorine atoms; R¹¹ and R¹², when present, are independently selected from the group consisting of H, hydroxy, nitro, R³, and R^{3a}; R¹⁰, when present, is selected from the group consisting of H; hydroxy, nitro, NHR¹³, and R³; or a pharmaceutically acceptable salt thereof.

Another embodiment of the present invention relates to a compound of formula I, wherein R² is a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, CM, amino, R¹⁵, hydroxy-(C₁-C₄)alkyl, R¹⁶O—(C₁-C₂)alkyl, cyano-(C₁-C₄)alkyl OR¹⁵, SR¹⁶, SO₂R¹⁵, and NR¹⁵R¹⁶; and wherein 1 to 2 substituents may be independently selected from halo, methyl, ethyl, n-propyl, methoxy, ethoxy, difluoromethyl, and trifluoromethyl; or a pharmaceutically acceptable salt thereof.

Another embodiment of the present invention relates to a compound of formula I, wherein R² is selected from the group consisting of pyridyl and a 5-membered heteroaryl containing 1 to 2 heteroatoms independently selected from N, O, and S; and wherein said group is optionally substituted with 1 to 2 substituents independently selected form chloro, fluoro, or methyl; or a pharmaceutically acceptable salt thereof.

Another embodiment of the present invention relates to a compound of formula I, wherein R² is selected from the group consisting of thienyl, thiazoyl, oxazolyl, 2-pyridyl, and 3-pyridyl; wherein said group is optionally substituted with 1 to 2 substituents independently selected from chloro, fluoro, or methyl; or a pharmaceutically acceptable salt thereof.

Another embodiment of the present invention relates to a compound of formula I, wherein the WXYZ ring is selected from the group consisting of a, c, d, e, f, and g; as defined above; wherein R² is a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, CN, amino, R¹⁵, hydroxy-(C₁-C₄)alkyl, R¹⁵O—(C₁-C₂)alkyl, cyano-(C₁-C₄)alkyl, OR¹⁵, SR¹⁵, SO₂R¹⁵, and NR¹⁵R¹⁶; and wherein 1 to 2 substituents may be independently selected from halo, methyl, ethyl, n-propyl, methoxy, ethoxy, difluoromethyl, and trifluoromethyl; or a pharmaceutically acceptable salt thereof.

Another embodiment of the present invention relates to a compound of formula I, wherein the WXYZ ring is selected from the group consisting of a, c, d, e, f, and g; as defined above; wherein R⁸-M-Q-R⁹ are taken together to form a ring; R¹¹ and R¹² when present, are independently selected from the group consisting of H, hydroxy, nitro, R³, and R^{3a}; and wherein R¹⁰, when present, is selected from the group consisting of H, hydroxy, nitro, NHR¹³, and R⁸; or optionally when n is zero, R⁹-Q-U—R¹¹ are taken together to form a ring; and R⁸ and R¹², when present, are independently selected from the group consisting of H, hydroxy, nitro, R³, and R^{3a}; or optionally when n is 1, R⁹-Q-T-R¹⁰ are taken together to form a ring; R⁸, R¹³, and R¹², when present, are independently selected from the group consisting of H, hydroxy, nitro, R³, and R^{3a}; wherein said rings are carbocyclic or heterocyclic; wherein said heterocyclic ring contains 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms; or a pharmaceutically acceptable salt thereof.

Another embodiment of the present invention relates to a compound of formula I, wherein R⁸-M-Q-R⁹ are taken together to form a ring; R¹¹ and R¹², when present, are independently selected from the group consisting of H, hydroxy, nitro, R³, and R^{3a}; and wherein R¹⁰, when present, is selected from the group consisting of H, hydroxy, nitro, NHR¹³, and R⁸; or optionally when n is zero, R⁹-Q-U—R¹¹ are taken together to form a ring; and R⁸ and R¹², when present, are independently selected from the group consisting of H, hydroxy, nitro, R³, and R^{3a}; or optionally when n is 1. R⁹-Q-T-R¹⁰ are taken together to form a ring; R⁸, R¹¹, and R¹² when present, are independently selected from the group consisting of H, hydroxy, nitro, R³, and R^{3a}; wherein said rings are 5 to 7 membered carbocyclic or heterocyclic rings; wherein said heterocyclic ring contains 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl; wherein said alkyl and alkoxy groups are optionally substituted with 1 to 5 fluorine atoms; wherein is a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, CN, amino, R¹⁵, hydroxy-(C₁-C₄)alkyl, R¹⁵O—(C₁-C₂)alkyl, cyano-(C₁-C₄)alkyl, OR¹⁵, SR¹⁵, SO₂R¹⁵, and NR¹⁵R¹⁶; and wherein 1 to 2 substituents may be independently selected from halo, methyl, ethyl, n-propyl, methoxy, ethoxy, difluoromethyl, and trifluoromethyl; or a pharmaceutically acceptable salt thereof.

Another embodiment of the present invention relates to a compound of formula I, wherein the WXYZ ring is selected from the group consisting of a, c, d, e, f, and g; as defined above; wherein R² is a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, CN, amino, R¹⁵, hydroxy-(C₁-C₄)alkyl, R¹⁵O—(C₁-C₂)alkyl, cyano-(C₁-C₄)alkyl, OR¹⁵, SR¹⁵, SO₂R¹⁵, and NR¹⁵R¹⁶; and wherein 1 to 2 substituents may be independently selected from halo, methyl, ethyl, n-propyl, methoxy, ethoxy, difluoromethyl, and trifluoromethyl; wherein R⁵-M-Q-R⁹ are taken together to form a ring; R¹¹ and R¹², when present, are independently selected from the group consisting of H, hydroxy, nitro, R³, and R^{3a}; and wherein R¹⁰, when present, is selected from the group consisting of H, hydroxy, nitro, NHR¹³, and R³; or optionally when n is zero, R⁹-Q-U—R¹¹ are taken together to form a ring; and R⁸ and R¹², when present, are independently selected from the group consisting of H, hydroxy, nitro, R³, and R^{3a}; or optionally when n is 1, R⁹-Q-T-R¹⁰ are taken together to form a ring; R⁸, R¹¹, and R¹², when present, are independently selected from the group consisting of H, hydroxy, nitro, R³, and R^{3a}; wherein said rings are carbocyclic or heterocyclic; wherein said heterocyclic ring contains 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms; or a pharmaceutically acceptable salt thereof.

Another embodiment of the present invention relates to a compound of formula I, wherein the WXYZ ring is selected from the group consisting of a, c, d, e, f, and g; as defined above; wherein R⁸-M-Q-R⁹ are taken together to form a 6-membered aromatic or heteroaromatic ring; wherein said heteroaromatic ring contains 1 to 4 heteroatoms selected independently from the group consisting of N, O, and S; and wherein said ring optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms; R¹¹ and R¹², when present, are independently selected from the group consisting of H, hydroxy, nitro, R³, and R^{3a}; R¹⁰, when present, is selected from the group consisting of H, hydroxy, nitro, NHR¹³, and R³; or a pharmaceutically acceptable salt thereof. A more preferred embodiment includes compounds of formula I, as defined in this paragraph, wherein R² is selected from the group consisting of pyridyl and a 5-membered heteroaryl containing 1 to 2 heteroatoms independently selected from N, O, and S; and wherein said group is optionally substituted with 1 to 2 substituents independently selected form chloro, fluoro, or methyl; or a pharmaceutically acceptable salt thereof.

Another embodiment of the present invention relates to a compound of formula I, wherein R⁸-M-G-R⁹ are taken together to form a 6-membered aromatic or heteroaromatic ring; wherein said heteroaromatic ring contains 1 to 4 heteroatoms selected independently from the group consisting of N, O, and S; and wherein said ring optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms; R¹¹ and R¹², when present, are independently selected from the group consisting of H, hydroxy, nitro, R³ and R^{3a}; R¹³, when present, is selected from the group consisting of H, hydroxy, nitro, NHR¹³ and R³; wherein W and Z are carbon; X is nitrogen; Y is CR²⁰; and R² is selected from the group consisting of pyridyl and a 5-membered heteroaryl containing 1 to 2 heteroatoms independently selected from N, O, and S; and wherein said group is optionally substituted with 1 to 2 substituents independently selected form chloro, fluoro, or methyl; or a pharmaceutically acceptable salt thereof.

Another embodiment of the present invention relates to a compound of formula I, wherein R⁸-M-Q-R⁹ are taken together to form a 6-membered aromatic or heteroaromatic ring; wherein said heteroaromatic ring contains 1 to 4 heteroatoms selected independently from the group consisting of N, O, and S; and wherein said ring optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl; wherein said alkyl and alkoxy groups are optionally substituted with 1 to 5 fluorine atoms; R¹¹ and R¹², when present, are independently selected from the group consisting of H, hydroxy, nitro, R³, and R^{3a}; R¹³, when present, is selected from the group consisting of H, hydroxy, nitro, NHR¹³, and R³; wherein W and Z are carbon; X is nitrogen; Y is CR²⁹; and R² is selected from the group consisting of thienyl, thiazoyl, oxazolyl, 2-pyridyl, and 3-pyridyl; wherein said group is optionally substituted with 1 to 2 substituents independently selected from chloro, fluoro, or methyl; or a pharmaceutically acceptable salt thereof.

Another embodiment of the present invention relates to a compound of formula I, wherein M, Q, U, and V are independently selected from the group consisting of carbon and nitrogen; R⁸, R⁹, R¹¹, and R¹², when present are independently selected from the group consisting of H, hydroxy, nitro, R³, and R^{3a}; and R¹⁰, when present, is selected from the group consisting of H, hydroxy, nitro, NHR¹³, and R³; and wherein the WXYZ ring is selected from the group consisting of a, c, d, e, f, and g; as defined above; or a pharmaceutically acceptable salt thereof. Another embodiment includes a compound formula I, as defined in this paragraph, but wherein W, X, and Z are carbon and Y is NR²¹; or a pharmaceutically acceptable salt thereof.

Another embodiment of the present invention relates to a compound of formula I, wherein R² is a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, CN, amino, R¹⁵, hydroxy-(C₁-C₄)alkyl, R¹⁵O—(C₁-C₂)alkyl, cyano-(C₁-C₄)alkyl, OR¹⁵, SR¹⁵, SO₂R¹⁵, and NR¹⁵R¹⁶, and wherein 1 to 2 substituents may be independently selected from halo, methyl, ethyl, n-propyl, methoxy, ethoxy, difluoromethyl, and trifluoromethyl; and R⁸-M-Q-R⁹ are taken together to form a ring and R¹¹—U—V—R¹² are taken together to form another ring; or optionally when n is 1, R⁸-M-Q-R⁹ are taken together to form a ring and R¹⁰-T-U—R¹¹ are taken together to form another ring; and wherein the WXYZ ring is selected from the group consisting of a, c, d, e, f, and g; as defined above; or a pharmaceutically acceptable salt thereof. Another embodiment includes a compound formula I, as defined in this paragraph, but wherein W, X, and Z are carbon and Y is NR²¹; or a pharmaceutically acceptable salt thereof.

Another embodiment of the present invention is directed to a compound of formula I, wherein E, F, G, and J are carbon; wherein E, F, G, and J are optionally independently substituted with fluorine, chlorine, or methyl; W and Z are carbon; X is nitrogen; Y is CR²⁰; wherein R²⁰ is hydrogen or halo; R² is selected from the group consisting of thienyl, thiazoyl, oxazolyl, 2-pyridyl, and 3-pyridyl; wherein R² is optionally substituted with 1 to 2 substituents selected from fluorine, chlorine, and methyl; R⁸-M-Q-R⁹ are taken together to form a 6-membered aromatic or heteroaromatic ring; wherein said heteroaromatic ring contains 1 to 4 heteroatoms selected independently from the group consisting of N, O, and S; wherein said ring is optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms; R¹¹ and R¹², when present, are independently selected from the group consisting of H, hydroxy, nitro, R³, and R^{3a}; R¹⁰ when present, is selected from the group consisting of H, hydroxy, nitro, NHR¹³, and R³; or a pharmaceutically acceptable salt thereof. Another aspect of the invention includes a compound of formula I, as defined in this paragraph, wherein n is zero; or a pharmaceutically acceptable salt thereof. Yet another aspect of the invention includes a compound of formula I, as defined in this paragraph, wherein n is zero and wherein R¹ is selected from the group consisting of pyridyl, pyrimidinyl, and phenyl; wherein R¹ is optionally substituted with 1 to 3 substituents independently selected from the group consisting of halo, (C₁-C₃)alkyl, and (C₁-C₃)alkoxy; or a pharmaceutically acceptable salt thereof.

Another embodiment of the present invention is directed to a compound of formula I, wherein R¹ is pyridyl optionally substituted with one or two substituents independently selected from (C₁-C₅)alkyl and halo; R² is thiazolyl, oxazolyl, or thienyl optionally substituted 1or 2 substituents independently selected from methyl, chloro, and fluoro; E, F, G, and J are carbon; R⁴ R⁵, R⁶, and R⁷ are independently selected from the group consisting of hydrogen, halo, and methyl; L is nitrogen; n is zero; V is carbon; U is carbon or nitrogen; R⁵-M-Q-R⁹ are taken together to form a 6-membered aromatic or heteroaromatic ring; optionally substituted with one or two substituents independently selected from the group consisting of halo, cyano, (C₁-C₄)alkyl, and (C₁-C₃)alkoxy; and wherein said heteroaromatic ring contains one nitrogen atom; R¹¹ is absent or selected from hydrogen, halo, (C₁-C₅)alkyl, CF₂H, CF₃, CF₂CF₃, cyano, and (C₁-C₅)alkoxy; R¹² is selected from the group consisting of hydrogen, halo, (C₁-C₅)alkyl, CF₂H, CF₃, CF₂CF₃, cyano, (C₁-C₅)alkoxy(C₃-C₇)cycloalkyl, (C₃-C₇)cycloalkyl-(C₁-C₃)alkyl, (C₁-C₃)alkoxy(C₁-C₃)alkyl, phenyl, pyridyl, phenoxy, pyridyloxy, benzyl, and pyridylmethyl; wherein said phenyl, pyridyl, phenoxy, pyridyloxy, benzyl, and pyridylmethyl are optionally substituted with 1 or 2 substituents independently selected from halo and methyl; or a pharmaceutically acceptable salt thereof. Another embodiment includes a compound of formula I, as defined in this paragraph, wherein W and Z are carbon; X is nitrogen; and Y is CR²⁰; or a pharmaceutically acceptable salt thereof.

Examples of specific compounds of formula I, include the following:
1-(4-(1-(4-methoxyphenyl)-4-(thiophen-2-yl)-1H-imidazol-2-yl)phenyl)-1H-pyrrolo[2,3-b]pyridine,
1-(4-(1-(4-methoxyphenyl)-4-(thiazol-2-yl)-1H-imidazol-2-yl)phenyl)-1H-pyrrolo[2,3-b]pyridine,
1-(4-(1,4-di(thiazol-2-yl)-1H-imidazol-2-yl)phenyl)-1H-pyrrolo[2,3-b]pyridine,
1-(4-(4-(pyridin-2-yl)-1-(pyrimidin-5-yl)-1H-imidazol-2-yl)phenyl)-1H-pyrrolo[2,3b]pyridine,
1-(4-(1-(2-methylpyridin-4-yl)-4-(pyridine-2-yl)-1H-imidazol-2-yl)phenyl)-1H-pyrrolo[2,3-b]pyridine,
1-(4-(1-(6-methylpyridin-3-yl)-4-(pyridin-2-yl)-1H-imidazol-2-yl)phenyl)-1H-pyrrolo[2,3-b]pyridine,
1-(4-(4-(pyridin-2-yl)-1-(pyridin-3-yl)-1H-imidazol-2-yl)phenyl)-1H-pyrrolo[2,3-b]pyridine,
1-(4-(1-(6-(1H-imidazol-1-yl)pyridin-3-yl)-4-(pyridin-2-yl)-1H-imidazol-2-yl)phenyl)-1H-pyrrolo[2,3-b]pyridine,
1-(4-(1-(6-methoxypyridin-3-yl)-4-(pyridin-2-yl)-1H-imidazol-2-yl)phenyl)-1H-pyrrolo[2,3-b]pyridine,
N,N-dimethyl-2-(1-(4-(4-(pyridin-2-yl)-1-(pyridin-3-yl)-1H-imidazol-2-yl)phenyl)-1H-pyrrolo[2,3-b]pyridin-3-yl)ethanamine,
1-(3-fluoro-4-(4-(pyridin-2-yl)-1-(pyridin-3-yl)-1H-imidazol-2-yl)phenyl)-1H-pyrrolo[2,3-b]pyridine,
1-(2-methyl-4-(1-(6-methylpyridin-3-yl)-4-(pyridin-2-yl)-1H-imidazol-2-yl)phenyl)-1H-pyrrolo[2,3-b]pyridine,
1-(3-methyl-4-(1-(6-methylpyridin-3-yl)-4-(pyridin-2-yl)-1H-imidazol-2-yl)phenyl)-1H-pyrrolo[2,3-b]pyridine.
1-(4-(4-(pyridin-2-yl)-1-(1-oxido-pyridin-3-yl)-1H-pyrrolo[2,3-b]pyridine,
1-(4-(1-(1-oxido-methylpyridin-3-yl)-4-(1-oxido-pyridin-2-yl)-1H-imidazol-2-yl)phenyl)-1H-indole,
1-(4-(1-(6-methylpyridin-3-yl)-4-(1-oxido-pyridin-2-yl)-1H-imidazol-2-yl)phenyl)-1H-pyrrolo[2,3-b]pyridine,
9-[4-(4-pyridin-2-yl-1-pyridin-3-yl-1H-imidazol-2-yl)phenyl]5,7,8,9-tetrahydrothiopyrano[3′,4′,4,5]pyrrolo[2,3]pyridine,
N,N-dimethyl(1-(4-(4-(pyridin-2-yl)-1-(pyridin-3-yl)-1H-imidazol-2-yl)phenyl)-1H-pyrrolo[2,3-b]pyridin-3-yl)methanamine,
9-(4-(4-(pyridin-2-yl)-1-(pyridin-3-yl)-1H-imidazol-2-yl)phenyl)-9H-pyrido[2,3-b]indole,
5-chloro-1-(4-(4-(pyridin-2-yl)-1-(6-methylpyridin-3-yl)-1H-imidazol-2-yl)phenyl)-1H-pyrrolo[2,3-b]pyridine,
5-fluoro-1-(4-(1-(6-methylpyridin-3-yl)-4-(pyridin-2-yl)-1H-imidazol-2-yl)phenyl)-1H-pyrrolo[2,3-b]pyridine,
5-methyl-1-(4-(1-(6-methylpyridin-3-yl)-1-(pyridin-2-yl)phenyl)-1H-pyrrolo[2,3-b]pyridine,
1-(4-(1-pyridin-3-yl)-4-(thiazol-2-yl)-1H-imidazol-2-yl)phenyl)-1H-pyrrolo[2,3-b]pyridine,
1-(4-(1-(pyridin-2-yl)-4-(thiazol-2-yl)-1H-imidazol-2-yl)phenyl)-1H-pyrrolo[2,3-b]pyridine.
1-(4-(1-(pyridin-4-yl)-4-(thiazol-2-yl)-1H-imidazol-2-yl)phenyl)-1H-pyrrolo[2,3-b]pyridine,
1-(4-(1-(pyrimidin-5-yl)-4-(thiazol-2-yl)-1H-imidazol-2-yl)phenyl)-1H-pyrrolo[2,3-b]pyridine,
1-(4-(1-(6-methylpyridin-3-yl)-4-(thiazol-2-yl)-1H-pyrrolo[2,3-b]pyridine,
1-(4-(1-(2-methylpyridin-3-yl)-1H-(thiazol-2-yl)-1H-imidazol-2-yl)phenyl)-1H-pyrrolo[2,3-b]pyridine,
1-(4-(1-(6-methoxypyridin-3-yl)-1H-imidazol-3-yl)-1H-imidazol-1-yl)-N,N-pyrrolo[2,3-b]pyridine,
5-(2-(4-(1H-pyrrolo[2,3-b]pyridin-1-yl)phenyl)-4-(thiazol-2-yl)-1H-imidazol-1-yl)-N,N-dimethylpyridin-2-amine,
2-(4-(2-(4-(1H-pyrrolo[2,3-b]pyridin-1-yl)phenyl)-4-(thiazol-2-yl)-1H-imidazol-1-yl)phenyl)-N-methylethanamine,
1-(4-(1-(6-(trifluoromethyl)pyridin-3-yl)-1H-(thiazol-2-yl)-1H-imidazol-2-yl)phenyl)-1H-pyrrolo[2,3-b]pyridine,
(4-(2-(4-(1H-pyrrolo[2,3-b]pyridin-1-yl)phenyl)-4-(thiazol-2-yl)-1H-imidazol-1-yl)phenyl)-N-methylmethanamine,
1-(4-(1-(6-morpholinopyridin-3-yl)-4-(thiazol-2-yl)-1H-imidazol-2-yl)phenyl)-1H-pyrrolo[2,3-b]pyridine,
1-(4-(4-(pyridin-2-yl)-1-(pyridin-3-yl)-1H-imidazol-2-yl)phenyl)-1H-indazole,
1-(4-(4-(pyridin-2-yl)-1-(pyridin-yl)-1H-imidazol-2-yl)phenyl)-1H-indole,
7-fluoro-1-(4-(4-(pyridin-2-yl)-1-(pyridin-3-yl)-1M-imidazol-2-yl)phenyl)-1H-indole,
4,5,6,7]-tetrafluoro-1-(4-(4-(pyridin-2-yl)-1-(pyridin-3-yl)-1H-imidazol-2-yl)phenyl)-1H-indole,
4-chloro-1-(4-(4-(pyridin-2-yl)-1-(pyridin-3-yl)-1H-imidazol-2-yl)phenyl)-1H-indole,
H4-(4-(pyridin-2-yl)-1-(pyridin-3-yl)-1H-imidazol-2-yl)phenyl)-1H-indole-4-carbonitrile,
3-(2-(4-(4-methyl-1H-imidazol-1-yl)phenyl)-4-(pyridin-2-yl)-1H-imidazol-1-yl)pyridine,
1-(4-(4-(pyridin-2-yl)-1-(pyridin-3-yl)-1H-imidazol-2-yl)phenyl)-1H-benzo[d][1,2,3]triazole,
2-(pyridin-2-yl)-1-(4-(4-(pyridin-2-yl)-1-(pyridin-3-yl)-1H-imidazol-2-yl)phenyl)-1H-benzo[d]imidazole,
3-(2-(4-(1H-imidazol-1-yl)phenyl)-4-(pyridin-2-yl)-1H-imidazol-1-yl)pyridine,
1-(4-(4-(pyridin-2-yl)-1-(pyridin-3-yl)-1H-imidazol-2-yl)phenyl)-1H-benzo[d]imidazole,
1-(4-(4-pyridin-2-yl)-1-(pyridin-3-yl)-1H-imidazol-2-yl)phenyl)-1H-imidazo[4,5-b]pyridine.
3-(4-(4-(pyridin-2-yl)-1-(pyridin-3-yl)-1H-imidazol-2-yl)phenyl)-3H-imidazo[4,5b]pyridine;
1-(4-(1-(6-methylpyridin-3-yl)-4-(pyridin-2-yl)-1H-imidazol-2-yl)phenyl)-1H-imidazo[4,5-b]pyridine,
3-(4-(1-(6-methylpyridin-3-yl)-4-(pyridin-2-yl)-1H-imidazol-2-yl)phenyl)-3H-imidazo[4,5-b]pyridine,
5-(4-(1-(6-methylpyridin-3-yl)-4-(pyridin-2-yl)-1H-imidazol-2-yl)phenyl)-5H-pyrrolo[3,2-b]pyrazine,
3-(4-(4-(pyridin-2-yl)-1-(pyridin-3-yl)-1H-imidazol-2-yl)phenyl)-3H-[1,2,3]triazolo[4,5-b]pyridine.
1-(4-(1-(6-methylpyridin-3-yl)-4-(pyridin-2-yl)-1H-imidazol-2-yl)phenyl)-1H-pyrrolo[3,2-b]pyridine,
1-(4-(1-(6-methylpyridin-3-yl)-4-(pyridin-2-yl)-1H-imidazol-2-yl)phenyl)-1H-pyrrolo[2,3-c]pyridine.
1-(4-(1-(6-methylpyridin-3-yl)-4-(pyridin-2-yl)-1H-imidazol-2-yl)phenyl)-1H-pyrrolo[3,2-e]pyridine,
9-(4-(1-(6-methylpyridin-3-yl)-4-(pyridin-2-yl)-1H-imidazol-2-yl)phenyl)-9H-purine,
1-(4-(1-(6-methylpyridin-3-yl)-4-(pyridin-2-yl)-1H-imidazol-2-yl)phenyl)-7H-purine,
1-(4-(1-(6-methylpyridin-3-yl)-4-(pyridin-2-yl)-1H-imidazol-2-yl)phenyl)-1H-pyrazolo[3,4-c]pyridine,
2-methyl-3-(4-(1-(6-methylpyridin-3-yl)-4-(pyridin-2-yl)-1H-imidazol-[4,5-b]pyridin,
2-(trifluoromethyl)-3-(4-(1-(6-methylpyridin-3-yl)-4-(pyridin-2-yl)-1H-imidazol-2-yl)phenyl)-3H-imidazo[4,5-b]pyridine,
2-isopropyl-3-(4-(1-(6-methylpyridin-3-yl)-4-(pyridin-2-yl))phenyl)-3H-imidazo[4,5-b]pyridine,
2-methoxy-3-(4-(1-(6-methylpyridin-3-yl)-4-(pyridin-2-yl)-1H-imidazo[4,5-b]pyridine,
1-(4-(1-(6-methylpyridin-3-yl)-4-(5-methylthiazol-2-yl)-1H-pyrrolo[2,3-b]pyridine,
1-(4-(4-(5-chlorothiophen-2-yl)-1-(pyrimidin-5-yl)-1H-imidazol-2-yl)phenyl)-1H-pyrrolo[2,3-b]pyridine,
1-(4-(4-(4-methylthiazol-2-yl)-1-(pyrimidin-5-yl)-1H-imidazol-2-yl)phenyl)-1H-pyrrol[2,3-b]pyridine,
1-(4-(4-(5-fluorothiophen-2-yl)-1-(6-methylpyridin-3-yl)-1H-imidazol-2-yl)phenyl)-1H-pyrrolo[2,3-b]pyridine,
1-(4-(4-(4,5-dimethylthiazol-2-yl)-1-(pyrimidin-5-yl)-1H-imidazol-2-yl)phenyl)-1H-pyrrolo[2,3-b]pyridine,
1-(4-(4-(1-methyl-1H-imidazol-2-yl)-1-(2-methylpyridin-4-yl)-1H-imidazol-2-yl)phenyl)-1H-pyrrolo[2,3-b]pyridine,
1-(4-(4-(1-methyl-1H-imidazol-2-yl)-1-(pyrimidin-5-yl)-1H-imidazol-2-yl)phenyl)-1H-pyrrolo[2,3-b]pyridine,
1-(4-(1-(2-methylpyridin-4-yl)-4-(pyridin-3-yl)-1H-imidazol-2-yl)phenyl)-1H-pyrrolo[2,3-b]pyridine,
1-(4-(1-(2-methylpyridin-4-yl)-1H-(pyridin-yl)-1H-imidazol-2-yl)phenyl)-1H-pyrrolo[2,3-b]pyridine,
5-(2-(4-(3,4-dichlorophenyl)phenyl)-4-(pyridin-2-yl)-1H-imidazol-1-yl)pyrimidine,
5-(2-(4-(4-chlorophenyl)phenyl)-4-(pyridin-2-yl)-1H-imidazol-1-yl)pyrimidine,
5-(4-(pyridin-2-yl)-2-(4-(pyridin-3-yl)phenyl)-1H-imidazol-1-yl)pyrimidine,
5-(4-(pyridin-2-yl)-2-(4-(pyridin-4-yl)phenyl)-1H-imidazol-1-yl)pyrimidine,
7-(4-(1-(6-methylpyridin-3-yl)-4-(pyridin-2-yl)-1H-imidazol-2-yl)phenyl)-7H-pyrrolo[2,3-d]pyrimidine,
7-methyl-5-(4-(1-(6-methylpyridin-3-yl)-4-(pyridin-2-yl)-1H-imidazol-2-yl)phenyl)-5H-pyrrolo[2,3-b]pyrazine.
1-(4-(4-(benzo[d]thiazol-2-yl)-1-(pyridin-3-yl)-1H-imidazol-2-yl)phenyl)-1H-pyrrolo[2,3-b]pyridine,
4-methoxy-6-methyl-8-(4-(4-(pyridin-2-yl)-1-(pyridin-3-yl)-1H-imidazol-2-yl)phenyl)quinoline,
8-(4-(4-<pyridin-2-yl)-1-(pyridin-3-yl)-1H-imidazol-2-yl)phenyl)-1,7-naphthyridine,
8-(4-(4-(pyridin-2-yl)-1-(pyridin-yl)-1H-imidazol-2-yl)phenyl)quinoline
6-methoxy-8-(4-(4-(pyridin-2-yl)-1-(pyridin-3-yl)-1H-imidazol-2-yl)phenyl]quinoline,
2-methoxy-3-(4-(1-(6-methylpyridin-3-yl)-4-(thiazol-2-yl)phenyl)-3H-imidazo[4,5-b]pyridine,
2-ethyl-3-(4-(1-(6-methylpyridin-3-yl)-4-(5-methylthiazol-2-yl)-1H-imidazol-2-yl)phenyl)-3H-imidazo[4,5-b]pyridine,
2-ethyl-3-(4-(1-(6-methylpyridin-3-yl)-4-(5-methylthiazol-2-yl)-1H-imidazol-2-yl)phenyl)-3H-imidazo[4,5-b]pyridine,
2-(difluoromethyl)-3-(4-(1-(6-methylpyridin-3-yl)-4-(thiazol-2-yl)-1H-imidazol-2-yl)phenyl)-3H-imidazo[4,5-b]pyridine,
2-ethyl-3-(4-(1-(6-methylpyridin-3-yl)-4-(thiazol-2-yl)-1H-imidazol-2-yl)phenyl)-3-H-imidazo[4,5-b]pyridine,
2-isopropyl-3-(4-(1-(6-methylpyridin-3-yl)-4-(5-methylthiazol-2-yl)-1H-imidazol-2-yl)phenyl)-3H-imidazo[4,5-b]pyridine,
2-(difluoromethyl)-3-(4-(1-(6-methylpyridin-3-yl)-4-(thiazol-2-yl)-1H-imidazol-2-yl)phenyl)-3H-imidazo[4,5-b]pyridine,
3-(4-(3-(pyridin-2-yl)-5-(pyridin-3-yl)-1H-1,2,4-triazol-1-yl)phenyl)-1H-imidazo[4,5-b]pyridin-2-(3H)-one,
2-methoxy-1-(4-(1-(6-methylpyridin-3-yl)-4-(pyridin-2-yl)-1H-imidazol-2-yl)phenyl)-1H-imidazo[4,5-c]pyridine,
2-methoxy-3-(4-(1-(6-methylpyridin-3-yl)-4-(4-methylthiazol-2-yl)-1H-imidazol-2-yl)phenyl)-3H-imidazo[4,5-b]pyridine,
3-(4-(1-(6-methylpyridin-3-yl)-1H-imidazol-2-imidazo[4,5-b]pyridine,
2-(methoxymethyl)-3-(4-(1-(6-methylpyridin-3-yl)-4-(thiazol-2-yl)-1H-imidazol-2-yl)phenyl)-3H-imidazo[4,5-b]pyridine,
2-methoxy-3-(4-(1-(6-methylpyridin-3-yl)-4-(thiophen-2-yl)-1H-(imidazol-2-yl)phenyl)-3H-imidazol-4,5-b]pyridine,
2-ethoxy-3-(4-(1-(6-methylpyridin-3-yl)-4-(pyridin-2-yl)-1H-imidazol-2-yl)phenyl)-3H-imidazo[4,5-b]pyridine,
3-(4-(1-(6-methylpyridin-3-yl)-4-(pyridin-2-yl)-1H-imidazol-2-yl)phenyl-1H-imidazo[4,5-b]pyridin-2-(3H)-one,
2-methoxy-3-(4-(1-(6-methylpyridin-3-yl)-4-(thiazol-4-yl)-1H-imidazol-2-yl)-1H-imidazol-2-yl)phenyl)-3H-imidazo[4,5-b]pyridine,
2-isopropyl-3-(4-(1-(6-methylpyridin-3-yl)-4-(thiazol-5-yl)-1H-imidazol-2-yl)phenyl)-3H-imidazo[4,5-b]pyridine,
2-isopropyl-3-(4-(1-(6-methylpyridin-3-yl)-4-(thiazol-4-yl)-1H-imidazol-2-imidazo[4,5-b]pyridine,
2-(trifluoromethyl)-3-(4-(1-(6-methylpyridin-3-yl)-4-(thiazol-4-yl)-1H-imidazol-2-yl)phenyl)-3H-yl)phenyl)-3H-imidazo[4,5-b]pyridine,
2-ethoxy-3-(4-(1-(6-methylpyridin-3-yl)-4-(thiazol-4-yl)-1H-imidazol-2-yl)phenyl)-3H-imidazo[4,5-b]pyridine,
3-(4-(5-(4-methoxyphenyl)-2-(thiophen-2-yl)-1H-imidazol-4-yl)phenyl-3H-imidazo[b]pyridine,
1-(4-(5-(4-methoxyphenyl)-2-(thiophen-2-yl)-1H-imidazol-4-yl)phenyl)-1H-imidazo[4,5-b]pyridine,
5-methoxy-1-(4-(5-(4-methoxyphenyl)-2-(thiophen-2-yl)-1H-imidazol-4-yl)phenyl)-1H-indole,
1-(4-(5-(4-methoxyphenyl)-2-(thiophen-2-yl)-1H-imidazol-4-yl)phenyl)-1H-pyrrolo[2,3-b]pyridine,
1-(4-(1-hydroxy-5-(pyrazin-2-yl)-2-(thiophen-2-yl)-1H-imidazol-4-yl)phenyl)-1H-pyrrolo[2,3-b]pyridine.
1-(4-(5-(pyrazin-2-yl)-2-(thiophen-2-yl)-1H-imidazol-4-yl)phenyl)-1H-pyrrolo[2,3-b]pyridine;
1-(4-(5-(4-methoxyphenyl)-2-(thiophen-2-yl)-1H-imidazol-4-yl)phenyl)-1H-imidazole,
1-(4-(5-(6-(4-methylpiperazin-1-yl)pyridin-3-yl)-2-(thiazol-5-yl)-1H-imidazol-4-yl)phenyl) 1H-pyrrolo[2,3-b]pyridine,
1-(4-(5-(4-methoxyphenyl]-2-(thiophen-2-yl)-1H-imidazol-yl)phenyl)-4-phenyl-1H-imidazole.
1-(4-(1-hydroxy-5-(pyrazin-2-yl)-2-(thiophen-2-yl)-1H-imidazol-4-yl)-2-methylphenyl)-1H-pyrrolo[2,3-b]pyridine,
1-(4-(1-hydroxy-5-(pyrazin-2-yl)-2-(thiophen-2-yl)-1H-imidazol-4-yl)-2-methylphenyl)-1H-pyrrolo[2,3-b]pyridine,
1-(4-(1-hydroxy-5-(pyrazin-2-yl)-2-(thiophen-2-yl)-1H-imidazol-4-yl)-2-methylphenyl)-1H-pyrrolo[2,3-b]pyridine,
1-(2-methyl-4-(5-(pyrazin-2-yl)-2-(thiazol-5-yl)-1H-imidazol-4-yl)phenyl)-1H-pyrrolo[2,3-b]pyridine,
1-(2-methyl-4-(5-(pyrazin-2-yl)-2-(thiazol-5-yl)-1H-imidazol-4-yl)phenyl)-1H-pyrrolo[2,3-b]pyridine,
1-(4-(2-(pyridin-2-yl)-4-(pyridin-3-yl)-1H-imidazol-5-yl)phenyl)-1H-pyrrolo[2,3-b]pyridine,
1-(4-(3-(pyridin-2-yl)-5(-pyridin-3-yl)-1H-1,2,4-triazol-1-yl)phenyl)-1H-pyrrolo[2,3b]pyridine,
2-methoxy-3-(4-(1-(6-methylpyridin-3-yl)-4-(thiazol-5-yl)-1H-pyrrolo[2,3-imidazo[4,5-b]pyridine,
2-(trifluoromethyl)-3-(4-(1-(6-methylpyridin-3-yl)-4-(thiazol-5-yl)-1H-imidazol-2-yl)phenyl)-2-yl)phenyl)-3H-imidazo[4,5-b]pyridine,
3-(4-(1-(6-methylpyridin-3-yl)-4-(thiazol-2-yl)-1H-imidazol-2-yl)phenyl)-1 H)imidazo[4,5-b]pyridin-2-(3H)-one,
1-(4-(2-(pyridin-2-yl)-5-(pyridin-3-yl)-2H-1,2,3-triazol-4-yl)phenyl)-1H-pyrrolo[2,3-b]pyridine,
1-(4-(1-(pyridin-2-yl)-4-(pyridin-3-yl)-1H-pyrazol-3-yl)phenyl)-1H-pyrrolo[2,3-b]pyridine,
1-(4-(3-(pyridin-2-yl)-5-(pyridin-3-yl)-1H-pyrazol-1-yl)phenyl)-1H-pyrrolo[2,3-b]pyridine,
1-(4-(5-(pyridin-2-yl)-3-(pyridin-3-yl)-1H-pyrazol-1-yl)phenyl)-1H-pyrrolo[2,3-b]pyridine, and
1-(4-(5-(pyridin-2-yl)-2-(pyridin-3-yl)-2-(pyridin-3-yl)-2H-1,2,4-triazol-3-yl)phenyl)-1H-pyrrolo[2,3-b]pyridine,

and pharmaceutically acceptable salts thereof.

Compounds of the Formula I may have optical centers and therefore may occur in different enantiomeric and diastereomeric configurations. The present invention includes all enantiomers, diastereomers, and other stereoisomers of such compounds of the Formula I, as well as racemic compounds and racemic mixtures and other mixtures of stereoisomers thereof.

Pharmaceutically acceptable salts of the compounds of formula I include the acid addition and base salts thereof.

Suitable acid addition salts are formed from acids which form non-toxic salts. Examples include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable base salts are formed from bases which form non-toxic salts. Examples include, but are not limited to, the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of Formula I may be prepared by one or more of three methods:

(i) by reacting the compound of Formula I with the desired acid or base;

(ii) by removing an acid- or base-labile protecting group from a suitable precursor of the compound of Formula I or by ring-opening a suitable cyclic precursor, for example, a lactone or lactam, using the desired acid or base; or

(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (“glass transition”). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H, G. Brittain, Marcel Dekker. 1995). Isolated site hydrates are ones in which the water molecules are isolated from direct contact with each other by intervening organic molecules, in channel hydrates, the water molecules lie in lattice channels where they are next to other water molecules. In metal-ion coordinated hydrates, the water molecules are bonded to the metal iron.

When the solvent or water is tightly bound, the complex will have a will-defined stoichiometry independent of humidity. When, however, the solvent or water is weakly bound, as in channel solvates and hygroscopic compounds, the water/solvent content will be dependent on humidity and drying conditions, in such cases, non-stoichiometry will be the norm.

The compounds of the invention may also exist in a mesomorphic state (mesophase or liquid crystal) when subjected to suitable conditions. The mesomorphic state is intermediate between the true crystalline state and the true liquid state (either melt or solution), Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphillic’ and consist of molecules which possess an ionic (such as —COO⁻Na⁺, —COO⁻K⁺, or —SO₃Na⁺) or non-ionic (such as —N⁻N⁺(CH₃)₃) polar head group. For more information, see Crystals and the Polarizing Microscope by N. H, Hartshorne and A. Stuart, 4^{th} Edition (Edward Arnold, 1970).

Hereinafter all references to compounds of Formula I or a specific compound of Formula I, unless otherwise indicated, are meant to encompass all salts, solvates, multi-component complexes and liquid crystals of said compounds or compound including but not limited to solvates, multi-component complexes and liquid crystals of said salts.

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

As indicated, so-called ‘prodrugs’ of the compounds of Formula I are also within the scope of the invention. Thus certain derivatives of compounds of Formula I which may have little or no pharmacological activity themselves can, when administered into or onto the body, be converted into compounds of Formula I having the desired activity, for example, by hydrolytic cleavage Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H, Bundgaard (Elsevier, 1985).

Some examples of prodrugs in accordance with the invention include, but are not limited to,

(i) where the compound of Formula I contains a carboxylic acid functionality (—COOH), an ester thereof, for example, a compound wherein the hydrogen of the carboxylic acid functionality of the compound of Formula (I) is replaced by (C₁-C₈)alkyl;

(ii) where the compound of Formula I contains an alcohol functionality (—OH), an ether thereof, for example, a compound wherein the hydrogen of the alcohol functionality of the compound of Formula I is replaced by (C₁-C₅)alkanoyl oxymethyl; and

(iii) where the compound of Formula I contains a primary or secondary amino functionality (—NH₂ or —NHR where R≠H), an amide thereof, for example, a compound wherein, as the case may be, one or both hydrogens of the amino functionality of the compound of Formula I is/are replaced by (C₁-C₁₀)alkanoyl.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include, but are not limited to,

(i) where the compound of Formula I contains a methyl group, an hydroxymethyl derivative thereof (—CH₃->—CH₂OH):

(ii) where the compound of Formula I contains an alkoxy group, an hydroxy derivative thereof (—OR->—OH):

(iii) where the compound of Formula I contains a tertiary amino group, a secondary amino derivative thereof (—NR¹R²->—NHR¹ or —NHR²);

(iv) where the compound of Formula I contains a secondary amino group, a primary derivative thereof (—NHR¹->—NH₂);

(v) where the compound of Formula I contains a phenyl moiety, a phenol derivative thereof (-Ph->-PhOH); and

(vi) where the compound of Formula I contains an amide group, a carboxylic acid derivative thereof (—CONH₂->COOH);

(vii) where the compound contains an aromatic nitrogen atom or an tertiary aliphatic amine function, an N-oxide derivative thereof.

It is to be understood that reference to the term “when present,” as used in the claims, means a ring atom's substituent may be absent. This may occur, for example, when a ring atom is N, O, or S. For example, when M in formula I is N, O, or S, then R⁸ may be absent because all of M's available bonding sites are used to form the heteroaromatic ring.

Included within the scope of this invention are compounds of Formula I wherein a nitrogen atom in an aromatic or non-aromatic tertiary amine functional group (e.g. pyridyl nitrogen, piperidinyl nitrogen, etc.) may be further substituted with oxygen (i.e., an N-oxide), such that a compound of formula I may have one or more N-oxides.

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of Formula I containing, for example, an imino, keto, or oxime group. Tautomerism can also take the form of so-called valence tautomerism in compounds that contain an aromatic moiety. It follows that a single compound may exhibit more than one type of isomerism.

This invention also relates to those stereoisomers of compounds of the formula I that are atropisomers, Atropisomers are isomeric compounds that are chiral, i.e., each isomer is not superimpossible on its mirror image and the isomers, once separated, rotate polarized light in equal but opposite directions. Atropisomers are distinguished from enantiomers in that atropisomers do not possess a single asymmetric atom. Such compounds are conformational isomers which occur when rotation about a single bond in the molecule is prevented or greatly slowed as a result of steric interactions with other parts of the molecule and the substituents at both ends of the single bond are unsymmetrical A detailed account of atropisomers can be found in Jerry March, Advanced Organic Chemistry, 101-102 (4th ed. 1992) and in Oki, Top Stereochem, 14, 1-81(1983). Included within the scope of the present claims are all stereoisomers, atropisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or l-lysine, or racemic, for example, di-tartrate or di-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamide. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L Eliel and S. H, Wilen (Wiley, 1994).

Accordingly, references herein to a specific compound of Formula I, unless otherwise indicated, are meant to include any tautomer, pure or substantially pure enantiomer, or racemic mixture of said compound.

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include, but are not limited to, isotopes of hydrogen, such as ²H and ³H, carbon, such as ¹¹C, ¹³C and ¹⁴C, chlorine, such as 36Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of Formula I, for example, those Incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₅-DMSO.

Specific embodiments of the present invention include the compounds exemplified in the Examples below and their pharmaceutically acceptable salts, complexes, solvates, polymorphs, steroisomers, metabolites, prodrugs, and other derivatives thereof:

This invention also pertains to a pharmaceutical composition for treatment of certain psychotic disorders and conditions such as schizophrenia, delusional disorders and drug induced psychosis; to anxiety disorders such as panic and obsessive-compulsive disorder; and to movement disorders including Parkinson's disease and Huntington's disease, comprising an amount of a compound of formula I effective in inhibiting PDE 10.

in another embodiment, this invention relates to a pharmaceutical composition for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising an amount of a compound of formula I effective in treating said disorder or condition.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to selected from Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other tic disorders.

In another embodiment, this invention relates to a method for treating an anxiety disorder or condition in a mammal which method comprises administering to said mammal an amount of a compound of formula I effective in inhibiting PDE 10.

This invention also provides a method for treating an anxiety disorder or condition in a mammal which method comprises administering to said mammal an amount of a compound of formula I effective in treating said disorder or condition.

Examples of anxiety disorders that can be treated according to the present invention include, but are not limited to, panic disorder; agoraphobia; a specific phobia; social phobia; obsessive-compulsive disorder; post-traumatic stress disorder, acute stress disorder, and generalized anxiety disorder.

This invention further provides a method of treating a drug addiction, for example an alcohol, amphetamine, cocaine, or opiate addiction, in a mammal, including a human, which method comprises administering to said mammal an amount of a compound of formula I effective in treating drug addiction.

This invention also provides a method of treating a drug addiction, for example an alcohol, amphetamine, cocaine, or opiate addiction, in a mammal, including a human, which method comprises administering to said mammal an amount of a compound of formula I effective in inhibiting PDE 10.

A “drug addiction”, as used herein, means an abnormal desire for a drug and is generally characterized by motivational disturbances such a compulsion to take the desired drug and episodes of intense drug craving.

This invention further provides a method of treating a disorder comprising as a symptom a deficiency in attention and/or cognition in a mammal, including a human, which method comprises administering to said mammal an amount of a compound of formula I effective in treating said disorder.

This invention also provides a method of treating a disorder or condition comprising as a symptom a deficiency in attention and/or cognition in a mammal, including a human, which method comprises administering to said mammal an amount of a compound of formula I effective in inhibiting PDE 10.

This invention also provides a method of treating a disorder or condition comprising as a symptom a deficiency in attention and/or cognition in a mammal, including a human, which method comprises administering to said mammal an amount of a compound of formula effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects, for example as occurs in age-related cognitive decline.

Examples of disorders that comprise as a symptom a deficiency in attention and/or cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline.

This invention also provides a method of treating a mood disorder or mood episode in a mammal, including a human, comprising administering to said mammal an amount of a compound of formula I effective in treating said disorder or episode.

This invention also provides a method of treating a mood disorder or mood episode in a mammal, including a human, comprising administering to said mammal an amount of a compound of formula I effective in inhibiting PDE10.

Examples of mood disorders and mood episodes that can be treated according to the present invention include, but are not limited to, major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression, major depressive disorder; dysthymic disorder; minor depressive disorder; premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder.

This invention further provides a method of treating a neurodegenerative disorder or condition in a mammal, including a human, which method comprises administering to said mammal an amount of a compound of formula I effective in treating said disorder or condition.

This invention further provides a method of treating a neurodegenerative disorder or condition in a mammal, including a human, which method comprises administering to said mammal an amount of a compound of formula I effective in inhibiting PDE10.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons. The term “neurotrophic agent” as used herein refers to a substance or agent that has some or all of these properties.

Examples of neurodegenerative disorders and conditions that can be treated according to the present invention include, but are not limited to, Parkinson's disease; Huntington's disease; dementia, for example Alzheimer's disease, multi-infarct dementia, AIDS-related dementia, and Fronto temporal Dementia; neurodegeneration associated with cerebral trauma, neurodegeneration associated with stroke, neurodegeneration associated with cerebral infarct; hypoglycemia-induced neurodegeneration; neurodegeneration associated with epileptic seizure; neurodegeneration associated with neurotoxin poisoning; and multi-system atrophy.

In one embodiment of the present invention, the neurodegenerative disorder or condition comprises neurodegeneration of striatal medium spiny neurons in a mammal, including a human.

In a further embodiment of the present invention, the neurodegenerative disorder or condition is Huntington's disease.

This invention also provides a pharmaceutical composition for treating psychotic disorders, delusional disorders and drug induced psychosis; anxiety disorders, movement disorders, mood disorders, neurodegenerative disorders, obesity, and drug addiction, comprising an amount of a compound of formula I effective in treating said disorder or condition.

This invention also provides a method of treating a disorder selected from psychotic disorders, delusional disorders and drug induced psychosis; anxiety disorders, movement disorders, obesity, mood disorders, and neurodegenerative disorders, which method comprises administering an amount of a compound of formula I effective in treating said disorder.

This invention also provides a method of treating disorders selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features, a depressive episode with melancholic features; a depressive episode with catatonic features, a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temporal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, obesity, inhalants, opioids, or phencyclidine; personality disorder of the paranoid type; and personality disorder of the schizoid type, which method comprises administering an amount of a compound of Formula I effecting in said disorders.

This invention also provides a method of treating psychotic disorders, delusional disorders and drug induced psychosis; anxiety disorders, movement disorders, mood disorders, neurodegenerative disorders, obesity, and drug addiction which method comprises administering an amount of a compound of formula I effective in inhibiting PDE10.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl”, as used herein, unless otherwise indicated, includes monovalent hydrocarbon radicals having at least one carbon-carbon double bond wherein alkyl is as defined above. Examples of alkenyl include, but are not limited to, ethenyl and propenyl.

The term “alkynyl”, as used herein, unless otherwise indicated, includes monovalent hydrocarbon radicals having at least one carbon-carbon triple bond wherein alkyl is as defined above. Examples of alkynyl groups include, but are not limited to, ethynyl and 2-propynyl.

The term “alkoxy”, as used herein, unless otherwise indicated, as employed herein alone or as part of another group refers to an alkyl, groups linked to an oxygen atom.

The term “alkylthio” as used herein, unless otherwise indicated, employed herein alone or as part of another group includes any of the above alkyl groups linked through a sulfur atom.

The term “halogen” or “halo” as used herein alone or as part at another group refers to chlorine, bromine, fluorine, and iodine.

The term “haloalkyl” as used herein, unless otherwise indicated, refers to at least one halo group, linked to an alkyl group. Examples, of haloalkyl groups include, but are not limited, to trifluoromethyl, trifluoroethyl, difluoromethyl and fluoromethyl groups.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

The term “aryl”, as used herein, unless otherwise indicated, includes an organic radical derived from an aromatic hydrocarbon by removal of one hydrogen, such as phenyl, naphthyl, indenyl, and fluorenyl, “Aryl” encompasses fused ring groups wherein at least one ring is aromatic.

Unless otherwise indicated, the term “heterocycloalkyl”, as used herein, refer to non-aromatic cyclic groups containing one or more heteroatoms, prefereably from one to four heteroatoms, each preferably selected from oxygen, sulfur and nitrogen. The heterocycloalkyl groups of this invention can also include ring systems substituted with one or more oxo moieties. Examples of non-aromatic heterocycloalkyl groups are aziridinyl, azetidinyl, pyrrolidinyl, piperidinyl, azepinyl, piperazinyl, 1,2,3,8-tetrahydropyridinyl, oxiranyl, oxetanyl, tetrahydrofuranyl, tetrahydrothienyl, tetrahydropyrenyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

Unless otherwise indicated, the term “heteroaromatic ring” as used herein, refers to an aromatic ring containing one or more heteroatoms (preferably oxygen, sulfur and nitrogen), preferably from one to four heteroatoms.

Unless otherwise indicated, the term “heteroaryl”, as used herein, refers to a radical derived from a heteroaromatic ring. Examples of 5 to 5 membered heteroaryls are pyridinyl, pyridazinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, triazinyl, purinyl, oxadiazolyl, thiadiazolyl, furazanyl. A ring nitrogen in a double bond in a heteroaryl or a heteroaromatic ring may be substituted with oxygen (as in N-oxide). In the instant application, heteroaryl groups are hereby defined to include heterocyclic rings substituted on carbon with one or more oxo moieties, if a tautomer of said ring can be drawn wherein the double bond of each oxo moiety can be moved within the ring and a ring proton, usually on nitrogen, is moved to the oxygen of each said oxo moiety, giving a tautomeric form having one or more hydroxy substituents on an aromatic ring as defined above. Examples of said heterocyclic ring substituted with one oxo moiety where a proton tautomer can be drawn include an imidazol-2-one group which can be drawn as a 2-hydroxyimidazole, and the same imidazol-2-one group of a benzimidazol-2-one which can be represented as a 2-hydroxyimidazole fused to a benzene ring as in 2-hydroxybenzimidazole. The terms “heterocyclic ring” and “heterocycle” include heteroaryl and heteroaromatic rings as well as non-aromatic heterocyclic rings containing zero or more double bonds. Tertiary nitrogen atoms in heterocycles which are not heteroaromatic may also be substituted by oxygen (as in N-oxide).

Unless otherwise indicated, the term “carbocyclic ring”, as used herein, includes aryl and alicyclic rings (e.g. cycloalkyl, cycloalkenyl, cycloalkadienyl).

Unless otherwise indicated, the term “heterocyclic ring”, as used herein, includes heteroaryl, heterocycloalkyl, heterocycloalkenyl, and heterocycloalkadienyl rings.

Unless otherwise indicated, the term “one or more” substituents, or “at least one” substituent as used herein, refers to from one to the maximum number of substituents possible based on the number of available bonding sites.

Unless otherwise indicated, all the foregoing groups derived from hydrocarbons may have up to about 1 to about 20 carbon atoms (e.g. C₁-C₂₅ alkyl, C₂-C₂₀ alkenyl, C₃-C₂₀ cycloalkyl, 3-20 membered heterocycloalkyl; C₆-C₂₀ aryl, 5-20 membered heteroaryl, etc.) or 1 to about 15 carbon atoms (e.g., C₁-C₁₅ alkyl, C₂-C₁₅ alkenyl, C₃-C₁₅ cycloalkyl, 3-15 membered heterocycloalkyl, C₆-C₁₅ aryl, 5-15 membered heteroaryl, etc.), or 1 to about 12 carbon atoms, or 1 to about 8 carbon atoms, or 1 to about 6 carbon atoms.

Unless otherwise indicated, the term “oxo”, as used herein, refers to a double-bonded oxygen atom attached to carbon or sulfur. For example, an oxo-substituted carbon atom is a carbonyl (as in a ketone or amid functional group); and an oxo-substituted sulfur (S═O) can be present in a sulfoxide, sulfone, sulfinamide, or sulfonamide.

“Neurotoxin poisoning” refers to poisoning caused by a neurotoxin. A neurotoxin is any chemical or substance that can cause neural death and thus neurological damage. An example of a neurotoxin is alcohol, which, when abused by a pregnant female, can result in alcohol poisoning and neurological damage known as Fetal Alcohol Syndrome in a newborn. Other examples of neurotoxins include, but are not limited to, kainic acid, domoic acid, and acromelic acid; certain pesticides, such as DDT; certain insecticides, such as organophosphates; volatile organic solvents such as hexacarbons (e.g. toluene); heavy metals (e.g. lead, mercury, arsenic, and phosphorous); aluminum; certain chemicals used as weapons, such as Agent Orange and Nerve Gas; and neurotoxic antineooplastic agents.

As used herein, the term “selective PDE 10 inhibitor” refers to a substance, for example an organic molecule, that effectively inhibits an enzyme from the PDE10 family to a greater extent than enzymes from the PDE 1-9 families or PDE11 family. In one embodiment, a selective PDE10 inhibitor is a substance, for example an organic molecule, having a Kᵢ for inhibition of PDE10 that is less than or about one-tenth the K; that the substance has for inhibition of any other PDE enzyme, in other words, the substance inhibits PDE10 activity to the same degree at a concentration of about one-tenth or less than the concentration required for any other PDE enzyme.

In general, a substance is considered to effectively inhibit PDE10 activity if it has a Kᵢ of less than or about 10 μM, preferablyless than or about 0.1 μM.

A “selective PDE10 inhibitor” can be identified, for example, by comparing the ability of a substance to inhibit PDE10 activity to its ability to inhibit PDE enzymes from the other PDE families. For example, a substance may be assayed for its ability to inhibit PDE10 activity, as well as PDE1A, PDE1B, PDE1C, PDE2, PDE3A, PDE3B, PDE4A, PDE4B, PDE4C, PDE4D, PDE5, PDE6, PDE7, PDE8, PDE9, and PDE11.

The term “treating”, as in “a method of treating a disorder”, refers to reversing, alleviating, or inhibiting the progress of the disorder to which such term applies, or one or more symptoms of the disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffective disorders include disorganized speech, affective flattening, alogia, anhedonia, inappropriate affect, dysphoric mood (in the form of, for example, depression, anxiety or anger), and some indications of cognitive dysfunction.

The term “mammal”, as used herein, refers to any member of the class “Mammalia”, including, but not limited to, humans, dogs, and cats

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration, or in a form suitable for administration by inhalation or insufflation.

The dissolution rate of poorly water-soluble compounds may be enhanced by the use of a spray-dried dispersion, such as those described by Takeuchi, H., et al. in “Enhancement of the dissolution rate of a poorly water-soluble drug (tolbutamide) by a spray-drying solvent deposition method and disintegrants” J. Pharm, Pharmacol. 39, 768-773 (1987).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates sorbic acid).

For buccal administration, the composition may take the form of tablets or lozenges formulated in conventional manner.

The compounds of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

When a product solution is required, it can be made by dissolving the isolated inclusion complex in water (or other aqueous medium) in an amount sufficient to generate a solution of the required strength for oral or parenteral administration to patients. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compounds of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound Capsules and cartridges (made e.g. from gelatin) for use in an inhaler or insufflator may be formulated containing a powder mix of a compound of the invention and a suitable powder base such as lactose or starch.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

A proposed daily dose of the compound of the invention for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

Compounds of the present invention were evaluated for ability to inhibit PDE10 enzyme with the following Assay procedure

The enzyme used in the procedure was cloned rat PDE10A full-length enzyme grown in transfected Sf9 insect cells. Cloned enzyme was extracted from homogenized cell pellets and stored frozen in homogenizing buffer until use. Compounds were initially dissolved in 100% DMSO and diluted out in 20 percent DMSO/water solution. Final concentration of DMSO in the assay was 2 percent as compounds were tested in triplicate in 96 well plates. Compound solution was placed in well, then tritiated cyclic AMP (New England Nuclear NET275) in assay buffer was added at 20 nM concentration. Then PDE10 enzyme in assay buffer of 50 mM Tris, 8.3 mM MgCl₂, pH 7.5 at room temperature was added for a final assay volume of 100 ul. Concentration of enzyme was added such that less than 10 percent of [3H]cAMP at 20 nM was converted to detectable end product, [3H]AMP bound to SPA (Scintillation Proximity Assay) beads. Phosphodiesterase scintillation proximity yttrium silicate beads from Amersham Biosciences (RPNQ0150) were added (50 ul at 20 mg/ml) after a 20 minute incubation at room temperature. Zinc sulphate as a component of the beads stops the phosphodiesterase reaction. Plates were let stand 12 to 16 hours and then counted in a Trilux plate reader to allow calculation of IC₅₀'s.

Non-specific binding to SPA beads was determined by addition of 1 uM papaverine. Total conversion by enzyme without inhibitor of [3H]cAMP to [3H]AMP, as detected by scintillation of [3H]AMP bound to yttrium silicate beads, was determined in the presence of vehicle-only.

### DETAILED DESCRIPTION OF THE INVENTION

When synthesizing compounds of formula I or their precursors, one skilled in the art may wish to choose reaction conditions which are not comparable with all functionality present in the reactants. Examples of said functionality are a more reactive primary amine, when the intended reaction involves another amine, or a carboxy group, where the intended reaction involves a different carboxy group. In such a case, the practitioner may determine that use of a protecting group is advantageous to avoid side reactions involving said functionality, and choose to protect said functionality, or transform said functionality into a protected, unreactive form, by use of an appropriate protecting group. One well-known reference to practitioners for choosing chemistry to introduce and remove protecting groups, estimating the need for and nature of said protecting groups, and choosing reactions compatable with specific protecting groups is that of T. W. Greene and P. G. M. Wuts. Protective Groups in Organic Synthesis, John Wiley & Sons, New York, 1999. Instances of using protecting groups in the synthesis of compounds of Formula I are given in the Examples and serve as illustrations. Examples 31 and 33 show the protection of amine functionality in the synthesis and use of a protected intermediate of formula R₁—NH₂, and subsequent removal of the protecting group to provide a compound of formula I.

Unless otherwise noted, variables present in the structures in the Schemes are as defined for formula I in the “Summary of the Invention” section.

For clarity, the radical containing the EFGJ and WXYZ rings and corresponding substituents of Formula I is illustrated as structure II in the Schemes shown below. The radical containing the LMQ(T)ₙUV ring and its substituents is illustrated as structure III. The radical containing the EFGJ and LMQ(T)ₙUV ring is illustrated as structure IV and that containing the WXYZN ring and its substituents as structure V.

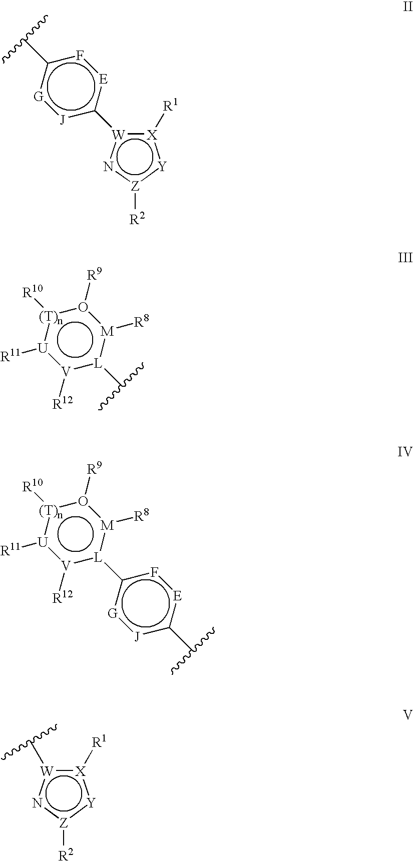

Also for brevity and clarity, nine subtypes of ring WXYZN contained in formulae I, II, and V are shown and illustrated as subtypes a-i of said formulae (attachment point is to W). Thus, a compound of formula Ia in the Schemes is refers to a compound of formula I having W=C, X=N, Y=C(R²⁰), and Z=C. Likewise, a synthetic intermediate containing the radical Va (for example R²²-Va as shown in Scheme IV) in the Schemes, refers to a compound of formula R²²-V wherein R²² is attached at W, W=C, X=N, Y=C(R²⁰), Z=C, and so forth.

### Subtypes a-i of I, II, and V:

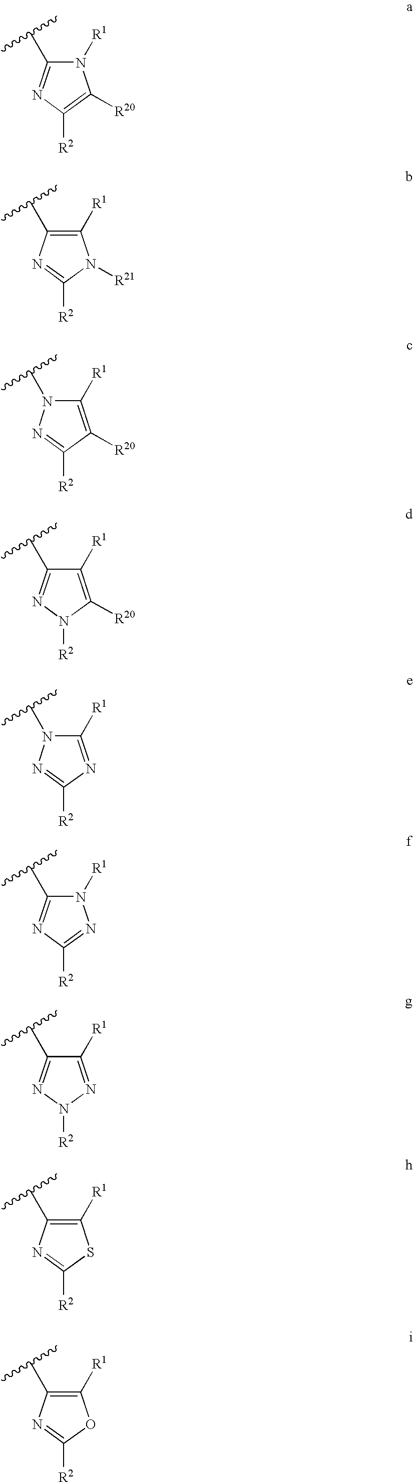

Scheme I illustrates four reaction types to prepare compounds of formula I by coupling compounds containing the LMQ(T)ₙUV ring portion of I (H-III, M₁-III, or X₁-III) with compounds containing the remaining atoms of I (X₁-II or M₁-II). If not otherwise designated, X₁ is an atom or group which renders electrophilic the atom in II or III to which it is attached, also making the group suitable for the coupling reactions discussed below, and is preferably selected from the group consisting of halogen, arylsulfonate (including tosylate and bromobenzenesulfonate), alkysulfonate (including mesylate), or perfluoroalkylsulfonate (including triflate and nonaflate), and more preferably is bromine, iodine, or triflate. M₁ is a metal atom or metal atom with attached ligands which renders nucleophilic the atom in II or III to which if is attached, which is also suitable for the coupling reactions discussed below, and is preferably selected from boron, tin, magnesium or zinc, together with attached ligands which include halide atoms and alkyl groups. One skilled in the art of chemical synthesis will recognize that the reactions represented in Scheme I represent different types of reactions and are generally described in the chemical literature as aryl-aryl, bi-aryl, heteroaryl-aryl, or heteroaryl-heteroaryl coupling reactions, arylation or heteroarylation reactions of aryl and/or heteroaryl halides, triflates or sulfonates, and aryl and heteroaryl amination reactions of aryl or heteroaryl halides and sulfonates (for example when L=N in H-III) or direct C—H arylation (for example when L=C in H-III). One skilled in the art will also recognize that each said type of reaction is effected and optimized by correct selection of appropriate metal atoms or metal-containing ligands (herein M₁), activating group X₁, and reaction conditions including solvent, concentration, catalysts, ligands, bases, temperature, presence of other reagents, and that there is extensive guidance given in the chemical literature to choose these conditions based on the chemical structures of the coupling partners including the identify of M₁ and X₁ which are readily available to one skilled in the art to locate and assist in the choice of optimal conditions.

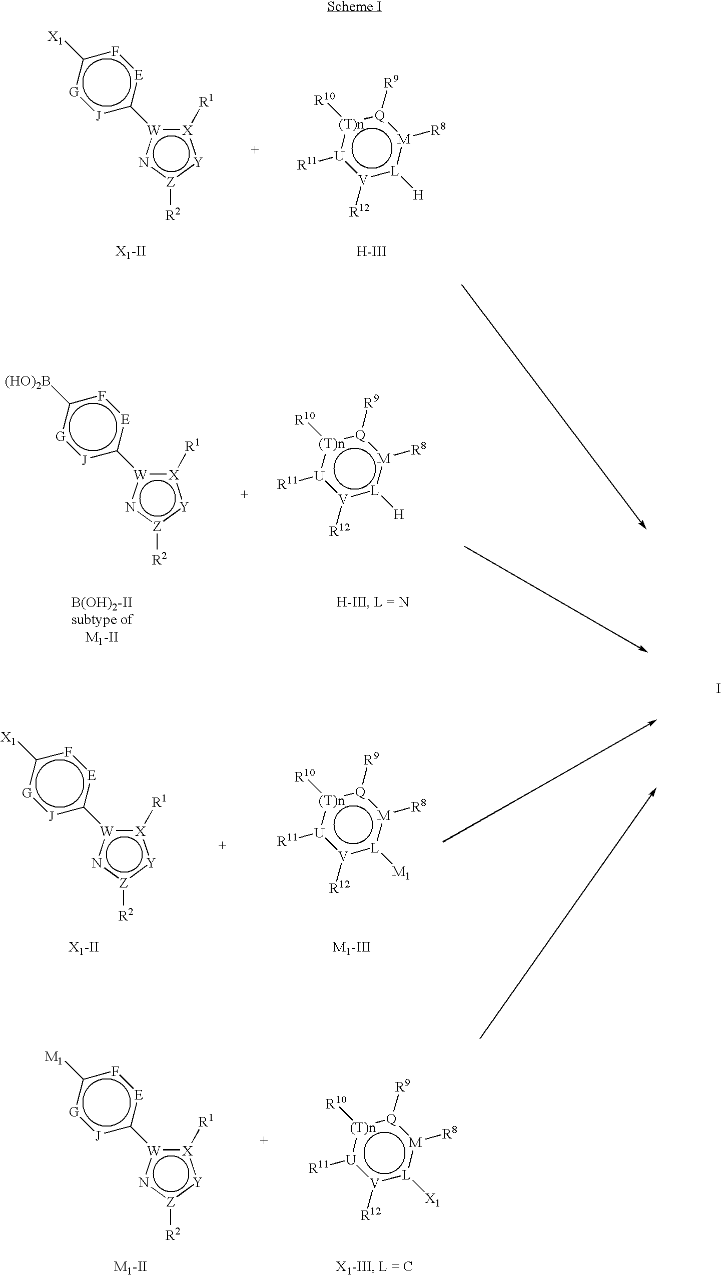

The first reaction shown in Scheme I is useful for preparing compounds of Formula I (L=N), by N-heteroarylation or N-arylation of a heterocycle of formula H-III (L=N) with a compound of formula X₁-II. In this reaction X₁ is more preferably iodine, Br, or Cl. A particularly useful method illustrated by many Examples provided in the instant application, and described in the literature by Buchwald (for example Antilla, J. Org. Chem. 2004, vol 69, p. 5578 and J, Am. Chem. Soc. 2001, vol 123, p. 7727) is that of combining X₁-II (X₁ is preferably iodine or bromine, more preferably iodine) and H-III with a catalytic amount of cuprous iodide (usually 5-10 mol %), 5-10 mot % of a 1,2-diamine ligand (e.g. trans-N,N′-dimethyl-cyclohexane-1,2-diamine, trans-cyclohexane-1,2-diamine, N,N′-dimethylethylenediamine, or 1,10-phenanthroline), and 1-3 equivalents, preferably about 2 equivalents of a base such as potassium phosphate, potassium carbonate or cesium carbonate and heating the mixture at between 80-160° C., usually 100-120° C. for an optimal time. A solvent, preferably dioxane, dimethylformamide, or toluene, is usually employed. Heating by microwave irradiation may be advantageous. Variations of these conditions such as use of CuO, potassium carbonate, and dimethylformamide solvent without diamine ligands may also be successful. A second method is that described by Hartwig (J. Am. Chem. Soc. 1998, vol. 120, p. 827 and J. Org. Chem. 1999, vol. 64, p 5575) wherein certain heterocycles III(L=N) are coupled to aryl iodides, bromides, and chlorides using catalytic amounts (3-5 mol %) of Pd(dba)₂(bis(dibenzylidineacetone)palladium(0)), 0.8-1 equiv tributylphosphine in toluene at about 100° C. Said Hartwig method may be applied to synthesis of I (L=N), by substituting a compound of formula X₁-II for the aryl halide cited therein. A third method described by Holmes et al. (WO 2005/090283), that of N-arylation of N-trialkylsilyl derivatives of certain compounds of formula III, by heating said derivative with an aryl halide in the presence of cesium carbonate, palladium acetate, and di-t-butylbiphenylphosphine at 100° C. in a pressure vessel containing sufficient carbon dioxide to generate a pressure of about 3000 p.s.i., may be used to prepare compounds of formula I by employing a trialkylsilyl derivative of H-III (namely R₃Si-III wherein L is N and R is preferably methyl), and substituting a compound of formula X₁-II (X₁ preferably iodine or Br), for said aryl halide. Yet other conditions suitable for coupling H-III and X₁-II to give I where L is nitrogen, are those of Kuil (Tetrahedron Lett. 2005, vol. 46, p. 2405, N-methylpyrrolidone solvent, catalytic cuprous salt (preferably iodide), and 1.1 equiv cesium carbonate at 110-125° C. in the presence of 10 mol % 4,7-dichloro-1,10-phenanthroline) or those of Cristau (Eur. J. Org. Chem. 2004, p. 695, and Chem. Eur. J. 2004, vol. 10, p 5607) using cuprous salts, cesium carbonate, and oxime ligands, and those of Cai (Synthesis 2004, p. 498) using cuprous iodide, amino acid ligands (e.g. proline), potassium carbonate, and solvents including dimethylsulfoxide and dimethylacetamide. The review of Ley (Angew. Chem. Int. Ed. 2003, 42, 5400-5449, especially Section 3, pp. 5418-5431 therein) contains references to other useful methods for coupling X₁-II and H-III using copper salts). Reviews citing other useful methods are given by Jiang (Metal-Catalyzed Cross-Coupling Reactions. 2^{nd} Edition. A. de Meijere, F. Diederich, Eds. Copyright 2004, Wiley-VCH Verlag GmbH & Co. KGaA. Weinhem, Germany), and Muci (Top. Curr. Chem. 2002, vol. 219, pp 133-209).

The first route show in Scheme I may also be employed for preparing compounds of formula I from compounds of formula H-III wherein L is carbon in this instance the reaction is described in the literature as a direct CH-arylation or a direct arylation of aromatic carbon, in the instant application of compound H-III by a compound X₁-II. Sames (Org. Lett 2004, vol. 6, p. 2897) provides methods for the direct CH-arylation of heterocycles of formula H-III such as indoles, imidazoles and benzimidazoles by aryl halides using conditions including 0.05 equiv palladium (II) acetate, 0.02 equiv triphenylphosphine, 2 equiv cesium acetate in dimethylformamide at 125° C. which may be adapted to the formation of compounds of formula I by substituting a compound of formula X₁-II wherein X₁ is more preferably iodine for said aryl halide of Sames. Additional methods which may also be employed to achieve coupling of H-III with X₁-II to give formula I compounds wherein L=carbon are described (J. Am. Chem. Soc. 2003, vol 125, p. 10580; Org. Lett 2003, vol 5, p. 3607; Abstracts of Papers, 230th ACS National Meeting, Washington, D.C., United States, Aug. 28-Sep. 1, 2005 (2005), ORGN-270; WO 2004/069394; J. Am. Chem. Soc. 2005, vol, 127, p. 3648, 4996, and 5284 and references therein).

The second reaction shown in Scheme I is employed for synthesizing a compound of formula I by coupling B(OH)₂-II with H-III (where L=N). A preferred method is their reaction in the presence of 1-2 equiv cupric acetate, triethylamine or pyridine, and molecular selves in dichloromethane at room temperature for an appropriate period. This method is described by Chan (Tetrahedron Lett. 1998, 39, 2933-2936) and Lam (Tetrahedron Lett. 1998, 39 2941). Many applications by other workers and modifications useful for optimizing said coupling of B(OH)₂-II with H-III (where L=N) are cited in a review by Ley (Angew. Chem. Int. Ed. 2003, 42, 5400-5449, pp. 5408-5417 therein) including methods employing additional ligands and co-oxidants which permit use of catalytic quantities of copper salts.

Compounds of formula I, wherein L is carbon, may be prepared by coupling either X₁-II and M₁-III or X₁-III and M₁-II (third and fourth reactions, respectively of Scheme I). Two reactions are especially useful for performing this coupling, the Suzuki or Suzuki-Miyaura reaction and the Stille reaction. The Suzuki or Suzuki-Miyaura reaction is the reaction of organoboron derivatives with organic electrophiles in the presence of a base. In application of this reaction to synthesis of compounds of formula I, is M₁ is (OH)₂, borate ester B(OR)₂, or M₁-III may be a triaryl- or tri-heteroarylboroxine also described by the formulae (III-B(−)—O—)₃ or (III)₃-boroxine. Likewise M₁-II may be a boroxine derivative described by formulae (II-B(−)—O—)₃ or (II)₃-boroxine (these formulae are shown for clarity.

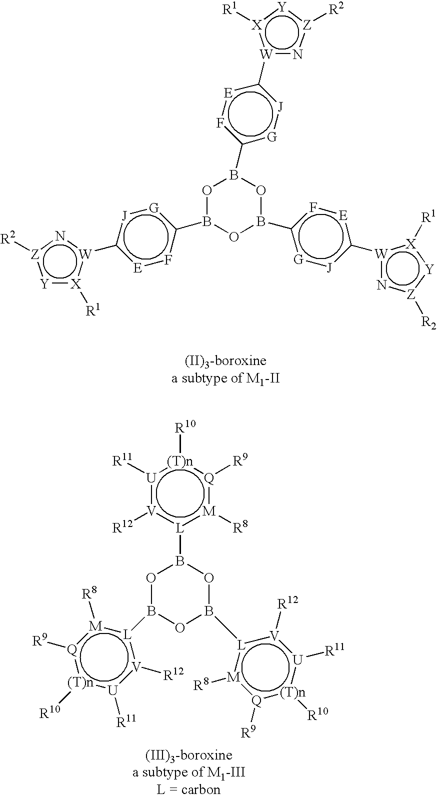

In said borate ester, R is usually a C₁-C₅ linear or branched alkyl group, or the two R groups are taken together with the oxygen and boron atoms which they are attached to form a 5-6 membered ring containing two or three carbon atoms which may be further substituted by alkyl groups or by fusion of a benzene ring to two of said carbons when the ring is 5-membered. For clarity, said cyclic borate ester is a borate ester of a diol such as ethylene glycol, propylene glycol, 2,2,3,3-tetramethyl-1,2-ethanediol (pinacol), or ortho-catechol, respectively. X₁ is preferably iodide, Br, Cl, or triflate. In a typical application, X₁-II and M₁-III, or X₁-III and M₁-II are combined with a palladium catalyst (0.01-0.1 mol equiv) and a base (usually 1-3 equiv) in a suitable solvent and heated at 20-220° C. preferably 80-150° C. for an optimal period. Palladium catalysts include Pd(OAc)₂, Pd₂(dba)₃ (tris(dibenzylidineacetone)dipalladium(0)), PdCl₂, PdCl₂(1,1-bis(diphenylphosphino)ferrocene) and Pd(PPh₃)₄. Palladium catalysts which contain phosphine-based ligands that are more stable on heating (such as Pd(PPh₃)₄), may be advantageous. Additional ligand may be added separately in an optimal amount. Catalyst selection for the Suzuki reaction has been reviewed by Bellina (Synthesis (2004), vol. 15, p. 2418). Suitable bases include Na₂CO₃, K₃PO₄, TI₂CO₃, NaHCO₂, (n-Bu)₄NF, Ba(OH)₂ and CsF. Suitable solvents include water, toluene, dioxane, dichloromethane, dimethoxyethane, dimethylformamide, tetrahydrofuran and ethanol. Mixtures of two or more solvents may be employed. Heating by microwave may shorten reaction time and improve yield. The Suzuki reaction may also be performed without catalyst (Leadbeater, Chem. Commun, 2005, vol 23, p, 2881). Reviews of the Suzuki/Suzuki-Miyaura reaction which contain additional guidance to the skilled artisan as to the selection of appropriate reaction conditions are given by Suzuki (Journal of Organometallic Chemistry (2002), 853(1-2), 83-90; Handbook of Organopalladium Chemistry for Organic Synthesis (2002), 1 249-262. Publisher: John Wiley & Sons, inc., Hoboken, N. J; J. Organomet. Chem. 1989, vol. 576, 147-168), Miyaura (Chem. Rev. 1895, vol. 95, pp. 2457-2483) and Li (Organic Syntheses (2005), vol. 81, pp. 89-97). Exemplary preparations of compounds I using the Suzuki reaction are provided in the Examples section of this application.

The Stille reaction is another reaction particularly useful for synthesizing compounds of formula I (LL=C), by coupling either X₁-II and M₁-III or X₁-III and M₁-II (third and fourth reactions, respectively of Scheme I). In this application of the Stille reaction, M₁ is a tin-containing group attached at tin (including SnMe₃, SnCl₃, or preferably Sn(n-Bu)₃ or SnR₃ where R is a longer alkyl chain), and X₁ is more preferably iodine, Br, triflate, or Cl and most preferably iodine, Br or triflate. The coupling is effected by combining these reactants in the presence of a palladium catalyst, preferably a Pd(0) or Pd(II) catalyst with attached ligands such as Pd(PPh₃)₄, bis(dibenzylideneacetone)palladium, bis(acetonitrile)palladium(II) dichloride, bis(triphenylphosphine)palladium(II) chloride, benzyl[bis(triphenylphosphine)]palladium(II) chloride, 1,1′-bis(diphenylphosphino)ferrocenepalladium(II) dichloride, and allylpalladium(II) chloride dimer, in an inert solvent such as toluene, tetrahydrofuran, xylene, benzene, dioxane, dichloroethane, dimethylformamide or N-methylpyrrolidone, at a suitable temperature (typically 80-150° C., including heating by microwave). Examples of suitable conditions are heating the coupling partners with 1-5% Pd(PPh₃)₄ or Pd(PPh₃)₂Cl₂ in tetrahydrofuran, dimethylformamide, dioxane or xylene solvent at reflux temperature. Specific illustrations of this method to synthesize compounds of formula I by the copper-assisted Stille reaction are given in Examples 77-80 herein. When the palladium catalyst is a palladium(II) catalyst the addition of an excess amount of M₁-II or M₁-III may be desirable. Additional ligand may also be added if beneficial. Addition of a salt such as LiCl and bases such as triethylamine, diisopropylethylamine, pyridine, sodium carbonate, and lithium carbonate may be beneficial. Other additives such as cuprous iodide (Farina, J. Org. Chem. 1994, vol. 59, p. 5905), cuprous oxide, or silver oxide may be added to improve the yield and rate of the Stille reaction leading to compounds of formula I. Guidance to the skilled artisan useful for conducting and optimizing the Stifle reaction to prepare compounds of formula I are provided in reviews by Stille (Angew. Chem. Intl. Ed. Engl. 1986, vol 25, p. 508) and Farina (Org. Reactions 1997, vol. 50, pp. 1-652, and in particular the tabular survey tables therein which contains many examples of couplings of M₁-III or X₁-III). One method for preparing a compound of formula I is that of heating a mixture, preferably by microwave, of M₁-II (wherein M₁ is trimethylstannyl or tri-(n-butyl)stannyl), 0.7-1.3 equiv X₁-III wherein X₁ is Br, I, or triflate, 1-3% mot equiv tetrakis-(triphenylphosphine)palladium(0), and 0.1-0.4 equiv cuprous iodide, in dioxane at 140-170° C. for 1-4 h.

The review of Hassan on aryl-aryl coupling (Chem. Rev. 2002, vol. 102, pp 1359-1469) is a further guide to the skilled artisan to prepare compounds of formula I wherein L is carbon, by the Stifle and Suzuki reactions of Scheme I. Said review additionally presents a other aryl-aryl coupling methods which are useful for coupling M₁-II to X₁-III, and M₁-III to X₁-II, including methods wherein M₁ is selected from groups containing and attached to Zn, Mg. Mn, Hg, Si, Ge, Pb, Bi, Zr, In, and Sb, using catalysts containing the metals Cu, Ni, and Pd or mixtures thereof, and provides references to specific methods for effecting said couplings.

Scheme II shows reactions by which one skilled in the art can prepare intermediates M₁-II and M₁-III (L=carbon) wherein M₁ is a boron- or tin-containing group used for a reaction of Scheme I. One skilled in the art may employ established methods to prepare these from X₁-II and X₁-III (L=carbon), respectively, where X₁ is more preferably triflate, nonaflate, iodide, Br or Cl. One method is heating said triflate with a tetraalkoxydiboron compound ((RO)₂B)₂ in dioxane at 80° C. with catalytic quantities of [1,1′bis(diphenylphosphino)ferrocene]dichloropalladium(II) and 1,1′-bis(diphenylphosphino)ferrocene and excess potassium acetate (Ishiyama, Tetrahedron Lett. 1997, vol. 38, p. 3447 and Thompson, Synthesis 2005, p. 547) to give borate ester (RO)₂B-II or (RO)₂B-III. Dimethylformamide or dimethylsulfoxide may be substituted for dioxane as solvent. Another is heating said triflate, nonaflate, iodide, or bromide in dioxane for an optimal time at 80-100° C. with 1.5 equiv H—B(OR)₂, for example pinacolborane, and 3% [1,1′(bis(diphenylphosphino)ferrocene]dichloropalladium(II) (or PdCl₂(PPh₃)₂ for said bromide), and 3 equiv triethylamine (Murata, J. Org. Chem. 2000, vol 65, p. 164). Another is heating said chloride with 1.1 equiv bis-(pinacolato)diboron, 3 mol % Pd(dba)₂ (bis(dibenzylidineacetone)palladium(0)), 7 mol % tricyclohexylphosphine, and 1.5 equiv potassium acetate in dioxane or dimethylsulfoxide at 80° C. for an optimal time to give the corresponding pinacolborate ester (Ishayama, Tetrahedron 2001, vol 57, p 9813).

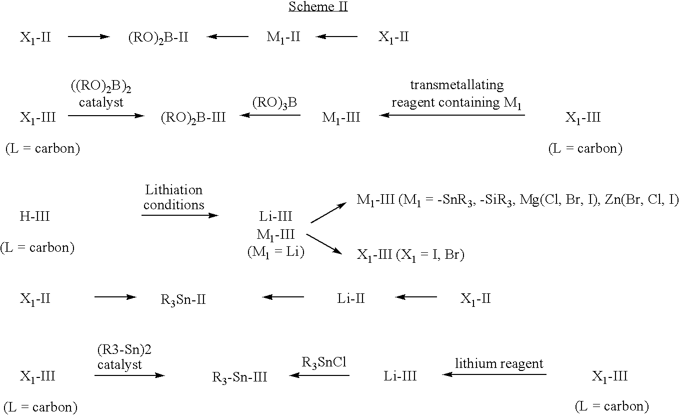

Another method which may be used to prepare (RO)₂B-II or (RO)₂B-III from X₁-II or X₁-III wherein X₁ is iodide or bromide, is by transmetallation of said iodide or bromide to give M₁-II or M₁-III wherein M₁ is lithium or magnesium halide, and reaction of the latter metallated species with a borate ester of formula (RO)₃B wherein R is preferablylower alkyl. An example of this method used to make a compound of formula M₁-III is given by Li (Organic Syntheses (2005), 81 89-97), and said method may be used to prepare other boron compounds (RO)₂B-II or (RO)₂B-III from said iodide or bromide. Said transmetallation of bromide or iodide to give M₁=lithium is generally accomplished by treating the bromide or iodide at −100° C. to 0° C. in tetrahydrofuran or ether with an organolithium reagent such as n-butyllithium, sec-butyllithium or t-butyllithium (methods cited by Sotomayor (Curr. Org. Chem. (2003), 7(3), 275-300)). Said transmetallation of bromide or iodide to give M<=magnesium halide is generally accomplished by treatment of said bromide or iodide with an organomagnesium halide such as isopropylmagnesium bromide at −78° C. to 65 in a suitable solvent such as tetrahydrofuran or ether. These conditions may be highly tolerant of other functional groups in the molecule and preferable in minimizing need for protecting groups. References to magnesation conditions including use to prepare boronic acid derivatives, useful for preparing compounds of formulae M₁-II and M₁-III where M₁ is magnesium halide, are provided by Knochel (Angewandte Chemie. International Edition (2003), 42(38), 4302-4320). Boronic acids (M₁=B(OH)₂) are prepared from the borate esters by hydrolysis and may be in equilibrium with the boroxine trimers shown above; when such is the case said mixture of boronic acid and trimer may be employed in the Suzuki reaction to form I. Alternate methods which may be used to prepare (RO)₂B-II or (RO)₂B-III and the corresponding boronic acids from X₁-II or X₁-III are given by Miyaura (Synthesis of Organometallic Compounds; Komiya, S., Ed.; Wiley: New York, 1997: p. 345), Vaultier (Comprehensive Organometallic Chistry II: Abel, et at, Eds.; Pergamon: Oxford 1995: Vol. 11, p 191) and Matteson (The Chemistry of the Metal-Carbon Bond; Hartley, et al., Eds; Wiley: New York, 1987: Vol. 4, p. 307).

Also shown in Scheme II, compounds of formula M₁-III wherein L is carbon may be prepared bylithiation (deprotonation at L) of the corresponding compound of formula H-III. Lithiation reagents include n-butyllithium, lithium diisopropylamide, n-butyllithium/tetramethylethylenediamine. Solvents include tetrahydrofuran, ether, hexane, and toluene. Methods for lithiation and guidance to use of this method to prepare compounds of formula M₁-III (LL=C) wherein M₁ is lithium are reviewed by Gschwend (Organic Reactions, vol 26 (Wiley: NY, 1979)). The reaction is especially useful when one or both of M and V is selected from N, O, and S. Examples given therein include lithiation of substituted and unsubstituted pyrroles, indoles, pyrazoles, furans, thiophenes, imidazoles, benzimidazoles, triazoles, tetrazoles, pyridines (as N-oxide), pyrimidines, oxazoles, and benzothiophenes. Additional guidance to methods is given by Iddon in reviews of lithiation of heterocycles of formula H-III (LL=C) (Heterocycles (1995), 41(7), 1525-74; Heterocycles (1995), 41(3), 533-93; Heterocycles (1994), 38(11), 2487-568; Heterocycles (1994), 37(3), 2087-147; Heterocycles (1994), 37(3), 2087-147). Lithiation of compounds of formula III (LL=C) is also especially useful when a substituent R⁸ or R¹² contains a heteroatom selected from O, N, or S which may coordinate lithium, or R⁸ or R¹² is a substituent which directs Lithiation of atom L in H-III (LL=C). Examples of said substituents include dialkylaminomethyl, carboxylic acid, carboxamide, ketone, sulfone, sulfonamide, alkoxyalkyl, and alkoxy. Such lithiations are referred to in the literature as “directed ortho metallation” and these methods which have been extensively developed by Snieckus are readily available to one skilled in the art (for example Snieckus, Metal-Catalyzed Cross-Coupling Reactions (2nd Edition) (2004), 2, 761-813). Li-III thus prepared may be converted to other M₁-III as shown by treatment with transmetallating reagents such as chlorotrialkylstannane, chlorotrialkylsiiane, magnesium halide or zinc halide. Li-III is also converted to X₁-III (X₁ is bromine or iodine) by treatment with bromine or iodine, respectively or other bromine- or iodine-containing reagents which brominate or iodinate organometallic reagents.

Also shown in Scheme II, previously mentioned stannane derivatives M₁-III (LL=C) and M₁-II wherein M₁ is a group connected at tin, which are used for the Stifle coupling discussed above in the context of Scheme I, are prepared from X₁-III (LL=C) and X₁-II (X₁ includes iodide, Br, Cl or triflate) by heating with a suitable fin derivative for example hexamethylditin or hexabutylditin and a suitable palladium catalyst, for example Pd(PPh₃)₄ in dioxane at 100-150° C. Another method to prepare said stannanes is the treatment of Li-II or Li-III with tributylstannyl chloride or trimethylstannyl chloride. These and other methods for preparing tin derivatives, applicable to the preparation of M₁-III and M₁-II wherein M₁ is a group connected at tin, are reviewed by Stille (Angew Chem. Intl. Ed. Engl. 1986, vol 25, p, 508) Said tin derivatives are preferably purified by silica gel chromatography before use in a reaction of Scheme I.

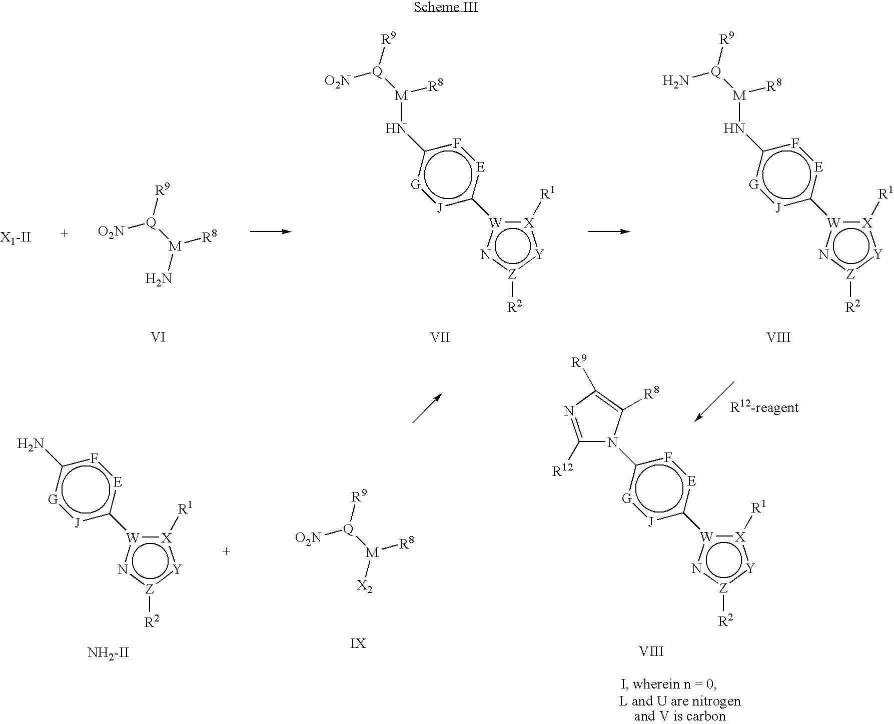

Certain heterocycles of formula H-III having L=nitrogen may contain additional nitrogens in conjugation with L, and in coupling with X₁-II according to Scheme I may give a mixture of isomeric compounds I. Such compounds H-III include for example an unsymmetrically substituted benzimidazole (such as 5-methylbenzimidazole, or 4(7)-azabenzimidazole) where the nitrogens in the 5-membered ring may both be reactive under the chosen coupling conditions. One skilled in the art may choose to separate such isomers by chromatography or crystallization, or may instead employ an alternate route for synthesis of I which gives only one isomer. Scheme III shows routes for preparation of compounds of formula I wherein n is zero, L and U are nitrogen, and V is carbon, which are particularly well-suited for preparing compounds of formula I where R⁸ and R⁹ are taken together to form a 5or 6-membered aromatic or heteroaromatic ring. A compound of formula X₁-II wherein X₁ is more preferably triflate, iodo, bromo, or chloro is coupled with a compound of formula VI using suitable coupling conditions to give a nitro compound of formula VII. Suitable coupling conditions include those suitable for amination of an aryl halide or triflate or heteroaryl halide or triflate with a primary aryl- or heteroarylamine. Particularly suitable coupling conditions include heating X₁-II and VI in toluene or tetrahydrofuran with 1-2.5 equiv of a base including lithium bis-(trimethylsilyl)amide, sodium t-butoxide, or potassium phosphate, 1-3% tris(dibenzylideneacetone)dipalladium(0), and 4-10% of a ligand, preferably an electron rich biaryl phosphine ligand, at 60-120° C. for an experimentally determined period up to about 24 hours. A more specific description of the foregoing particularly suitable coupling method, and references to other suitable coupling methods, are given by Charles (Org, Lett. 2005, vol 7, pp 3965-3968). A second particularly suitable coupling method, illustrated by Examples provided in the instant application, and in the publication of Yin (Org Lett 2002, vol. 4, pp. 3481-3484, and references therein), consists of combining X₁-II and VI, a catalytic amount (e.g. 1-3%) tris(dibenzylideneacetone)dipalladium(0), 4,5-bis(diphertylphosphino)-9,9-dimethylxanthene (2-3 equiv relative to the palladium catalyst), and cesium carbonate (1.2-1.5 equiv relative to X₁-II) in dioxane or other solvent and heating the mixture at 80-150° C. for a suitable period. In the Examples herein, when using this method, heating by microwave is advantageous. Descriptions of other methods useful for the coupling of X₁-II and VI are given by Kataoka (J. Org. Chem. 2002, vol 67., pp 5553-5566), Wolfe (J. Org, Chem. 65, 1144-1157), Old (J. Am. Chem. Soc. 1998, vol 120, pp 9722-9723), Wolfe (J. Org. Chem. 2000, vol 65, pp. 1158-1174), Muci (Top. Curr. Chem. 2002, vol. 219, pp 133-209), Shen (Angew. Chem. Int. Ed. 2005, 44, 1371-1375), and Jiang (Metal-Catalyzed Cross-Coupling Reactions. 2^{nd} Edition. A. de Meijere, F. Diederich, Eds. Copyright 2004, Wiley-VCH Veriag GmbH & Co. KGaA. Weinhem, Germany). Nitro compound VII is reduced to give a diamino compound of formula VIII using suitable reducing conditions. Suitable reducing conditions include one of the commonly known methods for reducing an aromatic or heteroaromatic nitro compound to the corresponding amine, including catalytic hydrogenation, catalytic transfer hydrogenation, or chemical reduction. A preferred method is that of combining VII with 10% palladium-on-carbon (for example 5-25 weight percent), in methanol or ethanol, and shaking the resultant mixture under 40-60 p.s.i hydrogen pressure for a suitable period determined by analysis of the mixture by TLC or HPLC-MS which typically shows formation and disappearance of an intermediate N-hydroxy compound and formation of the desired amine VIII. Compound VIII and a suitable R¹²-containing reagent are coupled and cyclized using suitable coupling and cyclizing conditions to give a compound of formula I wherein n is zero and L and U are both nitrogen and V is carbon. Suitable coupling and cyclizing conditions may comprise one or more separate chemical operations or steps. When the R¹² atom attached to ring atom V is carbon, the R¹²-reagent is preferably R¹²—COOH, (R¹²CO)₂O, or R¹²COCl. In this case, suitable coupling and cyclizing conditions comprise heating the diamine VIII in an excess of R¹²COOH, or with an excess of R¹²COOH and 1-1.5 equiv (R¹²)₂CO, or with an excess of R¹²COOH and 1-1.5 equiv R¹²COCl at a temperature usually between 80 and 150° C. as determined by experimentation. For example heating VIII with trifluoroacetic acid at about 90-100° C. produces a compound of formula I wherein R¹² is CF3. If it is desirable to avoid using excess R¹²COOH, heating with a slight excess of R¹²COOH in a high-boiling solvent such as o-dichlorobenzene gives a compound of formula I. Alternatively, a two-step procedure may be employed wherein VIII is first monoacylated on nitrogen by coupling with R¹²COOH and a suitable coupling agent for amide bond formation, or by reaction of R¹²COCl or (R¹²CO)₂O with VIII (for example in dichloromethane using triethylamine, or in pyridine), and the resultant amide is then cyclized with a suitable cyclizing condition for forming an imidazole ring by dehydrative cyclization of an amino amide. One suitable cyclizing condition is heating at 80-120° C. in phosphoryl chloride solvent. Another is heating with an acid catalyst such as sulfuric acid or p-toluenesulfonic acid at reflux in a suitable solvent such as toluene or xylene optionally with removal of water. Yet another suitable coupling and cyclizing condition is heating VIII with a nitrite R¹²CN under such acidic dehydrative conditions, including mixing said reactants with polyphosphoric acid and heating at 150-200° C. When the atom of R¹² attached to ring atom V is oxygen, a suitable coupling and cyclizing condition consists of heating VIII with an excess of orthocarbonate (R¹²O)₄C using an acid catalyst such as propionic acid, usually at a temperature between 80 and 160° C. to give a compound of formula I. When VIII is treated with carbonyldiimidazole in dichloromethane, one obtains a compound of formula I wherein R¹² is OH, Treatment of VIII with 1-1.5 equiv (1-ethoxyethylidene)malononitrile in refluxing acetic acid, or with excess triethylorthoformate and an acid catalyst (e.g., p-toluenesulfonic acid) produces a compound of formula I wherein R¹² is H, Other procedures are useful for converting VIII to a compound of formula I wherein R¹²=CN (Konstantinova, Tetrahedron 1998, p 9639), amino (Wu, J. Het Chem. 2003, p 191), alkyl (Spencer, J, Organomet. Chem. 1985, p 357), alkoxycarbonyl (Musser, Synth. Commun. 1984, p 947), aryl (Hendrickson, J. Org, Chem. 1987, p 4137) and is used by one skilled in the art. When R⁸ and R⁹ are not taken together to form an aromatic or heteroaromatic ring, an oxidation step may be included to aromatize the LMQUV ring (for example from an imidazoline to an imidazole ring). One such suitable oxidation step is stirring with activated manganese dioxide in an inert solvent such as dichloromethane.

Also shown in Scheme III is an alternative method where a nitro compound of formula VII is prepared by coupling an amine of formula NH₂-II with a halo nitro compound of formula IX (X₂=halogen) where R⁸ and R⁹ are taken together to form an aromatic or heteroaromatic ring, using suitable coupling conditions. Said coupling conditions may include those described above for coupling X₁-II and VI, and also include displacement conditions wherein NH₂-II and IX are heated together with or without a suitable solvent. Suitable solvents include dimethylformamide, dimethylsulfoxide, acetonitrile, ethanol, isopropyl alcohol and n-butanol. An organic base such as triethylamine, DBU (1,8-diazabicyclo[5.4.0]undec-7-ene), DBN (1,5-diazabicyclo[4.3.0]non-5-ene), sodium acetate, potassium t-butoxide, or an inorganic base or base mixture containing potassium carbonate or potassium fluoride may be added. Microwave heating may also be beneficial.

For brevity in these Schemes in describing alternative syntheses of the aforementioned compounds of formula I, X₁-II, and NH₂-II, a radical R²² refers to a radical which is selected from the group of radicals consisting of IV, X; and XI. When radical IV is attached to V, said IV-V is a compound of formula I. X₂ in X is as described for Scheme I, and when radical X is attached to radical V, said compound X-V is a compound of formula X₁-II. P₁ is a protecting group for a phenolic or heteroaryloxy hydroxy I group, and a compound wherein XI is attached to V (XI-V) is a compound of formula P₁O-II. Said compound containing XI is a precursor to a compound containing X as described below for Scheme XIX.

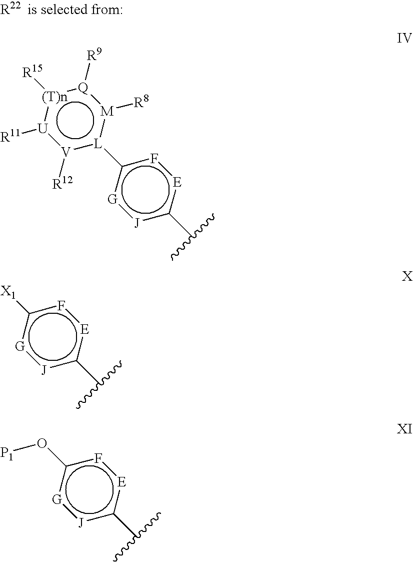

An imidazole compound of formula R²²-Va is prepared as shown in Scheme IV, by cyclization of an amidine XIII with a ketone derivative of formula XIV (X₂ is halogen or other leaving group, preferably bromine or chlorine), under suitable cyclizing conditions, which may include a step to dehydrate a hydroxy-imidazoline intermediate to the desired imidazole (such as heating in acetic acid, or heating with catalytic p-toluenesulfonic acid or sulfuric acid in toluene with removal of water. Suitable cyclizing conditions include heating XIII with XIV in a suitable solvent such as isopropyl alcohol or tert-butanol at 80-100° C. with 2-4 equiv of a base such as sodium or potassium bicarbonate, or adding IX at 0-25° C. to a mixture formed from treating XIII with a slight excess (or 2.2 equiv if XIV is a salt) of lithium bis-trimethylsilylamide in tetrahydrofuran at 0° C., subsequently adding XIV, and after subsequent workup, treating the crude product so obtained with acetic acid at 60-100° C. The latter procedure is given in the Example section of this application as “General Procedure 2”.

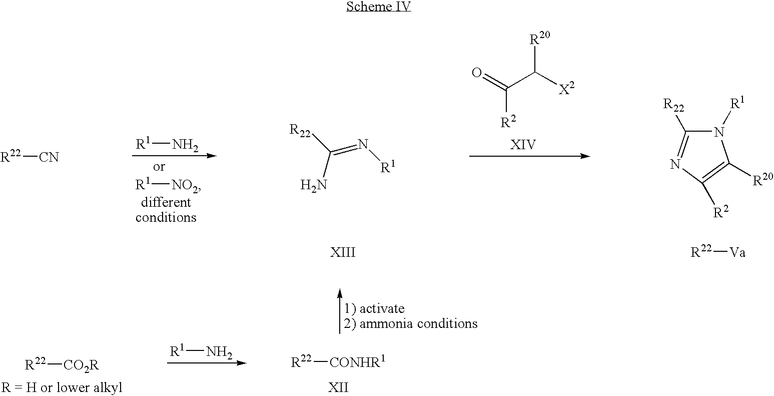

An amidine of formula XIII is formed by treatment of a nitrile R²²—CN with an aryl- or heteroarylamine of formula R₁—NH₂ under suitable amidine-forming conditions including those reported in the literature for forming N-aryl or N-heteroarylbenzamidine derivatives. Suitable amidine-forming conditions include adding 1-1.5 equiv of sodium hydride oil dispersion to a mixture of R²²—CN and R¹—NH₂ in dimethylsulfoxide and heating the resulting mixture at 50-65° C. for 1-4 h (this procedure is given in the experimental section as “General Procedure 1” and a closely related method given by Redhouse (Tetrahedron, 1992, vol. 48, pp. 7619-7628)). Other suitable amidine-forming conditions include converting R²²—CN to a methyl imidate hydrochloride with anhydrous hydrogen chloride in methanol, or to an S-methyl imidate hydriodide by stepwise conversion first to a thioamide R²²—C(S)NH₂ by treatment of R²²—CN with hydrogen sulfide in pyridine and subsequent methylation of R²²—C(S)NH; with methyl iodide in acetonitrile, and treatment of said methyl imidate hydrochloride or S-methylthioimidate hydriodide with R₁—NH₂ in a suitable solvent such as methanol or dimethylformamide. Amidine XIII is also be prepared by treating R²²—NH₂ with a trialkylaluminum reagent such as trimethylaluminum in a suitable inert solvent and adding R²²—CN, as described by Garigipati (Tetrahedron Lett. 1990, p. 1969) and also applied by Khanna (J. Med. Chem. 1997, vol. 40, p 1634-1647). Suitable amidine-forming conditions also include heating with aluminum chloride in an inert solvent, and conditions wherein a nitro compound R¹—NO₂ is reduced by samarium diiodide in tetrahydrofuran in the presence of R²²—CN, presumably to a metal complex of R¹—NH₂ which gives amidine XIII (examples provided by Zhou, J. Chem. Soc. Perkin 1, 1998, p. 2899). One skilled in the art will already know or readily find and implement a satisfactory method for converting R¹—NO₂ to R¹—NH₂ from the literature.

Also shown in Scheme IV, amidine XIII is alternatively prepared from amide XII in a two step sequence wherein said amide is first converted under suitable amide activating conditions to an activated intermediate, which is then treated with ammonia under suitable ammonia conditions to give the amidine XIII. Said amide activating and ammonia conditions include those reported in the literature for transforming an amide into an amidine by activation and addition of ammonia to the activated intermediate. One suitable amide activating condition is treatment of said amide with 1-1.5 equiv of phosphorus pentachloride in phosphorus oxychloride solvent at about 100° C. for 18 h and removing said solvent by evaporation or dissolution in hexanes. The residue or filtered solid is an activated intermediate which is then added portionwise to an excess of ammonia in ethanol or isopropyl alcohol at −20 to −10°G to give the amidine. Another method is treatment of XII in dichloromethane at −40° C. with 3 equiv pyridine and 1.3 equiv triflic anhydride to generate an activated pyridinium intermediate which is then treated with ammonia to give XIII (Charette, Tetrahedron Lett. 2000, pp 1677-1680). Other methods of forming amidines applicable to the synthesis of XIII are cited in this reference. Also shown in Scheme IV, XII is prepared by a suitable amidation method from R₁—NH₂ and the corresponding ester or acid. Said amidation methods are available in the literature to one skilled in the art, including preparing an acid chloride R²²—COCl by heating R²²—COOH in thionyl chloride solvent or by treatment of R²²—COOH with a slight excess of oxalyl chloride and a catalytic amount of dimethylformamide in an inert solvent such as dichloromethane, and reaction said acid chloride thus formed with R₁—NH₂ in a suitable solvent such as pyridine or dichloromethane containing a suitable amount of an appropriate organic base such as triethylamine at about room temperature, or by heating said acid chloride and amine in an inert solvent such as benzene or toluene. Another well-known amidation method is treating R²²COOH and R₁—NH₂ with a coupling agent such as 1-ethylamino-3-((3-dimethylamino)propyl) carbodiimide hydrochloride or N,N′-dicyclohexylcarbodiimide in an inert solvent, optionally with an additive such as 1-hydroxybenzotriazole. Other coupling agents which may be employed are diethylphosphoryl cyanide, carbonyl diimidazole, cyanuric fluoride (to form an acid fluoride), alkyl chloroformates (to form the mixed anhydride of the acid) and propanephosphonic anhydride. One skilled in the art will determine whether to activate the acid prior to adding R¹—NH₂, and what base, solvent and other conditions to employ. Another suitable amidation method is heating an ester R²²—COOR(R=C₃-C₄alkyl) in an inert solvent such as toluene, xylene, dichlorobenzene, or diphenyl ether with R¹—NH₂ optionally with a catalytic amount of sodium cyanide. Another is to treat R¹—NH₂ with trimethylaluminum in an inert solvent or solvent mixture to give the corresponding aluminum amide R¹—NH—AlMe₂, or with a Grignard reagent in a suitable solvent to give R1NHMgX, then adding R²²—COOR and allowing mixture to react for an appropriate time and temperature to give XII. Acid R²²—COOH is prepared from the corresponding ester R²²—COOR by saponification in aqueous alcohol or another organic solvent such as tetrahydrofuran containing water.

Scheme V shows an alternative method for preparing compounds of formula R²²-Va, wherein the ring R¹ is added fast. Primary amide R²²—CONH₂ is converted to the corresponding primary amidine R²²—C(═NH)NH₂ by sequential treatment under amide activation conditions and suitable ammonia conditions as described above for Scheme IV. Alternatively R²²—CN is added to dimethylaluminum amide (from trimethylaluminum and ammonium chloride in a suitable inert solvent such as toluene, dichloromethane or hexanes as described in the literature) to give R²²—C(NH)NH₂. R²²CONH₂ is formed by amidation of ester R²²—COOR(R=lower alkyl) by heating with ammonia in a suitable solvent such as ethanol, preferably in a sealed vessel at 60-100° C., by reaction of R²²COCl, prepared as described above, with ammonia, by coupling R²²—COOH with ammonia or ammonium chloride under suitable coupling conditions as described above for coupling to R¹—NH₂, or by partial hydrolysis of nitrite R²²—CN by a literature method for conversion of an aromatic or heteroaromatic nitrile to the corresponding amide. Amidine R²²—C(═NH)NH₂ and ketone derivative XIV are converted to imidazole XV under suitable conditions such as those described for conversion of XIII and XIV to R²²-Va in Scheme IV. XV is then arylated or heteroarylated on nitrogen, with R¹-X₁, predominantly on the nitrogen furthest from R² by suitable N-arylation or N-heteroarylation conditions. If an undesired isomer forms from non-selective arylation or heteroarylation on the nitrogen nearest R², it may be removed in a purification step. This route is preferred for R²²-Va having R²⁰=H over other R²⁰ substituents, when it is desirable to avoid said purification step. Suitable N-arylation or N-heteroarylation conditions include those set forth above for the first reaction of Scheme I (compounds of formula I wherein L is nitrogen), wherein R¹-X₁ is substituted for X₁-II, and XV is substituted for H-III. Other methods in the literature for N-arylating or N-heteroarylating imidazoles or benzimidazoles with either aryl- or heteroaryl halides and inflates, or with aryl- or heteroaryl boronic acids may be adapted to N-arylate or N-heteroarylate XV with either R₁-X₁ or R¹—B(OH)₂ to give R²²-Va. Included in these methods are those discussed in Scheme I for the couplings of H-III (L=N) with X₁-II and B(OH)₂-II. Also included is a method of N-arylating or N-heteroarylating a nitrogen heterocycle (in this case XV) with an electrophilic aryl or heteroaryl R¹ species such as N-fluoropyridinium triflate as shown in Example 119 for N-pyridinylation of a triazole nitrogen.

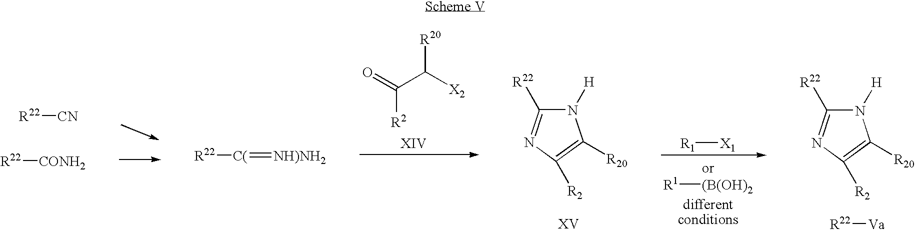

Scheme VI depicts another method for preparing R²²-Va, where R²⁰=H, An amino ester XVI (R′=methyl) is coupled to R²²—COOH under suitable coupling conditions for forming an amide from an acid and an amine, including those previously set forth for forming XII, to give an amido ester XVII. Said amido ester is converted to aldehyde XVIII by a suitable procedure including treatment with diisobutylaluminum hydride in a nonpolar solvent such as hexanes or toluene at −78° C., or by reduction of the ester to the to the corresponding alcohol XX (for example with lithium borohydride in methanol, or lithium aluminum hydride in tetrahydrofuran), and subsequent oxidation of the alcohol XX to aldehyde to XVIII with a selective oxidant (for example with pyridine-sulfur trioxide in dimethylsulfoxide, or by the Swern oxidation). Alternatively XX is prepared by coupling R²²—COOH with amino alcohol XIX. Another suitable method for preparing said aldehydes is to start with the N-methoxy-N-methyl amide corresponding to amino ester XVI (wherein OR′ is N(Me)OMe). Said amide is prepared by protection of the amino function of XVI with a suitable protecting group, hydrolysis of the ester to the acid (R′=H), coupling with N-methoxy-N-methylamine, and removing the protecting group. Said N-methoxy-N-methyl amide is then coupled with R²²—COOH to give the analog of XVII wherein OR′ is N(Me)OMe. Said analog is then reduced by the method of Fehrentz and Castro (Synthesis 1983, pp 676-677) with an excess of lithium aluminum hydride in tetrahydrofuran or ether giving aldehyde XVIII. Said aldehyde is combined with R¹—NH₂ and heated in a suitable solvent under acidic and dehydrating conditions such as heating with p-toluenesulfonic acid in toluene with azeotropic removal of water (Adams, WO 93/14082 (PCT/US93/00675), including Example 1 therein), giving a substance of formula R²²-Va wherein R²⁰ is H,

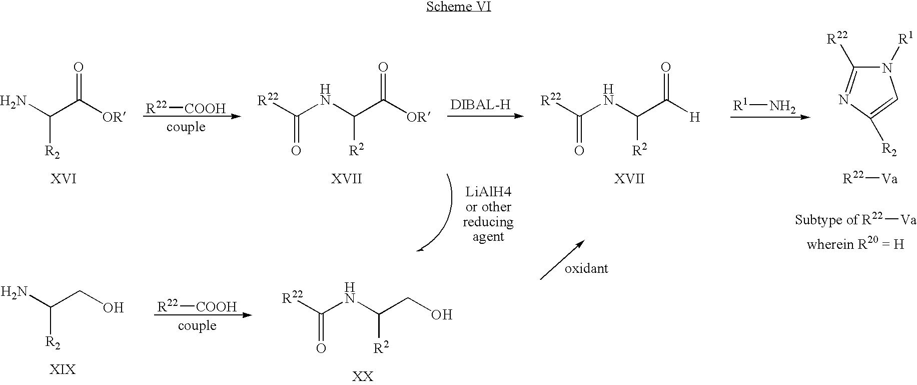

Scheme VII depicts another method for the synthesis of R²²-Va (wherein R²⁰ is H). Amide XXII is first activated under suitable amide activation conditions as previously described in Scheme IV to give activated intermediate XXI (X₂= preferably Cl, triflate, or pyridinium), which is then treated alternatively with an aminonitrile XXII to give XXIII. Aminonitrile XXII is a Strecker synthesis intermediate available from aldehyde R²—CHO by treatment with ammonium chloride and potassium or sodium cyanide in methanol or ethanol, optionally with added sodium bisulfite. XXIII is reduced with diisobutylaluminum hydride in a nonpolar solvent such as toluene or hexanes generating the imine intermediate. Cyclization of this imine preferably under acidic conditions including by heating with excess ammonium chloride in acetic acid or other suitable solvent yields R²²-Va (wherein R²⁰ is H).

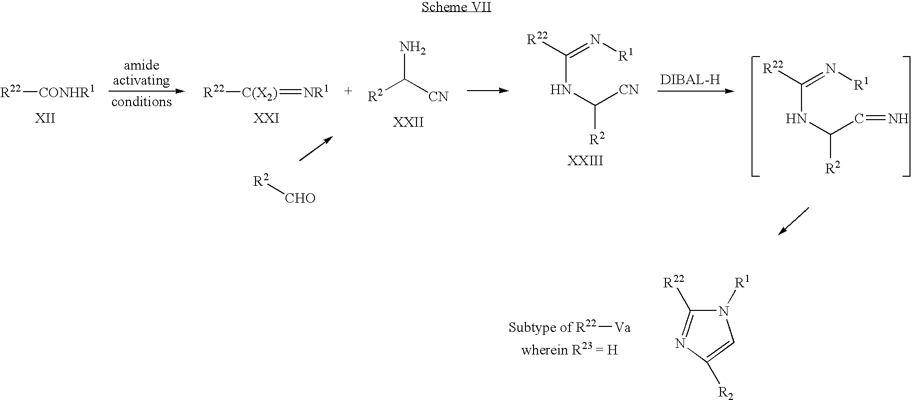

Another method of preparing R²²-Va is shown in Scheme VIII. Amido-ketone XXVII is heated with ammonia or an ammonia source under conditions suitable for imidazole formation. Said conditions may include a second step to dehydrate or aromatize a hydroxyimidazoline intermediate, usually including heating with an acid and optionally with removal of water Ammonia sources include ammonium hydroxide, ammonium acetate, ammonium chloride, and formamide. Solvents include acetic acid, ethanol and dimethylformamide. Preferred conditions are heating XXVII with excess ammonium acetate in acetic acid at reflux. XXVII is prepared by coupling XXVI with R²²—COOH in analogous fashion as described above for formation of XII. Alternatively XXVII is prepared by reaction of R²-M₁ (wherein M₁ is lithium or magnesium halide) and XXV (prepared by coupling R²²—COOH and XXIV) in a suitable solvent such as tetrahydrofuran or ether. Alternatively XXVII is prepared by oxidation of XXIX (obtained by coupling XXVIII with R²²—COOH), with a suitable oxidant such as pyridine-sulfur trioxide in dimethylsulfoxide, the reagents which effect the Swern oxidation, the Dess-Martin periodinane, a chromium (VI) reagent, or reagents which effect the Pfitzner-Moffatt oxidation or variants thereof.

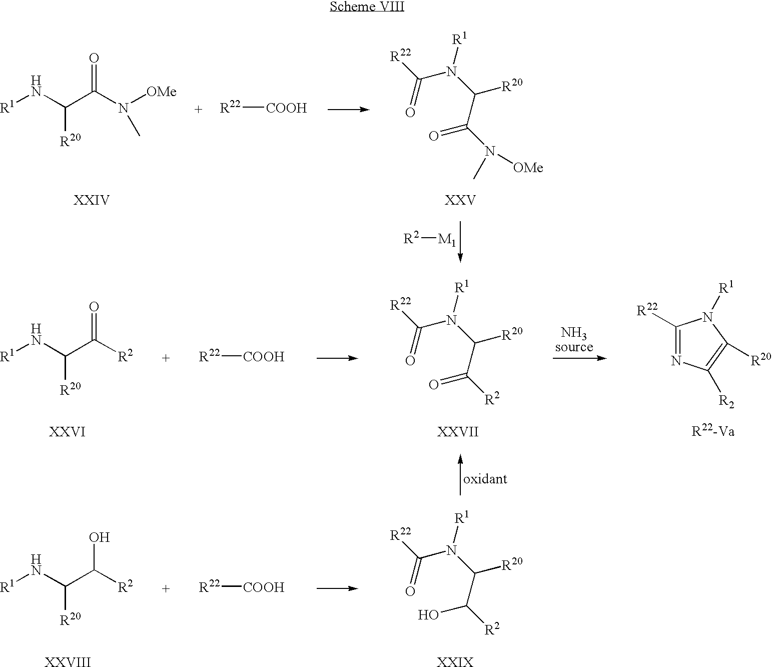

A compound of formula R²²—Vb (which includes Ib), wherein R²¹=H, is prepared as shown in Scheme IX. Aldehyde R²²—CHO is converted to cyanohydrin XXX (R═H), for example by treatment with sodium or potassium cyanide in a mixture of water and optional cosolvent such as dioxane or tetrahydrofuran, or to the O-trialkylsilyl cyanohydrin derivative such as the O-trimethylsilylcyanohydrin XXX (R=TMS) by treatment with a cyanotrialkylsilane such as cyanotrimethylsilane and an optional catalyst for silylcyanohydrin formation such as zinc iodide in an appropriate inert solvent such as dichloromethane, XXX is allowed to react with an organometallic derivative R¹-M₁ wherein M₁ is a metal atom or metal containing ligand linked at the metal atom, capable of adding R¹ to the nitrile function, to give the hydroxy ketone XXXI after a workup which includes acidic conditions to effect hydrolysis of the imine intermediate and cleavage of the silyl group if present. Preferred includes magnesium halide and lithium. Preferred conditions are combining R¹—M₁ (which is also generated from R¹—Br or R¹—I in said solvent at −100 to 0° C. from isopropylmagnesium halide or alkyllithium reagent) and XXX in ether or tetrahydrofuran, at −50 to 50° C. followed by addition of aqueous hydrochloric acid after consumption of XXX. XXXI is combined with aldehyde R²—CHO in the presence of an ammonia source and an oxidant, preferably a copper(II) salt under suitable imidazole-forming conditions giving R²²-Vb (wherein R²¹—H). Preferred conditions include mixing XXXI with 1.2 equiv R²—CHO, 2 equiv cupric acetate and 5-10 equiv ammonium acetate in acetic acid and heating at reflux temperature for a suitable period. When R²² is IV, R²²-Vb is a compound of formula Ib. A second general method for synthesis of R²²-Vb (including Ib where R²² is IV), is shown in the second reaction sequence of Scheme IX and relies on cyclization of diketone mono-oxime XXXIII and aldehyde R²—CHO under suitable conditions (including heating with an ammonia source under acidic conditions) to give N-hydroxyimidazole R²²—Vb (R²¹=OH). Preferred conditions are heating at reflux in acetic acid with 5-10 equiv ammonium acetate. Another preferred method is heating XXXIII and R²—CHO by microwave with methanol and acetic acid which can be used to produce R²²-Vb(R²¹=H), directly (Sparks, Org. Lett. 2004, vol. 6, pp. 2473-2476). N-hydroxy imidazole R²²-Vb(R²¹=OH) is reduced to NH-imidazole R²²-Vb (R²¹=H) by heating with triethyl phosphite at 80-110° C. in a suitable solvent such as dimethylformamide. Mono-oxime XXXIII is prepared by reaction of ketone XXXII with about 1.5 equiv sodium nitrite in acetic acid at room temperature. Ketone XXXIIa is prepared by reaction of R²²—COX₃ wherein X₃ is a leaving group (including halide, OR wherein R is lower alkyl, and N(Me)OMe), or R²²—CN, with a metallated species of formula R¹—CH₂-M₂ wherein M₂ is a metal or metal-containing ligand attached at the metal atom which is useful for synthesis of ketones via addition to R¹—COX₃. Preferable M₂ includes lithium, and a procedure for generating R¹—CH₂-M₂ which is often employed when R1 is a heterocycle having a suitably acidic CH₃, is that of treating R¹—CH₃ with lithium diisopropylamide or other organolithium or organosodium base in tetrahydrofuran, adding R²²—COOR(R=lower alkyl) after a suitable deprotonation period, and stirring the mixture at room temperature for a suitable period determined by experimentation. Another is heating R²²—COOR(R=lower alkyl) with R¹CH₃ in an alcohol containing potassium or sodium alkoxide, and yet another procedure is heating these reactants or R²²—CN in tetrahydrofuran with sodium hydride. When R₁—CH₃ is not suitably acidic, then R¹CH₂ M₂ wherein M₂ is MgBr is prepared by bromination of R¹CH₃ (for example with bromine or N-bromosuccinimide and a radical initiator in a suitable solvent such as carbon tetrachloride) and then reacting the R¹—CH₂Br with magnesium in tetrahydrofuran or ether to give R¹CH₂MgBr. A third closely related method for forming R²²-Vb (R²¹=H) is that of heating the diketone XXXVI with the aldehyde R²—CHO under suitable imidazole formation conditions, preferably heating these reactants in acetic acid with excess ammonium acetate, Diketone XXXVI is prepared by hydration of acetylene XXXV, by oxidation of XXXI (for example with copper sulfate in pyridine-water) or by heating monoketone XXXIIa or monoketone XXXIIb with SeO₂ in dioxane or acetic anhydride. Acetylene XXXV is hydrated to XXXVI by a literature method for hydration of diaryl acetylenes such as heating with iodine or palladium dichloride in dimethylsulfoxide at 120-160° C., by treatment with sulfur trioxide in dioxane, or by oxidation with potassium permanganate under aqueous conditions (such as with dichloromethane, aqueous sodium bicarbonate, and triethylammonium bromide). Acetylene XXXV is obtained by Sonogashira reaction (K, Sonogashira, Handbook of Organopalladium Chemistry for Organic Synthesis (2002), 1, 493-529) of either XXXIVa with R¹-X₁, or XXXIVb with R²²-X₁ (X₁ is most preferably iodo, bromo, or triflate). XXXIVa and XXXIVb are prepared by Sonogashira reaction of trimethylsilylacetylene and R²²-X₁, or R¹-X₁, respectively. Ketone XXXIIb is obtained from R¹COOH or R¹—CN and R²²CH₂-M₂ by the procedures given for preparing XXXIIa.

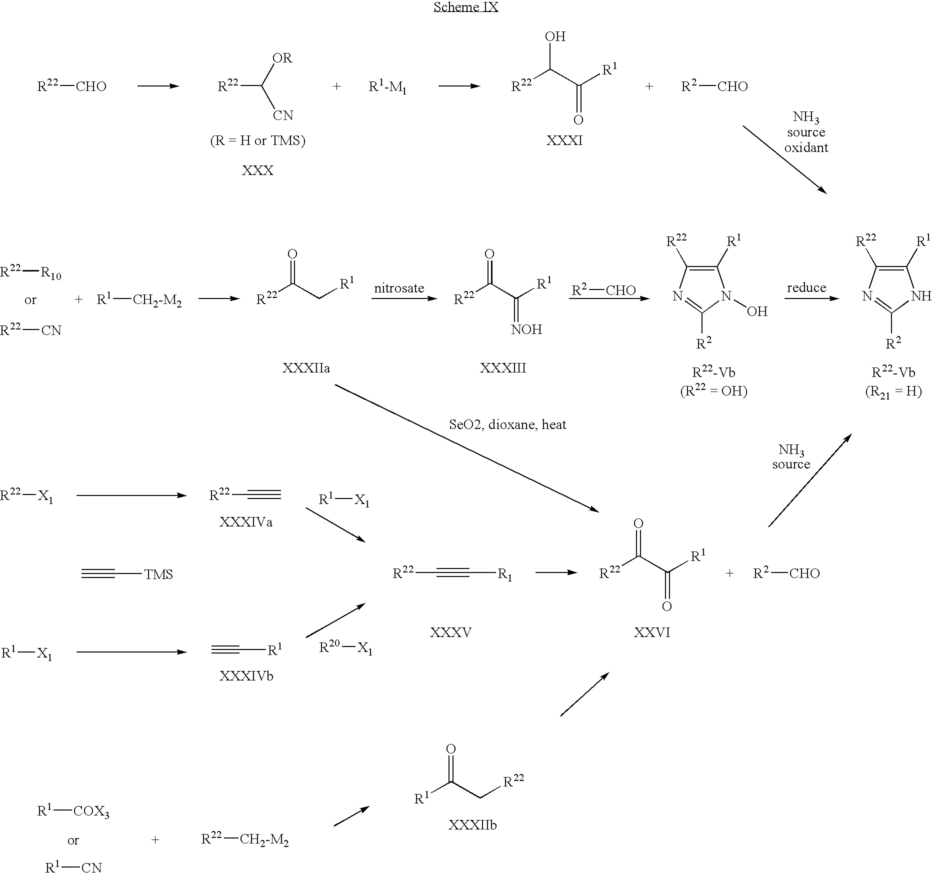

Scheme X shows routes to pyrazoles of formula R²²—Vc which includes Ic when R²² is IV. A preferred route to R²²-Vc (when R²⁰ is H) is that of heating acetylenic ketone XXXVIII with R²²—NH—NH₂ in ethanol (Bishop, Synthesis 2004, p. 43). XXXVIII is prepared by reaction of R¹-X₁ (X₁ is preferably iodine) with R²-acetylene XXXVII, catalytic palladium acetate, catalytic diphenylphosphinoferrocene and triethylamine in tetrahydrofuran at 70° C. in a sealed vessel under carbon monoxide pressure (40 bar), as described by Bishop (Synthesis 2004, p. 43, and references therein), or by cuprous iodide-catalyzed reaction of R¹—COCl with R²-acetylene in toluene and triethylamine as used by Bishop in said reference and as described by Chowdhury (Tetrahedron 1999, vol. 55, p. 7011), R²-acetylene is prepared by the Sonogashira reaction of R²-X₁ and trimethylsilylacetylene followed by cleavage of the trimethylsilyl group with acid or fluoride ion. Another route to R²²-Vc is that of heating a diketone XLI with R²²—NHNH₂ in a suitable solvent such as ethanol. Separation of the desired product may be required and, if so, effected by chromatography. XLI is prepared by acylating the enolate of XXXIX with R²²—COX₃, or that of XL with R¹—COX₃, (X₃ includes Cl, imidazo-1-yl, and OR′ where R′ is lower alkyl) effected by treating these reactants in tetrahydrofuran or dimethylformamide with sodium hydride or other organosodium or organolithium base (examples are sodium or lithium bis-(trimethylsilyl)amide, or when X₂ is OR′, in ethanol with sodium methoxide or ethoxide). R²²—NHNH₂ is prepared from R²²-X₁ (X₁ is preferably halogen or triflate) in some instances where R²²-X₁ is reactive enough for the halide to be displaced directly by hydrazine in a suitable solvent such as ethanol or tetrahydrofuran usually at 20-100° C. Alternatively, R²²-X₁ is allowed to react with benzophenone hydrazone or other protected hydrazine derivative, a palladium catalyst and a strong base (Arteburn, Org. Lett. 2001, p. 1351) giving protected R²²—NHNH₂ which is liberated by acid hydrolysis or other deprotection method. Alternatively, R²²—NH₂ is aminated by diazotization (example treatment with sodium nitrite and hydrochloric acid followed by reduction for example with stannous chloride in aqueous hydrochloric acid. Alternatively R²²-X₁ (X₁=triflate, nonaflate, halogen) may be aminated to give R²²—NH₂ by other procedures (reviews given by Buchwald in Metal-Catalyzed Cross-coupling reactions, 2^{nd} ed: De Meijere, A., Diederich, F. Eds.; Wiley-VCH: Weinheim, Germany, 2004 p 699 and Hartwig, J. F. in Handbook of Organopalladium Chemistry for Organic Synthesis; Negishi, E., Ed, Wiley-Interscience: New York, 2002; p 1051) including palladium catalyzed reaction with titanium-nitrogen complexes (conditions described by Hori, J. Am. Chem., Soc, 1938, p 7651), lithium hexamethyldisilazide (Huang, Org. Lett. 2001, vol. 3, pp. 3417-3419) or benzophenone imine (conditions described by Yang, Coll. Czech. Chem. Comm. 2000, p 549) and Tundel. J. Org. Chem., 2006, vol. 71, p. 430). Also many R²²-X₁ which are suitably activated may be displaced directly with ammonia to give R²²—NH₂ and then further aminated to give R²²—NHNH₂. Alternatively, pyrazole compounds of formula R²²-Vc are prepared by oxidation of pyrazoline XLIII with eerie ammonium nitrate in methanol optionally with heating by microwave, by 1,3-dibromo-5,5-dimethylhydantoin oxidation on silica get with microwave heating (Azarifar, Synthesis 2004, 1744).

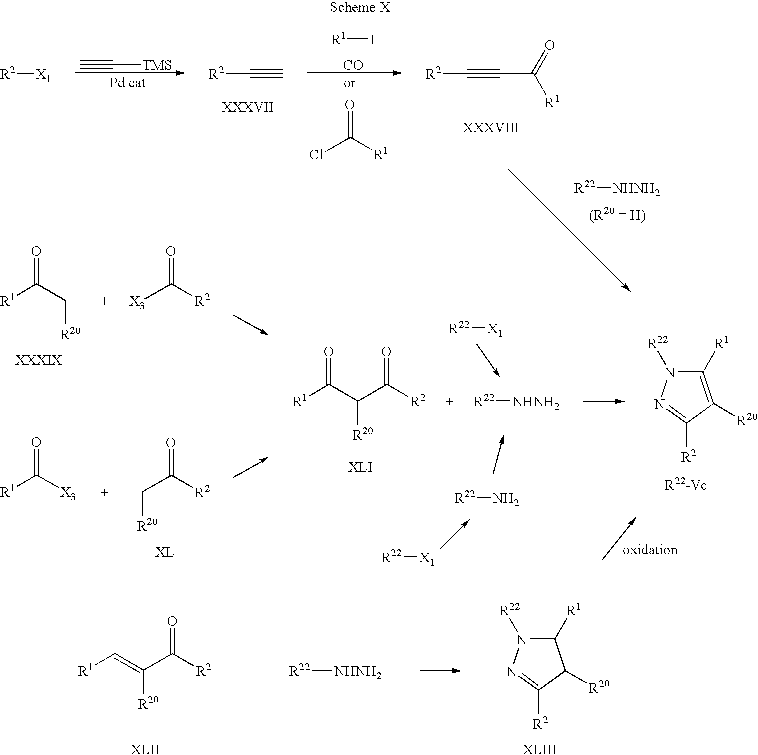

Scheme XI shows routes to pyrazoles of formula R²²-Vd which Includes compounds of formula Id when R²² is IV. Diketone XLIV is prepared from either R²²—COCH₂—R¹ and R²⁵—COX₃ or R²⁰—COCH₂—R₁ and R²²—COX₃ in analogous fashion to the preparation of XLI in the preceding Scheme. XLIV is condensed with R²—NHNH₂ under standard conditions for preparing a pyrazole from a diketone and a substituted hydrazine derivative, such as heating the reactants at reflux in ethanol, to give R²²-Vd. in the event that an undesired isomer forms it is removed in a purification step. R²—NHNH₂ is prepared from R²-X₁ or R²—NH₂ by one of the methods given in the previous Scheme for preparing R²²—NHNH₂ from R²²—X; or R²²—NH₂. Alternatively, R²²-Vd is prepared by N-arylation or M-heteroarylation of XLVI with R²-X₁. If an undesired isomer forms it is removed by chromatography or other purification method. A preferred method is selected from one of those given in the discussion of Scheme I for N-arylation or N-heteroarylation of H-III with X₁-II (particularly preferred are those of Cristau). A preferred method is the method of Example 120 herein which is a method for N-heteroarylation of XLVI with 2-iodopyridine, a diamine ligand, catalytic cuprous iodide, and potassium carbonate by heating in toluene. A preferred method for N-arylation or H-heteroarylation of XLVI with R²-X₁ is one of those described in the literature for N-arylation or N-heteroarylation of a pyrazole, including displacement of suitably activated R₂-X₁ by heating with potassium carbonate in dimethylformamide. Also included is a method of N-arylating or N-heteroarylating XLVI with an electrophilic aryl or heteroaryl R²-containing species such as N-fluoropyridinium triflate as shown in Example 119 for N-pyridinylation of a triazole nitrogen.

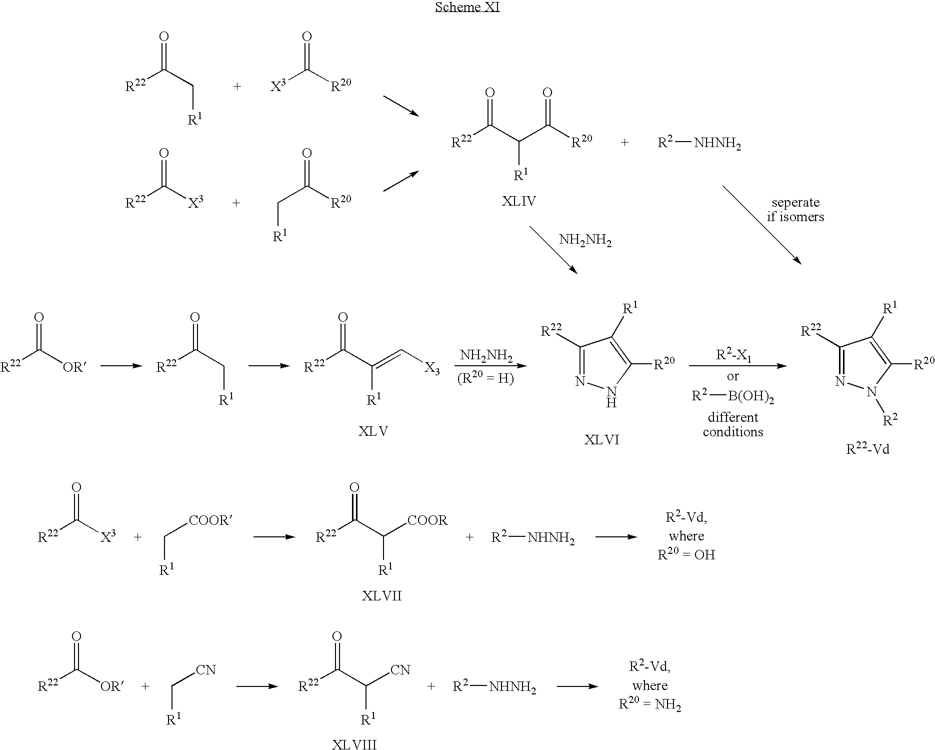

Another preferred method is N-arylation or N-heteroarylated of XLVI by R²—B(OH)₂ by one of the copper salt-mediated methods described in the discussion of the second reaction of Scheme 1 above (particularly those of the Lam, Chan, and Ley review citations). A compound of formula R²²-Vd (R²⁰=OH), or a compound of R²²−Vd(R²⁰=NH₂) is prepared by condensation of keto ester XLVII or keto nitrile XLVIII, respectively with R²—NHNH₂. Conditions for said condensations include heating the reactants in ethanol. XLVII is prepared by reaction of R²²—COX₃ with the enolate of R¹—CH₂COOR′ (R′ is lower alkyl) formed for example by reaction with lithium bis-(trimethylsilyl)amide or sodium hydride in tetrahydrofuran (X₃ is Cl, 1-imidazolyl, or OR′). XLVIII is also prepared by treating the reactants shown in the Scheme with lithium bis-(trimethylsilyl)amide or sodium hydride in tetrahydrofuran (R′ is also lower alkyl).

Compounds of formula R²²-Ve (including le when R²²=IV) and R²²-Vf (which includes if, when R²²=IV) are prepared as shown in Scheme XII. The route of Buzykin (Synthesis, 1993, p. 59) with modifications (described below) is effectively adapted for this purpose. For synthesis of R²²-Ve, the hydrazine R²²—NHNH₂ and aldehyde R¹—CHO are condensed to give a hydrazone XLIX, under any of many standard hydrazone condensing conditions know to one skilled in the art, such as mixing these reactants in ethanol or benzene for a suitable period. XLIX is then halogenated to give a hydrazonyl chloride L, prepared for example by treatment of XLIX with N-chlorosuccinimide-dimethylsulfide complex (Patel, Tetrahedron 1996, vol. 52, p 661) or a hydrazonyl bromide, prepared for example by treatment of XLIX with pyridinium perbromide in tetrahydrofuran (as in Preparation 88b herein). L is then treated with an amine R¹—CH₂NH₂ under suitable conditions such as in acetonitrile with excess triethylamine to provide an intermediate hydrazonyl halide displacement product which is subsequently oxidized by a suitable oxidizing method giving triazole R²²-Ve. in addition to hydrogen peroxide, potassium permanganate, and silver oxide described by Buzykin (above), suitable oxidizing methods including use of silver carbonate, sodium hypochlorite, calcium hypochlorite, Dess-Martin periodinane, or TPAP/NMO at room temperature in acetonitrile. R²²-Vf is prepared analogously, starting with R¹NHNH₂, R²CHO, and R²²—CH2NH₂. Examples of said oxidizing methods and use of this route to synthesize 1,2,4-triaryl triazoles are given by Paulvannan (Tetrahedron 2001, vol 57, p. 9677 and Tetrahedron 2000, vol 56, 8071, and references therein), and one skilled in the art, by choosing the starting materials as described in Scheme XII, may use said method to synthesize R²²-Ve or R²²-Vf. Alternatively hydrazonyl chloride L or LII (X₁=Cl) is heated with nitrile R²²—CN or R²²—CN, respectively, and catalytic ytterbium triflate in chlorobenzene at reflux (Su, Synth. Commun. 2005, vol 35, p. 1435) to give a compound of formula R²²-Ve or R²²-Vf, respectively.

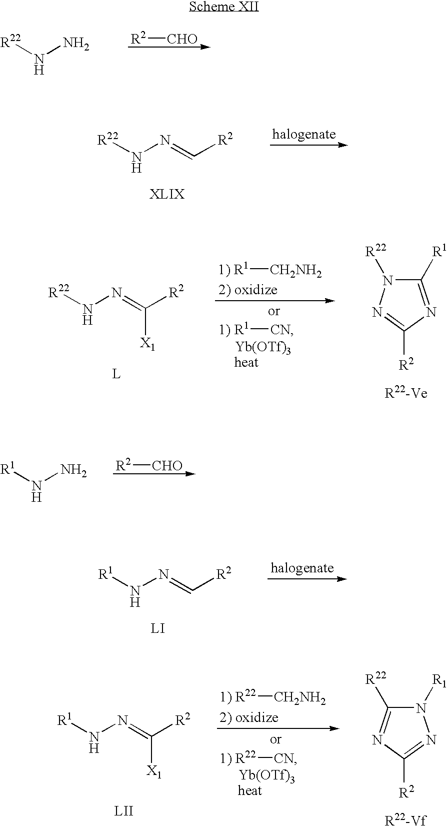

1,2,3-Triazoles R²²-Vg are prepared by the routes outlined In Scheme XIII. Triazole LII is N-arylated on the least hindered nitrogen atom by a suitable method for N-arylation or N-heteroarylation of a nitrogen-containing heterocycle selected from a literature method by one skilled in the art. Said methods include those set forth above for the first reaction of Scheme I, wherein R²-X₁ is substituted for X₁-II, and LIII is substituted for H-III (L=N). One preferred of said methods is that of Example 120 herein. Said methods also include those set forth above for the second reaction of Scheme I, wherein R²—B(OH)₂ is substituted for B(OH₂)-II and LIII is substituted for H-III (L=N). Also included is a method of N-arylating or N-heteroarylating a nitrogen heterocycle (in this case Lilt) with an electrophilic aryl or heteroaryl R² species such as N-fluoropyndinium inflate as in Example 119. Additional examples of said methods include heating LIII with an aryl bromide or iodide and potassium carbonate in dimethylsulfoxide at 150° C. (Kim, Bioorg. Med. Chem. Lett. 2004, vol, 14, p. 2401), and reaction of LIII with R²—B(OH)₂ and cupric acetate in pyridine (Tullis, Bioorg. Med. Chem. Lett. 2003, vol. 13, p. 1665) Triazole LIII is prepared by heating acetylene LIII with cyanotrimethylsilane, preferably neat but an inert solvent may be employed, typically in a sealed vessel at 130-180° C., preferably around 150° C. Acetylene XXXV is constructed by a method for preparation of diaryl or heteroaryl-aryl or bis-heteroaryl acetylenes in the literature. A method of choice is the Sonogashira reaction of R²²-acetylene and R¹-X₁ or R¹-acetylene and R²²-X₁ (X₁ is most preferably bromine, iodine or triflate). These acetylenes are themselves prepared by the Sonogashira reaction of R²²-X₁ and R¹-X₁, respectively with trimethylsilylacetylene. Alternatively, R²²-Vg is prepared by cyclization of bis-hydrazone LIV upon treatment with a suitable oxidizing agent such as potassium dichromate in acetic acid (El Khadem, J. Chem. Soc. Chem. C, 1968, p 949) or manganese dioxide (Bhatnagar, J. Org. Chem. 1967, vol. 32, p. 2252). Alternatively R²²-Vg is obtained by forming a monohydrazone LV of diketone R²²—CO—CO—R¹ (prepared as discussed for Scheme IX) with R²—NHNH₂ and heating said monohydrazone or mixture thereof (LV) with hydroxylamine hydrochloride in a suitable solvent at 100-200° C., or by forming the oxime of LV and heating said oxime with acetic anhydride. Alternatively, either of two ketones R¹—CH₂CO—R²² or R²²—CH₂CO—R¹ is converted to the corresponding monoxime (for example by treatment with sodium nitrite in acetic acid), and said monoxime is heated with R²²—NHNH₂ in a suitable solvent such as dimethylformamide to form R²²-Vg.

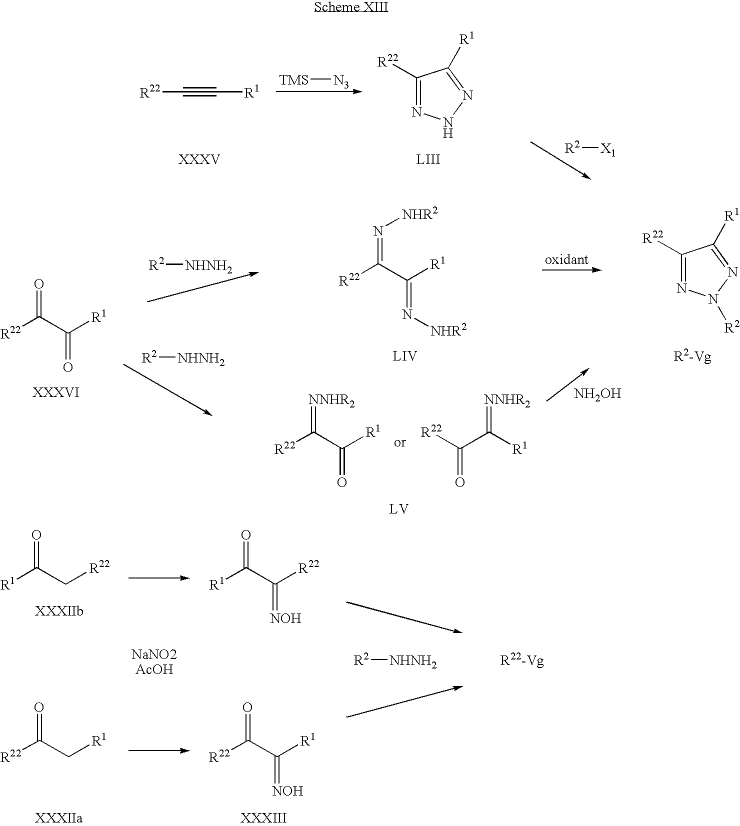

A compound of formula R²²-Vh (which includes a compound of formula Ih when R²² is IV) is prepared by one of the methods of Scheme XIV. Heating thioamide R²—C(S)NH₂ with bromoketone LVI under literature conditions for cyclizing a bromoketone and a thioamide to give a thiazole provides R²²-Vh. Suitable conditions include heating in a suitable solvent such as acetone, acetonitrile, isopropyl alcohol or dimethylformamide optionally in the presence of an organic or inorganic base. Suitable inorganic bases include sodium bicarbonate, potassium bicarbonate, potassium carbonate and cesium carbonate. Suitable organic bases include hindered bases which will not easily alkylate such as diisopropylethylamine. Bromo ketone LVI is prepared by bromination of ketone XXXIIa using cupric bromide in ethyl acetate at reflux, bromine in dioxane at 20° C., pyridinium perbromide, optionally polymer supported, in tetrahydrofuran at 0-25° C., by treatment with bromine in acetic acid containing hydrogen bromide, bromine in chloroform with heating, or n-bromosuccinimide in carbon tetrachloride with benzoyl peroxide initiator. Alternatively, amido ketone LVIII is heated with phosphorus pentasulfide or Lawesson's reagent in pyridine or chloroform to give R²²-Vh. Amido-ketone LVIII is prepared by addition of a rhodium (II) catalyst to a mixture of amide R²CONH₂ and diazoketone LVII according to the method of Davies (Tetrahedron 2004, vol. 80, pp. 3967-3977, or by coupling of amino ketone LIX with R²COON using a peptide coupling reagent or by first converting activating R²COOH as its acid chloride by analogy to methods described above for other amide bond formations. LIX is prepared by alpha-arylating or heteroarylating a protected glycine enolate with R²²-X₁ according to the method of Hartwig (J. Am. Chem. Soc. 2001, vol 123, p 8410) or by a similar non-palladium-catalytic method as illustrated by Bardel (J. Med. Chem. 1994, vol. 37, pp. 4567-4571) and converting the resultant amino acid to ketone LIX via established methodology of M-protection, Weinreb amide formation, Grignard addition of ring R¹, and deprotection. Diazo ketone LVII is prepared by subjecting XXXIIa to diazo transfer reaction conditions reported in the literature which are suitable for converting a ketone of formula Ar—CH₂CO—Ar′ to the corresponding diazo ketone of formula Ar—C(N₂)CO—Ar′ including treating XXXIIa with methanesulfonylazide in 1,2-dichloroethane and aqueous sodium hydroxide (Kuman, Syn Commun. 1991, p. 2121), with methanesulfonylazide and 1,8-diazabicyclo[5.4.0]undec-7-ene in acetonitrile, sequentially with lithium diethylamide and diphenylphosphorylazide, respectively, in tetrahydrofuran (Helv, Chim. Acta. 1995, p 1983), with p-toluenesulfonylazide and potassium or sodium ethoxide in ethanol (Tetrahedron 1970, p. 5557; Tetrahedron 1999, p. 11537), and with sodium hydride and tris-(diethylamino)azidophosphonium bromide in tetrahydrofuran (J. Org. Chem. 1999, p 4079). Finally, R²²-Vh is prepared starting with bromo chloro thiazole LX, R¹-M₁, R¹-M₁, and R²²-M₁ by Kershaw's sequence (Org, Lett, 2002, vol 4, pp. 1363-1365), using intermediates LXI, LXII, and LXIII, wherein M₁ is preferably independently B(OH)₂ or ZnBr, and using palladium catalyzed coupling methods given therein. Alternatively M, may also be selected from a metal or metal containing ligand, attached at the metal atom, such as SnR³ (R³ is lower alkyl), which is useful in aryl-aryl, heteroaryl-aryl or heteroaryl-aryl couplings including the Suzuki and Stille methods cited in connection with Scheme I or in the literature, and accompanying coupling conditions applicable to thiazole LX, LXI, and LXIII.

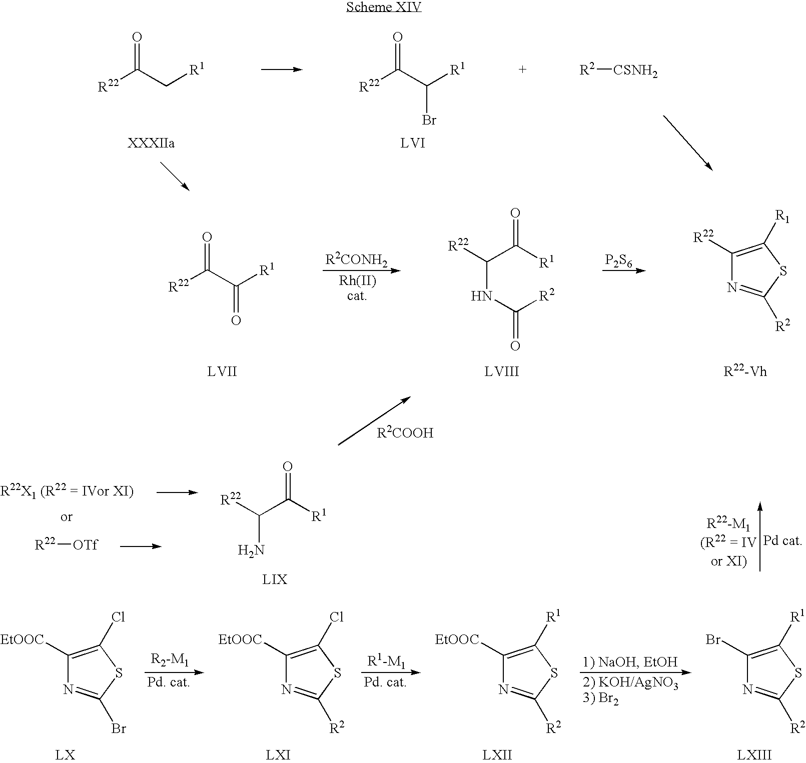

Compounds of formula R²²-Vi are prepared as shown in Scheme XV. Heating a mixture of R²²—COCH₂—R1 (XXXIIa) with [hydroxy(2,4-dimitrobenzene)sulfonyloxyiodo]benzene by microwave produces LXIV, and subsequent addition of R²—CONH₂ and further heating by microwave according to the method of Lee (Tetrahedron Lett. 2003, vol 44, p. 123) gives R²²-Vi. Alternatively, heating ester LXV with ammonium acetate or urea in acetic acid, or in formamide with catalytic sulfuric acid (Pei, Synthesis 1998, p 1298) produces R²²-Vi Ester LXV is obtained by esterification of alcohol XXXI with R²COOH using a suitable esterification method, for example treating a mixture of the XXXI and R²COOH with N,N′-dicyclohexylcarbodiimide and dimethylaminopyridine in a suitable solvent, or by formation of R²COCl from the acid as previously described, and reaction of this acid chloride, triethylamine and XXXI in dichloromethane. Alternatively, R²²-Vi is formed in a well-established cyclization of a 1,4-dicarbonyl compound LVIII, some literature methods being heating LVI in a suitable solvent with catalytic sulfuric acid, heating in thionyl chloride, or heating with phosphorus pentachloride in chloroform.

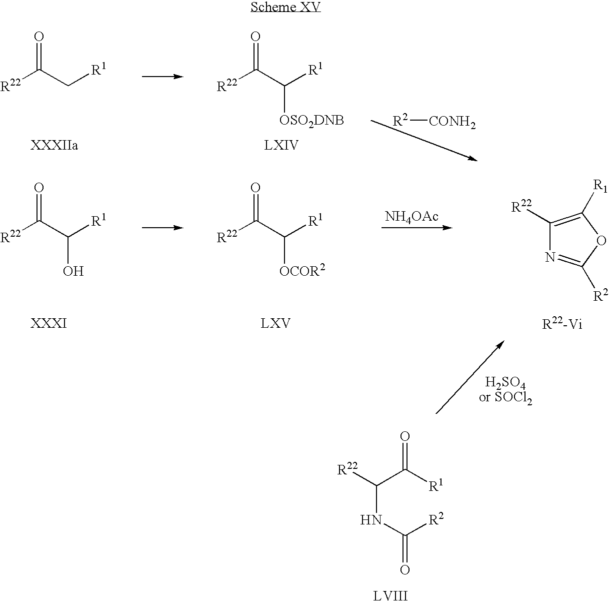

An alternative, preferred method to synthesize compounds of formula R²²-Vb, which includes a compound of formula Ib when R²²=IV, is shown in Scheme XVI. Amido-ketone LVIII, methods for whose preparation are given above, is heated with ammonium acetate in acetic acid to give R²²-Vb (R²¹=H), or with amine R²¹—NH₂ to give R²²-Vb. Other ammonia sources such as ammonia, ammonium chloride, or formamide may be substituted for ammonium acetate, and other solvents and acid catalysts may be employed for both reactions shown in Scheme XVI. One skilled in the art wilt be able to readily identify these alternative cyclization methods for formation of imidazoles from 1,4-dicarbonyl compounds such as LVIII from the literature. Also shown in Scheme XVI is an alternative method for preparation of LVIII. Sulfone LXVI (Ar is an optionally substituted aryl group, usually p-methylphenyl) is allowed to react with about 1.1 equiv R¹—CHO, 15 equiv triethylamine, 10 mol % of the thiazolium salt shown in Scheme XVI in chloroform at 35° C. to give LVIII (after the method of Murry, J. Am. Chem. Soc. 2001, vol 123, pp. 9696-9697). Sulfone LXVI is prepared from R²—CONH₂, R²²—CHO, and Ar—SO₂H according to a literature method by heating these reactants in formic acid (Morton, Tetrahedron Lett. 1982, vol 23, pp. 1123-6) or with trimethylsilylchloride in a suitable solvent (Sisko, Tetrahedron Lett, 1996, vol. 37, pp 8113-6; see also Method B in Sisko, Org, Synth. 1999, vol, 77, p. 198-205). The reaction of LXVI and R¹CHO to give LVIII, and the reaction of LVIII with R²¹—NH₂ to give R²²-Vb may be performed in a “one-pot” manner, using the method of Frantz (Org. Lett. 2004, vol. 8, pp. 843-845).

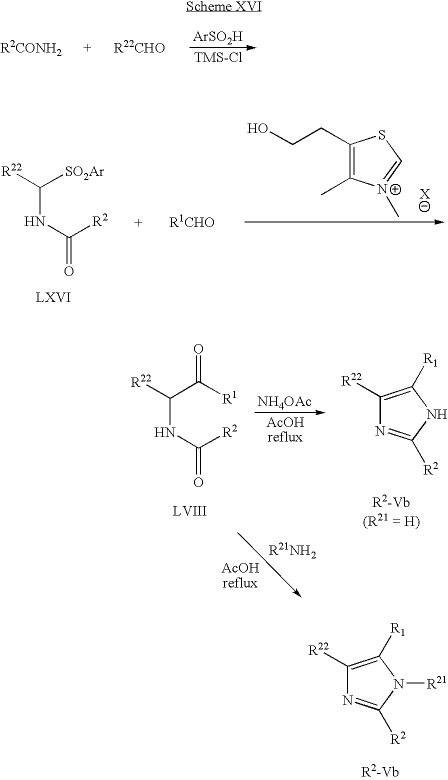

Scheme XVII describes a method for preparation of amino-esters XVI (R′=lower alkyl) and other intermediates used in preceding Schemes. Friedel-Crafts reaction of R²—H with ethyl oxalyl chloride and a suitable catalyst such as aluminum chloride in a suitable solvent such as carbon disulfide, nitrobenzene, chloroform, or dichloroethane at 0-120° C. produces keto-ester LXVII. If R²—H does not react or react with the desired regioehemistry in the Friedel-Crafts reaction, alternatively LXVII is also prepared by bromination of R²—H to give R²—Br and lithiation of R²—Br to give R²—Li. Alternatively direct lithiation of R²—H to give R²—Li, and subsequent reaction of R²—Li with ethyl oxalyl chloride gives LXVII. Suitable conditions for lithiation of R²—H and R²—Br are those given in Scheme I and discussion of Scheme I for lithiation of III and X₁-III. Also suitable preparations of certain LXVII with wide utility for other LXVII preparation are given by Castagnetti (Eur. J. Org. Chem. 2001, 691). Reaction of LXVII with hydroxylamine hydrochloride in ethanol or ethanol-water optionally containing a suitable base such as sodium bicarbonate provides oxime LXVIII. Alternatively oxime LXVIII is prepared by nitrosation of LXIX with sodium nitrite and an acid such as acetic or sulfuric acid in a suitable solvent or solvent mixture such as acetic acid and water. Reduction of oxime LXVII under suitable oxime reducing conditions produces amino ester XVI. XVI may also be prepared from R¹-X₁ by the route described to convert R²²-X₁ to LIX in Scheme XIV. Suitable oxime reducing conditions include hydrogenation with palladium on carbon in ethanol and transfer hydrogenation with ammonium formate, a palladium catalyst in methanol or ethanol. Reduction of XVI is accomplished using lithium aluminum hydride in tetrahydrofuran or ether.

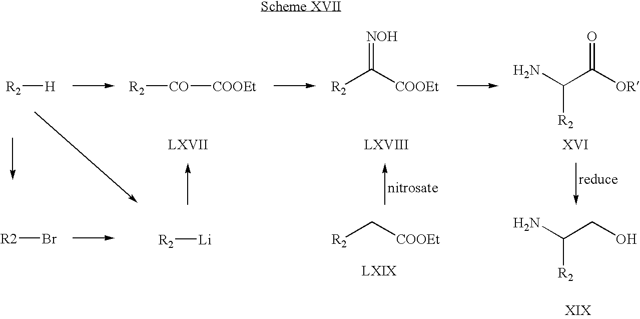

Scheme XVIII describes preparation of XIV, XXVI and other intermediates used in preceding Schemes. R²—COOH is converted to R²—CON(Me)OMe (Weinreb amide) by formation of the acid chloride (from thionyl or oxalyl chloride under standard conditions) and coupling to N,O-dimethylhydroxylamine, or by direct coupling using standard coupling agents for amide bond formation, R²—CON(Me)OMe subsequently treated with a slight excess of organometallic reagent R²⁰—CH₂-M₁ in a suitable solvent such as ether or tetrahydrofuran, typically at −78 to 25° C. to give ketone R²⁰—CH₂—CO—R². M₁ is preferablylithium or magnesium (halide), for example R²⁰—CH₂-M₁ (where R²⁰ is H) is methylmagnesium bromide or methyllithium. Ketone R²⁰—CH2—CO—R² is brominated to give XIV using a suitable literature monobromination method for an aryl- or heteroaryl ketones including treatment with a quaternary ammonium perbromide reagent in methanol, dichloromethane or tetrahydrofuran, heating with cupric bromide in chloroform or ethyl acetate, treatment with bromine in acetic acid, or treatment with bromine and a Lewis acid such as aluminum trichloride in a suitable solvent. A preferred monobrominating condition is treatment of the ketone with pyridinium bromide perbromide in acetic acid containing 5-10 equiv of hydrogen bromide. Preparation 96B-96D is exemplary of said sequence for converting R²COOH to XIV(X₂ is bromine, chlorine or triflate). Alternatively, certain bromoketones XIV are prepared by reaction of R²—Li with bromo or chloroester LXX at −100 to −70° C. and quenching at said low temperature where the tetrahedral adduct is stable. Said preparation is illustrated where LXX is methyl bromoacetate, and R² is 2-thiazolyl, 2-imidazolyl and other heterocycles having a ring nitrogen adjacent to the lithiation site, in the Examples herein. Amino-ketone XXVI is prepared by alkylation of R¹NH2 with XIV, wherein X₂ is a leaving group, preferably Br or Cl, under suitable amine alkylation conditions. Said alkylation is optionally conducted in the presence of a solvent and/or a base. Suitable solvents include C₁-C₄ alcohols including ethanol, and bases selected from carbonates and bicarbonates of sodium and potassium, at temperatures of 0-100° C., preferably 20-80° C. Lithium bromide or sodium iodide may also be included when beneficial. Alternatively XL is prepared from R²⁰—CH₂—COOH by coupling to N,O-dimethylhydraxylamine to give the corresponding Weinreb amide (for example by refluxing R²0-CH₂—COOH with thionyl chloride, or treating it with oxalyl chloride and a catalytic amount of dimethylformamide in a suitable inert solvent, to give the acid chloride, and treating said chloride with N,O-dimethylhydroxylamine and triethylamine in a suitable solvent such as dichloromethane. Said Weinreb amide is then added to R²—Li under suitable conditions to give XL. Alternatively XXVI may be prepared by condensing R¹NH₂ and an alkyl glyoxylate derivative LXXI to give an imine LXXII, for example by reaction in toluene or dichloroethane at 20-120° C. in the presence of a drying agent such as magnesium sulfate or activated molecular seives. LXXII is reduced to amine LXXIII by catalytic hydrogenation using palladium on carbon and hydrogen, by transfer hydrogenation using a palladium catalyst and ammonium formate, or by reducing with sodium borohydride, sodium thiacetoxyborohydride, or sodium cyanoborohydride in a suitable solvent such as methanol, acetic acid or dichloroethane or a mixture thereof. Amine LXXIII is protected with a suitable protecting group such as N-t-butoxycarbonyl or N-carbobenzyloxy. The resulting protected analog of LXXIII is then transformed into XXVI by any of the available literature methods for converting esters to ketones such as hydrolysis, coupling to form the Weinreb amide (protected form of XXIV), reaction with an organolithium reagent R₂—Li prepared as described above, and deprotection using suitable deprotection conditions to give XXVI.

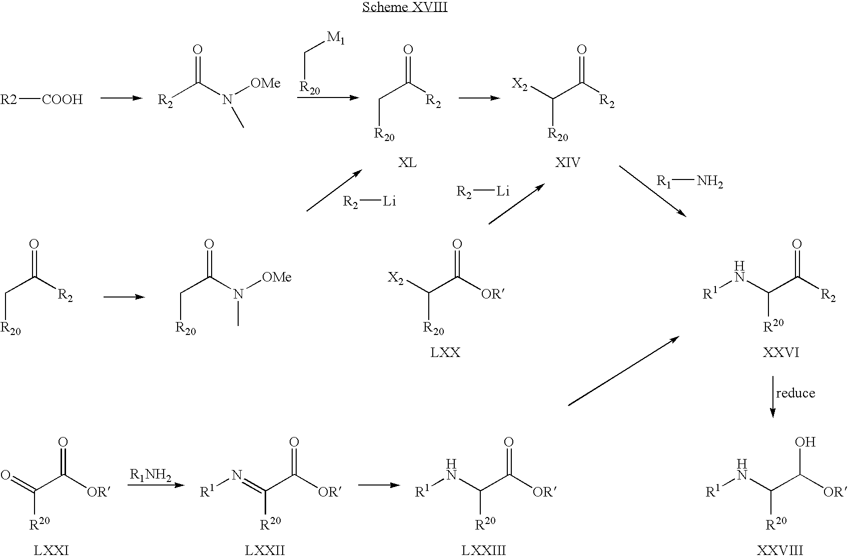

Scheme XIX shows the preparation and use of LXXV, an optional starting material in Schemes IV-XVII, which contains a protected aryloxy or heteroaryloxy radical (specifically where R²² is radical XI). Y₂ is chosen from the group consisting of H, CN, COOR′ (wherein R′ is lower alkyl), COOH, CONH₂, CHO, halogen, CH₃, CH₂NH₂, NH₂, NHNH₂, CH₂-M₁ and M₁ (wherein M₂ is selected from lithium, magnesium halide, zinc halide, B(OH)₂, B(OR₂) wherein R is as defined for Scheme lit, and SnR₃(R is methyl or n-butyl)).

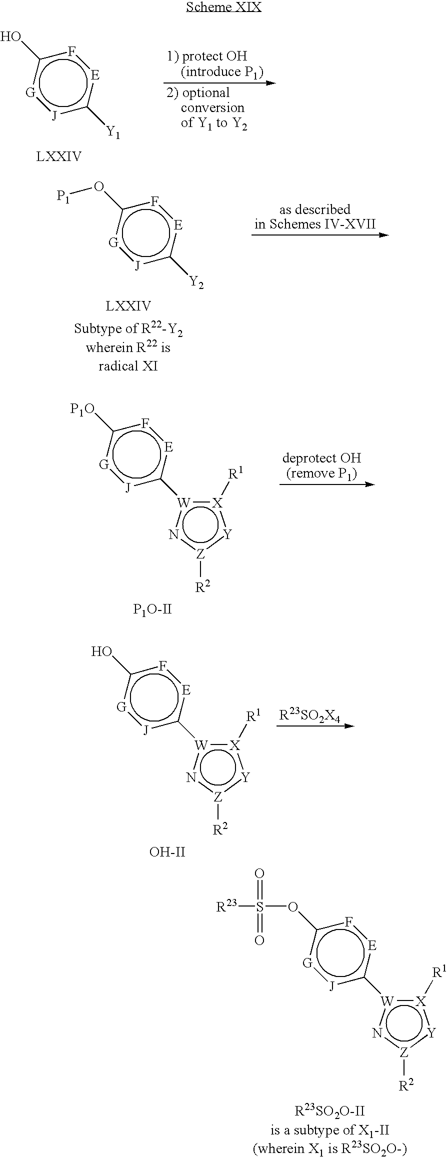

LXXV may be chosen as the R²²-containing starting material for Scheme IV-XVII based on its availability (or availability of its precursor LXXIV), or based on suitability for the intended reaction sequence. For example LXXV where Y₂ is iodide is an appropriate starting material for preparation of R²²-acetylene XXXIVa in Scheme IX by a Sonogashira reaction. LXXV is prepared by protecting LXXIV with a suitable protecting group, and by converting Y₁ to Y₂ if Y₁ and Y₂ are different. In LXXIV, Y₁ is chosen from the group consisting of H, CN, COOR′ (wherein R′ is lower alkyl), CHO, halogen, CH₃, NH₂, NH₂NH₂, and SnR₃ (R is methyl or n-butyl). Said protecting group P₁ is chosen to be stable to the reaction conditions to which it is subjected, except for those conditions intended for deprotection. More specifically P₁ is a protecting group for a phenolic or heteroaryloxy hydroxyl group which is chosen to be stable to the reaction conditions for the conversion of LXXIV to LXXV (when Y₂ is different from Y₁) and to the reaction conditions for conversion of LXXV to P₁—O—II. Protecting group P₁ is also chosen to be introduced under conditions where only the hydroxy function of LXXIV reacts, and to be removed by conditions which do not alter other features of P₁—O—II or cause adverse reaction of OH-II. A suitable group P₁ may be chosen, with the aforementioned considerations, from those described T. W. Greene and P. G. M. Wuts. Protective Groups in Organic Synthesis. John Wiley & Sons, New York, 1999. This reference also describes methods for introducing and removing said group, and charts describing the stability of said group under many different common reaction conditions which are employed in the Schemes herein. An exemplary set of radicals from which P₁ may be chosen is benzyl, methyl and triisopropylsilyl. Benzyl and methyl ethers LXXV of LXXIV (P₁=Bn or Me, respectively) are prepared for example by treatment of LXXIV with sodium hydride and benzyl bromide, or sodium hydride and methyl iodide, respectively, in dimethylformamide or tetrahydrofuran. Also said ethers may be prepared by alkylation of LXXIV with benzyl bromide, methyl iodide, or dimethyl sulfate under aqueous basic conditions or with cesium, sodium, or potassium carbonates in acetone, ethanol, or dimethylformamide. Alternatively said ethers may be made by the Mitsunobu reaction of LXXIV with benzyl alcohol or methanol. Also said methyl ether may be prepared by methylation of LXXIV with diazomethane in a suitable inert solvent. The triisopropylsilyl ether LXXV (P₁=i-Pr)₃Si) is prepared treating LXXIV (P₁=(i-Pr)₃Si) with triisopropylsilyl chloride and imidazole in dimethylformamide or acetonitrile. When Y₁ and Y₂ are different, P₁-protected LXXIV is converted to LXXV by an appropriate functional group interconversion reaction. For example in said protected intermediate wherein is COOMe, saponification conditions produce the corresponding product LXXV wherein Y₂ is COOH, As another example, reduction of said protected intermediate where Y₁ is CN with lithium aluminum hydride or by borane in tetrahydrofuran produces LXXV wherein Y₁ is CH₂NH₂. As a third example, transmetallation with t-butyllithium of said protected intermediate wherein Y₁ is iodide produces LXVII wherein Y₁ is lithium, and so forth. An intermediate of formula LXXV (R²²—Y₂ wherein R²² Z is radical XI) thus prepared is then converted to P₁O-II by a reaction sequence selected from Scheme IV-XVII. P₁O-II is then converted to HO-II by an appropriate deprotection reaction selected for the protecting group P₁. Exemplary deprotection conditions are treating methyl or benzyl ethers P₁O-II (P₁=Me or Bn, respectively) with boron tribromide in dichloromethane followed by treatment with aqueous sodium hydroxide to give OH—H, Alternatively catalytic hydrogenation deprotects said benzyl ether. Treating triisopropylsilyl ether P₁O-II (P₁=(i-Pr)₃Si) with tetrabutylammonium fluoride in tetrahydrofuran or heating with aqueous hydrochloric acid in an inert organic cosolvent deprotects said triisopropylsilyl ether giving OH-II. Said compound OH-II is converted to a compound of formula R²³SO₂O-II as shown, by reaction with a reagent of formula R²³—SO₂-X₄ or (R²³⁻SO₂)O under suitable conditions. R²³ is methyl, perfluoro-(C₁-C₄)-alkyl, or phenyl optionally monosubstituted with methyl or halogen. Preferred R²³ are p-methylphenyl and trifluoromethyl. X₄ is a suitable leaving group and is preferably halogen. Exemplary preferred reagents and conditions for conversion of LXXIV to LXXV are p-toluenesulfonyl chloride and either triethylamine or pyridine in a cosolvent such as dichloromethane, and treatment with trifluoromethanesulfonic anhydride and triethylamine in dichloromethane. Said compound of formula R²³SO₂O-II thus produced is a compound of formula X₁-II wherein X₁ is R²³SO₂O.

Scheme XX shows how X₁-II is converted to NH₂-II. A method is selected from those given for conversion of R²³-X₁ to R²².NH₂ in discussion of Scheme X, wherein X₁-II is substituted for R²²-X₁.

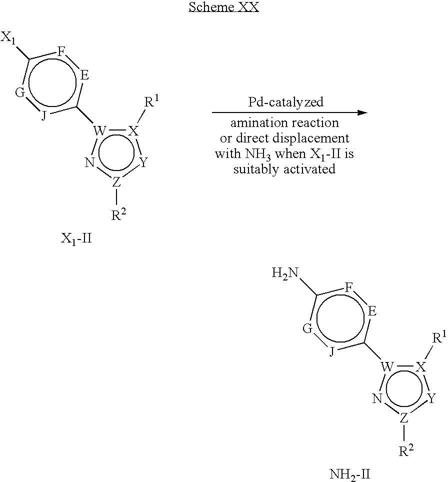

Scheme XXI depicts methods for preparing compounds containing the radical IV, a subtype of radical R²², which are used as intermediates to prepare compounds of formula I in preceding Schemes. In Scheme XXI, Y₃ is CN, COOR (wherein R is (C₁-C₄)alkyl or benzyl), CH₃ or OP₁, wherein P₁ is a protecting group as defined for Scheme XIX. X₁ and M₁ are as defined for Scheme I. M₂ is B(OH)₂, B(OR)₂ and R₃Sn where R is as defined for Scheme I.

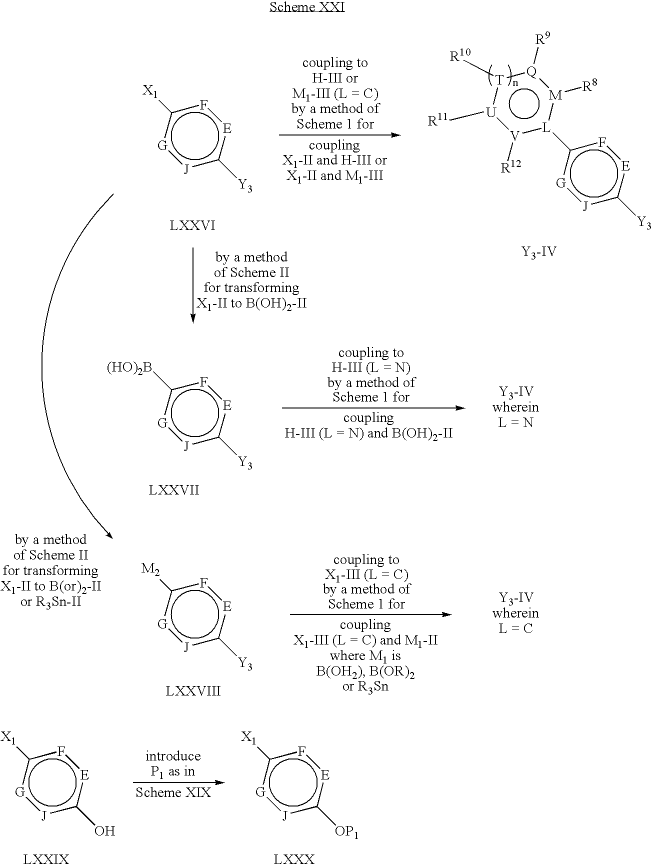

As shown in detail in Scheme XXI, intermediates LXXVI, LXXVII, and LXXVIII are converted, by coupling with H-III, M₁-III (LL=C), or X₁-III (LL=C), by analogy to and with the methods of Scheme I, to intermediates of formula Y₃-IV. Also, LXXVII and LXXVIII are optionally prepared by methods described in Scheme I for borylation or stannylation of X₁-II, as shown in Scheme XXI. Also shown is the formation of LXXX, a subtype of LXXVII, from a para-substituted hydroxy compound of formula LXXIX, by a method of Scheme XIX for protection of LXXIV therein.

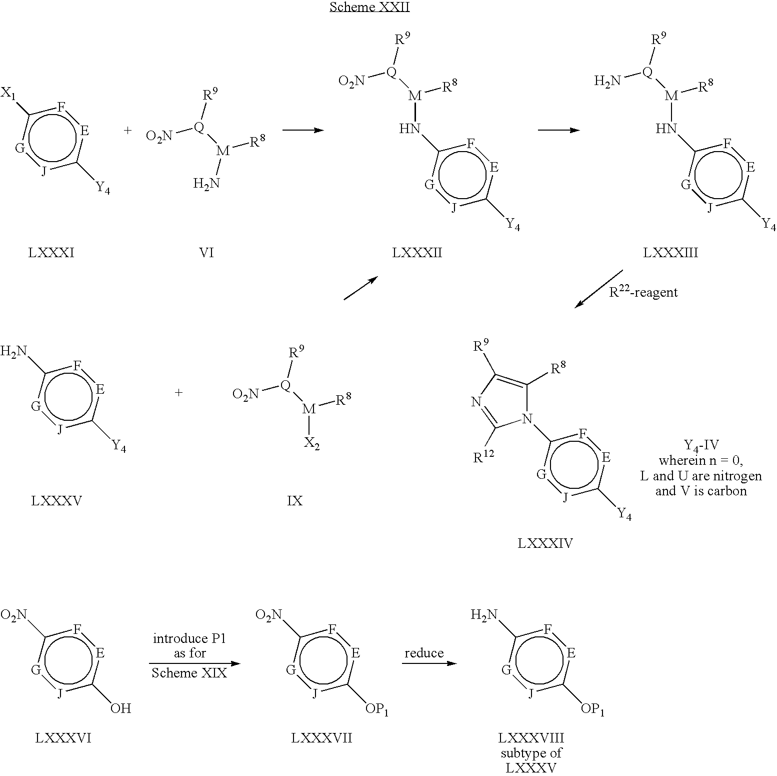

Scheme XXII depicts alternative methods for the preparation of intermediates LXXXIV which contain a subtype of radicals IV and R²², which may be used to prepare compounds of formula I by methods outlined in preceding Schemes, in Scheme XXII, Y₄ is CN, CH₃ or OP₁ (P₁ is as defined for Scheme XIX), X₁ is as defined for Scheme I. Intermediates VI and IX and R¹²-reagent are as defined for Scheme III. In Scheme XXIII, R⁸ and R⁹ are taken together to form an aromatic or heteroaromatic ring but are otherwise as defined for claim 1. Reactions shown in Scheme XXII are accomplished by the methods of Scheme III. More specifically, LXXXI is substituted for X₁-II in Scheme III. LXXXII is substituted for VII, LXXXIII for VIII, and LXXXV for NH₂-II of Scheme III, to give a product of formula LXXXIV. Intermediate LXXXI is a subtype of LXXVI in the preceding Scheme. Preparation of LXXXVIII, a subtype of LXXXV, is accomplished by protecting nitro compound LXXVII with P₁ by the method described for introducing P₁ in Scheme XIX, and reducing the resultant protected nitro compound LXXVII by a suitable method (such as hydrogenation with palladium on carbon in methanol, or with stannous chloride if P₁ is benzyl).

Scheme XXIII

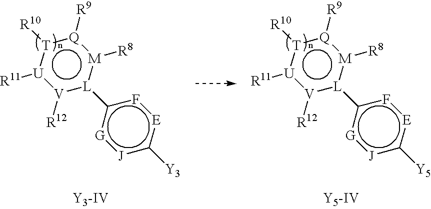

| Y₃ in Y₃-IV | Y₅ in Y₅-IV | Exemplary Method(s) |
|---|---|---|
| CN | COOH | complete hydrolysis: NaOH or H₂SO₄ in |
| | | H₂O/opt. cosolvent |
| CN | CO₂Me | above plus cat. H₂SO₄, MeOH or K₂CO₃, |
| | | MeI, DMF |
| CN | CONH₂ | H₂O₂, NaOH, EtOH—H₂O, RT or |
| | | NaBO₃/H₂O/MeOH |
| CN | CH₂NH₂ | 1) BH₃—Me₂S/THF 2)HCl—H₂O or |
| | | LiAlH₄/THF or H₂, Pd/C, NH₃—EtOH |
| CN | CHO | 1) i-Bu₂AlH, solvent, −78° C.; |
| | | 2) H₂SO₄—H₂O |
| COOCH₃ | COOH | NaOH, THF—H₂O |
| OP₁ | OSO₂R²³ | analogy to conversion of P₁O-II to R²³SO₂-II |
| | | (Scheme XIX) |
| Y₅ in Y₅-IV | Y₅ in Y₅-IV | Exemplary interconversions of Y₅ in Y₅-IV |
| OSO₂R²³ | NH₂ | described in Scheme X |
| OSO₂R²³ | B(OH)₂ | analogy to conversion of X₁-II to B(OH)₂-II |
| | | (Scheme II) |
| OSO₂R²³ | R₃Sn | analogy to conversion of X₁-II to R₃Sn-II |
| | | (Scheme II) |

The first part of Scheme XXIII shows methods for transforming compounds containing the radical IV (Y₃-IV) formed in the preceding Schemes XXII and XXI to other intermediates used in preceding Schemes (Y₃-IV) for preparing compounds of formula I. These are exemplary methods which are well known to one skilled in the art and for which there is extensive literature precedent. Many other methods are also available for accomplishing said transformations. The second part of Scheme XXIII shows standard functional group transformations known to one skilled in the art, whereby said compounds Y₅-IV in the first part of the Scheme are converted to yet other compounds Y₅-IV also used in preceding schemes to synthesize compounds of formula I.

### EXAMPLES

### General Experimental Procedures

Abbreviations used include SGC (silica gel chromatography), DCM (dichloromethane), THF (tetrahydrofuran), EtOAc (ethyl acetate), TFA (trifluoroacetic acid), DMF (dimethylformamide). LiHMDS (lithium bis-(trimethylsilyl)amide), Bn (benzyl), Ar (aryl). RT (room temperature), and equiv (equivalents). NH₄OH refers to the concentrated aqueous reagent containing 28-30% ammonia. Ratios of liquids are specified using volume measures (e.g. 5:1 DCM: 2-propanol, or 0.5% MeOH in DCM, 0.5% NHOH (where the fatter means 0.5 ml MeOH and 0.5 mL cone. NH₄OH per 100 ml. DCM). Proton NMR were obtained at 400 mHz, and ¹³C NMR at 100 mHz on Varian Unity 400 spectrometers. Chemical shifts are expressed in parts per million downfieid from trimethylsilane (external reference). Mass spectral data were obtained using a Micromass ZMD spectrometer operating with an atmospheric pressure chemical ionization (APCl) source (when AP+ is designated), or (when ES+ is designated) an electrospray source. Reactions heated by microwave were conducted using either a Personal Chemistry SmithCreator™ microwave reactor (for 2-5 mL solvent volumes) or (larger volumes) a Personal Chemistry (Biotage) Optimizer™ microwave reactor with pressure limits set at approximately 200 p.s.i. Melting points were obtained on a Thomas-Hoover melting point apparatus and are uncorrected. HPLC-MS analysis was performed on a Hewlett Packard (Agilent Technology) 1100 series system at a flow rate of 1.0 mL/minute using diode array and mass detectors with acetonitrile (solvent A) and 0.1% (v/v) formic acid in water (solvent B). When ratios or purities are specified the A₂₈₀ signal is used. If not otherwise specified, the method used a linear binary gradient of 10:90 A:B to 90:10 A:B over 10 min on a Zorbax Bonus-RP™ column, 5 μM particle size, 150 mm×4.6 mm i.d. Method 2 used the same column but a linear gradient of 3:7 A:B to 95:5 A:B over 15 min. Method 3 used a 5 μM Kromasil™ 150×4.6 mm column with an isocratic ratio of A:B as specified (e.g. 60/40 means 60% A, 40% B). RP-HPLC purification was performed using a Shimadzu preparative HPLC equipped with X-Terra™ 50×50 mm column, linear gradient of 25%-85% (over 10 min) acetonitrile:water, each containing either 0.1% TFA (“acidic conditions”) or 0.1% NH₄OH (“basic conditions”). Organic solutions were dried over MgSO₄ or Na₂SO₄, unless otherwise specified. When a reaction mixture is described below to be filtered and concentrated, unless otherwise specified, the filtered solids are washed with either more of the reaction solvent with DCM, or with a mixture of DCM and 2-propanol and the filtrates are combined and concentrated. The term “concentrated” refers to removal of solvent at reduced pressure on a rotary evaporator at a temperature between room temperature and 70° C. “Drying” or “dried” refers to drying at high vacuum (0.5-0.05 Torr) between room temperature and 100° C.

### General Procedure 1

### Amidine Formation from Aryl-Nitrile and Aryl- or Heteroarylamine with Sodium Hydride in Dimethylsulfoxide

Sodium hydride dispersion (60% in oil, about 1.5 equiv NaH) is added to a solution of the aromatic nitrite (1.0 equiv) and aryl- or heteroarylamine (usually 1.0 equiv) in anhydrous dimethylsulfoxide at RT and the resulting mixture heated at 50-60° C. for 2-18 h, usually 3-4 h. The cooled mixture is quenched with water, or more usually, poured onto ice, and the resulting mixture extracted with EtOAc and the EtOAc extracts dried, concentrated, and purified as indicated. On some occasions, as indicated, the amidine precipitated and was filtered and processed as indicated.

### General Procedure 2

### Formation of an Imidazole by Sequential Treatment of an the Intermediate Hydroxyimidazoline in Hot Acetic Acid

A 1.0 M solution of LiHMDS in THF (Aldrich Chemical Co., 1.0-1.2 equiv, or 2.2 equiv when the heteroaryl-halomethylketone is a hydrobromide salt) is added dropwise to a solution of the amidine (1.0 equiv) in anhydrous THF (generally 2-4 ml/mmol amidine) at −20° C. to 5° C. under nitrogen and the resulting solution stirred at about 0° C. for 10-30 min, A solution of the haloketone (1.0-1.5 equiv, in equal or greater amount relative to the lithium base) in anhydrous THF (1-3 mL per mmol) is added in one portion. The resulting mixture is stirred in an ice bath for 10-30 min and then at RT for at least 30 min. Water and organic solvent (usually EtOAc or DCM) are added and the product is isolated by extraction into the organic layer which is dried and concentrated. The resulting crude product, which generally contains hydroxy-imidazoline, the target imidazole, and unreacted amidine (HPLCMS analysis) is dissolved in acetic acid (5-25 ml/mmol) and heated at 60-100° C. for 20-60 min (HPLCMS showing disappearance of the hydroxy-imidazoline peak). This mixture is concentrated, and the crude product isolated by extraction using aqueous NaOH and organic solvent (usually EtOAc or DCM), and residual amidine removed by washing with aqueous citric acid. If not otherwise specified, the product was purified by SGC (gradient of MeOH in DCM, 0.5% NH₄OH). In the following Example section, compounds of formula I are designated as Example 1, Example 2, and so on, whereas the corresponding synthetic intermediates are designated Preparation 1A, Preparation 1B, or Preparation 2A and so on.

### Example 1

### 1-(4-(1-(4-methoxyphenyl)-4-(thiophen-2-yl)-1H-imidazol-2-yl)phenyl)-1H-pyrrolo[2,3-b[pyridine

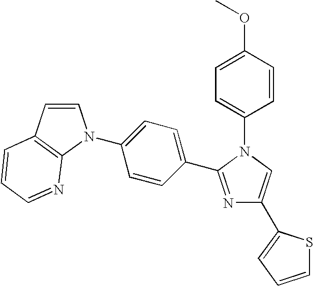

N′-(4-methoxyphenyl)-4-(1H-pyrrolo[2,3-b]pyridin-1-yl)benzamidine (300 mg, 0.876 mmol), 2-chloroacetylthiophene (210 mg, 131 mmol), and NaHCO₃ (147 mg, 1.75 mmol), were combined in 6 mL 2-propanol and the resulting mixture heated at 76° C. for 16 h. The mixture was concentrated and the residue purified by SGC (EtOAc-hexanes) giving 261 mg (66%) of the title substance. ¹H NMR (CDCl₃) δ 8.33 (dd, 1H, J=1.5, 4.5). 7.93 (dd, 1H, J=1.7, 7.9), 7.78 (m, 2H), 7.65 (m, 2H), 7.51 (d, 1H, J=3.7), 7.31 (s, 1H), 7.25-7.23 (m, 4H), 7.12 (dd, 1H, J=4.6, 7.9), 7.07 (dd, 1H, J=3.7, 5.0), 6.94 (m, 2H), 6.61 (d, 1H, J=3.7), 3.83 (s, 3H). MS (AP+) m/e 449 (MH+). IC₅₀=3.35 nM.

### Preparation 1A

### 4-(1H-pyrrolo[2,3-b]pyridin-1-yl)benzonitrile

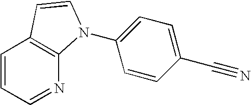

A mixture of 7-azaindole (37.5 g, 0.317 mol), 4-iodobenzonitrile (80 g, 0.349 mol CuI (0.91 g, 4.75 mmol), K₃PO₄ (136 g, 0.634 mol), and (±)-trans-1,2-diaminocyclohexane (3.62 g, 31.7 mmol) in p-dioxane (120 mL) was stirred vigorously in a 250 mL flask equipped with a reflux condenser at 120° C. (oil bath temperature) for 19 h. The mixture was cooled and filtered and the solids washed sequentially with EtOAc (200 mL) and DCM (200 mL). The filtrate was concentrated, the residue was dissolved in EtOAc, and the resulting solution washed with water (3×100 mL), brine, dried over Na₂SO₄, and concentrated. The solid so obtained was dissolved in 75 mL boiling DCM and 300 mL, hexanes was added. The resulting white suspension was filtered at RT and the filtered solid washed twice with 1:5 DCM-hexanes and dried (colorless solid, 43.3 g). The filtrates were concentrated and the residue recrystallized in the same manner giving a second crop, also pure by NMR (15.3 g, 84% total yield). ¹H NMR (CDCl₃) δ 8.37 (dd, 1H, J=1.3.5), 8.06-8.02 (m, 2H), 7.97 (dd, J=1.7, 7.9), 7.81-7.77 (m, 2H), 7.55 (d, 1H, J=3.7 Hz), 7.17 (dd, 1H, J=5, 7.9), 6.68 (d, 1H, J=3.7). MS (AP+) 220 (MH+).

### Preparation 1B

### (E)-N′-(4-methyoxyphenyl)-4-(1H-pyrrolo[2,3-b]pyridin-1-yl)benzamadine

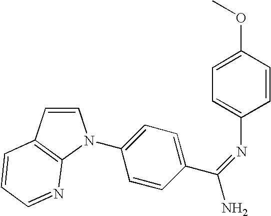

A mixture of 4-methoxyaniline (1.12 g, 9.11 mmol) in toluene (50 mL) was treated at 0° C. with a solution of trimethylaluminum (6.4 mL of 2.0 M in toluene, 12.8 mmol) and the mixture was stirred 3 h at RT. A solution of (1H-pyrrolo[2,3-b]pyridin-1-yl)benzonitrile (2.0 g, 9.1 mmol) in toluene (25 mL) was added dropwise and the resulting mixture heated at 73° C. for 18 h and 90° C. for 4 h. The mixture was poured into a stirred mixture of silica gel, MeOH, and DCM, filtered and the fitter cake washed with 300 mL of 2:1 DCM-MeOH, The filtrate was concentrated giving an orange solid which was recrystallized from 0.5:2:1 EtOAc-hexanes-ether giving 1.92 g (62%) of a brown solid. ¹H NMR (DMSO-d₆) δ 8.32 (dd, 1H, J=1.7, 4.6). 8.10-8.01 (m, 6H), 7.20 (dd, 1H, J=4.8, 7.9), 6.87 (m, 2H), 6.82-6.76 (m, 2H), 6.73 (d, 1H, J=3.7), 6.27 (br, 2H), 3.70 (s, 3H), IC50.

### Example 2

### 1-(4-(1-(4-methoxyphenyl)-4-(thiazol-2-yl)-1H-imidazol-2-yl)phenyl)-1H-pyrrolo[2,3-b]pyridine

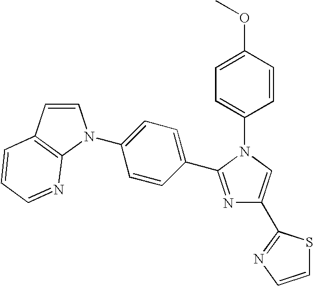

N′-(4-methoxyphenyl)-4-(1H-pyrrolo[2,3-b]pyridin-1-yl)benzamidine (300 mg, 0.876 mmol), 2-bromoacetylthiazole (270 mg, 1.31 mmol, Dondoni et al, J. Am. Chem. Soc. 1994, 116, 3324-3336), and NaHCO₃ (147 mg, 1.75 mmol), were combined in 6 mL 2-propanol and the resulting mixture heated at 76° C. for 16 h. The mixture was concentrated and the residue purified by SGC (EtOAc-hexanes) and the product triturated with ether giving an orange solid (75 mg, 19%), ¹H NMR (CDCl₃, partial) δ 8.34 (dd, 1H, J=1.4, 4.3), 7.93 (dd, 1H, J=1.6, 7.8), 7.80 (d, 1H, J=3.3), 7.75 (m, 3H), 7.59 (m, 2H), 7.49 (d, 1H, J=3.8), 7.28 (d, 1H, J=3.3), 7.23 (m, 2H), 7.12 (dd, 1H, J=5.0, 7.9), 7.92 (m, 2H). 6.61 (d, 1H, J=3.7), 3.83 (s, 3H). MS (AP+) m/e 450 (MH+). IC₅₀=156 nM.

### Preparation 2A

### 2-Bromoacetylthiazole

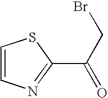

The following is a large-scale adaptation of the Dondoni procedure cited above. A solution of bromoacetyl bromide (57.6 g, 0.285 mol) in dry DCM (100 ml) was added at 0-5° C. to a stirred solution of 2-trimethylsilylthiazole (37.4 g, 0.238 mol) in DCM (300 ml). After 2 h at 0° C., aqueous saturated NaHCO₃ (1 L) was added and the resulting mixture was extracted with DCM (2×500 ml). The extracts were stirred with decolorizing carbon (Darco KB™, 10 g) and filtered through Celite, and concentrated. The residue was purified by SGC (1.2 kg silica, 1:3 to 1:1 DCM-hexanes) giving 25.2 g of colorless crystalline solid (41%). NMR (CDCl₃, 400 mHz) δ 8.02 (d, 1H, J=3.3 Hz), 7.74 (d, 1H, J=3 Hz), 4.69 (s, 2H). An alternate preparation was also achieved as follows. A solution of n-butyllithium (13.1 mL of 2.5 M in hexanes) was added at −78° C. to a stirred solution of 2-thiazole (2.66 g, 31.25 mmol) in ether (26 mL). After 15 min, methyl bromoacetate (3.11 mL, 32.8 mmol) was added giving a light brown slurry which was warmed to RT and treated with acetic acid (3.6 mL). Water (50 mL) and ether (30 mL) were added and the ether layer was separated, dried, and concentrated. The residue was suspended in hexanes (50 mL) at reflux and the hexanes decanted from a heavy oil. This was repeated and the hexanes combined and concentrated giving 4.9 g of light yellow needles (76%) having NMR identical to that described above plus minor impurities which could be removed by one trituration with 10 mL hexanes at RT.

### Example 3

### 1-(4-(1,4-di(thiazol-2-yl)-1H-imidazol-2-yl)phenyl)-1H-pyrrolo[2,3-b)pyridine

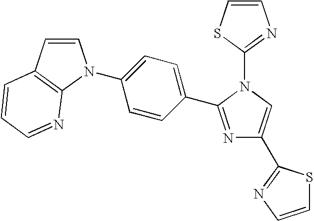

4-(1H-pyrrolo[2,3-b]pyridin-1-yl)-N′-(thiazol-2-yl)benzamidine (191 mg, 0.60 mmol), 2-bromoacetylthiazole (247 mg, 1.2 mmol), and NaHCO₃ (135 mg) were combined in 5 mL 2-propanol and the mixture was heated at 95° C. for 24 h. More 2-bromoacetyl thiazole (100 mg) was added and heating was continued for 5 h. The mixture was concentrated and the residue (743 mg) purified by preparative reversed-phase HPLC on an X-Terra™ 50×50 mm column eluted with a linear gradient of 25%-85% acetonitrile in aqueous 0.1% TFA over 10 min giving 27 mg of an oil which was further purified by SGC (EtOAc-hexanes) giving 10 mg (4%) of the title substance. ¹H NMR (DMSO-d₆) δ 8.31 (dd, 1H, J=4.6, 1.7). 8.22 (d, 1H, J=4.6), 8.05 (dd, 1H, J=1.7, 7.9), 8.03 (d, 1H, J=3.1), 7.95 (d, 1H, J=3.7), 7.86 (d, 2H, J=8.7), 7.7-7.6 (m, 2H), 7.55 (d, 2H, J=8.7), 7.54 (d, 1H, J=4.3), 7.19 (dd, 1H, J=7.8, 4.7), 6.71 (d, 1H, J=3.7). MS (AP+) m/e 427 (MH+). IC₅₀=1610 nM.

### Preparation 3A

### Methyl 4-(1H-pyrrolo[2,3-b]pyridin-1-yl)benzimidate hydrochloride

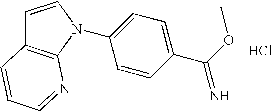

Anhydrous HCl was introduced into a suspension of 4-(1H-pyrrolo[2,3-b]pyridin-1-yl)benzonitrile (2.0 g, 9.11 mmol) in ether (20 mL) at 0° C. for about 20 min, during which time the initial solid dissolved, and a precipitate subsequently formed. The vessel was capped and stored at RT for 5 h, at which time no nitrile remained (TLC analysis). The product was filtered, washed with ether and dried giving 2.60 g of a light yellow solid (99%), which was used within 1 day. NMR indicated about 15% of an impurity was present. For the major substance, ¹H NMR (DMSO-d₆) (δ 8.33 (m, 2H), 8.36 (dd, 1H, J=1.7, 4.6), 8.30 (m, 2H), 8.17 (d, 1H, J=3.7), 8.10 (dd, 1H, J=1.7, 7.9), 7.26 (dd, 1H, J=4.8, 7.7), 6.81 (d, 1H, J=3.7), 6.17 (or, 2-3H), 4.29 (s, 3H). MS (AP+) m/e 252 (MH+). In a second preparation on 10 g scale, the impurity was present in about 30% amount and did not appear to have a methoxyl resonance.

### Preparation 3B

### 4-(1H-pyrrolo[2,3-b]pyridin-1-yl)-N′-(thiazol-2-yl)benzamidine

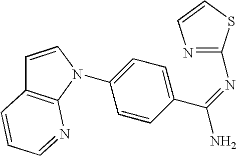

Methyl 4-(1H-pyrrolo[2,3-b]pyridin-1-yl)benzimidate hydrochloride (250 mg, 0.87 mmol), 2-aminothiazole (87 mg, 0.87 mmol) and triethylamine (178 mg, 1.74 mmol) were combined in 2 mL MeOH and heated at 75° C. for 72 h. The mixture was concentrated, the residue partitioned between DCM and saturated aqueous NaHCO₃. The aqueous layer was withdrawn and extracted twice with DCM. The organic layers were combined, dried over Na₂SO₄, filtered and concentrated giving an orange product which was used without purification (191 mg). MS (AP+) m/e 320 (MH+).

### Example 4

### 1-(4-(4-(pyridin-2-yl)-1-(pyrimidin)-5-yl-)-1H-imidazol-2-yl)phenyl)-1H-pyrrolo[2,3-b]pyridine

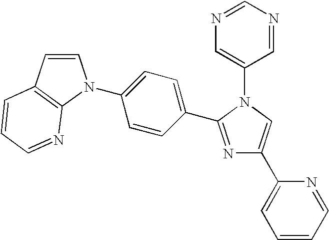

According to General Procedure 2, N′-(pyrimidin-5-yl)-4-(1H-pyrrolo[2,3-b]pyridin-1-yl)benzamidine (447 mg, 1.42 mmol) and 2-bromo-1-(pyridin-2-yl)ethanone hydrobromide (400 mg, 1.42 mmol) gave the title substance as a yellow solid. Yield 80 mg, 13.5% of theory. ¹H NMR (CDCl₃) δ 9.24 (s, 1H), 8.77 (s, 2H), 8.58 (m, 1H), 8.35 (dd, 1H, J=1.7, 4.6), 8.13 (d, 1H, J=7.9), 7.95 (dd, 1H, J=1.5, 7.7), 7.90 (s, 1H), 7.87 (m, 2H), 7.77 (m, 1H), 7.57 (m, 2H), 7.52 (d, 1H, J=3.7), 7.20 (m, 1H), 7.13 (dd, 1H, J=5.0, 7.9), 6.64 (d, 1H, J=3.7). MS (AP+) m/e 416 (MH+). IC₅₀=13.6 nM.

### Preparation 4A

### N′-(pyrimidin-5-yl)-4-(1H-pyrrolo[2,3-b]pyridin-1-yl)benzamidine

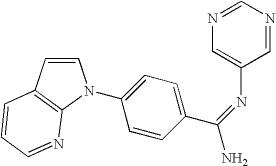

Sodium hydride oil dispersion (6.85 g of 60%) was added to a solution of 4-(1H-pyrrolo[2,3-b]pyridin-1-yl) benzonitrile (25.0 g, 114 mmol) and 5-aminopyrimidine (10.8 g, 114 mmol, prepared as described by Philips et al, Can. J. Chem 1999, 77, 216-222) in anhydrous dimethylsulfoxide (200 mL), and the resulting suspension was heated at 50-60° C. for 4-8 h. The cooled mixture was poured onto ice (1 kg), and the yellow suspension stirred with 150 mL EtOAc and 150 mL hexanes for 15 min and filtered. The solid was washed with water (1 L in 3 portions), and dried at 78° C. in vacuo overnight. The dried solid (30.0 g) was suspended in 400 mL 1N HCl and the resulting aqueous solution extracted with EtOAc (5×125 mL). DCM (100 mL) and aqueous NaOH (110 mL of 6N were added to the aqueous layer giving a flocculent suspension which was filtered and the solid washed with water (2×200 mL) and dried at 78° C. and 0.1 mm giving the title substance (22.7 g). ¹H NMR (CDCl₃) δ 8.78 (br, 1H), 8.33 (m, 3H), 8.2-8.0 (m, 6H), 7.21 (dd, 1H, J=4.6, 7.9), 6.95 (br, 2H), 6.75 (d, 1H, J=3.7). MS (AP+) m/e 315 (MH+).

### Preparation 48

### 2-bromo-1-(pyridin-2-yl)ethanone hydrobromide

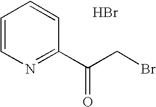

30% HBr in acetic acid (100 mL) was added at RT to a stirred solution of 2-acetylpyridine (40 g, 0.33 mol) in acetic acid (100 mL). Pyridinium tribromide (118 g) was added and the resulting mixture was stirred 23 h at RT and filtered. The solid was washed with acetic acid (3×100 mL) and dried at 78° C. in vacuo until sublimation began, then at RT in vacuo, giving 88.0 g (95%) of the title substance ¹H NMR (CD₃OD, 400 mHz) δ 8.82 (ddd, 1H, J=0.8, 1.7, 4.6 Hz), 8.73 (td, 1H, J=1.5, 8.0 Hz). 8.28 (ddd, 1H, J=1.1.8 Hz), 8.14 (ddd, 1H, J=1, 5, 8 Hz), 3.91 (A of AB, 1H, J=11.6 Hz), 3.81 (B of AB, 1H, J=11.6 Hz). The species observed by NMR was presumed to be a hemiketal adduct of the title substance and d4-methanol.

### Example 5

### 1-(4-(1-(2-methylpyridin-4-(pyridin-2-yl)-1H-imidazol-2-yl)-1H-imidazol-2-yl)phenyl)-1H-pyrrolo[2,3-b]pyridine

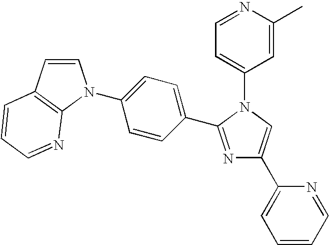

According to General Procedure 2, N′-(2-methylpyridin-4-yl)-4-(1H-pyrrolo[2,3-b]pyridin-1-yl)benzamidine (500 mg, 1.53 mmol) and 2-bromo-1-(pyridin-2-yl)ethanone hydrobromide (430 mg, 1.53 mmol) gave the title substance as an off-white solid. Yield 300 mg, 46% of theory. ¹H NMR (CDCl₃) δ 8.6 (d, 1H, J=4.6 Hz), 8.55 (d, 1H, J=5.4 Hz), 8.38 (dd, 1H, J=1.7, 4.6 Hz), 8.15 (d, 1H, J=7.9 Hz), 7.99-7.96 (m, 2H), 7.87 (m, 2H). 7.80 (m, 1H), 7.63 (m, 2H), 7.55 (d, 1H, J=3.7 Hz), 7.22 (m, 1H), 7.19 (m, 1H), 7.16 (dd, 1H, J=4.6, 7.9 Hz), 7.04 (dd, 1H, J=2.1, 5.4 Hz), 6.66 (d, 1H, J=3.7 Hz), 2.60 (s, 3H). MS (AP+) m/e 429 (MH+). IC₅₀=4.62 nM.

### Preparation 5A

### N′-(2-methylpyridin-4-yl)-4-(1H-pyrrolo[2,3-b]pyridin-1-yl)benzamidine

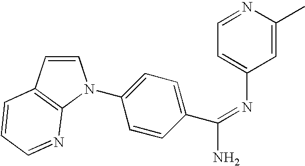

According to General Procedure 1, 4-(1H-pyrrolo[2,3-b]pyridin-1-yl)benzonitrile (5.06 g, 23.1 mmol and 4-amino-2-picoline (2.5 g, 23.1 mmol) gave a reaction mixture which was poured onto about 400 g ice and 100 ml 1:1 EtOAc-hexanes and the solid product was filtered, washed thoroughly with water (1 liter in 4 portions) and dried giving the title substance. Yield 5.79 g, 76% of theory. ¹H NMR (CDCl₃) δ 8.39 (br, 1H), 8.36 (dd, 1H, J=1.5, 4.8 Hz), 8.00 (m, 2H), 7.97 (dd, 1H, J=1.7, 7.9 Hz), 7.92 (m, 2H), 7.55 (d, 1H, J=3.7 Hz), 7.15 (dd, 1H, J=4.6, 7.9 Hz), 8.79 (br, 1H), 6.74 (br, 1H), 6.66 (d, 1H, J=3.7 Hz), 4.89 (br, 2H), 2.52 (s, 3H). MS (AP+) m/e 328 (MH+).

### Example 6

### 1-(4-(1-(6-methylpyridine-3-yl)-4-(pyridin-2-yl)-1H-imidazol-2-yl)phenyl)-1H-pyrrolo[2,3-b]pyridine

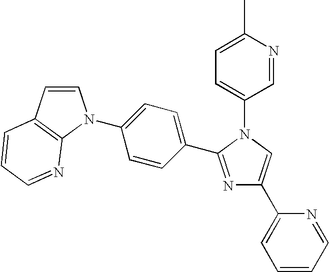

A mechanically stirred suspension of N′-(6-methylpyridin-3-yl)-4-(1H-pyrrolo[2,3-b]pyridin-1-yl)benzamidine (49.6 g, 152 mmol) in anhydrous THF (1 L) was treated over 30 min at less than 4° C. with a solution of LiHMDS (350 mL of 1M in THF). After 15 min at 0° C. the clear brown solution was treated portionwise at 3-6° C. with 2-bromo-1-(pyridin-2-yl)ethanone hydrobromide (42.6 g, 152 mmol) over 20 min. After being stirred 30 min at 0° C. and the mixture was warmed to 25° C. over 1 h and stirred at 25° C. for 30 min. Water (500 mL) and EtOAc (1 L) were added and the organic layer was separated, washed with brine, dried over Na₂SO₄, and concentrated. The residue was dissolved in 200 mL acetic acid and the resulting solution heated at 95° C. for 20 min and concentrated. The residue was dissolved in EtOAc (1 L) and 2N HCl (450 mL). The organic layer was separated and washed with water (150 mL) and aqueous 10% citric acid (250 mL). The citric acid layer was extracted with EtOAc (2×100 mL). The combined organic layers were washed with water, brine, dried, and concentrated giving 42 g of crude product as a brown oil which was purified by SGC (1% MeOH in DCM, 0.5% NH₄OH), giving the title substance in several fractions contaminated with 1-7% of the corresponding amide (4-(1H-pyrrolo[2,3-b]pyridin-1-yl)benzamide) as determined by HPLC (280 nM absorption ratio). Yield 15 g, 31%. The material was efficiently further purified by recrystallization as illustrated: a 4.5 g fraction containing 3.5% amide impurity was dissolved in 98:2 acetonitrile:water and the resulting solution stirred at RT from 40 min. The crystalline precipitate was filtered, washed with fresh acetonitrile and dried giving 2.9 g of the title substance containing 0.3% amide, in this manner the remaining fractions were purified and the recrystallized solids combined giving 9.35 g of the title substance containing less than 1% amide impurity. ¹H NMR (CDCl₃) δ 8.58 (m, 2H), 8.37 (dd, 1H, J=1.5, 4.8 Hz), 8.18 (d, 1H, J=7.9 Hz), 7.97 (dd, 1H, J=1.7, 7.9 Hz), 7.91 (br, 1H), 7.82 (m, 2H), 7.79 (id, 1H, J=1.7, 7.9 Hz), 7.62 (m, 2H), 7.53-7.50 (m, 2H), 7.24-7.19 (m, 2H), 7.15 (dd, 1H, J=4.6, 7.9 Hz), 6.65 (d, 1H, J=3.7 Hz), 2.64 (s, 3H). MS (AP+) m/e 429 (MH+). Anal. Calcd for C₂₇H₂₀N₈: C, 75.68; H, 4.70; N, 19.61. Found: C, 75.39; H, 4.52; N, 19.64. IC₅₀=3.21 nM.

### Preparation 6A

### N′-(6-methylpyridin-3-yl)-4-(1H-pyrrolo[2,3-b]pyridin-1-yl)benzamidine

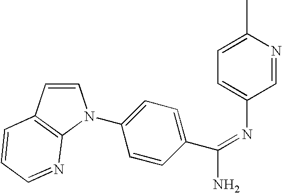

According to General Procedure 1, 4-(1H-pyrrolo[2,3-b]pyridin-1-yl) benzonitrile (51.5 g, 0.235 mol), 3-amino-6-methylpyridine (25.45 g, 0.235 mol), 60% sodium hydride oil dispersion (14.1 g, 0.353 mol) in dimethylsulfoxide (200 mL) for 3 h at 55° C. gave a reaction mixture which was poured onto ice together with 150 mL EtOAc and 150 ml hexanes, the mixture stirred 30 min and filtered, and the solid washed repeatedly with water and hexanes and partially dried. The solid (91 g) was dissolved in 590 mL of 2 N HCl and the resulting solution extracted with EtOAc (3×300 mL). Aqueous 2N NaOH (450 mL) was added to the aqueous layer and the resulting solution was extracted repeatedly with EtOAc removing a small quantity of (4-(1H-pyrrolo[2,3-b]pyridin-1-yl)benzamide). The aqueous layer was fully basified (ca 450 mL 2N NaOH) and the resulting precipitate filtered, washed with water (2×200 mL) and 1:1 hexane-EtOAc (200 mL) and dried. Yield 62 g (80%). ¹H NMR (CDCl₃) δ 8.36 (dd, 1H, J=1.7, 5.0), δ 8.21 (d, 1H, J=2.0), 8.02 (m, 2H), 7.96 (dd, 1H, J=1.7, 7.9), 7.91 (m, 2H), 7.55 (d, 1H, J=3.7), 7.23 (dd, 1H, J=2.5, 7.9), 7.16-7.12 (m, 2H), 6.65 (d, 1H, J=3.7), 4.93 (br, 2H), 2.52 (s, 3H). MS (AP+) m/e 328 (MH+).

### Example 7

### 1-(4-(4-(pyridin-2-yl)-1-(pyridin-3-yl)-1H-imidazol-2-yl)phenyl)-1H-pyrrolo[2,3-b]pyridine

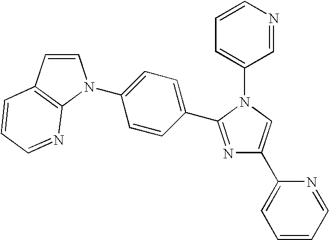

According to General Procedure 2, N′-(pyridin-3-yl)-4-(1H-pyrrolo[2,3-b]pyridin-1-yl)benzamidine (25.1 g, 80.0 mmol), 176 mL 1M LiHMDS in THF, and 2-bromo-1-(pyridin-2-yl)ethanone hydrobromide (22.5 g, 80.0 mmol) gave crude product which was purified by SGC (0.5%-5% ethanol in DCM, 0.5% aqueous NH₄OH), giving 12.7 g product in 5 fractions contaminated with between 2-8% of 4-(1H-pyrrolo[2,3-b]pyridin-1-yl)benzamide by HPLC (280 nM detection). A portion of this material (11.9 g) was recrystallized at RT in 75 mL acetonitrile containing 2% water using previously obtained seed crystals and dried at 100° C. Yield 8.0 g. M.P. 174-176° C. ¹H NMR (CDCl₃) δ8.68-8.66 (m, 2H), 8.57 (m, 1H), 8.35 (dd, 1H, J=1.7, 4.7 Hz), 8.19 (m, 1H), 8.05 (br, 1H), 7.95 (dd, 1H, J=1.7, 7.9 Hz), 7.85-7.80 (m, 3H), 7.65 (m, 1H), 7.58 (m, 2H), 7.51 (d, 1H, J=3.7 Hz), 7.39 (dd, 1H, J=4.8, 8.1 Hz), 7.13 (dd, 1H, J=5.0, 7.9 Hz), 6.63 (d, 1H, J=3.7). One resonance was not clearly seen and presumed to be under the chloroform peak. MS (AP+) m/e 415 (MH+). Anal. Calcd for C₂₈H₁₈N₈, C, 75.35; H, 4.38; N, 20.28. Found: C. 74.68; H, 4.01; N. 20.11. IC₅₀=6.84 nM.

### Preparation 7A

### N′-(pyridin-3-yl)-4-(H-pyrrolo[2,3-b]pyridin-1-yl)benzamidine

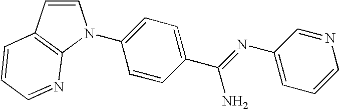

4-(1H-pyrrolo[2,3-b]pyridin-yl)benzonitrile (25.0 g, 114 mmol) and 3-aminopyridine (10.73 g, 114 mmol) were dissolved in anhydrous dimethylsulfoxide at RT and sodium hydride oil dispersion (5.5 g of 60% NaH by weight, 137 mmol) was added in one portion. After moderate foaming had subsided, the mixture was heated to 57° C. for 2.5 h. The mixture was cooled to 0° C. and 200 g of ice, 100 ml water, and 400 ml EtOAc were added sequentially. The organic layer was separated and the aqueous layer was extracted twice with EtOAc. The organic layers were combined, dried over MgSO₄, and concentrated. The residue was dissolved in 1N HCl (350 mL) and extracted with EtOAc (2×250 mL). DCM (200 mL) and aqueous 6N NaOH (80 mL) were added to the aqueous layer and the organic layer was separated. The aqueous layer was extracted successively with portions of DCM (800 mL total). EtOAc (150 mL) was added to the aqueous layer and the mixture was filtered to remove 0.9 g of a solid. The aqueous layer was separated and extracted with about 300 mL DCM. The organic layers were combined, 100 mL isopropyl alcohol was added, the resulting solution dried over Na₂SO₄ and concentrated giving 33.1 g of an orange foam. This material was dissolved in 150 mL DCM, and the resulting solution heated at reflux while 150 mL hexanes and seed crystals of the title substance were added. The mixture was stirred at RT for 30 min and filtered. The solid was washed twice with 50 mL of 1:1 DCM-hexanes (v/v) and dried giving 22.8 g (65%) of the title substance as a beige solid. ¹H NMR (CDCl_{3) δ8.37} (dd, 1H, J=1.7, 4.6), 8.32 (m, 2H), 8.01 (br, 2H), 7.97 (dd, 1H, J=1.7, 7.9), 7.91 (d, 2H, J=8.3), 7.56 (d, 1H, J=3.7), 7.30 (m, 2H), 7.14 (dd, 1H, J=5, 7.9), 6.65 (d, 1H, J=3.7), 5.0 (br, 2H). MS (AP+) m/e 314 (MH+).

### Example 8

### 1-(4-(1-(6-(1H-imidazol-1-yl)pyridin-3-yl)-4-(pyridin-2-yl)-1H-imidazol-2-yl)phenyl)-1-H-pyrrolo[2,3-b]pyridine

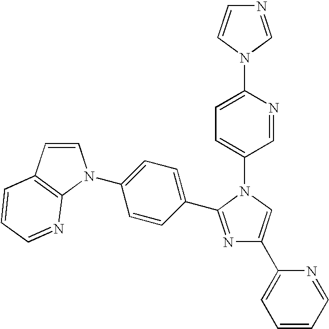

According to General Procedure 2, N′-(6-(1H-imidazol-1-yl)pyridin-3-yl)-4-(1H-pyrrolo[2,3-b]pyridin-1-yl)benzamidine (3.00 g, 7.9 mmol) and 2-bromo-1-(pyridin-2-yl)ethanone hydrobromide (2.22 g, 7.9 mmol) gave 700 mg of a chromatographed solid which was triturated with ether and dried. Yield 471 mg, 12% ¹H NMR (CDCl₃) δ 8.57 (m, 1H), 8.52 (d, 1H, J=2.5 Hz), 8.36 (s, 1H), 8.33 (dd, 1H, J=1.7, 4.5 Hz), 8.14 (d, 1H, J=7.9 Hz), 7.94 (dd, 1H, J=1.7, 7.9 Hz), 7.91 (s, 1H), 7.86 (m, 2H), 7.78 (dd, 1H), 7.75 (m, 1H), 7.63 (m, 2H), 7.60 (m, 1H), 7.51 (d, 1H, J=3.7 Hz), 7.41 (d, 1H, J=3.7 Hz), 7.21-7.18 (m, 2H), 7.13 (dd, 1H, J=4.6, 7.9 Hz), 6.63 (d, 1H, J=3.7 Hz). MS (AP+) m/e 481 (MH+). IC₅₀=1.48 nM.

### Preparation 8A

### N′-(6-(1H-imidazol-1-yl)pyridin-3-yl)-4-(1H-pyrrolo[2,3-b]pyridin-1-yl)benzamidine

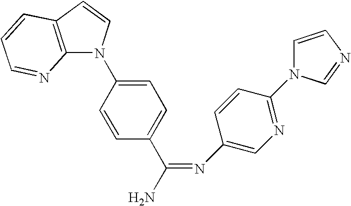

According to Procedure 1, 6-(1H-imidazol-1-yl)pyridin-3-amine (5.00 g, 31.2 mmol), 4-(1H-pyrrolo[2,3-b]pyridin-1-yl)benzonitrile (6.83 g), 2.74 g of 60% sodium hydride dispersion gave, after pouring the reaction mixture onto ice, a precipitate which was filtered, washed with water (5×200 mL) and ether, and dried at 100° C. in vacuo. Yield 9.21 g (78%). ¹H NMR (DMSO-d₆) δ 8.45 (s, 1H), 8.33 (m, 1H), 8.15-8.01 (m, 7H), 7.89 (s, 1H), 7.73 (d, 1H, 8.3 Hz), 7.45 (d, 1H, J=7.9 Hz), 7.21 (dd, 1H, J=4.6, 7.9 Hz), 7.08 (s, 1H), 6.79 (br, 2H), 6.75 (d, 1H, J=3.7 Hz). MS (AP+) m/e 380 (MH+).

### Preparation 8B

### 6-(1H-imidazol-1-yl)pyridin-3-amine

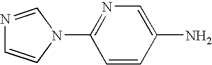

A mixture of 2-(1H-imidazol-1-yl)-5-nitropyridine (10.0 g, 52.6 mmol), 10% palladium-on-carbon (3 g), 1N HCl (105 mL) in MeOH (200 mL) was shaken under 45 p.s.i, hydrogen pressure for 2 h, filtered, and the filtrate evaporated. The residue was partitioned between 110 mL 2N NaOH and 150 mL DCM. The aqueous layer was separated and extracted twice with 150 mL 4:1 (v/v) DCM 2-propanol. The organic layers were dried and concentrated giving 7.43 g (89%) of the title substance. ¹H NMR (CDCl₃) δ 8.14 (s, 1H), 7.92 (m, 1H), 7.49 (m, 1H), 7.15-7.13 (m, 2H), 7.10 (dd, 1H, J=2.7, 8.5 Hz), 3.79 (br, 2H). MS (AP+) m/e 161 (MH+).

### Preparation 8C

### 2-(1H-imidazol-1-yl)-5-nitropyridine

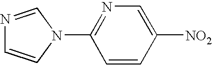

A mixture of 2-chloro-5-nitropyridine (50 g, 0.315 mol), imidazole (21.4 g, 0.315 mol), and potassium carbonate (33.4 g, 0.315 mol) in anhydrous dimethylsulfoxide (300 mL) was stirred at 100° C. for 1.5 h and poured into 500 mL ice water. The precipitate was filtered, washed with cold water (4×100 mL) and dried in vacuo Yield 42.3 g, 70.6%, ¹H NMR (DMSO-d₆) δ 9.27 (s, 1H), 8.76 (m, 1H), 8.66 (s, 1H), 8.05 (m, 2H), 7.16 (s, 1H).

### Example 9

### 1-(4-(1-(6-methoxypyridin-3-yl)-4-(pyridin-2-yl)-1H-imidazol-2-yl)phenyl)-1H-pyrrolo[2,3-b]pyridine

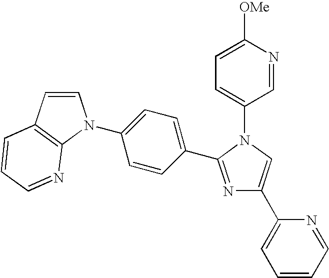

According to General Procedure 2, N′-(6-methoxypyridin-3-yl)-4-(1H-pyrrolo[2,3-b]pyridin-1-yl)benzamidine (460 mg. 1.34 mmol) and 2-bromo-1-(pyridin-2-yl)ethanone hydrobromide (376 mg, 1.34 mmol) gave 100 mg of the title substance which was triturated with ether-hexanes to give an off-white solid. Yield 60 mg, 10%. ¹H NMR (CDCl₃) δ 8.57 (m, 1H), 8.35 (dd, 1H, J=1.7, 4.6 Hz), 8.20 (d, 1H, J=2.9 Hz), 8.14 (m, 1H), 7.94 (dd, 1H, J=1.7, 7.9 Hz), 7.82-7.72 (m, 4H), 7.62 (m, 2H), 7.51-7.47 (m, 2H), 7.18 (m, 1H), 7.12 (dd, 1H, J=4.6, 7.9 Hz), 6.78 (d, 1H, J=8.7 Hz), 8.62 (d, 1H, J=3.7 Hz), 3.97 (s, 3H). MS (AP+) m/e 445 (MH+). IC₅₀=3.07 nM.

### Preparation 9A

### N′-(6-methoxypyridin-3-yl)-4-(1H-pyrrolo[2,3-b]pyridin-1-yl)benzamidine

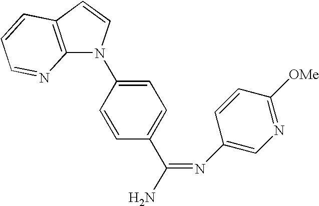

According to General Procedure 1, 6-methoxy-3-aminopyridine (14.1 g, 114 mmol) and 4-(1H-pyrrolo[2,3-b]pyridin-1-yl)benzonitrile (25 g, 114 mmol) gave a reaction mixture which was poured onto ice and stirred with 100 mL brine, 100 mL hexanes, and 100 mL EtOAc for 30 min The product was filtered and washed with water (5×200 mL) and hexanes (2×150 mL) and dried in vacuo with heat overnight. Yield 36 g off-white solid, 92%). ¹H NMR (CDCl₃) δ 8.32 (dd, 1H, J=12, 4.6), 8.12-8.02 (m, 6H), 7.68 (m, 1H), 7.24 (br, 1H), 7.20 (dd, 1H, J=4.6, 7.9), 6.76-6.73 (m, 2H), 6.54 (br, 2H). MS (AP+) m/e 344 (MH+).

### Example 10

### N,N-dimethyl-2-(1-(4-(4-(pyridin-2-yl)-1-(pyridin-3-yl)-1H-imidazol-2-yl)phenyl)-1H-pyrrolo[2,3-b]pyridin-3-yl)ethanamine

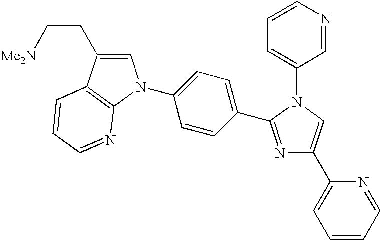

A mixture of 2-(2-(4-iodophenyl)-1-(pyridin-3-yl)-1H-imidazol-4-yl)pyridine (200 mg. 0.47 mmol), N,N-dimethyl-2-(1H-pyrrolo[2,3-b]pyridin-3-yl)ethanamine dihydrochloride (123 mg, 0.47 mmol, Eur. Pat. Appl. EP870768), copper iodide (4.5 mg, 0.024 mmol), K₃PO₄ (418 mg, 1.98 mmol), trans-1,2-diaminocyclohexane (6 mg, 0.047 mmol) and p-dioxane (1.5 ml) was heated at 110° C. with stirring in a screw cap vial for 22 h. The mixture was filtered through a short plug of silica eluting with DCM-MeOH, The (filtrate was concentrated and the resulting yellow solid triturated with ether to give 130 mg of an off-white solid. This material was recrystallized from DCM-ether giving the title substance (45 mg, 20%). ¹H NMR (CDCl₃) δ8.67 (m, 2H), 8.58 (m, 1H), 8.37 (m, 1H), 8.10 (m, 2H), 7.87 (s, 1H), 7.79-7.77 (m, 3H), 7.64 (m, 1H), 7.56 (m, 2H), 7.48 (s, 1H), 7.39 (dd, 1H, J=4.6, 7.9 Hz), 7.23-7.16 (m, 2H), 3.45 (m, 2H), 3.31 (m, 2H), 2.85 (s, 6H). MS (AP+) m/e 486 (MH+). IC₅₀=8.99 nM.

### Preparation 10A

### 4-iodo-N′-(pyridin-3-yl)benzamidine

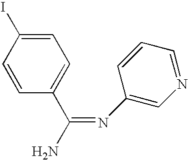

According to General Procedure 1, 4-iodobenzonitrile (11.45 mol), 3-aminopyridine (5.18 g, 55 mol), and 60% sodium hydride dispersion (2.6 g, 65 mmol) in 100 ml anhydrous dimethylsulfoxide at 55° C. for 3 h gave a reaction mixture which was treated with 100 ml water at less than 35° C. and extracted with 3×100 ml EtOAc. The organic layers were concentrated, and the residue dissolved in 100 mL EtOAc and 100 mL 1N HCl. The aqueous layer was separated and 100 mL EtOAc and 30 mL 6N NaOH were added. The organic layer was separated and combined with two further EtOAc extracts of the aqueous layer. These combined organic layers were dried and concentrated giving 9.64 g of a yellow solid which was the title substance contaminated with 3-aminopyridine. This material was dissolved with heating in 200 mL DCM and the resulting solution washed with water (3×30 mL), dried over MgSO₄, and concentrated. The solid was suspended in 2:1 DCM-hexanes, filtered, and washed with more of the same solvent mixture giving the title substance as a light yellow solid, Yield 6.8 g, 42%. ¹H NMR (CDCl₃) δ 8.30-8.26 (m, 2H), 7.78 (d, 2H, J=83 Hz), 7.58 (br, 2H), 7.27 (m, 2H), 4.9 (br, 2H). MS (AP+) m/e 324 (MH+)

### Preparation 10B

### 2-(2-(4-iodophenyl)-1-(pyridin-3-yl)-1H-imidazol-4-yl)pyridine

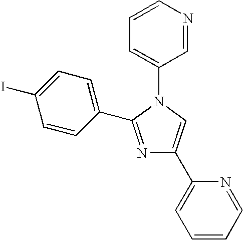

A solution of 4-iodo-N′-(pyridin-3-yl)benzamidine (6.0 g, 18.6 mmol) in anhydrous THF (100 mL) was treated at 0° C. with a THF solution of LiHMDS (41 mL of 1M). After being stirred at 0° C. for 30 min, 25° C. for 30 min, and 35° C. for 30 min, the solution was treated with water (100 mL) and EtOAc (100 mL). The organic layer was separated, dried, and concentrated and the residue was dissolved in 60 mL acetic acid. The resulting solution was heated at 90° C. for 30 min and concentrated. The residue was dissolved in 100 mL DCM and water, and the pH of the aqueous layer adjusted to >11 with 6N NaOH, The organic layer was separated and washed with aqueous 10% citric acid (3×30 mL), water, dried, and concentrated. SGC purification (gradient of 0.5-2% MeOH in DCM, 0.5% NH₄OH) of the residue (3.0 g) gave 2.0 g of the title substance as a light brown solid (25% yield), containing about 3% of the corresponding des-iodo analog by HPLCMS. ¹H NMR (CDCl₃) δ 8.63 (dd, 1H, J=1.5, 4.8 Hz), 8.57 (d, 1H, J=2.5 Hz), 8.54 (m, 1H). 8.07 (d, 1H, J=8.3 Hz), 7.85 (s, 1H). 7.73 (m, 1H). 7.62 (m, 2H), 7.55 (m, 1H), 7.36 (dd, 1H, J=4.6, 7.9 Hz), 7.18-7.11 (m, 3H). MS (AP+) m/e 425 (MH+).

### Example 11

### 1-(3-fluoro-4-(4-(pyridin-2-yl)-1-(pyridin-3-yl)-1H-imidazol-2-yl)phenyl)-1H-pyrrolo[2,3-b]pyridine

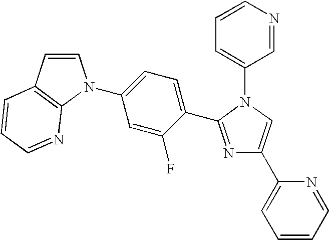

A mixture of 2-(2-(4-bromo-2-fluorophenyl)-1-(pyridin-3-yl)-1H-imidazol-4-yl)pyridine (200 mg, 0.51 mmol), 7-azaindole (72 mg, 0.61 mmol), CuI (5 mg, 0.03 mmol), trans-N,N′-dimethyl-cyclohexane-1,2-diamine (Strem Chemicals, 14.5 mg, 0.10 mmol), and K₃PO₄ (225 mg, 1.06 mmol) in toluene (5 mL) was heated at 120° C. for 48 h. HPLC analysis showed mostly starting bromide. The mixture was filtered and the filtrate evaporated and the residue was redissolved in p-dioxane (1 mL) and additional portions (the amounts specified above) of 7-azaindole, K₃PO₄. CuI, and trans-N,N′-dimethyl-cyclohexane-1,2-diamine were added and the resulting mixture irradiated in a microwave apparatus at 150° C. for 1 h, 180° C. for 5 h, and 200° C. for 2 h giving a mixture which was filtered, concentrated and purified by preparative RP-HPLC giving the product, an off-white solid, presumed to be the bis-TFA salt. Yield 47 mg, 21%. NMR (CDCl₃) δ 8.93 (s, 1H), 8.86 (d, 1H, J=4.6 Hz), 8.68 (dd, 1H, J=1, 5 Hz), 8.58-8.53 (m, 2H), 8.37 (dd, 1H, J=1.7, 4.6 Hz), 8.30 (m, 1H), 7.97 (dd, 1H, J=1.7, 7.9 Hz), 7.83-7.76 (m, 3H), 7.73 (dd, 1H, J=2, 11.6 Hz), 7.64 (m, 1H), 7.53 (d, 1H, J=3.7 Hz), 7.50 (dd, 1H, J=5.2, 8.1 Hz), 7.18 (dd, 1H, J=5.0, 7.9 Hz), 8.67 (d, 1H, J=3.7 Hz). MS (AP+) m/e 433 (MH+). IC₅₀=4.14 nM.

### Preparation 11A

### 4-Promo-2-fluoro-N-(pyridin-3-yl)benzamide

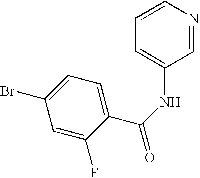

A mixture of 2-fluoro-4-bromobenzoic acid (3.09 g, 14.1 mmol) in thionyl chloride (7 mL) was stirred at RT for 18 h. The suspension was treated with dichoromethane (20 mL) and DMF (5 drops) and the mixture was heated at reflux 4 h, and concentrated to a yellow oil which was dissolved in chloroform (10 mL) and cooled to 0° C. This solution was treated with a mixture of 3-aminopyridine (1.33 g, 14.1 mmol) and pyridine (2.3 mL. 28.2 mmol) in chloroform (15 mL), and the resulting suspension stirred at RT for 3 days. The solid was filtered, washed with DCM and dried (1.27 g). The mother liquors were extracted with aqueous NaHCO₃ dried, concentrated and the residue purified by SGC giving another 2.0 g. Combined yield 1.27 g, 79%. ¹H NMR (CDCl₃) δ 8.68 (d, 1H, J=2.5 Hz), 8.44 (br, 1H), 8.39 (dd, 1H, J=1.5, 4.8 Hz), 8.26 (d, 1H, J=8.3 Hz), 8.03 (t, 1H, J=8.5 Hz), 7.47 (dd, 1H, J=1.7, 8.3 Hz), 7.39 (dd, 1H, J=1.7, 11.6 Hz), 7.32 (dd, 1H, J=4.6, 8.3 Hz). MS (AP+) m/e 295/297 (1:1, MH+).

### Preparation 11B

### 4-bromo-2-fluoro-N′-(pyridin-3-yl)benzamidine

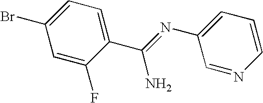

A suspension of 4-bromo-2-fluoro-N-(pyridin-3-yl)benzamide (1.25 g, 4.24 mmol) in toluene (15 mL) was treated with 970 mg (4.7 mmol) phosphorus pentachloride and the resulting mixture heated at reflux for 18 h. cooled and the solid filtered. A portion (1.0 g) of the solid was dissolved at RT in a saturated solution of ammonia in ethanol and heated at reflux for 16 h and the solution concentrated. SGC (1:1 EtOAc-hexanes, then EtOAc) gave the title product as a yellow solid. Yield 0.58 g. ¹H NMR (CDCl₃) δ 8.27-8.23 (m, 2H), 8.01 (br, 1H), 7.38-7.23 (m, 4H), 5.27 (br, 2H). MS (AP+) m/e 294/296 (1:1, MH+).

### Preparation 11C

### 2-(2-(4-bromo-2-fluorophenyl)-1-(pyridin-3-yl)-1H-imidazol-4-yl)pyridine

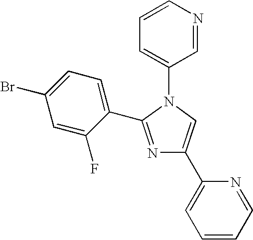

According to General Procedure 2, 4-bromo-2-fluoro-N′-(pyridin-3-yl)benzamidine (545 mg, 1.85 mmol), lithium bis-(trimethylsilylamide) (4.26 mmol of 1M in THF), and 2-bromo-1-(pyridin-2-yl)ethanone hydrobromide (519 mg, 1.85 mmol) gave after SGC a brown solid. Yield 220 mg, 30%. ¹H NMR (CDCl₃) δ 8.60 (dd, 1H, J=1.5, 4.8 Hz). 8.55 (m, 1H), 8.49 (d, 1H, J=2.1 Hz), 8.05 (dd, 1H, J=7.9 Hz), 7.95 (s, 1H), 7.74 (dd, 1H, J=1.9, 7.8 Hz), 7.60-7.56 (m, 1H), 7.55 (dd, 1H, J=1.5, 2.7, 8 Hz), 7.38 (dd, 1H, J=1.2, 8.3 Hz), 7.33 (m, 1H), 7.17 (ddd, 1H, J=12, 4.9, 7.6 Hz), 7.12 (dd, 1H, J=1.7, 9.5 Hz). MS (AP+) m/e 395/397 (1:1, MH+).

### Example 12

### 1-(2-methyl-4-(1-(6-methylpyridin-3-yl)-4-(pyridin-2-yl)-1H-imidazol-2-yl)phenyl)-1H-pyrrolo[2,3-b]pyridine

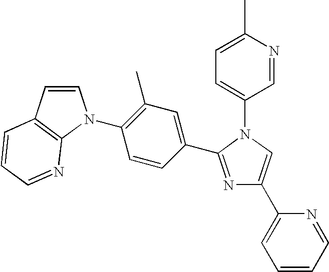

According to General Procedure 2, 3-methyl-4-(1H-pyrrolo[2,3-b[pyridin-1-yl)benzonitrile (450 mg, 1.32 mmol), LiHMDS (3.03 mL of 1M in THF), and 2-bromo-1-(pyridin-2-yl)ethanone hydrobromide (371 mg, 1.32 mmol) gave a chromatographed solid (103 mg) which was further purified by RP-HPLC to give the product as a light yellow solid, presumed to be a TFA salt. Yield 87 mg, 12%. ¹H NMR (CDCl₃) δ 8.87 (d, 1H, J=48 Hz), 8.71 (s, 1H), 8.61 (d, 1H, J=25 Hz), 8.58 (d, 1H, J=8.3 Hz), 8.35 (dd, 1H, J=1.7, 5.0 Hz), 8.31-8.27 (m, 1H), 8.14 (dd, 1H, J=1.6, 7.9 Hz), 7.76 (dd, 1H, J=2.5, 8.3 Hz), 7.64-7.60 (m, 2H), 7.39 (d, 1H, J=8.3 Hz), 7.3-7.2 (m, 4H), 6.72 (d, 1H, J=3.7 Hz), 2.89 (s, 3H), 2.08 (s, 3H). MS (AP+) m/e 443 (MH+). About 10% of another unidentified substance appearing to have two methyl groups (2.6, s, and 2.1, s) and a possible mass of 505 (MH+506 observed as a minor peak) was also present. IC₅₀=31.7 nM.

### Preparation 12A

### 3-methyl-4-(1H-pyrrolo[2,3-b]pyridin-1-yl)benzonitrile

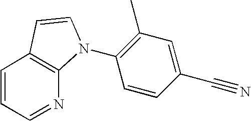

A mixture of 4-bromo-3-methylbenzonitrile (5.45 g, 27.8 mmol), N,N′-dimethylethylenediamine (0.6 mL, 5.56 mmol), CuI (530 mg, 2.78 mmol), sodium iodide (7.9 g, 52.8 mmol), 7-azaindole (3.28 g, 27.8 mmol), and K₃PO₄ (12.3 g, 58.4 mmol) in toluene (40 mL was heated at reflux for 36 h. The mixture was filtered, the filtrate evaporated, and the residue purified by SGC (5% and 10% EtOAc in hexane) giving a white solid. Yield 780 mg, 12%. ¹H NMR (CDCl₃) δ 8.30 (s, 1H), 7.99 (d, 1H, J=7.5 Hz). 7.88 (s, 1H), 7.82 (d, 1H, J=7.9 Hz), 7.46 (d, 1H, J=7.9 Hz), 7.25 (m, 1H), 7.13 (m, 1H), 6.67 (m, 1H), 218 (s, 3H). MS (AP+) m/e 234 (MH+)

### Preparation 12B

### 3-methyl-N′-(6-methylpyridin-3-yl)-4-(1H-pyrrolo[2,3-b]pyridin-1-yl)benzamidine

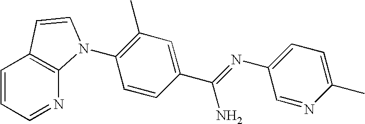

According to Procedure 1, 3-methyl-4-(1H-pyrrolo[2,3-b]pyridin-1-yl)benzonitrile (1.06 g, 4.61 mmol), sodium hydride dispersion (240 mg, 6 mmol) and 3-amino-6-methylpyridine (500 mg, 4.61 mmol) gave a reaction mixture which was poured onto ice and stirred with 30 mL 1:1 EtOAc-hexane giving a solid which was filtered, washed with water and hexanes and dried. Yield 850 mg, 54% ¹H NMR (CDCl₃) δ 8.28 (dd, 1H, J=1.2, 4.6 Hz), 8.20 (br, 1H), 7.98 (dd, 1H, J=1.7, 7.9 Hz), 7.9-7.7 (m, 2H), 7.40 (dd, 1H, J=2.9, 7.9 Hz), 7.27-7.22 (m, 2H), 7.14 (br, 1H), 7.10 (dd, 1H, J=4.8, 7.7 Hz), 6.64 (d, 1H, J=3.7 Hz), 4.9 (br, 2H), 2.52 (s, 3H), 2.14 (s, 3H). MS (AP+) m/e 342 (MH+).

### Example 13

### 1-(3-methyl-4-(1-(6-methylpyridin-3-yl)-4-(pyridin-2-yl)-1H-imidazol-2-yl)phenyl)-1H-pyrrolo[2,3-b]pyridin

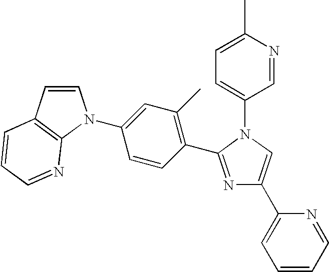

According to General Procedure 2, 2-methyl-N′-(6-methylpyridin-3-yl)-4-(1H-pyrrolo[2,3-b]pyridin-1-yl)benzamidine (900 mg, 2.64 mmol), LiHMDS (6.1 mL of 1M in THF) and 2-bromo-1-(pyridin-2-yl)ethanone hydrobromide (741 mg, 2.64 mmol) gave a chromatographed product (195 mg) which was further purified by RP-HPLC (basic conditions) giving a yellow solid. Yield 51 mg, 4.3%. ¹H NMR (CDCl₃) δ 8.58 (d, 1H, J=5 Hz), δ 8.46 (br, 1H), δ 8.36 (d, 1H, J=5 Hz), 8.16 (br, 1H), 7.95 (d, 1H, J=7.9 Hz), 7.80 (br, 1H), 7.70 (m, 2H), 7.51 (d, 1H, J=3.7 Hz), 7.43-7.40 (m, 2H), 7.25-7.21 (m, 2H), 7.15-7.11 (m, 2H), 6.62 (d, 1H, J=3.7 Hz), 2.55 (s, 3H), 2.25 (s, 3H), MS (AP+) m/e 443 (MH+). IC₅₀=37.1 nM.

### Preparation 13A

### 2-methyl-4-(1H-pyrrolo[2,3-b]pyridin-1-yl)benzonitrile

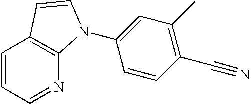

A mixture of 4-bromo-2-methylbenzonitrile (5.45 g, 27.8 mmol), N,N′-dimethylethylenediamine (0.6 mL, 5.56 mmol), CuI (530 mg, 2.78 mmol), and sodium iodide (7.9 g, 52.8 mmol) in toluene (50 mL) was heated at reflux for 28 h. K₃PO₄ (12.3 g, 58.4 mmol) and 7-azaindole (3.28 g, 27.8 mmol) were added and the mixture was heated at reflux for another 48 h, cooled, filtered, and concentrated. SGC (5% and 10% EtOAc-hexane) of the residue gave the title product as a colorless solid. Yield 2.8 g, 43%, ¹H NMR (CDCl₃) δ 8.37 (br, 1H), 7.96 (d, 1H, J=7.5 Hz), 7.86 (s, 1H), 7.80 (d, 1H, J=8.3 Hz), 7.71 (d, 1H, J=8.37 Hz), 7.51 (d, 1H, J=3.7 Hz), 7.17 (br, 1H), 6.67 (br, 1H), 2.62 (s, 3H) MS (AP+) m/e 234 (MH+).

### Preparation 13B

### 2-methyl-N′-(6-methylpyridin-3-yl)-4-(1H-pyrrolo[2,3-b)]pyridin-1-yl)benzamidine

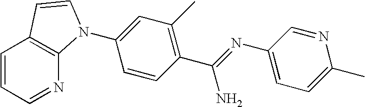

According to General Procedure 1, 2-methyl-4-(1H-pyrrolo[2,3-b]pyridin-1-yl)benzonitrile (1.83 g, 7.82 mmol), 3-amino-6-methylpyridine (845 mg. 7.82 mmol), and sodium hydride dispersion (407 mg, 10.2 mmol) gave a reaction mixture which was poured onto ice and 1:1 EtOAc-hexane (20 mL). A sticky solid was filtered and triturated with DCM-hexanes giving the title substance as a dark solid Yield 1.68 g, 63%). NMR (CDCl₃) δ 8.30 (d, 1H, J=4.6 Hz), 7.95 (d, 1H, J=7.9 Hz), 7.7-7.6 (m, 2H), 7.48 (br, 1H), 7.12 (dd, 1H, J=4.7, 7.7 Hz), 6.63 (d, 1H, J=3.7 Hz), 2.8-2.2 (br, 6H). MS (AP+) m/e 342 (MH+).

### Example 14

### 1-(4-(4-(pyridin-2-yl)-1-(1-oxido-pyridin-3-yl)-1H-imidazol-2-yl)phenyl)-1H-pyrrolo[2,3-b]pyridine

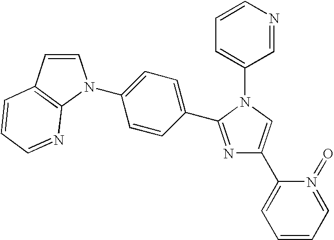

1-(4-(4-(pyridin-2-yl)-1-(pyridin-3-yl)-1H-imidazol-2-yl)phenyl)-1H-pyrrolo[2,3-b]pyridine (100 mg, 0.24 mmol) and 80% m-chloroperoxybenzoic acid (68 mg) were combined with 2 mg 3-t-butyl-4-hydroxy-5-methylphenyldisulfide in 2 mL chloroform and heated at reflux for 4 h. Another 48 mg m-chloroperoxybenzoic acid was added and the mixture heated 30 min then stirred at RT overnight. The mixture was dissolved in DCM and extracted with a 1:1 mixture of aqueous 1M sodium thiosulfate and aqueous 1M NaHCO₃, dried, and concentrated. SGC (1-8% ethanol in DCM) gave 46 mg of a yellow-brown foam. A single crystal X-ray analysis on a crystal obtained by allowing a portion of this material to stand in 98:2 acetonitrile-water confirmed the structure. ¹H NMR (CDCl₃) δ 8.87 (s, 1H), 8.68-8.67 (m, 2H), 8.51 (dd, 1H, J=2.1, 8.3 Hz), 8.34 (dd, 1H, J=1.5, 4.8 Hz), 8.32 (dd, 1H, J=1, 7 Hz), 7.95 (dd, 1H, J 1.7, 7.9 Hz), 7.83-7.80 (m, 2H), 7.67 (ddd, 1H, J=1.7, 2.6, 8.2 Hz). 7.60-7.56 (m, 2H), 7.50 (d, 1H, J=3.7 Hz), 7.42-7.35 (m, 2H), 7.16 (m, 1H), 7.13 (dd, 1H, J=4.8, 7.7 Hz), 6.63 (d, 1H, J=3.7 Hz). HPLCMS 7.288 min, m/e 431/883 (MH+, M₂Na⁺). IC₅₀=11.5 nM.

### Example 15

### 1-(4-(1-(1-oxido-6-methylpyridin-3-yl)-4-(1-oxido-pyridin-2-yl)-1H-imidazol-2-yl)phenyl)-1H-indole

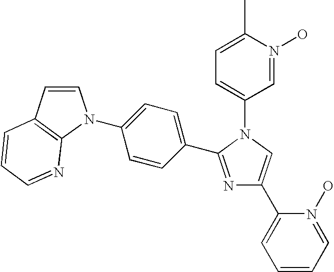

1-(4-(1-(6-methylpyridine-3-yl)-4-(pyridin-2-yl)-1H-imidazol-2-yl)phenyl)-1H-pyrrolo[2,3-b]pyridine (250 mg, 0.58 mmol), 3-t-butyl-4-hydroxy-5-methylphenyldisulfide (2 mg), and 77% m-chloroperoxybenzoic acid (302 mg. 1.75 mmol) were stirred in chloroform at RT for 18 h. A little MeOH was added to the resulting suspension and the resulting mixture was purified by SGC (1-2% MeOH in DCM, 0.5% NH₄OH) giving two substances. The more polar substance was identified as the title structure by single crystal X-ray analysis of a crystal obtained from acetonitrile containing 2% water. Yield 24 mg. ¹H NMR (CDCl₃) δ 8.85 (s, 1H), 8.49 (dd, 1H, J= 1.9, 8.1 Hz), 8.38 (d, 1H, J=21 Hz), 8.35 (dd, 1H, J=1.5, 4.8 Hz), 8.32 (d, 1H, J=6.6 Hz), 7.96 (dd, 1H, J=1.7, 4.9 Hz), 7.87 (m, 2H), 7.63 (m, 2H), 7.54 (d, 1H, J=3.7 Hz), 7.38 (m, 1H), 7.32 (d, 1H, J=8.7 Hz), 7.18 (dd, 1H, J=2.0, 6.6 Hz), 7.16-7.13 (m, 2H), 6.65 (d, 1H, J=3.7 Hz), 2.55 (s, 3H). MS (AP+) m/e 461 (MH+). IC₅₀=16.4 nM.

### Example 16

### 1-(4-(1-(6-methylpyridin-3-yl)-4-(1-oxido-pyridin-2-yl)-1H-imidazol-2-yl)phenyl)-1H-pyrrolo[2,3-b]pyridine

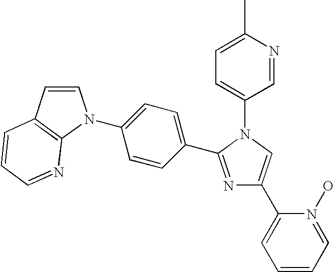

The less polar of two substances isolated from the metachloroperbenzoic acid oxidation of 1-(4-(1-(6-methylpyridin-3-yl)-4-(pyridin-2-yl)-1H-imidazol-2-yl)phenyl)-1H-pyrrolo[2,3-b]pyridine (preceding Example) was also isolated. Yield 23 mg. ¹H NMR (CDCl₃) δ 8.85 (s, 1H), 8.58 (d, 1H, J=2.5 Hz), 8.52 (dd, 1H) J=2.1, 8.3 Hz), 8.36 (dd, 1H, J=1.5, 4.8 Hz), 8.32 (d, 1H, J=6.6 Hz), 7.98 (dd, 1H, J=1.7, 7.9 Hz), 7.83 (m. 2H), 7.61 (m, 2H), 7.54 (dd, 1H, J=2.9, 7.5 Hz), 7.52 (d, 1H, J=3.7 Hz), 7.38 (m, 1H), 7.24 (d, 1H, J=8.7 Hz), 7.18-7.13 (m, 2H), 6.65 (d, 1H, J=3.7 Hz). 2.64 (s, 3H). MS (AP+) m/e 445 (MH+). IC₅₀=12.3 nM.

### Example 17

### 9-[4-(4-pyridin-2-yl-1-pyridin-3-yl-1H-imidazol-2-yl)phenyl]-5,7,8,9-tetrahydrothiopyrano[3′,4′,4,5]pyrrolo[2,3-b]pyridine

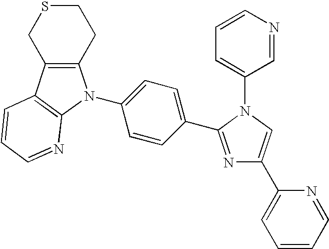

A mixture of 2-(2-(4-iodophenyl)-1-(pyridin-3-yl)-1H-imidazol-4-yl)pyridine (200 mg, 0.47 mmol), 5,7,8,9-tetrahydrothiopyrano[3′,4′,4, 5]pyrrolo[2,3-b]pyridine (90 mg, 0.47 mmol), CuI (4.5 mg, 0.024 mmol), K₃PO₄ (209 mg, 0.987 mmol) and trans-cyclohexanediamine (6 mg. 0.05 mmol) in p-dioxane (1 mL) was heated at 110° C. for 19 h, cooled, and filtered. The filtrate was concentrated and the residue purified by RP-HPLC giving 27 mg of the title substance. ¹H NMR (CDCl₃) δ 8.87 (d, 1H, J=5.8 Hz), 8.82 (br, 1H), 8.73 (dd, 1H, J=1.5, 4.8 Hz), 8.68 (d, 1H, J=2.5 Hz), 8.56 (d, 1H, J=8.3 Hz), 8.31-8.26 (m, 2H), 7.90 (dd, 1H, J=1.2, 7.9 Hz), 7.82 (m, 1H), 7.63-7.58 (m, 3H), 7.51 (dd, 1H, J=4.6, 8.3 Hz), 7.40 (m, 2H). 7.17 (dd, 1H, J=5.0, 7.9 Hz), 3.89 (m, 2H), 2.96 (m, 2H). 2.84 (m, 2H). MS (AP+) m/e 487 (MH+). IC₅₀=0.912 nm

### Example 18

### N,N-dimethyl(1-(4-(4-(pyridin-2-yl)-1-(pyridin-3-yl)-1H-imidazol-2-yl)phenyl)-1H-pyrrolo[2,3-b]pyridin-3-yl)methanamine TFA salt

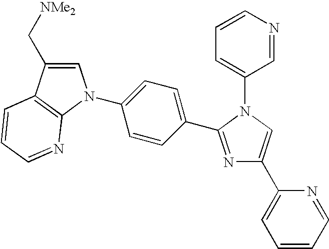

A mixture of 2-(2-(4-iodophenyl)-1-(pyridin-3-yl)-1H-imidazol-4-yl)pyridine (200 mg, 0.47 mmol), 3-(N,N-dimethylamino-methyl)-7-azaindole (83 mg, 0.47 mmol), CuI (5 mg, 0.024 mmol), K₃PO₄ (209 mg. 1 mmol) and trans-N,N′-dimethyl-cyclohexane-1,2-diamine (7 mg, 0.05 mmol) in p-dioxane (1 mL) was heated in an oil bath at 110° C. for 17 h and by microwave at 140° C. for 90 min. HPLC indicated about 50% conversion to the title substance. The mixture was filtered and concentrated. SGC (0.5% and 1% MeOH in DCM, 0.5% NH₄OH) gave 80 mg product which was further purified by RP-HPLC giving the title substance. Yield 28 mg. ¹H NMR (DMSO-d₆) δ 9.70 (br, 1H), 8.88 (m, 2H), 8.62 (d, 1H, J=5.0 Hz), 8.44 (br, 1H), 8.38 (dd, 1H, J=1.5, 4.8 Hz), 8.33 (dd, 1H, J=1.7, 7.9 Hz), 8.19 (s, 1H), 8.16-8.12 (m, 2H), 7.99-7.96 (m, 3H), 7.60-7.56 (m, 3H), 7.49 (m, 1H), 7.33 (dd, 1H, J=5.0, 7.9 Hz), 4.45 (d, 1H, J=4.6 Hz), 2.77 (s, 3H), 2.78 (s, 3H). MS (AP+) m/e 472 (MH+). IC₅₀=24.3 nM.

### Example 19

### 9-(4-(4-(pyridin-2-yl)-1-(pyridin-3-yl)-1H-imidazol-2-yl)phenyl)-9H-pyrido[2,3-b]indole

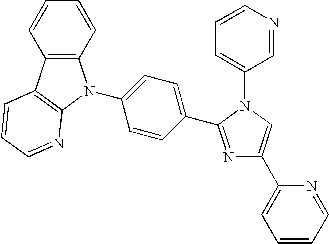

A mixture of 2-(2-(4-iodophenyl)-1-(pyridin-3-yl)-1H-imidazol-4-yl)pyridine (200 mg, 0.47 mmol), 9H-pyrido[2,3-b]indole (79 mg, 0.47 mmol), CuI (5 mg, 0.024 mmol), K₃PO₄ (209 mg, 1 mmol), and trans-N,N′-dimethyl-cyclohexane-1,2-diamine (7 mg, 0.05 mmol) in p-dioxane (1 ml) was heated in an oil bath at 110° C. for 18 h and filtered. Concentration and SGC (0.5% and 1% MeOH in DCM, 0.5% NH₄OH) gave the title substance as an off-white solid. Yield 103 mg. ¹H NMR (CDCl₃) δ 8.75 (d, 1H, J=2.5 Hz), 8.69 (dd, 1H, J=1.4, 4.8 Hz), 8.59 (d, 1H, J=4.1 Hz) 8.46 (dd, 1H, J=1.7, 5 Hz), 8.36 (dd, 1H, J=1.7, 7.9 Hz), 8.2 (d, 1H, J=7.5 Hz), 8.10 (d, 1H, J=7.9 Hz), 7.82 (br, 1H). 7.72-7.64 (m, 5H). 7.48-7.41 (m, 3H), 7.32 (m, 1H), 7.25-7.22 (m, 2H). MS (AP+) m/e 465 (MH+). IC₅₀=0.992 nM.

### Example 20

### 5-chloro-1-(4-(4-pyridin-2-yl)-1-(6-methylpyridin-3-yl-1H-imidazol-2-yl)phenyl)-1H-pyrrolo[2,3-b]pyridine

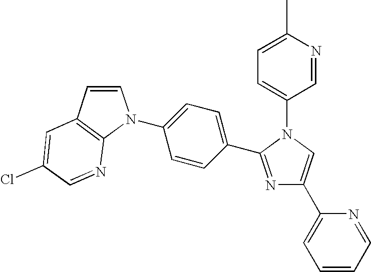

A mixture of 2-(2-(4-iodophenyl)-1-(6-methylpyridin-3-yl)-1H-imidazol-4-yl)pyridine (216 mg, 0.49 mmol), 5-chloro-1H-pyrrolo[2,3-b]pyridine (75 mg, 0.49 mmol), CuI (5 mg, 0.024 mmol), K₃PO₄ (218 mg, 1.03 mmol), and trans-N,N′-dimethyl-cyclohexane-1,2-diamine (7 mg, 0.05 mmol) In p-dioxane (1 mL) was heated in an oil bath at 110° C. for 18 h, cooled, filtered, and concentrated. SGC (0.5% and 1% MeOH in DCM, 0.5% NH₄OH) gave 107 mg of a yellow solid which was recrystallized from 98:2 acetonitrile-water. Yield 99 mg. ¹H NMR (CDCl₃) δ 8.57 (d, 1H, J=4 Hz), 8.54 (d, 1H, J=2.5 Hz), 8.27 (d, 1H, J=2.1 Hz), 8.15 (d, 1H, J=7.9 Hz), 7.91 (d, 1H, J=2.5 Hz). 7.78 (m, 1H), 7.75 (m, 2H), 7.60 (m, 2H). 7.53 (d, 1H, J= 3.7 Hz), 7.50 (dd, 1H, J=2.7, 8.1 Hz), 7.24-7.19 (m, 2H), 6.57 (d, 1H, J=3.7 Hz), 2.62 (s, 3H). MS (AP+) m/e 463 (MH+). IC₅₀=<2.72 nM.

### Preparation 20A

### 4-iodo-N-(6-methylpyridin-3-yl)benzamide

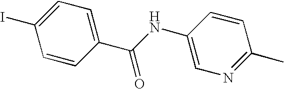

4-iodobenzoyl chloride (59 g, 0.22 mol) was added to a mixture of 6-methyl-3-aminopyridine (21.8 g, 0.201 mol) and triethylamine (56 g, 0.55 mol) in DCM (700 mL) at 0° C. and the mixture was warmed to RT. After 18 h the suspension was filtered and the solid washed with dichoromethane and dried giving 38 g of the title substance. The filtrate was was extracted with aqueous 5% NaOH (200 mL) and the organic layer which contained solid was filtered and dried (12.8 g of title substance). The organic layer was dried and concentrated. SGC of the residue (1% and 1.5% MeOH in DCM, 0.5% NH₄OH) gave 3.7 g of product. Also obtained was 4.7 g impure product which was triturated with ether giving 4.0 g of pure product. Yield 59.5 g, 87.5%. ¹H NMR (DMSO-d₆) δ 10.37 (s, 1H), 8.73 (d, 1H, J=2.5 Hz), 8.01 (dd, 1H, J=2.7, 8.5 Hz), 7.90 (m, 2H), 7.73 (m, 2H), 7.21 (d, 1H, J=83 Hz), 2.40 (s, 3H). MS (AP+) m/e 339 (MH+).

### Preparation 20B

### 4-iodo-N′-(6-methylpyridin-3-yl)benzamidine

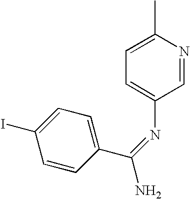

Phosphorus pentachloride (19.7 g, 95 mmol) was added to 4-iodo-N-(6-methylpyridin-3-yl)benzamide (30.5 g, 90.2 mmol) in phosphorus oxychloride (30 mL) and the resulting mixture heated at 105° C. (bath) for 18 h. The excess phosphorus oxychloride was removed by distillation at reduced pressure in a dry rotary evaporator. The residue, a tan solid, was added in portions to a solution of ammonia (40 g) in ethanol (1.3 L) at 0° C. Ammonia was bubbled into the resulting solution for 15 min, and the mixture was stirred at RT for 1.5 h and concentrated. The resulting solid (44 g) was dissolved in saturated aqueous NaHCO₃ and the resulting solution extracted twice with 200 mL portions of 5:1 DCM/2-propanol. The combined organic layers were dried and evaporated giving a yellow solid. Yield 29.3 g, 96%. ¹H NMR (CDCl₃) δ 8.14 (br, 1H), 7.77 (d, 2H, J=8.3 Hz), 7.56 (m, 2H), 7.19 (d, 1H, J=8 Hz), 7.11 (d, 1H, J=8 Hz), 4.9 (br, 2H), 2.50 (s, 3H). MS (AP+) m/e 338 (MH+).

### Preparation 20C

### 2-(2-(4-iodophenyl)-1-(6-methylpyridin-3-yl)-1H-imidazol-4-yl)pyridine

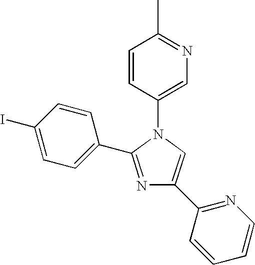

A solution of 4-iodo-N′-(6-methylpyridin-3-yl)benzamidine (23.0 g, 68.2 mmol) in anhydrous THF (150 mL) was treated at 0° C. with LiHMDS (150 mL of 1M in THF, 150 mmol). The resulting solution was treated after 15 min with 2-bromo-1-(pyridin-2-yl)ethanone hydrobromide (19.1 g, 68.2 mmol) and the resulting mixture stirred at RT for 18 h. Water (300 mL) and EtOAc (200 mL were added. The aqueous layer was separated and extracted with EtOAc (2×200 mL). The combined organic layers were dried and concentrated and the residue heated in acetic acid (200 mL) at 90° C. for 30 min. The mixture was concentrated and the residue partitioned between DCM (300 mL) and excess 2N NaOH, The aqueous layer was separated and extracted with DCM (3×200 mL). The combined organic layers were washed with aqueous 10% citric acid (3×100 mL), water, brine, dried, and concentrated. The residue was purified by SGC (0-1% MeOH in DCM, 0.5% NH₄OH) giving 7.6 g of product which was triturated with ether. Yield 6.5 g, 20%. ¹H NMR (CDCl₃) δ 8.56 (ddd, 1H, J=0.8, 1.7, 4.8 Hz). 8.48 (d, 1H, J=2.5 Hz). 8.10 (d, 1H, J=7.9 Hz), 7.89 (br, 1H). 7.77 (dt, 1H, J=1.7, 7.7 Hz), 7.63 (m, 2H), 7.43 (dd, 1H, J=2.5, 8.3 Hz), 7.21 (d, 1H, J=8.3 Hz), 7.2 (m, 1H), 7.16 (m, 2H), 2.62 (s, 3H). MS (AP+) m/e 439 (MH+).

### Preparation 20D

### 5-chloro-3-iodo-2-aminopyridine

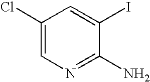

A mixture of 5-chloro-2-aminopyridine (54.7 mmol, 7.00 g), iodine (20.8 g, 82 mmol), and silver trifluoroacetate (14.5 g, 65.6 mmol) in chloroform (300 ml) was heated at reflux for 72 h. The mixture was filtered and the solid washed with DCM (150 ml). The filtrate was washed twice with aqueous 1M sodium thiosulfate, aqueous saturated NaHCO₃, dried, and concentrated giving 2.78 g of a crystalline solid which was triturated three times with 2:1 chloroform-hexanes (5 ml). The combined chloroform-hexane portions were concentrated to a dark oil yield 2.26 g, in which the title substance was the major component. ¹H NMR (CDCl₃) δ 7.96 (d, 1H, J=2.5 Hz), 7.81 (d, 1H, J=2.5 Hz), 4.95 (br, 2H). MS (AP+) m/e 255/257 (3:1, MH+).

### Preparation 20E

### 5-chloro-3-(2-(trimethylsilyl)ethynyl)pyridin-2-amine

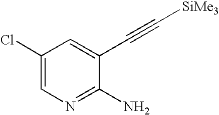

A mixture of 5-chloro-3-iodo-2-aminopyridine (2.23 g, 8.78 mmol), dichlorobis(triphenylphosphine)palladium(II) (184 mg, 0.26 mmol), CuI (50 mg, 0.03 mmol), and trimethylsilylethyne (1.29 g, 13.2 mmol) in DMF (3 mL) and triethylamine (3 ml) was heated 7 h at 55° C. (bath). The mixture was concentrated purified by SGC (loaded in DCM-triethylamine, eluted with 10-30% EtOAc-hexanes) giving the title substance. Yield 1.18 g, 59%. ¹H NMR (CDCl₃) δ 7.51 (s, 1H), 7.26 (s, 1H), 5.15 (br, 2H), 0.24 (s, 9H). MS (AP+) m/e 225/227 (MH+).

### Preparation 20F

### 5-chloro-3-ethynylpyridin-2-amine

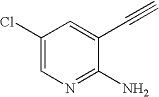

Tetrabutylammonium fluoride (1M in THF, 8 mL) was added to a solution of 5-chloro-3-(2-(trimethylsilyl)ethynyl)pyridin-2-amine (1.16 g, 5.16 mmol) in THF (10 ml) at RT. After 15 min the mixture was diluted with ether (125 ml) and the resulting solution extracted with water (2×30 mL), dried, and concentrated. The residue was purified by SGC (0-20% EtOAc-hexanes) giving the title substance as brown-yellow solid. Yield 515 mg, 65%. ¹H NMR (CDCl₃) δ 7.97 (d, 1H, J=2.1 Hz), 7.52 (d, 1H, J=2.1 Hz), 5.07 (br, 2H), 3.43 (s, 1H). HPLCMS 7.12 min, m/e 153 (MH+).

### Preparation 20G

### 5-Chloro-1H-pyrrolo[2,3-b]pyridine

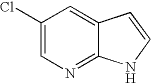

5-chloro-3-ethynylpyridin-2-amine (510 mg, 3.36 mmol), sodium gold tetrachloride dihydrate (51 mg, 0.13 mmol), 2 drops of water, and 10 mL absolute ethanol were combined and stirred 16 h at RT, 2.5 h at 65° C. and concentrated. The mixture was redissolved in 12 mL ethanol and 45 mg additional sodium gold chloride dihydrate was added and the mixture was heated at 80° C. for 16 h and concentrated. The solution was determined by NMR and HPLCMS to contain a 3.5:1 mixture of the title substance and 3-acetyl-2-amino-5-chloropyridine, a byproduct formed by hydration of the starting alkyne. The solution was concentrated and the residue purified by SGC (0-20% EtOAc-hexanes) giving the title substance. Yield 100 mg. An additional 240 mg of the title substance contaminated with the acetyl byproduct was also obtained, ¹H NMR (CDCl₃) δ 10.65 (br, 1H), 8.27 (d, 1H, J=2.1 Hz), 7.92 (d, 1H, J=2.1 Hz), 7.39 (m, 1H), 6.45 (dd, 1H, J=1.9, 3.5 Hz). HPLCMS 7.91 min, m/e 153/155 (3.1, MH+).

### Example 21

### 5-fluoro-1-(4-(1-(6-methylpyridin-3-yl)-4-(pyridin-2x-yl)-1H-imidazol-2-yl)phenyl)-1H-pyrrolo-[2,3-b]pyridine

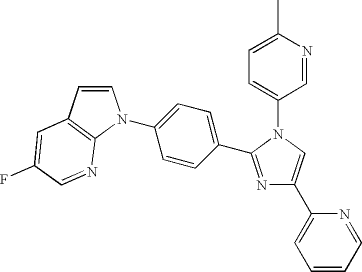

A mixture of 2-(2-(4-iodophenyl)-1-(6-methylpyridin-3-yl)-1H-imidazol-4-yl)pyridine (161 mg, 0.38 mmol), 5-fluoro-1H-pyrrolo[2,3-b]pyridine (50 mg assumed, 0.36 mmol), Cut (3 mg, 0018 mmol), K₃PO₄ (159 mg, 0.75 mmol), and trans-N,N′-dimethyl-cyclohexane-1,2-diamine (2 mg, 0.036 mmol) in p-dioxane (0.6 mL) was heated (microwave) at 150° C. for 3.5 h, 175° C. for 1 h, cooled and filtered. The filtrate was concentrated and a portion of this mixture purified by RP-HPLC (basic conditions). A yellow solid, 8 mg was obtained. By HPLCMS, 7% of the starting iodide was present in the sample. ¹H NMR (CDCl₃) δ 8.57 (m, 1H), 8.55 (m, 1H), 8.22 (m, 1H), 8.14 (d, 1H, J=7.5 Hz), 7.90 (br, 1H), 7.77 (m, 3H), 7.64-7.56 (m, 4H), 7.50 (dd, 1H, J=2.7, 8.1 Hz), 7.24-7.15 (m, 2H), 8.80 (d, 1H, J=3.7 Hz), 2.62 (s, 3H). HPLCMS 7.27 min (m/e 447, MH+). IC₅₀=2.82 nM.

### Preparation 21A

### 2-Amino-5-fluoro-3-iodopyridine

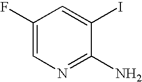

The following procedure is a modification of that of Dinnell (US2002 22624A1) for iodination of 5-chloro-2-aminopyridine. A mixture of 2-amino-5-fluoropyridine (5.0 g, 45 mmol), iodine (11.3 g, 45 mmol) and Ag₂SO₄ (14.0 g, 45 mmol) in ethanol was heated at reflux for 95 h, cooled, and filtered. The filtrate was concentrated and partitioned between 600 mL DCM and 200 mL 2N NaOH, The organic layer was separated, washed with water and dried giving a solid (4.6 g). The aqueous NaOH layer was extracted with 500 mL 4:1 DCM-2-propanol, dried and concentrated. The residue (1.1 g) was combined with the other solid. SGC (loaded in DCM, eluted with 20% EtOAc-hexanes) giving an orange solid. Yield 2.19 g, 20.4%. ¹H NMR (CDCl₃) δ 7.91 (d, 1H; J=2.7 Hz), 7.65 (dd, 1H, J=2.7, 7.3 Hz), 4.83 (br, 2H). MS (ES+) m/e 239 (MH+).

### Preparation 21B

### 5-fluoro-3-(2-(trimethylsilyl)ethynyl)pyridin-2-amine

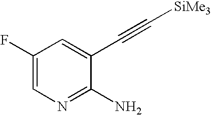

A mixture of 5-fluoro-3-iodo-2-aminopyridine (1.00 g, 4.2 mmol), dichlorobis(triphenylphosphine)palladium(II) (33 mg, 0.126 mmol), CuI (24 mg, 0.128 mmol), and trimethylsilylethyne (620 mg, 6.3 mmol) in DMF (2 mL) and triethylamine (4 mL) was heated 8 h at 50° C. (bath). The mixture was filtered, concentrated, and the residue purified by SGC (20% EtOAc-hexanes) giving a Sight brown solid. Yield 530 mg. 60%. ¹H NMR (CDCl₃) δ 7.90 (br, 1H), 7.28 (dd, 1H, J=2.5, 8.30 Hz), 4.89 (br, 2H). 0.24 (s, 9H),

### Preparation 21C

### 5-Fluoro-3-ethynyl-pyridine-2-amine

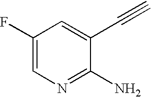

5-Fluoro-3-(2-(trimethylsilyl)ethynyl)pyridin-2-amine (281 mg, 1.35 mmol) was dissolved in 4 mL 1M tetrabutylammonium fluoride in THF at RT. After 1 h, the mixture was concentrated and the residue purified by SGC (10% and 20% EtOAc-hexanes) giving a light brown solid. Yield 51 mg, 28%. ¹H NMR (CDCl₃) δ 7.93 (br, 1H), 7.32 (dd, 1H, J=2.9, 8.3 Hz), 4.91 (br. 2H), 3.43 (s, 1H).

### Preparation 21D

### 5-Fluoro-1H-pyrrolo[2,3-b]pyridine

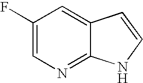

5-Fluoro-3-ethynyl-pyridine-2-amine (50 mg, 0.37 mmol) and sodium gold tetrachloride dihydrate (5 mg, 0.015 mmol) were combined in 1 ml ethanol and heated at 90° C. (bath) for 48 h. The mixture was concentrated and the residue used without purification. ¹H NMR (CDCl₃) δ (for the major substance) 9.79 (br, 1H), 8.20 (m, 1H), 7.72 (dd, 1H, J=2.9. 8.7 Hz), 7.44 (m, 1H), 6.53 (m, 1H). About 15% of another substance having 2.58 (s, 3H), 7.81 (dd, 1H, J=2.9, 8.3 Hz) and 7.53 (dd, 1H, J=2.9, 7.9 Hz) consistent with 2-amino-3-acetyl-5-fluoropyridine formed by hydration of the alkyne was also present.

### Example 22

### 5-methyl-1-(4-(1-[6-methylpyridin-3-yl)-4-(pyridin-2-yl)-1H-imidazol-2-yl)phenyl)-1H-pyrrolo[2,3-b]pyridine

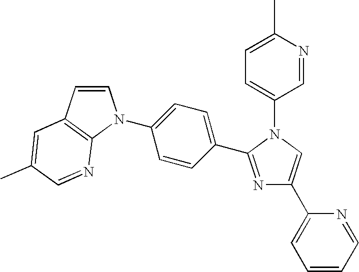

A mixture of 2-(2-(4-iodophenyl)-1-(6-methylpyridin-3-yl)-1H-imidazol-4-yl)pyridine (200 mg, 0.46 mmol), 5-methyl-1H-pyrrolo[2,3-b]pyridine (62 mg, 0.46 mmol), CuI (4 mg, 0.022 mmol), K₃PO₄ (210 mg, 1.0 mmol), and trans-N,N′-dimethyl-cyclohexane-1,2-diamine (12 mg, 0.05 mmol) in p-dioxane (1 mL) was heated by microwave at 150° C. for 1.5 h. Additional 5-methyl-7-azaindole (62 mg, 0.46 mmol), CuI (4 mg, 0.022 mmol), and diamine (12 mg) were added and the mixture heated by microwave at 160° C. for 1 h. The mixture was filtered, concentrated and the residue purified by SGC (1% and 2% MeOH in DCM, 0.5% NH₄OH) giving a solid which was triturated with ether and dried. Yield 18 mg. ¹H NMR (CDCl₃) δ8.57 (m, 2H), 8.18-8.12 (m, 2H), 7.90 (br, 1H), 7.82-7.73 (m, 4H), 7.59 (m, 2H), 7.51-7.47 (m, 2H), 7.23-7.18 (m, 2H), 6.54 (d, 1H, J=3.3 Hz), 2.62 (s, 3H), 2.43 (s, 3H). A second compound appearing to contain two methyl resonances was present in about 10% amount (s, 2.69), (s, 2.34). MS (ES+) m/e 443 (MH+). The material was homogeneous by HPLCMS: 6.85 min, m/e 443 (MH+). IC₅₀=12.1 nM.

### Example 22A

### 3-ethynyl-5-methylpyridin-2-amine

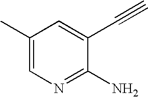

2-Amino-3-iodo-5-methylpyridine (8.95 g, 38.2 mmol), trimethylsilylacetylene (4.5 g, 45.9 mmol), 1,4-diazabicyclo[2.2.2]octane (7.27 g, 65 mmol), and dichlorobis(triphenylphosphine)palladium(II) (1.34 g, 1.91 mmol) were combined in DMF (45 mL) and the mixture heated at 110° C. for 16 h. The mixture was filtered, concentrated, and the residue purified by SGC (10%-30% EtOAc-hexanes) to isolate the more polar of two spots. A yellow solid (3.05 g) containing by NMR 3-trimethylsilylethynyl-2-amino-5-methylpyridine (identical to that reported by Abbiati (Synthesis 2002, vol 13, pp 191216)) and other aromatic substance(s) was obtained. Part of this material (2.07 g) was dissolved in 1M tetrabutylammonium fluoride in THF (30 mL) and stirred at RT for 1 h, concentrated, and the residue purified by SGC (10-30% EtOAc-hexanes). Yield 1.05 g, 60%, ¹H NMR (CDCl₃) δ 7.85 (d, 1H, J=1.7 Hz), 8.37 (d, 1H, J=2.1 Hz), 4.91 (br, 2H). 3.36 (s, 1H), 2.14 (s, 3H). IC₅₀=3.35 nM.

### Preparation 22B

### 5-Methyl-1H-pyrrolo[2,3-b]pyridine

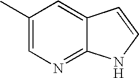

3-ethynyl-5-methylpyridin-2-amine (500 mg) and sodium gold tetrachloride dihydrate (68 mg, 0.2 mmol) were combined in 4 mL ethanol and the mixture heated at reflux for 18 h. filtered, concentrated, and the residue purified by SGC (5% MeOH in DCM, 0.5% NH₄OH) giving a yellow solid, in which the major substance was equivalent by NMR to that reported by Graczyk et al., (WO2004 078757). This was used without further purification. ¹H NMR (CDCl₃) δ 10.03 (br, 1H), δ 15 (d, 1H, J=2.1 Hz), 7.75 (m, 1H), 7.29 (d, 1H, J=3.7 Hz), 6.41 (d, 1H, J=3.7 Hz), 2.43 (s, 3H).

### Example 23

### 1-(4-(1-(pyridin-3-yl)-4-(thiazol-2-yl)-1H-imidazol-2-yl)phenyl)-1H-pyrrolo[2,3-b]pyridine

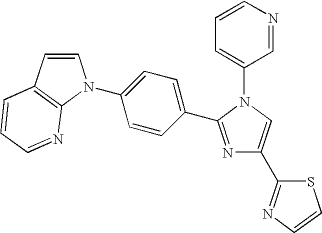

LiHMDS (70 mL of 1M in THF) was added to a solution of N′-(pyridin-3-yl)-4(1H-pyrrolo[2,3-b]pyridin-1-yl)benzamidine (21.0 g, 67.0 mmol) in THF (80 mL) at about −20° C. and the solution was stirred 10 min at 0° C. A solution of 2-bromoacetylthiazole (13.8 g, 67.0 mmol) in THF (85 mL) was added at about 0° C., The mixture was stirred at 10° C. for 30 min and at RT for 1 h Water (200 mL) and EtOAc (about 500 mL) were added, and the organic layer was separated, dried over Na₂SO₄, and concentrated giving 33.2 g of a foam which was dissolved in acetic acid (150 mL) and heated on a steam bath for 30 min. The mixture was concentrated and the residue dissolved in 600 mL EtOAc and extracted with 200 mL of 3N NaOH. The aqueous layer was separated and extracted with EtOAc (100 mL). The organic layers were combined, washed with brine, dried over MgSO₄ and concentrated giving 26.0 g of a brown foam. SGC (0%-16% ethanol in DCM, 0.5% NH₄OH) gave 8.1 g of the title substance. SGC of less pure fractions (66-100% EtOAc-hexanes, 0.5% triethylamine) gave a second pure batch of the title substance (2.8 g, 39% combined yield). This material dissolved readily in 150 mL acetone and quickly crystallized at RT. The suspension was concentrated at reflux to a volume of 90 mL, stirred at RT 30 min and at 0° C., filtered, and the solid washed with cold acetone and dried (6.65 g). MP 195-197° C. ¹H NMR (CDCl₃) δ 8.68 (m, 2H), 8.34 (dd, 1H, J=1.7, 4.6), 7.94 (dd, 1H, J=1.7, 7.9), 7.82-7.0 (m, 4H), 7.62 (m, 1H). 7.56 (m, 2H), 7.50 (d, 1H, J=3.7), 7.39 (dd, 1H, J=5.2, 8.5), 7.32 (d, 1H, J=3.3), 7.12 (dd, 1H, J=5.0, 7.9). 6.62 (d, 1H, J=3.7), MS (AP+) m/e 421 (MH+). Anal. Calcd for C₂₄H₁₆N₆S+0.2H₂O; C. 67.97; H, 3.90; N, 19.82. Found: C, 68.07; H, 3.80; N, 19.69. IC₅₀=3.32 nM.

### Example 24

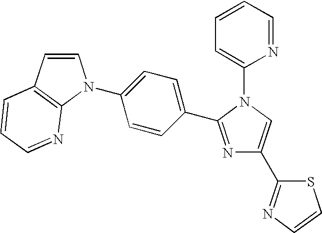

From N′-(pyridin-2-yl)-4-(1H-pyrrolo[2,3-b]pyridin-1-yl)benzamidine (930 mg, 2.97 mmol) and 2-bromoacetylthiazole (610 mg, 2.96 mmol) according to General Procedure 2. The chromatographed product was additionally triturated with ether-hexanes giving 89 mg of an off-white solid, ¹H NMR (CDCl₃) δ (partial) 8.59 (m, 1H). 8.35 (dd, 1H, J=1.7, 5.0), 8.09 (s, 1H), 7.95 (dd, 1H, J=1.7, 7.9), 7.83-7.80 (m, 3H), 7.78 (dt, 1H, J=1.9, 7.8), 7.61 (m, 2H), 7.51 (d, 1H, J=3.7), 7.33 (ddd, 1H, J=0.8, 5.0, 7.5), 7.29 (d, 1H, J=3.3), 7.29 (m, 1H), 6.63 (d, 1H, J=3.7). MS (AP+) m/e 421 (MH+). IC₅₀=13.4 nM.

### Preparation 24A

N′-(pyridin-2-yl)-4-(1H-(pyrrolo)[2,3-b]pyridin-1-yl)benzamidine

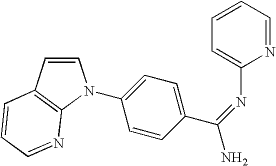

According to General Procedure 1, (1H-pyrrolo[2,3-b]pyridin-1-yl)benzonitrile (2.07 g, 9.45 mmol) and 2-aminopyridine (977 mg, 10.4 mmol) gave 3.3 g of a yellow solid which was boiled in 15 mL 1:1 DCM-hexanes, the suspension filtered at 0° C., and the resulting solid washed with cold 1:1 DCM-hexanes giving 1.95 g (66%) of the title substance as yellow crystals. ¹H NMR (CDCl₃) δ 8.37 (dd, 1H, J=1.5, 4.8). 8.33 (dd, 1H, J=1.2, 5.0). 8.08 (m, 2H), 7.96 (dd, 1H, J=1.7, 7.9). 7.91 (m, 2H), 7.65 (m, 1H), 7.55 (d, 1H, J=3.7), 7.29 (d, 1H, J=7.9), 7.14 (dd, 1H, J=5.0, 7.9), 6.93 (m, 1H), 6.65 (d, 1H, J=3.7). MS (AP+) m/e 314 (MH+).

### Example 25

### 1-(4-(1-(pyridin-4-yl)-4-(thiazol-2-yl)-1H-imidazol-2-yl)phenyl)-1H-pyrrolo[2,3-b]pyridine

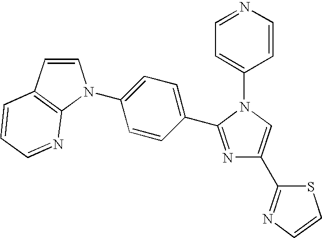

N′-(pyridin-4-yl)-4-(1H-pyrrolo[2,3-b]pyridin-1-yl)benzamidine (700 mg, 2.23 mmol) and 2-bromoacetylthiazole (460 mg, 2.24 mmol) were condensed according to Procedure 2, and the chromatographed product triturated with ether and dried (yellow solid, 85 mg). ¹H NMR (CDCl₃) δ 8.70 (m, 2H), 8.37 (dd, 1H, J=1.7, 4.6), 7.96 (dd, 1H, J=1.5, 7.7), 7.87-7.83 (m, 4H), 7.59 (m, 2H), 7.52 (d, 1H, J=3.7), 7.33 (d, 1H, J=3.3), 7.25 (m, 2H), 7.14 (dd, 1H, J= 5.0, 7.9), 6.65 (d, 1H, J=3.7). MS (AP+) m/e 421 (MH+). IC₅₀=4.46 nM.

### Preparation 25A

### N′-(pyridin-4-yl)-4-(1H-pyrrolo[2,3-b]pyridin-1-yl)benzamidine

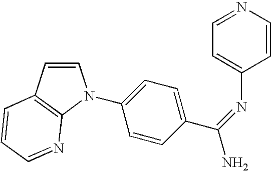

According to General Procedure 1. (1H-pyrrolo[2,3-b]pyridin-1-yl)benzonitrile (2.07 g, 9.45 mmol) and 2-aminopyridine (977 mg, 10.4 mmol) gave 1.4 g of a red solid which was dissolved in 5 ml DCM. Addition of 10 ml hexanes gave a precipitate which was filtered and dried (brown solid, 790 mg, 27%). ¹H NMR (CDCl₃) δ 8.50 (m, 2H), 8.37 (dd, 1H, J=1.7, 4.6), 8.0-7.9 (m, 5H, including 7.97 (dd, 1H, J=1.7, 7.9)), 7.55 (d, 1H, J=3.7), 7.15 (dd, 1H, J=4.8, 7.7), 6.91 (m, 2H), 6.66 (d, 1H, J=3.7), 4.95 (br, 2H). MS (AP+) m/e 314 (MH+).

### Example 26

### 1-(4-(1-(pyrimidin-5-yl)-4-(thiazol-2-yl)-1H-imidazol-2-yl)phenyl)-1H-pyrrolo[2,3-b]pyridine

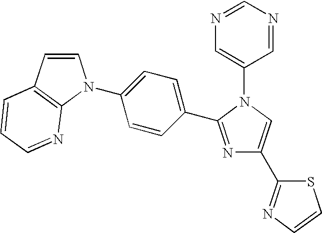

LiHMDS in hexanes (3.9 mL) was added to a solution of N′-(pyrimidin-5-yl)-4-(1H-pyrrolo[2,3-b]pyridin-1-yl)benzamidine (1.03 g, 3.28 mmol) in THF (8 mL) at 0-5° C. After 20 min, a solution of 2-bromoacetylthiazole (676 mg, 3.28 mmol) in THF (5 mL) was added at 0° C. The resulting solution was stirred 30 min at 0° C. and 30 min at RT. Water (20 mL) and EtOAc (90 mL) were added and the organic layer was separated, washed with water, dried over Na₂SO₄, concentrated, and the residue dissolved in acetic acid (15 mL), the solution heated at 80° C. for 30 min and concentrated. The residue was partitioned between EtOAc and 1N NaOH and the organic layer was separated, dried over Na₂SO₄, and concentrated giving a residue which was purified by SGC (2% MeOH in DCM, 0.5% NH₄OH). The product thus obtained was triturated with ether. Yield, 80 mg. ¹H NMR (CDCl₃) δ 9.07 (s, 1H), 8.77 (s, 2H), 8.35 (dd, 1H, J=1.5, 4.8), 7.95 (dd, 1H, J=1.5, 7.7), 7.90 (br, 1H), 7.90-7.86 (m, 2H), 7.85 (d, 1H, J=3.3), 7.57-7.54 (m, 2H), 7.51 (d, 1H, J=3.7). 7.35 (d, 1H, J=3.3), 7.14 (dd, 1H, J=5.0, 7.7), 6.64 (d, 1H, J=3.7). MS (AP+) m/e 422 (MH+). IC₅₀=4.40 nM.

### Example 27

### 1-(4-(1-(6-methylpyridin-3-yl)-4-(thiazol-2-yl)-1H-imidazol-2-yl)phenyl)-1H-pyrrolo[2,3-b]pyridine

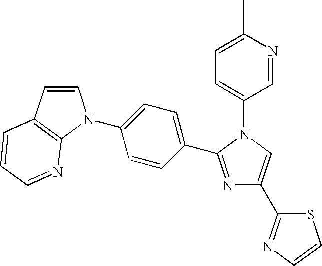

N′-(6-methylpyridin-3-yl)-4-(1H-pyrrolo[2,3-b]pyridin-1-yl)benzamidine (1.3 g, 3.97 mmol) and 2-bromoacetylthiazole (818 mg, 3.97 mmol) were condensed according to General Procedure 2 and the chromatographed product triturated with ether-hexanes giving 140 mg (8% yield) of the title substance. Another 320 mg of impure material was also obtained. ¹H NMR (CDCl₃) δ 8.65 (dd, 1H, J=1.5, 4.8), 8.33 (dd, 1H, J=1.7, 4.6), 793 (dd, 1H, J=1.7, 7.9), 7.82 (d, 1H, J=2.9), 7.77 (m, 2H), 7.65 (s, 1H), 7.61 (dd, 1H, J=1.2, 7.9), 7.55 (m, 2H), 7.48 (d, 1H, J=3.7), 7.32 (d, 1H, J=3.3), 7.29 (dd, 1H, J=5.0, 7.9), 7.11 (dd, 1H, J=4.8, 7.7), 6.61 (d, 1H, J=3.7), 2.35 (s, 3H). MS (AP+) m/e 435 (MH+). IC₅₀=1.48 nM.

### Example 28

### 1-(4-(1-(2-methylpyridin-3-yl)-4-(thiazol-2-yl)-1H-imidazol-2-yl)phenyl]-1H-pyrrolo[2,3-b]pyridine

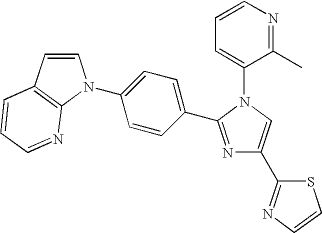

N′-(2-methylpyridin-3-yl)-4-(1H-pyrrolo[2,3-b]pyridin-1-yl)benzamidine (1.4 g, 4.28 mmol) and 2-bromoacetylthtazole (882 mg, 4.28 mmol) were condensed according to Procedure 2 and the chromatographed product triturated with ether-hexanes giving the pure title substance as a yellow solid (110 mg). Another lot of impure material (300 mg) was also obtained, ¹H NMR (CDCl₃) δ 8.66 (dd, 1H, J=1.5, 4.8), 8.33 (dd, 1H, J=1.7, 4.6), 7.93 (dd, 1H, J=1.7, 7.9), 7.82 (d, 1H, J=2.9), 7.77 (m, 2H), 7.65 (s, 1H), 7.61 (dd, 1H, J=1.2, 7.9), 7.55 (m, 2H), 7.48 (d, 1H, J=3.7), 7.32 (d, 1H, J=3.3). 7.29 (dd, 1H, J=5.0, 7.9), 7.11 (dd, 1H, J=4.8, 7.7), 6.61 (d, 1H, J=3.7), 2.35 (s, 3H), MS (AP+) m/e 435 (MH+). IC₅₀=44.1 nM.

### Preparation 28A

### N′-(2-methylpyridin-3-yl)-4-(1H-pyrrolo[2,3-b]pyridin-1-yl)benzamidine

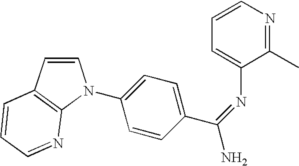

According to General Procedure 1, (1H-pyrrolo[2,3-b]pyridin-1-yl)benzonitrile (1.78 g. 8.14 mmol) and 2-methyl-3-aminopyridine (0.97 g, 8.9 mmol) gave 3.4 g of a yellow solid which was dissolved in DCM, precipitated with hexanes and filtered. This precipitation was repeated and the yellow solid dried (2.35 g, 88%).

¹H NMR (CDCl₃) δ 8.39 (dd, 1H, J=1.7, 4.6), 8.26 (dd, 1H, J=1.5, 4.8), δ 8.07 (m, 3H), 7.99 (dd, 1H, J=1.7, 7.9), 7.95 (m, 3H), 7.58 (d, 1H, J=3.7), 7.23 (m, 1H), 7.19-7.14 (m, 3H), 6.67 (m, 1H), 4.79 (br, 2H), 2.46 (s, 3H) MS (AP+) m/e 328 (MH+)

### Example 29

### 1-(4-(1-(6-methoxypyridin-3-yl)-4-(thiazol-2-yl)-1H-imidazol-2-yl)phenyl)-1H-pyrrolo[2,3-b]pyridine

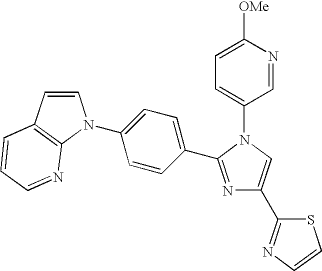

LiHMDS in THF (3.8 mL of 1M) was added at −20° C. to a suspension of N′-(6-methoxypyridin-3-yl)-4-(1H-pyrrolo[2,3-b]pyridin-1-yl)benzamidine (1.08 g, 3.15 mmol) in anhydrous THF (10 mL) and the resulting solution was stirred 20 min at −20° C. and 30 min at 0° C. A solution of 2-bromoacetylthiazole (650 mg, 3.15 mmol) in THF (5 mL) was added and the resulting mixture was stirred at 0° C. for 10 min and RT for 30 min. Water (20 mL) and EtOAc (90 mL) were added and the organic layer was separated, washed with water, dried over Na₂SO₄, concentrated. The residue was dissolved in acetic acid (15 mL) and the resulting solution heated at 80° C. for 35 min and concentrated. The residue was dissolved in EtOAc and water and the pH of the aqueous layer brought to 14 with aqueous NaOH, The aqueous layer was separated and extracted thrice with EtOAc. The combined organic layers were dried over Na₂SO₄ and concentrated. The residue was purified by SGC (0.5-1% MeOH in DCM, 0.5% NH₄OH) giving the title substance (145 mg). ¹H NMR (CDCl₃) δ 8.35 (dd, 1H, J=1.7, 5.0), 8.20 (d, 1H, J=2.0), 7.95 (dd, 1H, J=1.7, 7.9), 7.84-7.79 (m, 4H), 7.62-7.60 (m, 2H), 7.50 (d, 1H, J=3.7), 7.48 (dd, 1H, J=2.7, 8.9), 7.30 (d, 1H, J=2.9), 7.13 (dd, 1H, J=5.0, 7.9), 6.79 (d, 1H, J=8.7), 6.63 (d, 1H, J=3.7), 3.97 (s, 3H). MS (PP*) m/e 451 (MH+). IC₅₀=0.383 nM.

### Example 30

### 5-(2-(4-(1H-pyrrolo[2,3-b]pyridin-1-yl)phenyl)-4-(thiazol-2-yl)-1H-imidazol-1-yl)-N,N′-dimethylpyridin-2-mine

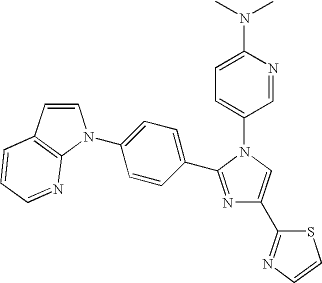

According to General Procedure 2, N′-(6-(dimethylamino)pyridin-3-yl)-4-(1H-pyrrolo[2,3-b]pyridin-1-yl)benzamidine (1.0 g, 2.81 mmol) and 2-bromoacetylthiazole (579 mg, 2.81 mmol) gave 130 mg of chromatographed product which was triturated with ether giving the title substance as a greenish solid (68 mg, 5% yield). ¹H NMR (CDCl₃) δ 8.34 (dd, 1H, J=1.7, 4.6), 8.17 (d, 1H, J=2.9), 7.94 (dd, 1H, J=1.7, 7.9), 7.80-7.77 (m, 3H), 7.68-7.65 (m, 3H), 7.51 (d, 1H, J=3.7), 7.30 (dd, 1H, J=2.5, 9.1), 7.28 (d, 1H, J=3.3). 7.11 (dd, 1H, J=5.0, 7.9), 6.61 (d, 1H, J=3.7), 6.48 (d, 1H, J=9.1), 3.12 (s, 6H). MS (AP+) m/e 464 (MH+). IC₅₀=0.607 nM.

### Preparation 30A

### N,N-dimethyl-5-nitropyridin-2-amine

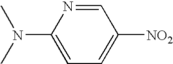

Dimethylamine gas (5 g) was introduced in to a solution of 2-bromo-5-nitropyridine (5 g, 24.6 mmol) in ethanol (20 ml) and the resulting solution was sealed in a thick wall glass vessel which was (CAUTION) heated for 17 h in a 150° C. oil bath behind a safety shield and concentrated to 6.4 g of a yellow solid. SGC (20-40% EtOAc-hexanes) giving 3.7 g (90%) of a yellow solid presumed to be the free base. ¹H NMR (CDCl₃) δ 9.02 (d, 1H, J=2.9 Hz), 8.16 (dd, 1H, J=2.9, 9.5 Hz), 6.43 (d, 1H, J=9.5), 3.20 (s, 6H),

### Preparation 30B

### N²,N²-dimethylpyridine-2,5-diamine

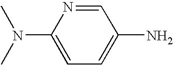

A mixture of N,N-dimethyl-5-nitropyridin-2-amine (3.5 g, 21 mmol) and 10% palladium on carbon (540 mg) in 25 mL MeOH and 25 mL EtOAc was shaken under 45 p.s.i. hydrogen pressure at RT for 1.5 h. The mixture was filtered through Celite and the filtrate concentrated to a red oil (2.8 g, 100%). ¹H NMR (CDCl₃) δ 7.75 (d, 1H, J=2.9), 6.98 (dd, 1H, J=2.9, 8.7), 6.43 (d, 1H, J=8.7), 3.17 (br, 2H), 2.97 (s, 6H).

### Preparation 30C

### N′-(6(dimethylamino)pyridin-3-yl)-4-(1H-pyrrolo[2,3-b]pyridin-1-yl)benzamidine

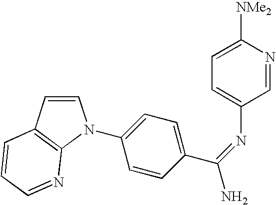

According to General Procedure 1, (1H-pyrrolo[2,3-b]pyridin-1-yl)benzonitrile (2.03 g, 9.27 mmol) and N²,N²-dimethylpyridine-2,5-diamine (1.39 g, 10.2 mmol) and the crude product obtained by EtOAc extraction was dissolved in 30 mL 2H HCl and 30 ml DCM. The aqueous layer was separated and basified to pH 14 with aqueous NaOH, and extracted with DCM (3×20 ml). The organic layers were dried and concentrated and the crude product purified by SGC (2%-5% MeOH in DCM, 0.5% NH₄OH) to give the title substance (1.0 g, 30%). ¹H NMR (CDCl₃) δ 8.39 (dd, 1H, J=1.7, 4.6), 8.04 (m, 2H), 7.98 (m, 2H), 7.92 (m, 2H), 7.58 (d, 1H, J=3.7), 7.25-7.23 (m, 1H), 7.16 (dd, 1H, J=4.6, 7.9), 6.67 (d, 1H, J=3.7), 6.6 (m, 1H), 4.9 (br, 2H), 3.09 (s, 6H). MS (AP+) m/e 357 (MH+).

### Example 31

### 2-(4-(2-(4-(1H-pyrrolo[2,3-b]-1-yl)phenyl-4-(thiazol-2-yl)-1H-imidazol-1-yl)phenyl)-N-methylethanamine

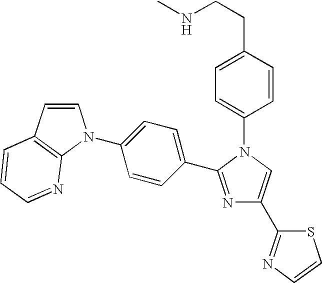

tert-Butyl 4-(2-(4-(1H-pyrrolo[2,3-b]pyridin-1-yl)phenyl)-4-(thiazol-2-yl)-1H-imidazol-1-yl)phenethylmethylcarbamate (50 mg, 0.14 mmol) was dissolved in 2 ml TFA at RT. After 15 min the mixture was concentrated and the residue was purified by SGC (loaded in DCM with 5 drops of triethylamine, eluted with 0.5-2% MeOH in DCM, 0.5% NH₄OH) giving a light brown solid. Yield 25 mg ¹H NMR (CDCl₃) δ 8.33 (dd, 1H, J=1.7, 4.6 Hz), 7.94 (dd, 1H, J=1.7, 7.9 Hz), 7.80 (d, 1H, J=3.3 Hz), 7.78-7.74 (m, 2H), 7.60-7.57 (m, 2H), 7.49 (d, 1H, J=3.7 Hz), 7.28-7.26 (m, 3H), 7.24-7.22 (m, 2H). 7.11 (dd, 1H, J=4.6, 7.9 Hz), 6.61 (d, 1H, J=3.7 Hz), 2.89 (m, 4H). 2.47 (s, 3H). MS (AP+) m/e 477 (MH+). IC₅₀=6.63 nM.

### Preparation 31A

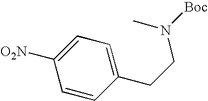

N-methyl-2-(4-nitrophenyl)ethanamine hydrochloride (8.00 g, 36.9 mmol), di-t-butyldicarbonate (8.86 g, 40.6 mmol), and triethylamine (4.11 g, 40.6 mmol) were combined in 100 ml THF, stirred for 1 h at RT, and concentrated. The residue was dissolved in EtOAc, the solution washed twice with aqueous 1N NaOH, dried and concentrated. Yield 10.1 g, 98%. ¹H NMR(CDCl₃) δ 8.15 (d, 2H, J=8.3 Hz), 7.34 (m, 2H), 3.47 (m, 2H), 2.95 (m, 2H). 2.84 and 2.79 (br s, 3H total)), 1.41 and 1.37 (br s, 9H total). The sample contained about 5% unreacted di-t-butyldicarbonate (s, 1.52).

### Example 31B

### tert-butyl 4-aminophenethylmethylcarbamate

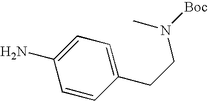

A mixture of tert-butyl 4-nitrophenethylmethylcarbamate (5.00 g, 17.6 mmol) and 10% palladium on carbon (2 g) in MeOH (100 ml) was shaken at 45 p.s.i. hydrogen pressure for 18 h, filtered, concentrated, and the residue purified by SGC (20% EtOAc-hexane, 0.5% Et₃N) giving a white solid. Yield 2.87 g, 65%. ¹H NMR (CDCl₃) δ 6.93 (m, 2H), 6.60 (m 2H), 3.55 (m, 2H), 3.31 (m, 2H), 2.82-2.60 (m, 5H), 1.39 (s, 9H),

### Preparation 31C

### tert-butyl 4-(2-(4-(1H-pyrrolo[2,3-b]pyridin-1-yl)phenyl)-4-(thiazol-2-yl)-1H-imidazol-1-yl)phenethylmethylcarbamate

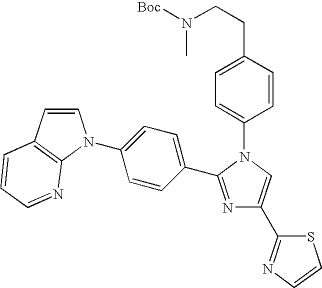

Sodium hydride oil dispersion (800 mg of 60%, 20 mmol) was added to a mixture of 4-(1H-pyrrolo[2,3-b]pyridin-1-yl)benzonitrile (2.00 g, 9.1 mmol) and tert-butyl 4-aminophenethylmethylcarbamate (2.30 g, 9.1 mmol) in anhydrous dimethylsulfoxide (20 mL). The resulting mixture was stirred at 55° C. for 3 h, cooled and poured onto ice, and extracted with EtOAc (2×300 mL). The organic layers were concentrated and the residue resolved in 1N HCl (50 mL), quickly extracted with EtOAc (3×) and the aqueous layer basified with 50 mL aqueous 2N NaOH and extracted with DCM (3×250 mL). The DCM layers were dried and concentrated to give 2.77 g of a solid whose NMR and MS were consistent with the desired amidine (estimated 70-80% purity). A portion of this material (1.00 g, 2.1 mmol) was cyclized according to General Procedure 2 employing 2.5 mL, of 1M LiHMDS in THF and 2-bromoacetylthiazole (2.1 mmol, 437 mg), extraction with EtOAc, dehydration of the crude product in acetic acid for 20 min at 80° C., extraction with DCM, and purification by SGC as specified therein. Yield 80 mg. ¹H NMR (CDCl₃, partial) δ 8.34 (dd, 1H, J=1.7, 4.6 Hz), 7.93 (dd, 1H, J=1.7, 7.5 Hz), 7.80 (d, 1H, J=3.3 Hz), 7.76-7.73 (m, 3H), 7.60-7.57 (m, 2H), 7.49 (d, 1H, J=3.3 Hz), 7.28 (d, 1H, J=3.3 Hz), 7.12 (dd, 2H, J=5, 79 Hz), 6.61 (d, 1H, J=3.7 Hz), 3.5-3.4 (m, 2H), 2.9-2.7 (m, 2H), 1.39 (s, 9H).

### Example 32

### 1-(4-(1-(6-(trifluoromethyl)pyridin-3-yl)-4-(thiazol-2-yl)-1H-imidazol-2-yl)phenyl)-1H-pyrrolo[2,3-b]pyridine

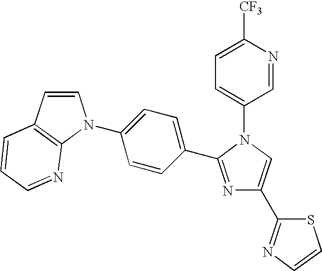

According to General Procedure 2, N′-(6(triftuoromethyl)pyridin-3-yl)-4-(1H-pyrrolo[2.3-b]pyridin-1-yl)benzamidine (500 mg, 1.3 mmol) and 2-bromoacetylthiazole (270 mg, 1.3 mmol) gave the crude title substance (460 mg), which was purified by SGC giving 23 mg of pure material and 206 mg of material contaminated with starting amidine. The impure material was dissolved in DCM and the solution washed with aqueous citric acid, dried and concentrated giving an additional 110 mg of pure product, ¹H NMR (CDCl₃) δ 8.78 (m, 1H), 8.35 (dd, 1H, J=1.7, 4.6), 7.95 (dd, 1H, J=1.7, 7.9), 7.87 (m, 2H), 7.84-7.83 (m, 2H), 7.78 (m, 2H), 7.56 (m, 2H). 7.52 (d, 1H, J=3.7), 7.34 (d, 1H, J=3.3). 7.14 (dd, 1H, J=4.6, 7.9). 6.64 (d, 1H, J=3.7). MS (AP+) m/e 489 (MH+), IC₅₀=2.13 nM.

### Preparation 32A

### N′-(6-(trifluoromethyl)pyridin-3-yl)-4-(1H-pyrrolo[2,3-b]pyridin-1-yl)benzamidine

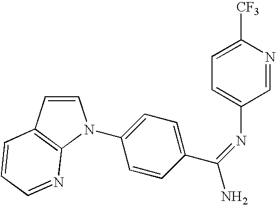

Sodium hydride oil dispersion (0.51 g of 60%) was added to a solution of 4-(1H-pyrrolo[2,3-b]pyridin-1-yl)benzonitrile (1.27 g, 5.8 mmol) and S-trifluoromethyl-2-methoxypyridine (0.95 g, 5.8 mmol) in anhydrous dimethylsulfoxide (12 mL) at RT and the mixture was heated at 55° C. for 1.5 h. The cooled mixture was poured onto ice and the aqueous mixture extracted with EtOAc (3×100 mL) The organic layers were dried, concentrated, and the residue dissolved in 15 mL of 1N HCl. The resulting solution was extracted twice with hexane, twice with ether, and the aqueous layer basified with NaOH (25 mL of 2N) and extracted with DCM (3×125 mL). The DCM extracts were dried, concentrated, and the residue purified by SGC (1-3% MeOH in DCM, 0.5% NH₄OH) giving 1.1 g of a brown paste which was triturated several times with 1:2 ether-hexanes giving 701 mg of a brown solid. ¹H NMR (CDCl₃) δ 8.42 (br, 1H), 8.37 (d, 1H, J=4), 8.03 (m, 2H), 7.98-7.93 (m, 3H), 7.67 (d, 1H, J=7.9). 7.57 (m, 1H), 7.46 (m, 1H), 7.15 (dd, 1H, J=4.6, 7.9), 6.67 (d, 1H, J=3.7), 4.96 (br, 2H) MS (AP+) m/e 382 (MH+).

### Example 33

### (4-(2-(4-(1H-pyrrolo[2,3-b]pyridin-1-yl)phenyl)-4-(thiazol-2-yl)-1H-imidazol-1-yl)phenyl-N-methylmethanamine Hydrochloride

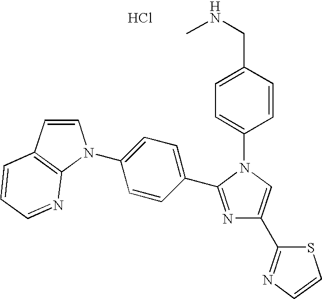

Tert-butyl 4-(2-(4-(1H-pyrrolo[2,3-b]pyridin-1-yl)phenyl)-4-(thiazol-2-yl)-1H-imidazol-1-yl)benzylmethylcarbamate (50 mg, 0.09 mmol) was dissolved in trifluoroacetic acid (1 ml) at RT. After 15 min the mixture was concentrated and the residue was dissolved in 0.5 mL 1N HCl. The resulting solution was concentrated and the residue triturated with 1:1 ether-hexanes and dried giving 40 mg of the title substance as a solid. NMR (DMSO-d₆, 400 mHz) δ 9.22 (br, 2H), 8.28 (dd, 1H, J=1.7, 4.5 Hz), 8.10 (s, 1H), 8.06 (dd, 1H, J=1.7, 8 Hz), 7.97 (d, 1H, J=3.7 Hz), 7.96 (m, 2H), 7.85 (d, 1H, J=3.3 Hz), 7.68 (d, 1H, J=3.3 Hz), 7.64 (A of AB, 2H, J=8-9 Hz), 7.53 (B of AB, 2H, J=8-9 Hz), 7.49 (m, 2H), 7.19 (dd, 1H, J=4.6, 7.9 Hz), 6.72 (d, 1H, J=3.7 Hz), 4.17-4.13 (m, 2H), 2.52-2.50 (t, 3H, J=5.4 Hz) MS (AP+) 483 (MH+), IC₅₀=14.1 nM.

### Preparation 33A

### N-methyl(4-nitrophenyl)methanamine

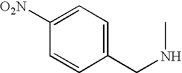

p-Nitrobenzaldehyde (15.0 g, 99.3 mmol) and 40% aqueous methylamine (17 ml) were combined in MeOH (250 mL) for 15 min at 0° C. for 15 min and then treated with sodium borohydride (3.77 g, 99.3 mmol). The mixture was stirred at RT for 2 h and concentrated. Water (50 mL) was added to the residue which was then extracted with DCM (3×250 mL). The extracts were dried and concentrated giving the title substance. Yield 15.4 g, 94%. ¹H NMR (CDCl₃) δ 8.10 (m, 2H), 7.43 (m, 2H), 3.79 (s, 2H), 2.39 (s, 3H). MS (AP+) m/e 167 (MH+).

### Preparation 33B

### tert-Butyl 4-nitrobenzylmethylcarbamate

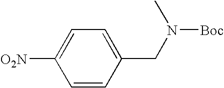

N-methyl(4-nitrophenyl)methanamine (14.3 g, 85.9 mmol) and di-t-butyldicarbonate (20.6 g. 94.5 mmol) were combined in THF at 0° C., stirred at RT for 1 h, and concentrated. The residue was dissolved in EtOAc (400 ml) and the solution washed with aqueous 1N NaOH (2×150 mL), dried, and concentrated. Yield 23.0 g. ¹H NMR (CDCl₃) δ 3.12 (d, 1H, J= 8 Hz), 7.33 (d, 8 Hz), 4.46 (br, 2H). 2.84 and 2.79 (br, 3H total), 1.43 and 1.37 (br, 9H total). MS (AP+) m/e 167 (MH-Boc).

### Preparation 33C

### tert-buty 14-aminobenzylmethylcarbamate

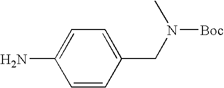

A mixture of tert-butyl 4-nitrobenzylmethylcarbamate (12.0 g, 45.1 mmol) and 10% palladium on carbon (5 g) in MeOH (120 ml) was shaken under 45 p.s.i. hydrogen pressure for 1 h at RT, filtered, concentrated, and the residue purified by SGC (0.5% and 1% MeOH in DCM, 0.5% NH₄OH). Yield 4.93 g, 46%. ¹H NMR (CDCl₃) δ 7.00 (br, 2H), 6.62 (m, 2H), 4.27 (br, 2H), 3.61 (br, 2H), 2.76 and 2.71 (br, 3H total), 1.45 (s, 9H)

### Preparation 33D

### tert-butyl 4-(4-(1H-pyrrolo[2,3-b]pyridin-1-yl)benzamido)benzylmethylcarbamate

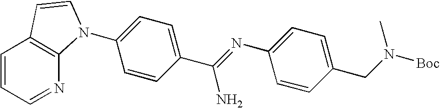

Sodium hydride oil dispersion (1.12 g of 60%) was added to a solution of 4-(1H-pyrrolo[2,3-b]pyridin-1-yl)benzonitrile (2.78 g, 12.7 mmol) and tort-butyl 4-aminobenzylmethylcarbamate (3.00 g, 12.7 mmol) in anhydrous dimethylsulfoxide (25 mL) at RT and the mixture was heated at 55° C. for 1.5 h. The cooled mixture was poured onto ice and the aqueous mixture extracted with EtOAc (2×300 mL). The EtOAc was concentrated and the residue purified by SGC (1% MeOH in DCM, 1% triethylamine) giving a yellow solid. Yield 1.81 g, 31%. ¹H NMR (CDCl_{3) δ} 8.36 (dd, 1H, J=1.7, 4.6 Hz), 7.99 (br, 2H), 7.96 (dd, 1H, J=1.7, 7.9 Hz), 7.88 (d, 2H, J=8.7 Hz), 7.54 (d, 1H, J=3.7 Hz), 7.20 (d, 2H. J=7.13 (dd, 1H, J=4.6, 7.9 Hz), 6.95 (dd, 2H, J=8.3 Hz). 6.64 (d, 1H, J=3.7 Hz), 4.9 (br, 2H), 4.37 (br, 2H). 2.82 and 2.79 (br s. 3H total), 1.47 (s, 9H). MS (AP+) m/e 456 (MH+).

### Preparation 33E

### tert-Butyl 4-(2-(4-(1H-pyrrolo[2,3-b]pyridin-1-yl)phenyl)-4-(thiazol-2-yl)-1H-imidazol-1-yl)benzylmethylcarbamate

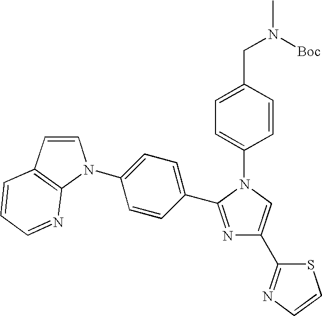

tert-Butyl 4-(4-(1H-pyrrolo[2,3-b]pyridin-1-yl)benzamido)benzylmethylcarbamate (162 mg, 0.36 mmol), 2-bromoacetylthiazole (109 mg, 0.53 mmol), and NaHCO₃ (61 mg, 0.72 mmol) were combined in 2-propanol (2 mL) and heated at 72° C. for 2 h. The mixture was filtered and concentrated. Another 62 mg NaHCO₅ and 5 mL 2-propanol was added and the mixture heated at 92° C. for 5 h. The mixture was filtered, concentrated, and the residue heated in 3 mL acetic acid at 60° C. for 10 min. The solution was concentrated and the residue digested in excess 1N NaOH and extracted with DCM (2×50 mL). The extracts were dried and concentrated and the residue purified by SGC (0.5% MeOH in DCM, 0.5% NH₄OH). Yield 51 mg. ¹H NMR (CDCl₃) δ 8.33 (dd, 1H, J=1.5, 4.8 Hz), 7.94 (dd, 1H, J=1.7, 7.9 Hz), 7.80 (d, 1H, J=3.3 Hz), 7.77-7.75 (m, 3H), 7.81-7.57 (m, 2H), 7.49 (d, 1H, J=3.7 Hz). 7.28 (m, 5H). 7.11 (dd, 1H, J=4.6, 7.9 Hz), 4.47 and 4.43 (br s, 2H total), 2.88 and 2.83 (br s, 3H total), 1.46 and 1.43 (br s, 9H total). MS (AP+) m/e 563 (MH+) and 463 (MH-C₄—H₈CO₂).

### Example 34

### 1-(4-(1-(6-morpholinopyridin-3-yl)-4-(thiazol-2-yl)-1H-imidazol-2-yl)phenyl)-1H-pyrrolo[2,3-b]pyridine

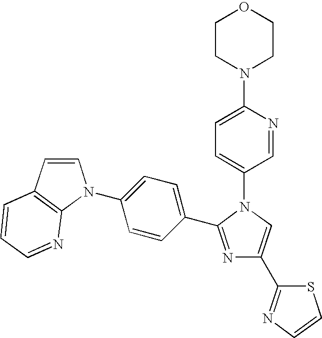

N′-(6-morpholinopyridin-3-yl)-4-(1H-(1H-pyrrolo[2,3-b]pyridin-1-yl)benzamidine (300 mg, 2.30 mmol) and 2-bromoacetylthiazole (484 mg, 2.30 mmol) were condensed according to Procedure 2, and the chromatographed product triturated with ether (91 mg, light brown solid). ¹H NMR (CDCl₃) δ 8.35 (dd, 1H, J=1.7, 4.6). 8.20 (d, 1H, J=2.9), 7.95 (dd, 1H, J=1.7, 7.9), 7.81-7.79 (m, 3H). 7.70 (br, 1H), 7.65 (m, 2H), 7.51 (d, 1H, J=3.7), 7.37 (dd, 1H, J= 2.7, 8.9), 7.29 (d, 1H, J=3.3), 7.13 (dd, 1H, J=4.8, 7.7), 6.63-6.60 (m, 2H), 3.82 (m, 4H). 3.56 (m, 4H). MS (AP+) m/e 506 (MH+). IC₅₀=0.436 nM.

### Preparation 34A

### N′-(6-morpholinopyridin-3-yl)-4-(1H-pyrrolo[2,3-b]pyridin-1-yl)benzamidine

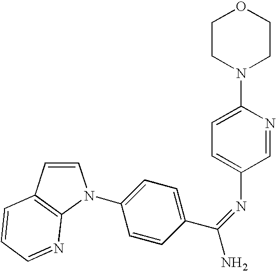

According to General Procedure 1, (1H-pyrrolo[2,3-b]pyridin-1-yl)benzonitrile (1-5 g, 8.4 mmol), 3-amino-6-morpholinopyridine (18 g, 8.4 mmol) and 740 mg (2.2 equiv sodium hydride dispersion) gave, after quenching with water and extraction with EtOAc, an aqueous layer which contained a suspended solid. This aqueous layer was tittered and the solid was dissolved in 150 mL 4:1 DCM/2-propanol. The resulting solution was dried and concentrated, and the resulting solid triturated with ether (1.04 g, 31%). ¹H NMR (CDCl₃) δ 8.38 (dd, 1H, J=1.7, 4.6), 8.06-7.89 (m, 6H), 7.55 (d, 1H, J=3.7), 7.27 (br, 1H), 7.14 (dd, 1H, J=4.6, 7.9), 6.71-6.68 (m, 1H), 6.65 (d, 1H, J=3.7), 4.9 (br, 2H). 3.83 (m, 4H), 3.44 (m, 4H). MS (AP+) m/e 399 (MH+).

### Example 35

### 1-(4-(4-(pyridin-2-yl)-1-(pyridin-3-yl)-1H-imidazol-2-yl)phenyl)-1H-indazole

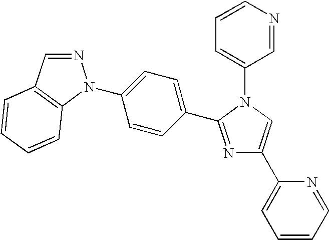

A mixture of 2-(2-(4-iodophenyl)-1-(pyridin-3-yl)-1H-imidazol-4-yl)pyridine (200 mg, 0.47 mmol), indazole (67 mg. 0.56 mmol), Cut (134 mg, 0.7 mmol). K₃PO₄ (199 mg, 0.94 mmol) and trans-1,2-cyclohexanediamine (6 mg, 0.05 mmol) in p-dioxane (6 mL) was heated at 115° C. (bath) for 18 h. Additional indazole (67 mg) was added and the mixture was heated by microwave at 150° C. for 20 h, filtered, concentrated, and the residue purified by SGC (0.5-1.5% MeOH in DCM, 0.5% NH₄OH). Yield 25 mg, 13%, an off-white solid. ¹H NMR (CDCl₃) δ 8.69 (d, 1H, J=2.5 Hz), 8.67 (dd, 1H, J=1.5, 4.8 Hz), 8.56 (d, 1H, J=4.5 Hz), 8.9 (s, 1H), 8.15 (d, 1H, J=79 Hz), 7.93 (s, 1H), 7.79 (d, 1H, J=8.3 Hz), 7.76 (m, 1H), 7.71 (m, 2H), 7.63 (m, 1H), 7.60 (m, 2H), 7.43 (m, 1H), 7.39 (dd, 1H, J=4.8, 8.1 Hz), 7.24 (m, 1H), 7.20 (m, 1H) MS (AP+) m/e 415 (MH+). IC₅₀=27.8 nM.

### Example 36

### 1-(4-(4-(pyridin-2-yl)-1-(pyridin-3-yl)-1H-imidazol-2-yl)phenyl)-1H-indole

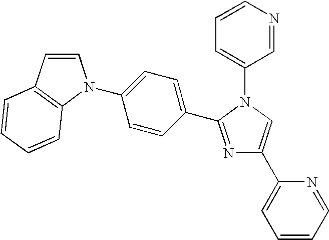

A mixture of 2-(2-(4-iodophenyl)-1-(pyridin-3-yl)-1H-imidazol-4-yl)pyridine (200 mg. 0.47 mmol), indole (66 mg, 0.56 mmol), CuI (134 mg, 0.7 mmol), K₃PO₄ (199 mg, 0.94 mmol) and trans-1,2-cyclohexanediamine (8 mg, 0.05 mmol) in p-dioxane (4 ml) was heated at 120° C. (bath) for 18 h, filtered, concentrated, and the residue purified by SGC (0.5-1.5% MeOH in DCM, 0.5% NH₄OH). Yield 60 mg, 31%, an off-white solid. ¹H NMR (CDCl₃) δ 8.68 (dd, 1H, J=1.5, 5 Hz), 8.58 (d, 1H, J=4 Hz), 8.14 (d, 1H, J=7.9 Hz), 7.91 (s, 1H), 7.77 (dt, 1H, J=1.7, 7.7 Hz), 7.68-7.65 (m, 2H), 7.59-7.53 (m, 3H), 7.46 (m, 2H), 7.42 (dd, 1H, J=5.0, 7.9 Hz), 7.31 (d, 1H, J=3.3 Hz), 7.21-7.14 (m, 4H), 6.67 (dd, 1H, J=˜1, 3.3 Hz). MS (AP+) m/e 414 (MH+). IC₅₀=281 nM.

### Example 37

### 7-fluoro-1-(4-(4(pyridin-3-yl)-1H-imidazol-2-yl)phenyl)-1H-indole

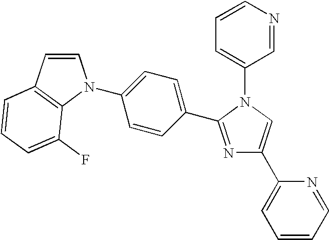

A mixture of 2-(2-(4-iodophenyl)-1-(pyridin-3-yl)-1H-imidazol-4-yl)pyridine (200 mg, 0.47 mmol), indole (76 mg, 0.56 mmol), CuI (134 mg, 0.7 mmol), K₅PO₄ (199 mg, 0.94 mmol) and trans-1,2-cyclohexanediamine (6 mg, 0.05 mmol) in p-dioxane (2 ml) was heated at 150° C. (bath) for 2 h, filtered, concentrated, and the residue purified by SGC (0.5 and 1% MeOH in DCM, 0.5% NH₄OH). Yield 80 mg, 39%. ¹H NMR (CDCl₃) δ 8.67 (m, 2H), 8.57 (m, 1H), 8.14 (d, 1H, J=7.9 Hz), 7.93 (s, 1H), 7.77 (dt, 1H, J=1.7, 7.7 Hz), 7.64 (m, 1H), 7.50 (m, 2H), 7.42-7.37 (m, 4H), 7.22-7.17 (m, 2H), 7.05 (m, 1H), 6.89 (dd, 1H, J=7.5, 13 Hz), 6.68 (dd, 1H, J=2.5, 3.3 Hz). MS (AP+) m/e 432 (MH+). IC₅₀=30.8 nM.

### Example 38

### 4,5,6,7-tetratfluoro-1-(4-(4-(pyridin-2-yl)-1-(pyridin-3-yl)-1H-imidazol-2-yl)phenyl)-1H-indole

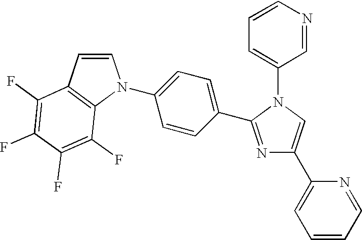

A mixture of 2-(2-(4-iodophenyl)-1-(pyridin-3-yl)-1H-imidazol-4-yl-pyridine (200 mg, 0.47 mmol), 4,5,6,7-tetrafluoroindole (106 mg, 0.56 mmol), CuI (134 mg, 0.7 mmol), K₃PO₄ (199 mg, 0.94 mmol) and trans-1,2-cyclohexanediamine (6 mg, 0.05 mmol) in p-dioxane (2 mL) was heated by microwave at 150° C. for in, filtered, concentrated, and the residue purified by SGC (0.5 and 1% MeOH in DCM, 0.5% NH₄OH). Yield 70 mg, 31%. ¹H NMR (COCl₃) δ 8.68 (dd, 1H, J=1.5, 4.9 Hz). 8.64 (d, 1H, J=2 Hz), 8.56 (ddd, 1H, J=1.2, 5 Hz), 8.13 (dt, 1H, J=˜1.8 Hz), 7.93 (s, 1H), 7.77 (dt, 1H, J=2, 7.9 Hz), 7.65 (ddd, 1H, J=1.7, 2.5, 8.2 Hz), 7.65 (m, 2H), 7.42 (ddd, 1H, J=1, 5, 8 Hz), 7.37-7.34 (m, 2H), 7.19 (m, 2H), 6.76 (dd, 1H, J=2, 3.3 Hz). MS (AP+) m/e 486 (MH+). IC₅₀=170 nM.

### Example 39

### 4-chloro-1-(4-(4-(pyridin-2-yl)-1-(pyridin-3-yl)-1H-imidazol-2-y TFA salt

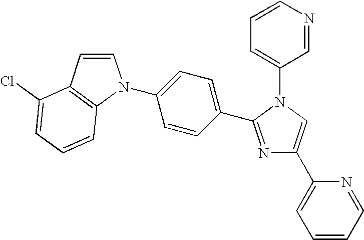

A mixture of 2-(2-(4-iodophenyl)-1-(pyridin-3-yl)-1H-imidazol-4-yl)pyridine (200 mg, 0.47 mmol), 4-chloroindole (85 mg, 0.56 mmol), Cut (134 mg, 0.7 mmol), K₃PO₄ (199 mg, 0.94 mmol) and trans-1,2-cyclohexanediamine (6 mg, 0.05 mmol) in p-dioxane (2 mL) was heated by microwave at 150° C. for 1.5 h, filtered, concentrated, and the residue purified by SGC (0.5 and 1% MeOH in DCM, 0.5% NH₄OH) giving 76 mg of solid containing starting iodide. This was purified by RP-HPLC giving a yellow solid. Yield 41 mg. ¹H NMR (CDCl₃) δ 8.89 (dd, 1H, J=1, 5.8 Hz). 8.75 (dd, 1H, J=1.5, 4.8 Hz). 8.71 (s, 1H), 8.61 (d, 1H, J=2 Hz), 8.58 (d, 1H, J=7.8 Hz), 8.39 (dt, 1H, J=1.7, 7.9 Hz), 7.29 (ddd, 1H, J=2, 3, 8 Hz). 7.65 (ddd, 1H, J=1, 8, 7 Hz), 7.59-7.54 (m, 3H), 7.48 (m, 2H), 7.41 (dt, 1H, J=1, 8 Hz), 7.34 (d, 1H, J=3.3 Hz), 7.18-7.11 (m, 2H), 6.80 (dd, 1H, J=1, 3 Hz), MS (AP+) m/e 448 (MH+), IC₅₀=113 nM.

### Example 40

### 1-(4-(4-(pyridin-2-yl)-1-(pyridin-3-yl)-1H-imidazol-2-yl)phenyl)-1H-indole-4-carbonitrile bis-TFA salt

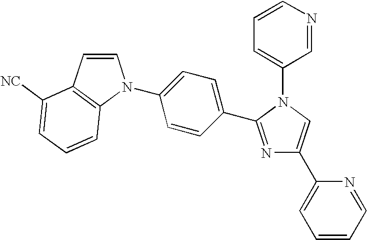

A mixture of 2-(2-(4-iodophenyl)-1-(pyridin-3-yl)-1H-imidazol-4-yl)pyridine (200 mg, 0.47 mmol), 4-cyanoindole (67 mg, 0.47 mmol), CuI (4.5 mg, 0.024 mmol), K₃PO₄ (199 mg, 0.94 mmol) and N,N-dimethyl-trans-1,2-cyclohexanediamine (7 mg, 0.05 mmol) in p-dioxane (1 mL) was heated by microwave at 140° C. for 2 h, filtered, concentrated, and the residue purified by RP-HPLC giving a yellow solid. Yield 87 mg, 42%. ¹H NMR (CDCl₃) δ 8.87 (dd, 1H, J=1, 5.5 Hz), 8.74 (dd, 1H, J=1.5, 4.8 Hz), 8.73 (s, 1H), 8.58 (d, 1H, J=2.1 Hz), 8.55 (d, 1H, J=7.9 Hz), 8.26 (dt, 1H, J=1.7, 7.9 Hz), 7.82 (ddd, 1H, 1, 2.6, 8 Hz), 7.71 (d, 1H, J=8.3 Hz), 7.82-7.58 (m, 3H), 7.55-7.52 (m, 2H), 7.48-7.45 (m, 3H), 7.28-7.24 (m, 1H), 6.90 (dd, 1H, J=0.8, 3.3 Hz). MS (AP+) m/e 439 (MH+). Anal. Calcd for C₂₈H₁₈N₈+2 CF₃COOH:C, 57.66; H, 3.02; N, 12.61. Found: C, 57.67; H, 3.09; N, 12.69. IC₅₀=65.4 nM.

### Example 41

### 3-(2-(4-(4-methyl-1H-imidazol-1-yl)phenyl)-4-(pyridin-2-yl)-1H-imidazol-1-yl)pyridine

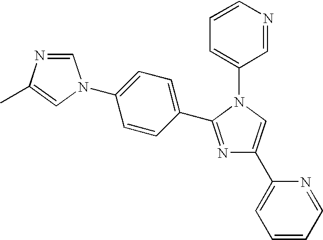

A mixture of 2-(2-(4-iodophenyl)-1-(pyridin-3-yl)-1H-imidazol-4-yl)pyridine (300 mg, 0.71 mmol), 4-methylimidazole (58 mg, 0.71 mmol). Cut (7 mg, 0.035 mmol), Cs₂CO³ (463 mg, 1.4 mmol), and N,N-dimethyl-trans-1,2-cyclohexanediamine (10 mg, 0.07 mmol) in DMF (1 mL) was heated at 110°G for 48 h, filtered, concentrated, and the residue purified by SGC (0.5-2% MeOH in DCM, 0.5% NH₄OH). Yield 172 mg, 64%. NMR showed two substances in approximately 4:1 ratio. NMR (CDCl₃, major isomer) d 8.67 (dd, 1H, J=1.7, 5.0 Hz), 8.61 (d, 1H, J=2.1 Hz), 8.56 (ddd, 1H, J=0.8, 1.7, 5.0 Hz). 8.10 (dt, 1H, J=1.0, 7.9 Hz), 7.88 (s, 1H), 7.78-7.73 (m, 2H), 7.62 (ddd, 1H, J=1.7, 2.6, 8.2 Hz), 7.53-7.49 (m, 2H), 7.40 (ddd, 1H), 7.31-7.27 (m, 2H), 7.18 (ddd, 1H, J=1, 4.8, 7.5 Hz), 6.98 (m, 1H), 2.26 (d, 3H, J=1 Hz). MS (AP+) m/e 379 (MH+). Minor isomer (partial) 2.15 (d, 3H, J=1 Hz), 6.98 (t, 1H), 7.66 (ddd, 1H, J=1.7, 2.6, 8.2 Hz), 8.11 (dt, 1H, J=1, 8 Hz). IC₅₀=293 nM.

### Example 42

### 1-(4-(4-(pyridin-2-yl)-1-(pyridin-3-yl)-1H-imidazol-2-yl)phenyl)-1H-benzo[d][1,2,3]triazole bis-TFA salt

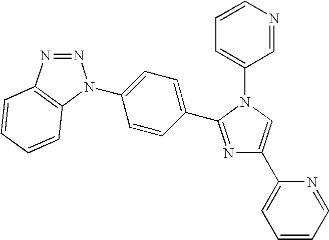

A mixture of 2-(2-(4-iodophenyl)-1-(pyridin-3-yl)-1H-imidazol-4-yl)pyridine (200 mg, 0.47 mmol), benzotriazole (56 mg, 0.47 mmol), CuI (4.5 mg, 0.024 mmol), K₃PO_{4 (199} mg. 0.94 mmol) and N,N-dimethyl-trans-1,2-cyclohexanediamine (7 mg, 0.05 mmol) in p-dioxane (1 mL) was heated at 110° C. for 48 h and by microwave at 140° C. for 1 h, filtered through silica, concentrated, and the residue purified by RP-HPLC giving a yellow solid. Yield 24 mg, 12%, ¹H NMR (CDCl₃) δ 8.84 (d, 1H, J=6.6 Hz), 8.80-8.78 (m, 2H), 8.68 (br, 1H), 8.61 (d, 1H, J=8.3 Hz), 8.37 (dt, 1H, J=˜2, 8 Hz), 8.16 (d, 1H, J=8.3 Hz), 7.96 (m, 1H), 7.85 (m, 2H), 7.77 (d, 2H, J=8.7 Hz), 7.72 (m, 1H), 7.67 (m, 2H), 7.60 (dt, 1H, J=˜1, 7 Hz), 7.47 (m, 1H). MS (AP+) m/e 416 (MH+). IC₅₀=67.8 nM.

### Example 43

### 2-(pyridin-2-yl)-1-(4-(4-(pyridin-2-yl)-1-(pyridin-3-yl)-1H-imidazol-2-yl)phenyl)-1H-benzo[d]imidazole

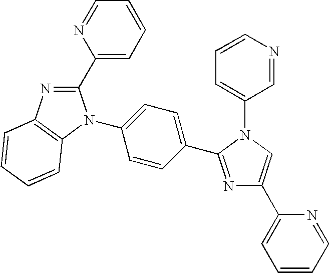

A mixture of 2-(2-(4-iodophenyl)-1-(pyridin-3-yl)-1H-imidazol-4-yl)pyridine (200 mg, 0.47 mmol), 2-(2-pyridyl)benzimidazole (92 mg, 0.47 mmol), Cut (4.5 mg, 0.024 mmol), Cs₂CO₃ (305 mg, 0.94 mmol) and N,N-dimethyl-trans-1,2-cyclohexanediamine (7 mg, 0.05 mmol) in DMF (1 mL) was heated at 110° C. for 5 days and by microwave at 140° C. for 1 h, concentrated, and the residue purified by SGC (0.5% and 1% MeOH in DCM, 0.5% NH₄OH) giving an off-white solid. Yield 50 mg. 22%. ¹H NMR (CDCb) δ 8.67 (dd, 1H. J=1.5, 4.8 Hz), 8.62 (d, 1H, J=2 Hz), 8.59 (ddd, 1H, J=1,-1, 5 Hz), 8.37 (ddd, 1H, J=1, 1, 5 Hz), 8.16 (br, 1H), 8.11 (d, 1H, J=7.9 Hz). 7.88 (d, 1H, J=7.5 Hz), 7.82 (br, 1H), 7.75 (dt, 1H, J=1.7, 7.7 Hz), 7.70 (m, 1H, J=8.3 Hz), 7.54 (m, 2H), 7.42 (ddd, 1H, J=˜1, 4.4, 8.5 Hz), 7.36-7.27 (m, 4H), 7.25-7.21 (m, 4H). MS (AP+) m/e 492 (MH+). IC₅₀=33.1 nM.

### Example 44

### 3-(2-(4-(1H-imidazol-1-yl)phenyl)-4-(pyridin-2-yl)-1H-imidazol-1-yl)pyridine

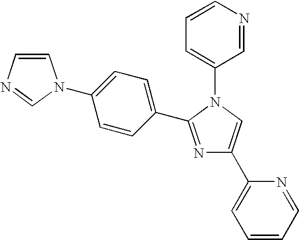

A mixture of 2-(2-(4-iodophenyl)-1-(pyridin-3-yl)-1H-imidazol-4-yl)pyridine (200 mg, 0.47 mmol), imidazole-2-carboxaldehyde (45 mg, 0.47 mmol), Cut (5 mg, 0.024 mmol), Cs₂CO₃ (306 mg, 0.94 mmol) and N,N-dimethyl-trans-1,2-cyclohexanediamine (7 mg, 0.05 mmol) in p-dioxane (1 mL) was heated at 110° C. for 46 h and by microwave at 140° C. for 1.5, filtered, concentrated, and the residue purified by SGC (1% and 2% MeOH in DCM, 0.5% NHOH) giving a yellow solid which was determined to be the title substance (decarbonylation had occurred). Yield 40 mg, 10%. ¹H NMR (CDCb) δ 8.68 (dd, 1H, J=1.5, 4.8 Hz), 8.61 (d, 1H, J=2 Hz). 8.58 (m, 1H, J=4 Hz), 8.13 (d, 1H, J=7.9 Hz), 7.95 (s, 1H), 7.86 (s, 1H), 7.78 (dt, 1H, J=1.7, 7.7 Hz), 7.84 (ddd, 1H, J=1.5, 2.5, 8 Hz), 7.54 (m, 2H), 7.41 (ddd, 1H, J=-1, 4.8, 8 Hz), 7.34 (m, 2H), 7.26 (t, 1H), 7.21 (ddd, 1H, J=1, 2.5, 7.5 Hz), 7.19 (br, 1N). MS (AP+) m/e 365 (MH+). IC₅₀=522 nM.

### Example 45

### 1-(4-(4-(pyridin-2-yl)-1-(pyridin-3-yl)-1H-imidazol-2-yl)phenyl)-1H-benzo[d]imidazole

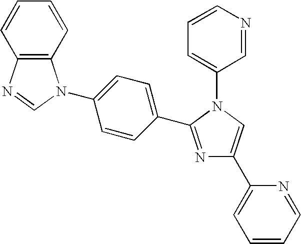

A mixture of 2-(2-(4-iodophenyl)-1-(pyridin-3-yl)-1H-imidazol-4-yl)pyridine (200 mg, 0.47 mmol), benzimidazole (55 mg, 0.47 mmol), CuI (5 mg, 0.024 mmol), Cs₂CO₃ (306 mg, 0.94 mmol) and N,N-dimethyl-(trans-1,2-cyclohexanediamine (7 mg, 0.05 mmol) in DMF (1 mL) was heated at 110° C. for 18 h, filtered, concentrated, and the residue purified by SGC (0.5 and 1% MeOH in DCM, 0.5% NH₄OH) giving a yellow solid. Yield 60 mg, 31%. ¹H NMR (CDCl₃) δ 8.71 (dd, 1H, J=1.5, 4.8 Hz), 8.65 (d, 1H, J=2.1 Hz), 8.59 (ddd, 1H, J=˜1, 1.5, 5 Hz), 8.20 (d, 1H, J=7.5 Hz). 8.10 (s, 1H), 7.88-7.83 (m, 2H), 7.71 (ddd, 1H, J=1.5, 2.5, 8 Hz), 7.64 (m, 2H), 7.53-7.44 (m, 4H), 7.35-7.25 (m, 4H). MS (AP+) m/e 415 (MH+). IC₅₀=97.8 nM.

### Example 46

### 1-(4-(4-(pyridin-2-yl)-1-(pyridin-3-yl)-1H-imidazol-2-yl)phenyl)-1H-imidazo[4,5-b]pyridine bis-trifluororoacetic acid salt

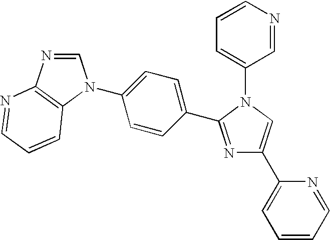

A mixture of 2-(2-(4-iodophenyl)-1-(pyridin-3-yl)-1H-imidazol-4-yl)pyridine (200 mg, 0.47 mmol), 4-azabenzimidazole (56 mg, 0.47 mmol), CuI (5 mg, 0.024 mmol), Cs₂CO₃ (306 mg, 0.94 mmol) and N,N-dimethyl-trans-1,2-cyclohexanediamine (7 mg, 0.05 mmol) in DMF (1 mL) was heated at 110° C. for 24 h and by microwave at 140° C. for 1.5, filtered, concentrated and the residue purified by RP-HPLC giving two isomers. For the first-eluting peak, yield 25 mg, light brown solid. HPLCMS 4.53 min (m/e 416, MH+). ¹H NMR (DMSO-d₆) δ 8.93 (s, 1H), 8.73 (d, 1H, J=2.5 Hz), 8.70 (dd, 1H, J=1.5, 4.8 Hz), 8.64 (dt. 1H, J=1, 5.4 Hz), 8.51 (m, 2H), 8.21 (m, 2H), 8.12 (dd, 1H, J=1.7, 8.3 Hz), 7.99 (ddd, 1H, J=1.5, 25, 8.1 Hz), 7.76 (m, 2H), 7.64 (m, 2H), 7.80 (dd, 1H, J=5.0, 8.3 Hz), 7.54 (m, 1H), 7.38 (dd, 1H, J=4.8, 8.3 Hz). A minor substance (˜15%) was also detected by NMR. MS (AP+) m/e 416 (MH+). IC₅₀=231 nM.

### Example 4

### 3-(4-(4-(pyridin-2-yl)-1-(pyridin-3-yl)-1H-imidazol-2-yl)phenyl)-3H-imidazol-[4,5-b]pyridine

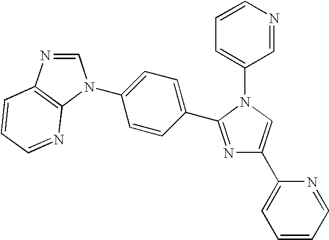

The second eluting isomer from the RP-HPLC purification of the preceding Example was isolated. Yield 45 mg, light brown solid. It was determined to be a mixture of the title substance and 2-(2-phenyl-(pyridin-3-yl)-1H-imidazol-4-yl)pyridine resulting from reduction of the iodide starting material: HPLCMS 4.87 min (m/e 416, MH+ of title substance) and 4.69 min (m/e 299 for MH+ of des-iodo derivative), approx 2:1 ratio by 280 nM. UV absorbance, respectively). ¹H NMR (DMSO-d₆) δ (partial) 8.95 (s, 1H). IC₅₀=14.7 nM.

### Example 48

### 1-(4-(1-(6-methylpyridin-3-yl)-4-(pyridin-2-yl)-1H-imidazol-2-yl))phenyl)-1H-imidazo[4,5-b]pyridine

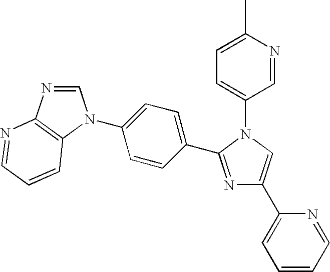

A mixture of 2-(2-(4-iodophenyl)-1-(8-methylpyridin-3-yl)-1H-imidazol-4-yl)pyridine (200 mg, 0.45 mmol), 4-azabenzimidazole (54 mg, 0.45 mmol), CuI (4 mg, 0.023 mmol), Cs₂CO3 (308 mg, 0.94 mmol) and N,N-dimethyl-trans-1,2-cyclohexanediamine (6 mg, 0.045 mmol) in DMF (0.3 ml) was heated by microwave at 150° C. for 2 h, filtered through a small silica plug, concentrated and the residue purified by RP-HPLC (basic conditions) giving two isomers. For the first-eluting peak, yield 15 mg. HPLCMS 4.69 min (m/e 430, MH+). ¹H NMR (CDCl₃) δ 8.63 (dd, 1H, J=1.5, 4.8 Hz), 8.59 (dq, 1H, J=< 1 Hz, 5.0 Hz), 8.50 (d, 1H, J=2.5 Hz), 8.34 (s, 1H), 8.17 (br, 1H), 7.85 (dd, 1H, J=1.7, 8.3 Hz), 7.86-7.81 (br, 1H), 7.69 (m, 2H), 7.58 (dd, 1H J=2.7, 8.1 Hz), 7.46 (m, 2H), 7.30-7.25 (m, 4H), 2.64 (s, 3H). IC₅₀=63.7 nM.

### Example 49

### 3-(4-(1-(6-methylpyridin-3-yl)-4-(pyridin-2-yl)-1H-imidazol-2-yl)phenyl)-3H-imidazo[4,5-b]pyridine

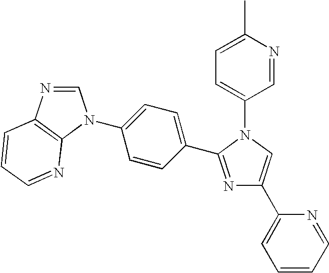

The second eluting isomer from the RP-HPLC purification in the preceding Example was isolated. Yield 25 mg. It was distinguishable from the first-eluting isomer of the preceding Example by HPLCMS retention time (5.10 min (m/e 430, MH+)). ¹H NMR (CDCl₃) δ 8.58 (dq, 1H, J=< 1 Hz, 5 Hz). 8.54 (d, 1H, J=2.5 Hz), 8.45 (dd, 1H, J=1.5, 4.8 Hz), 8.34 (s, 1H), 8.18 (br, 1H), 8.15 (dd, 1H, J=1.7, 7.9 Hz). 7.83 (br, 1H), 7.80 (m, 2H), 7.66 (m, 2H), 7.52 (dd, 1H, J=2.7, 8.1 Hz), 7.31 (dd, 1H, J=4.8, 8.1 Hz), 7.24 (d, 1H, J=8.3 Hz), 7.25-7.23 (m, 2H), 2.63 (s, 3H). IC₅₀=5.50 nM.

The title substance was independently synthesized by the following procedure. N²-(4-(1-(6-methylpyridin-3-yl)-1H-(pyridin-2-yl)-1H-imidazol-2-yl)phenyl)pyridine-2,3-diamine (42 mg, 0.1 mmol) and ethoxymethylenemaiononitrile (15 mg, 0.12 mmol) and acetic acid (0.2 ml) were combined, the solution heated at reflux for 45 min and concentrated. The residue was dissolved in 30 mL EtOAc and the solution washed with aqueous NaHCO₃ and dried. The residue was purified by SGC (1% MeOH in DCM, 0.5% NH₄OH). Yield 18 mg. HPLCMS 5.10 min (m/e 430, MH+). By ¹H NMR (CDCb), this material was identical to the second-eluting isomer described immediately above and distinguishable from the first-eluting isomer of the preceding Example.

### Preparation 49A

### N-(4-(1-(6-methylpyridin-3-yl)-4-(pyridin-2-yl)-1H-imidazol-2-yl)phenyl)-3-nitropyridin-2-amine

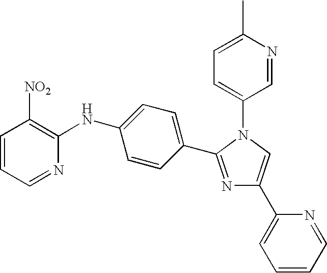

A mixture of 2-(2-(4-iodophenyl)-1-(6-methylpyridin-3-yl)-1H-imidazol-4-yl)pyridine (1.06 g, 2.4 mmol), 2-amino-3-nitropyridine (370 mg, 2.66 mmol), tris(dibenzylideneacetone)dipalladium(0) (21 mg, 0.023 mmol), 4,5-bis(diphenylphosphino) 9,9-dimethylxanthene (33 mg, 0.057 mmol), Cs₂CO₅ (1.04 g, 3.2 mmol) and p-dioxane (3 mL) was heated by microwave at 145° C. for 120 mm. The mixture was diluted with DCM, filtered, and combined with four other identically prepared crude products (together representing a total of 5.3 g. 11.6 mmol of starting iodide), for a total of 5.31 g of crude product. This was purified by SGC (1%-3% MeOH in DCM, 0.5% NH₄OH, giving a red solid. Yield 2.80 g, 56%. ¹H NMR (CDCl₃) δ 10.21 (s, 1H), 8.56 (ddd, 1H, J=< 1, 5 Hz), 8.51-8.46 (m, 3H), 8.11 (dt. 1H, J=< 1, 7.9 Hz), 7.84 (s, 1H), 7.74 (dt, 1H, J=1.7, 7.7 Hz), 7.67 (m, 2H), 7.47-7.43 (m, 3H), 7.19 (s, 1H), 7.16 (ddd, 1H, J=1, 4.7, 7.6 Hz), 6.84 (dd, 1H, J=4.6, 8.3 Hz), 2.60 (s, 3H). MS (AP+) m/e 450 (MH+).

### Preparation 49B

### N²-(4-(1-(6-methylpyridin-3-yl)-4-(pyridin-2-yl)-1H-imidazol-2-yl)phenyl)pyridine-2,3-diamine

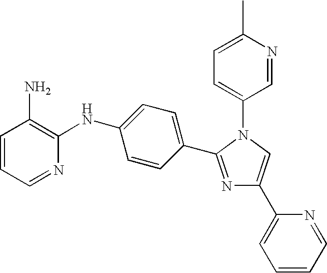

A mixture of N-(4-(1-(6-methylpyridin-3-yl)-4-(pyridin-2-yl)-1H-imidazol-2-yl)phenyl)-3-nitropyridin-2-amine (2.7 g, 6.01 mmol), 10% palladium on carbon (900 mg), and MeOH (100 ml) was shaken under 45 p.s.i. hydrogen pressure for 2 h, filtered, concentrated and the residue dried giving a dark pink solid which was used without further purification. Yield 2.15 g, 85%. ¹H NMR (CDCl₃) δ 8.55 (dq, 1H, J=<1, 5 Hz), 8.51 (d, 1H, J=2 Hz), 8.09 (d, 1H, J=8.3 Hz), 7.80-7.78 (m, 2H), 7.73 (dt, 1H, J=1.7, 7.7 Hz), 7.42 (dd, 1H, J=2.5, 8.3 Hz), 7.30 (m, 2H), 7.30 (m, 2H), 7.22 (m, 2H), 7.17 (s, 1H), 7.15 (m, 1H), 7.00 (dd, 1H, J=1.7, 7.5 Hz), 6.77 (dd, 1H, J=5, 7.9 Hz), 6.68 (br, 1H), 2.59 (s, 3H) MS (AP+) m/e 420 (MH+).

### Example 50

### 5-(4-(1-(6-methylpyridin-3-yl)-4-(pyridin-2-yl)-1H-imidazol-2-yl)phenyl)-5H-pyrrolo[3,2-b]pyrazine

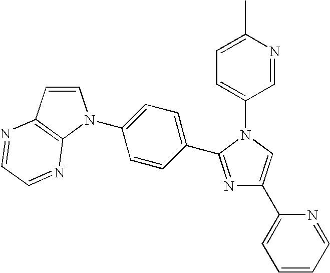

A mixture of 2-(2-(4-iodophenyl)-1-(6-methylpyridin-3-yl)-1H-imidazol-4-yl)pyridine (200 mg, 0.45 mmol), 5H-pyrrolo[3,2-b]pyrazine (54 mg, 0.45 mmol), CuI (4 mg, 0.023 mmol), K₃PO₄ (218 mg, 1.03 mmol) and N,N-dimethyl-trans-1,2-cyclohexanediamine (6 mg, 0.045 mmol) in p-dioxane (0.5 mL) was heated by microwave at 150° C. for 2 h, diluted with DCM, filtered, and concentrated and the residue purified by SGC (1% and 2% MeOH in DCM, 0.5% NH₄OH) giving an off-white solid. Yield 90 mg. 47%. ¹H NMR (CDCb) δ 8.57 (ddd, 1H, J=0.8, 1.6.5 Hz), 8.53 (d, 1H, J=2.9 Hz), 8.48 (d, 1H, J=2.9 Hz), 8.28 (d, 1H, J= 2.5 Hz), 8.14 (d, 1H, J=7.9 Hz), 7.92 (br, 1H), 7.82 (d, 1H, J=3.7 Hz), 7.81-7.79 (m, 3H), 7.62 (m, 2H), 7.51 (dd, 1H, J=2.5, 8.3 Hz), 7.24 and 7.20 (m, 2H total), 6.87 (d, 1H, J=3.7 Hz), 2.62 (s, 3H). MS (AP+) m/e 430 (MH+). IC₅₀=<3.47 nM.

### Example 51

### 3(4-(4-(pyridin-2-yl)-1-(pyridin-3-yl)-1H-imidazol-2-yl)phenyl)-3H-[1,2,3]triazole[4,5-b]pyridine bis-TFA salt

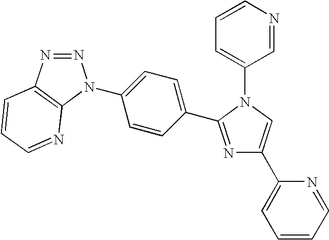

A mixture of 2-(2-(4-(iodophenyl)-1-(pyridin-3-yl)-1H-imidazol-4-yl)pyridine (200 mg, 0.47 mmol), 4-azabenzotriazole (57 mg, 0.47 mmol), CuI (5 mg, 0.024 mmol), K₃PO₄ (205 mg, 0.94 mmol) and N,N-dimethyl-trans-1,2-cyclohexanediamine (6 mg, 0.047 mmol) in DMF (1 ml) was heated by microwave at 140° C. for 2 h, filtered through silica eluting with MeOH-DCM, concentrated and the residue purified by RP-HPLC. Yield 18 mg. ¹H NMR (CDCl_{3) δ} 8.76 (dd, 1H, J=1.7, 4.8 Hz), 8.71-8.86 (m, 2H), 8.59 (ddd, 1H, J=0.8, 1.7, 5 Hz), 8.45 (dd, 1H, J=1.2, 8.3 Hz), 8.40-8.36 (m, 2H), 8.18 (m, 1H), 8.04 (br, 1H), 7.82 (m, 1H). 7.69-7.63 (m, 3H), 7.43 (dd, 1H, J=4.4, 8.5 Hz), 7.40 (dd, 1H, J=4.6, 7.9 Hz), 7.25-7.23 (m, 1H). Another substance was detected by NMR (˜20%, partial) 8.84 (dd, 1H, J=1.7, 4.1 Hz), 8.28 (dd, 1H, J=1.7, 8.7 Hz), which was not resolved by HPLCMS. IC₅₀=11.5 nM.

### Example 52

### 1-(4-(1-(6-methylpyridin-3-yl)-4-(pyridin-2-yl)-1H-imidazol-2-yl)phenyl)-1H-pyrrolo[3,2-b]pyridine

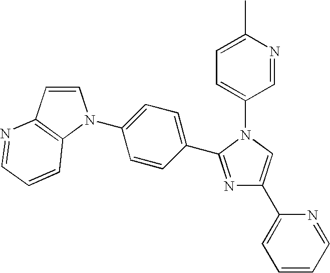

A mixture of 2-(2-(4-iodophenyl)-1-(6-methylpyridin-3-yl)-1H-imidazol-4-yl)pyridine (200 mg, 0.45 mmol), 1H-pyrrolo[1,3,2-b]pyridine (Chem. Pharm. Bull. 1987, 35(5)-1823-28, 53 mg, 0.45 mmol, CuI (5 mg, 0.025 mmol), K₃PO₄ (209 mg, 1 mmol) and N,N-dimethyl-trans-1,2-cyclohexanediamine (7 mg, 0.049 mmol) in p-dioxane (1 mL) was heated by microwave at 150° C. for 2.5 h, diluted with DCM, filtered, and concentrated and the residue purified by SGC (1%-4% MeOH in DCM, 0.5% NH₄OH) giving a yellow solid. Yield 35 mg. ¹H NMR (CDCl₃) δ 8.59 (d, 1H, J=4.5 Hz). 8.52 (m, 2H), 8.15 (d, 1H, J=7.9 Hz). 7.95 (br, 1H), 7.90 (d, 1H, J=8.3 Hz), 7.80 (dt, 1H, J=1, 8 Hz), 7.65-7.62 (m, 3H), 7.57 (dd, 1H, J=2.7, 8.1 Hz), 7.43 (m, 2H), 7.27 (d, 1H, J=8.3 Hz). 7.24-7.20 (m, 2H), 6.98 (d, 1H, J=3 Hz), 2.64 (s, 3H). HPLCMS 2.91 min, m/e 429 (MH+). IC₅₀=9.32 nM.

### Example 53

### 1-(4-(1-(6-methylpyridin-3-yl)-4-(pyridin-2-yl)-1H-imidazol-2-yl)phenyl)-1H-pyrrolo[2,3]-c]pyridine

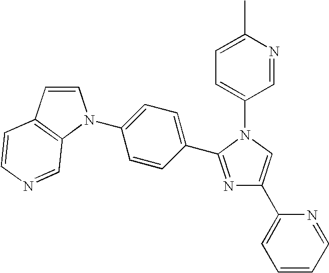

A mixture of 2-(2-(4-iodophenyl)-1-(6-methylpyridin-3-yl)-1H-imidazol-4-yl)pyridine (133 mg, 0.30 mmol), 1H-pyrrolo[2,3-c]pyridine (36 mg, 0.30 mmol), CuI (3 mg, 0.015 mmol), K₃PO₄ (193 mg, 0.91 mmol) and N,N-dimethyl-trans-1,2-cyclohexanediamine (4 mg, 0.030 mmol) in p-dioxane (3 mL) was heated by microwave at 150° C. for 2.5 h, diluted with DCM, concentrated and the residue purified by SGC (1-1.5% MeOH in DCM, 0.5% NH₄OH) giving a yellow solid. Yield 60 mg, 62%. ¹H NMR (CDCl₃) δ 8.93 (s, 1H), 8.58 (ddd, 1H, J=1, 2, 5 Hz), 8.55 (d, 1H, J=2.5 Hz), 8.30 (d, 1H, J=5.8 Hz), 8.13 (dt, 1H, J=0.8.8 Hz), 7.88 (s, 1H), 7.77 (dt, 1H, J=1.7, 7.7 Hz), 7.68-7.64 (m, 3H), 7.57 (d, 1H, J=2.9 Hz), 7.55 (dd, 1H, J= 2.9, 8.3 Hz), 7.47 (m, 2H), 7.28 (d, 1H, J=8.3 Hz), 7.20 (ddd, 1H, J=1.2, 5, 7.5 Hz), 6.76 (d, 1H, J=2.9 Hz), 2.65 (s, 3H). MS (AP+) m/e 429 (MH+). IC₅₀=5.69 nM.

### Example 54

### 1-(4-(1-(6-methylpyridine-3-yl)-4-(pyridin-2-yl)-1H-imidazol-2-yl)phenyl)-1H-pyrrolo[3,2-c]pyridine

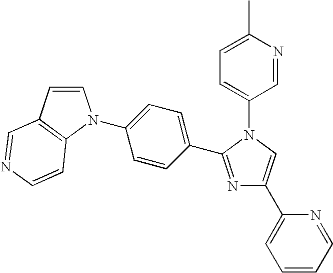

A mixture of 2-(2-(4-iodophenyl)-1-(6-methylpyridin-3-yl)-1H-imidazol-4-yl)pyridine (115 mg, 0.26 mmol), 1H-pyrrolo[3,2-c]pyridine (46 mg, 0.39 mmol), CuI (2.5 mg, 0.013 mmol), K₃PO₄ (165 mg, 0.78 mmol) and N,N-dimethyl-trans-1,2-cyclohexanediamine (4 mg, 0.030 mmol) in p-dioxane (3 ml) was heated by microwave at 150° C. for 2.5 h, diluted with DCM, concentrated and the residue purified by SGC (1-1.5% MeOH in DCM, 0.5% NH₄OH) giving a yellow solid which was further purified by RP-HPLC (basic conditions). Yield 22 mg. ¹H NMR (CDCl₃) δ 9.02 (s, 1H), 8.59 (m, 1H), 8.51 (d, 1H, J=2.9 Hz), 8.36 (d, 1H, J=6.2 Hz), 8.12 (dt, 1H, J=−1.8 Hz), 7.89 (s, 1H), 7.77 (dt, 1H, J=1.7, 7.7 Hz). 7.68 (m, 2H), 7.58 (dd, 1H, J=2.9, 8.3 Hz), 7.55 (d, 1H, J=6.2 Hz), 7.51 (d, 1H, J=3.3 Hz), 7.45 (m, 2H), 7.29 (d, 1H, J=8.3 Hz), 7.20 (ddd, 1H, J=1.2, 5, 7.5 Hz), 6.93 (d, 1H, J=3.3 Hz), 2.65 (s, 3H). MS (AP+) m/e 429 (MH+). IC₅₀=5.82 nM.

### Example 55

### 9-(4-(1-(6-methylpyridin-3-yl)-4-(pyridin-2-yl)-1H-imidazol-2-yl)phenyl-9H-purine and 7-(4-(1-(6-methylpyridin-3-yl)-4-pyridin-2-yl)-1H-imidazol-2-yl))phenyl)-7H-purine

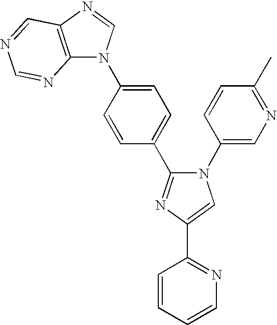

Analogously to the method used to prepare 1-(4-(1-(6-methylpyridin-3-yl)-4-(pyridin-2-yl)-1-imidazol-2-yl)phenyl)-1H-imidazo[4,5-b]pyridine, 2-(2-(4-iodophenyl)-1(6-methylpyridin-3-yl)-1H-imidazol-4-yl)pyridine and purine were coupled giving the title substance as a mixture of two isomers, approximately 4:1 ratio, ¹H NMR (CDCl₃, 400 mHz) δ (major isomer) 9.24 (s, 1H), 9.05 (s, 1H), 8.58 (ddd, 1H, J=0.8, 1.7, 5 Hz), 8.53 (d, 1H, J=2.5 Hz), 8.39 (s, 1H). 8.12 (dt, 1H, J=8 Hz), 7.89 (s, 1H). 7.80-7.75 (m, 3H), 7.69 (m, 2H), 7.53 (dd, 1H, J=2.7, 8.1 Hz), 7.26 (d, 1H, J=8.3 Hz), 7.20 (ddd, 1H, J=1.5, 7.5 Hz), 2.64 (s, 3H). For the minor isomer δ (partial) 8.48 (d, 1H, J=2 Hz), 8.25 (br, 1H), 8.00 (d, 1H, J=8.3 Hz), 7.45 (dd, 1H, J=2.5, 8.7 Hz), 2.60 (s, 3H). HPLCMS 4.54 min, m/e 431 (MH+). MS (AP+) m/e 431 (MH+). IC₅₀=10.2 nM.

### Example 56

### 1-(4-(1-(6-methylpyridin-3-yl)-4-(pyridin-2-yl)-1H-imidazol-2-yl)phenyl)-1H-pyrazolo[3,4-]pyridine

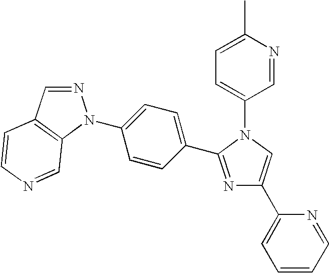

A mixture of 2-(2-(4-iodophenyl)-1-(6-methylpyridin-3-yl)-1H-imidazol-4-yl)pyridine (175 mg, 0.40 mmol), 1H-pyrazolo[3,4-c]pyridine (J. Chem. Soc. Perkin Transactions I, 1973, p. 2901, 0.48 mg, 0.40 mmol), Cut (3.8 mg, 0.020 mmol), K₃PO₄ (178 mg, 0.84 mmol) and N,N-dimethyl-trans-1,2-cyclohexanediamine (12 mg, 0.080 mmol) in p-dioxane (1 mL) was heated by microwave at 150° C. for 3 h, filtered through silica using DCM-MeOH, and concentrated and the residue purified by SGC (1% MeOH in DCM, 0.5% NHOH) giving a yellow solid. Yield 45 mg, 26%. ¹H NMR (CDCl₃) showed a 10:1 mixture of two substances which were not resolved by HPLCMS (4.23 min, m/e 430 (MH+). For the major substance δ 9.28 (s, 1H), 8.60 (d, 1H, J=5 Hz), 8.53 (d, 1H, J=2.5 Hz), 8.41 (d, 1H, J=5 Hz), 8.32 (br, 1H), 8.26 (s, 1H), 7.96 (br, 1H), 7.76 (m, 2H), 7.71 (m, 1H), 7.66 (m, 2H), 7.55 (m, 1H), 7.34 (br, 1H), 7.26-7.24 (m, 2H), 2.64 (s, 3H). For the minor substance (partial) 9.25 (s, 1H), 2.67 (s, 3H), IC₅₀=6.44 nM.

### Example 57

### 2-methyl-3-(4-(1-(6-methylpyridin-3-yl)-4-(pyridin-2-yl)-1H-imidazol-2-yl)phenyl)-3H-imidazo[4,5-b]pyridine

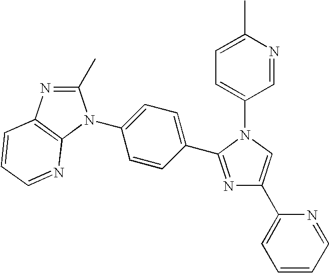

N²-(4-(1-(6-methylpyridin-3-yl)-4-(pyridin-2-yl)-1H-imidazol-2-yl)phenyl)pyridine-2,3-diamine (75 mg, 0.18 mmol), (1-ethoxyethylidene)malononitrile (29 mg, 0.22 mmol) and acetic acid (0.5 ml) were combined and heated at reflux for 1.5 h. The mixture was concentrated and the residue dissolved in DCM and washed with aqueous NaHCO₃. The organic layer was dried, concentrated, and the residue purified by SGC (1-4% MeOH in DCM, 0.5% NH₄OH) giving a pink solid, Yield 30 mg, 37%. ¹H NMR (CDCl₃) δ 8.60 (d, 1H, J=4 Hz), 8.57 (d, 1H, J=2 Hz), 8.29 (d, 1H, J=1.5, 4.8 Hz), 8.00 (dd, 1H, J=1.5, 8.1 Hz), 7.70 (m, 2H), 7.59 (dd, 1H, J=1.8, 8.1 Hz), 7.41 (m, 2H), 7.29-7.22 (m, 3H), 2.64 (s, 3H), 2.54 (s, 3H). A minor set (10%) of resonances was also present (partial description) 2.52 (s, 3H), 2.67 (s, 3H). HPLCMS was homogeneous (4.25 min, m/e 444 (MH+)). IC₅₀=1.89 nM.

### Example 58

### 2(trifluoromethyl)-3(4-(1-(6-methylpyridin-3-yl)-4-(pyridin-2-yl)-1H-imidazol-2-yl)phenyl)-3H-imidazo[4,5-b]pyridine

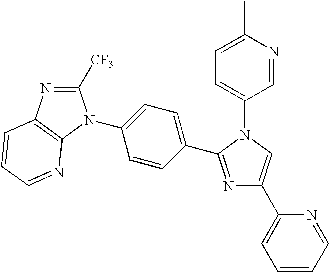

N²-(4-(1-(6-methylpyridin-3-yl)-1H-(pyridin-2-yl)-1H-imidazol-2-yl)phenyl)pyridine-2,3-diamine (75 mg, 0.18 mmol) was heated at reflux in 0.5 mL TFA for 1.5 h and concentrated. The residue was dissolved in 10 mL DCM and the solution extracted with aqueous NaHCO₃, The organic layer was dried, concentrated, and the residue purified by SGC (1:2 EtOAc-hexane) giving a solid. Yield 43 mg, 48%, ¹H NMR (CDCl₃) δ 8.60-8.58 (m, 2H), 8.51 (dd, 1H, J=1.5, 4.8 Hz), 8.24 (dd, 1H, J=1.7, 8.3 Hz), δ 22 (br, 1H), 8.15-7.95 (br, 1H), 7.86 (br, 1H), 7.71 (m, 2H), 7.54 (dd, 1H, J=2.9, 8.3 Hz), 7.42 (d, 2H, J=8.3 Hz), 7.41 (dd, 1H, J=4.6, 8.3 Hz), 7.26 (d, 1H, J=7.3 Hz), 7.25 (br, 2H). 2.64 (s, 3H). MS (AP+) m/e 498 (MH+). HPLCMS 5.95 min, m/e 498 (MH+). IC₅₀=1.06 nM.

### Example 59

### 2-isopropyl-3-(4-(1-(6-methylpyridin-3-yl)-4-(pyridin-2-yl)-1H-imidazol-2-yl)phenyl)-3H-imidazo[4,5-b]pyridine

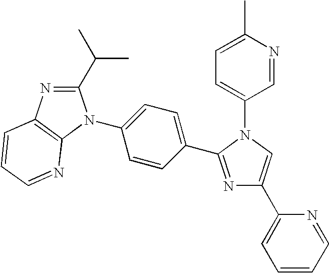

N²-(4-(1-(6-methylpyridin-3-yl)-4-(pyridin-2-yl)-1H-imidazol-2-yl)phenyl)pyridine-2,3-diamine (75 mg, 0.18 mmol) was combined with isobutyric anhydride (28 mg, 0.18 mmol; and isobutyric acid (0.5 ml) and heated at reflux for 2 h and concentrated. The residue was purified by SGC (1-4% MeOH in DCM, 0.5% NH₄OH) giving a pink solid. Yield 42 mg, 50%. ¹H NMR (CDCl₃) δ 8.81 (d, 1H, J=2.5 Hz), 8.59 (d, 1H, J=4 Hz), 8.28 (dd, 1H, J=1.7, 5 Hz), 8.17 (br, 1H), 8.05 (dd, 1H, J=1.7, 7.9 Hz), ˜8.0 (br, 1H), 7.83 (br, 1H), 7.71 (m, 2H), 7.56 (dd, 1H, J=2.3, 8.3 Hz), 7.37 (m, 2H), 7.27 (d, 1H, J=8.3 Hz), 7.22 (dd, 1H, J=5.0, 7.9 Hz), 7.24 (m, 1H), 3.10 (septet, 1H, J7 Hz). 2.64 (s, 3H), 1.32 (d, 6H, J=7 Hz). HPLCMS 5.06 min, m/e 472 (MH+). IC₅₀=0.831 nM.

### Example 60

### 2-methoxy-3-(4-(1-(6-methylpyridin-3-yl)-4-(pyridin-2-yl)-1H-imidazol-2-yl)phenyl)-3H-imidazo[4,5-b]pyridine

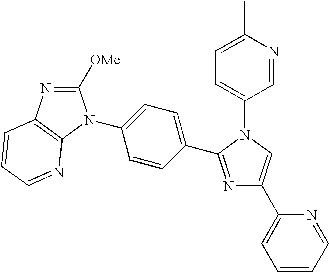

N²-(4-(1-(6-methylpyridin-3-yl)-4-(pyridin-2-yl)-1H-imidazol-2-yl)phenyl)pyridine-2,3-diamine (75 mg, 0.18 mmol) was combined with 0.5 mL tetramethylorthocarbonate and 2 mg propionic acid and heated at reflux for 1.5 h. The mixture was purified by SGC (1-4% MeOH in DCM, 0.5% NH₄OH) giving a colorless solid. Yield 49 mg, 59%. ¹H NMR (CDCl_{3) δ} 8.58-8.58 (m, 2H), 8.16 (dd, 1H, J=1.5, 5 Hz), 8.16 (br, 1H), 7.97 (br, 1H), 7.81 (dd, 1H, J=1.7, 7.9 Hz), 7.80 (br, 1H), 7.62 (m, 4H), 7.52 (dd, 1H, J=2.5, 8.3 Hz), 7.23 (d, 1H, J=8 Hz), 7.22 (m, 1H). 7.17 (dd, 1H, J=5, 7.9 Hz), 4.21 (s, 3H), 2.63 (s, 3H). MS (ES+) m/e 460 (MH+). IC₅₀=0.388 nM.

### Example 61

### 1-(4-(1-(6-methylpyridin-3-yl)-4(5-methylthiazol-2-yl)-1H-imidazol-2-yl)phenyl)-1H-pyrrolo[2,3-b]pyridine

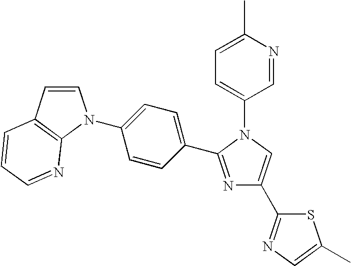

According to General Procedure 2, N′-(6-methylpyridin-3-yl)-4-(1H-(1H-pyrrolo[2,3-b]pyridin-1-yl)benzamidine (1.00 g, 3.06 mmol) and 2-bromo-1-(5-methyl-thiazol-2-yl)-ethanone (673 mg. 3.06 mmol) in 20 ml THF gave a light brown solid. Yield 152 mg. 11%. ¹H NMR (CDCb) δ 8.53 (d, 1H, J=2.5), 8.34 (dd, 1H, J=1.7, 5.0), 7.94 (dd, 1H, J=1.7, 7.9), 7.79 (m, 2H), 7.72 (br, 1H), 7.57 (m; 2H), 7.50 (d; 1H, J=3.7), 7.47 (dd, 1H, J=2.7, 8.1), 7.45 (m, 1H), 7.12 (d, 1H, J=8.3), 7.12 (dd, 1H, J=4.6, 7.9), 6.61 (d, 1H, J=3.7), 2.61 (s, 3H), 2.50 (s, 3H). MS (AP+) m/e 449 (MH+) IC₅₀=54.8 nM.

### Preparation 61A

### 2-Bromo-1-(5-methyl-thiazol-2-yl)-ethanone

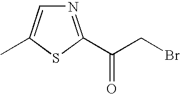

n-Butyllithium in hexanes (15.4 ml of 2.5M, 38.6 mmol) was added dropwise to a solution of 5-methylthiazole (3.65 g, 36.8 mmol) in ether (100 ml) at −78 to −65° C. and the mixture was stirred 15 min at −75° C. Methyl bromoacetate (3.85 ml. 5.9 g, 38.6 mmol) was added over 5 min (<−70° C.), and the mixture was stirred 25 min at −75° C. and treated with acetic acid (4 ml). Ether (100 ml) and water (50 mL) were added, the mixture brought to RT, and the organic layer washed with brine, dried, and concentrated giving the title substance as a yellowish solid (8.2 g, 100%) containing only small amounts of methyl bromoacetate and acetic acid by NMR. Recrystallization from hexane containing a little DCM gave a solid (3.15 g, 39%); ¹H NMR (CDCb) δ 7.67 (d, 1H, J=1 Hz), 4.62 (s, 2H), 2.56 (d, 1 Hz); ¹³C NMR (CDCb) δ 184.90, 162.14, 144.33, 143.68, 30.79, 12.89; MS 220/222 (100%, MH+). The NMR was consistent with that reported by R. W. Stevens, et al., PCT int. Appl. (1999) WO9905104A1, p. 121 for material prepared by bromination of 5-methyl-thiazol-2-yl-ethanone.

### Example 62

### 1-(4-(4-(5-chlorothiophen-2-yl)-1-(pyrimidin-5-yl)-1H-imidazol-2-yl)phenyl)-1H-pyrrolo[2,3-b]pyridine

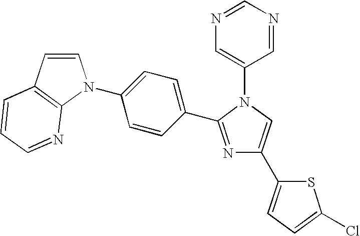

2-Bromo-1-(5-chlorothiophen-2-yl)ethanone (360 mg, 1.5 mmol), N′-(pyrimidin-5-yl)-4-(1H-pyrrolo[2,3-b]pyridin-1-yl)benzamidine (314 mg, 100 mmol), NaHCO₃ (166 mg, 2 mmol) and 2-propanol (4 mL) were combined and heated at reflux for 3.5 h, cooled, and diluted with DCM (10 mL). The mixture was filtered, concentrated, and the residue purified by SGC (EtOAc-hexanes giving a yellow solid. Yield 15 mg, 4%. ¹H NMR (CDCl₃) δ 8.24 (s, 1H), 8.74 (s, 2H), 8.35 (dd, 1H, J=1.5, 4.8 Hz). 7.95 (dd, 1H, J=1.7, 7.9 Hz), 7.85 (m, 2H), 7.53 (m. 2H), 7.50 (d, 1H, J=3.7 Hz), 7.33 (s, 1H), 7.16 (d, 1H, J=3.7 Hz), 7.13 (dd, 1H, J=4.6, 7.9 Hz), 6.89 (d, 1H, J=3.7 Hz), 8.83 (d, 1H, J=3.7 Hz). MS (AP+) m/e 455 and 457 (3:1. MH+). IC₅₀=65.1 nM.

### Example 63

### 1-(4-(4-(4-methylthiazol-2-yl)-1-(pyrimidin-5-yl)-1H-imidazol-2-yl)phenyl)-1H-pyrrolo-2,3-b]pyridine

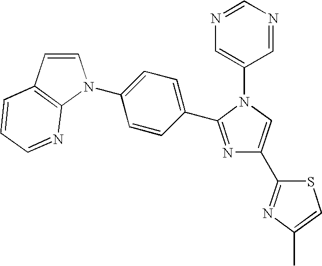

According to General Procedure 2, N′-(pyrimidin-5-yl)-4-(1H-pyrrolo[2,3-b]pyridin-1-yl)benzamidine (1.00 g, 3.18 mmol) and 2-bromo-1-(4-methylthiazol-2-yl)ethanone (700 mg, 3.18 mmol) gave 280 mg (20%) of the title substance. ¹H NMR (CDCb) δ 9.25 (s, 1H), 8.74 (s, 2H), 8.35 (dd, 1H, J=1.7, 5 Hz), 7.94 (dd, 1H, J=1.7.8 Hz), 7.87 (m, 2H). 7.81 (br, 1H). 7.55 (m, 2H), 7.51 (d, 1H, J=3.7 Hz), 7.13 (dd, 1H, J=4.7, 8 Hz), 6.68 (m, 1H), 6.63 (d, 1H, J=3.7 Hz), 2.49 (s, 3H). MS (AP+) m/e 436 (MH+). IC₅₀=13.8 nM.

### Preparation 63A

### 2-bromo-1-(4-methylthiazol-2-yl)ethanone

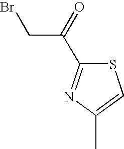

According to the procedure given for preparation of 2-bromo-1-(5-methyl-thiazol-2-yl)-ethanone, 4-methylthiazole (6.9 g, 69.6 mmol), n-butyllithium (73.1 mmol) and methyl bromoacetate (11.17 g,/3.1 mmol) gave crude product which was purified by SGC in EtOAc-hexanes followed by crystallization from 1:1 EtOAc-hexanes giving a colorless crystalline solid. Yield 4.3 g, 28%. ¹H NMR (CDCb) δ 7.33 (s, 1H). 4.71 (s, 2H), 2.54 (s, 3H).

### Example 64

### 1-(4-(4-(5-fluorothiophen-2-yl)-1-(6-methylpyridin-3-yl)-1H-imidazol-2-yl)phenyl)-1H-pyrrolo[2,3-b]pyridine

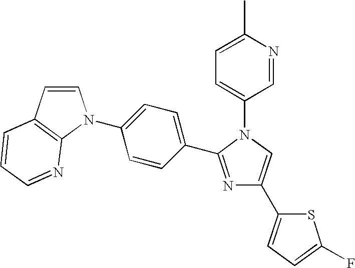

1(5-fluoro-thiophen-2-yl)-ethanone (522 mg, 238 mmol), NaHCO₂ (308 mg, 3.67 mmol, N′-(6-methylpyridin-3-yl)-4-(1H-pyrrolo[2,3-b]pyridin-1-yl)benzamidine (600 mg, 1.83 mmol) and 2-propanol were heated at reflux for 6 h. The mixture was filtered, concentrated, and the residue dissolved in acetic acid (5 ml), heated on a steam bath for 5 min, and concentrated. The residue was dissolved in EtOAc and the solution washed with aqueous NaOH, dried, and concentrated. The residue was purified by SGC (EtOAc-hexanes. Yield 290 mg, 35%. ¹H NMR (CDCl₃) δ 8.53 (d, 1H, J=2.5 Hz), 8.34 (dd, 1H, J=1.7, 4.7 Hz), 7.94 (dd, 1H, J=1.7, 7.9 Hz), 7.78 (m, 2H). 7.55 (m, 2H), 7.49 (d, 1H, J=3.7 Hz), 7.45 (dd, 1H, J= 2.5, 8.3 Hz), 7.25 (s, 1H), 7.20 (d, 1H, J=79 Hz), 7.12 (dd, 1H, J=4.6, 7.9 Hz), 6.97 (br, 1H), 6.62 (d, 1H, J=3.7 Hz), 6.42 (dd, 1H, J=2, 4 Hz). MS (AP+) m/e 452 (MH+). IC₅₀=6.59 nM.

### Preparation 64A

### 1-(5-fluoro-thiophen-2-yl)-ethanone

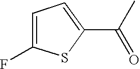

Methylmagnesium bromide in ether (28.3 ml of 3.0M, 85 mmol) was added to a solution of 5-fluorothiophene-2-carbonitrile (R. J. Chambers and A. Marfat, Synthetic Communications 2000, 30(19), 3629-3632, 9.0 g, 70.8 mmol) and the resulting mixture heated at reflux for 45 min. The mixture was poured into a mixture of ice and 20 mL cone. HCl. The resulting suspension was saturated with NaCl and filtered to remove a solid byproduct (2.7 g), and the filtrate extracted twice with DCM. The organic layers were dried and concentrated leaving a dark oil which was distilled (Kugelrohr, 10-20 mm) giving the product as a fight brown liquid (3.4 g, 33%). ¹H NMR (CDCl₃) δ 7.35 (dd, 1H, J=3, 4 Hz) 6.52 (dd, 1H, J=1, 4-5 Hz), 2.46 (s, 1H) was consistent with that reported (R. D. Schuetz and G. P. Nilles, J. Org. Chem. 1971, 36(15), 2188-2190).

### Preparation 64B

### 2-Bromo-1-(5-fluoro-thiophen-2-yl)-ethanone

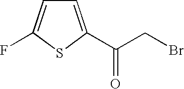

Pyridinium tribromide (1.63 g of 90% purity, 1.05 equiv) was added in one portion to a solution of 629 mg (4.37 mmol) 1-(5-fluoro-thiophen-2-yl)-ethanone in chloroform at RT. After 2 h, the solution was diluted with ether (50 mL) and the mixture washed with water, brine, dried, and concentrated. The resulting oil was purified by SGC (a gradient of DCM in hexanes) giving the title substance (684 mg, 70%) as an oily solid. ¹H NMR (CDCl₃) δ 7.49 (dd, 1H, J=3, 4 Hz), 6.56 (dd, 1H, J=1, 4.5 Hz), 4.25 (s, 2H); ¹³C NMR (CDCl₃) δ 184.58 (d, J=3 Hz). 173.10 (d, J=301 Hz), 132.27 (d, J=5 Hz), 130.37 (d, J=2 Hz), 110.30 (d, J=13 Hz), 29.11.

### Example 85

### 1-(4-(4-(4,5-dimethylthiazol-2-yl)-1-(pyrimidin-5-yl)-1H-imidazol-2-yl)phenyl)-1H-pyrrolo[2,3-b]pyridine

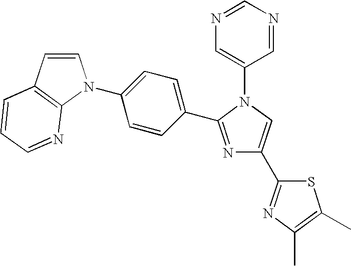

According to General Procedure 2,2-bromo-1-(4,5-dimethylthiazol-2-yl)ethanone (536 mg, 2.29 mmol), and N′-(pyrimidin-5-yl)-4-(1H-pyrrolo[2,3-b]pyridin-1-yl)benzamidine (600 mg, 1.91 mmol) gave a light red solid. Yield 90 mg, 10.5%. ¹H NMR (CDCl₃) δ 9.24 (s, 1H), 8.74 (s, 2H), 8.35 (dd, 1H, J=1.5, 4.8 Hz), 7.94 (dd, 1H, J=1.7, 7.9 Hz), 7.86 (m, 2H), 7.54 (m, 2H), 7.51 (d, 1H, J=3.7 Hz), 7.13 (dd, 1H, J=5.0, 7.9 Hz), 6.63 (d; 1H, J=3.7 Hz), 2.40 (s, 3H), 2.37 (s, 3H). MS (AP+) m/e 450 (MH+). IC₅₀=74.2 nM.

### Preparation 65A

### 2-bromo-1-(4,5-dimethylthiazol-2-yl)ethanone

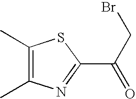

According to the procedure given for preparation of 2-bromo-1(5-methyl-4-thiazol-2-yl) ethanone, 4,5-dimethylthiazole (8.97 g, 79.4 mmol), n-butyllithium in hexanes (83.3 mmol), and methyl bromoacetate (12.7 g, 83.3 mmol) gave crude product which was crystallized from 4:1 EtOAc-hexanes. Yield 8.6 g, 48%. ¹H NMR(COCl₃) δ 4.64 (s, 2H), 2.44 (s, 3H), 2.39 (s, 3H). ¹³C NMR (CDCl₃) δ 184.72, 158.61, 152.30, 138.07, 31.16, 15.12, 12.48.

### Example 66

### 1-(4-(4-(1-methyl-1H-imidazol-2-yl)-1-(2-methylpyridin-4-yl)-1H-imidazol-2-yl)phenyl)-1H-pyrrolo[2,3-b]pyridine

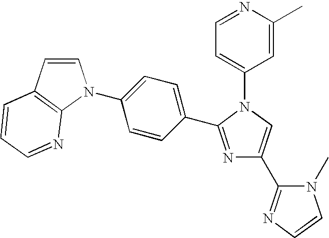

According to General Procedure 2, N′-(2-methylpyridin-4-yl)-4-(1H-pyrrolo[2,3-b]pyridin-1-yl)benzamidine (500 mg, 153 mmol) and 2-bromo-1-(1-methyl-1H-imidazol-2-yl)ethanone (372 mg, 1.83 mmol) gave chromatographed product which was further purified by RP-HPLC (basic conditions) giving an off-white solid. Yield 47 mg, 7%), ¹H NMR (CDCl₃) δ 8.55 (d, 1H, J=5.4 Hz), 8.36 (dd, 1H, J=1.7, 4.6 Hz), 8.02 (br, 1H), 7.96 (dd, 1H, J=17, 7.9 Hz), 7.82 (m, 2H), 7.56 (m, 2H) 7.51 (d 1H J=3.7 Hz), 7.16-7.12 (m, 3H), 7.02 (dd, 1H, J=1.9, 5.6 Hz), 6.92 (d, 1H, J=1 Hz), 6.64 (d, 1H, J=3.7 Hz), 4.18 (s, 3H) MS (AP+) m/e 432 (MH+). IC₅₀=112 nM.

### Preparation 66A

### 2-bromo-1-(1-methyl-1H-imidazol-2-yl)ethanone

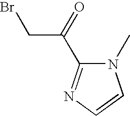

According to the procedure given for preparation of 2-bromo-1-(5-methyl-thiazol-2-yl)-ethanone, N-methylimidazole (1.80 g, 21.9 mmol), n-butyllithium in hexanes (23.0 mmol), and methyl bromoacetate (3.5 g, 23.0 mmol) gave crude product which was triturated with 1:2 EtOAc-hexanes giving a solid, which was suspended in hot DCM and filtered. Evaporation of the filtrate gave a yellow solid. Yield 1.2 g, 27%. ¹H NMR (CDCl₃) δ7.17 (s, 1H), 7.09 (s, 1H), 4.68 (s, 2H), 4.00 (s, 3H).

### Example 67

### 1-(4-(4-(1-methyl-1H-imidazol-2-yl)-1-(pyrimidin-5-yl)-1H-imidazol-2-yl)phenyl)-1H-pyrrolo2,3-b]pyridine

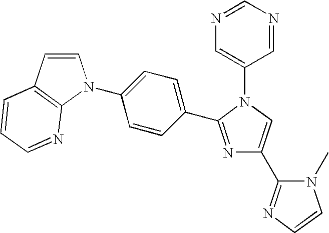

According to General Procedure 2,2-bromo-1-(1-methyl-1H-imidazol-2-yl)ethanone (342 mg, 1.69 mmol) and N′-(pyrimidin-5-yl)-4-(1H-pyrrolo-2,3-b]pyridin-1-yl)benzamidine (530 mg, 169 mmol) gave chromatographed product which was further purified by RP-HPLC (basic conditions). Yield 100 mg, 14%. ¹H NMR (CDCb) δ 9.27 (s, 1H), 8.78 (s, 2H), 8.35 (dd, 1H, J=1.7, 4.6 Hz), 8.26 (s, 1H), 8.20 (s, 1H), 7.96 (dd, 1H, J=1.7, 7.9 Hz), 7.85 (m, 2H), 7.53-7.50 (m, 3H), 7.18 (d, 1H, J=1 Hz), 7.14 (dd, 1H, J=4.6, 7.9 Hz), 6.64 (d, 1H, J=3.7 Hz), 4.21 (s, 3H). MS (AP+) m/e 419 (MH+). IC₅₀=274 nM.

### Example 68

### 1-(4-(1-(2-methylpyridin-4-yl)-4-(pyridin-3-yl)-1H-imidazol-2-yl)phenyl)-1H-pyrrolo[2,3-b]pyridine bis-TFA salt

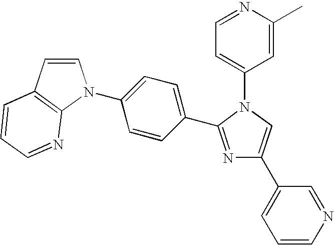

According to General Procedure 2, N′-(2-methylpyridin-4-yl)-4-(1H-pyrrolo[2,3-b]pyridin-1-yl)benzamidine (347 mg, 1.06 mmol) and 2-bromo-1-(pyridin-3-yl)ethanone hydrobromide (299 mg, 106 mmol) gave a yellow solid after RP-HPLC purification. Yield 30 mg, ¹H NMR (CDCb) δ 9.41 (d, 1H, J=2 Hz), 8.87 (dt, 1H, J=1.7, 8.3 Hz), 8.71 (d, 1H, J= 5.6 Hz), 8.62 (dd, 1H, J=1.7, 5.4 Hz). 8.40 (dd, 1H, J=15, 4.8 Hz), 8.04 (dd, 1H, J=1.7, 7.9 Hz), 7.96 (s, 1H), 7.91-7.85 (m, 3H), 7.59 (m, 2H), 7.54 (d, 1H, J=3.7 Hz). 7.39 (d, 1H, J= 2 Hz), 7.28 (dd, 1H, J=2, 6 Hz), 7.20 (dd, 1H, J=4.8, 7.7 Hz), 6.70 (d: 1H, J=3.7 Hz), 4.0 (br, >3H), 2.74 (s, 3H). MS (AP+) m/e 429 (MH+). IC₅₀=5.10 nM.

### Example 69

### 1-(4-(1-(2-methylpyridin-4-yl-4-(pyridin-4-yl)-1H-imidazol-2-yl)phenyl)-1H-pyrrolo-[2,3-b]pyridine bis-TFA salt

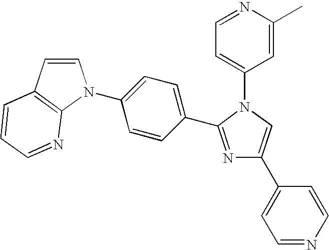

According to General Procedure 2, N′-(2-methylpyridin-4-yl)-4-(1H-pyrrolo[2,3-b]pyridin-1-yl)benzamidine (347 mg, 1.06 mmol) and 2-bromo-1-(pyridin-4-yl)ethanone hydrobromide (298 mg, 106 mmol) gave a solid after RP-HPLC purification. Yield 54 mg. ¹H NMR (CDCb) 8.80 (d, 2H, J=7 Hz), 8.76 (d, 1H, J=5.6 Hz), 8.40 (dd, 1H, J=1.7, 5.0 Hz), 8.32 (6, 2H, J=7 Hz), 8.13 (s, 1H), 8.08 (dd, 1H, J=1.7, 7.9 Hz), 7.90 (m, 2H), 7.62 (m, 2H), 7.55 (d, 1H, J=3.7 Hz), 7.41 (d, 1H, J=17 Hz), 7.32 (dd, 1H, J=17, 5.8 Hz), 7.25 (dd, 1H, J=5, 7.9 Hz), 6.74 (d, 1H, J=3.7 Hz), 2.74 (s, 3H). MS (AP+) m/e 429 (MH+). IC₅₀=89.7 nM.

### Example 70

### 5-(2-(4-(3,4-dichloropheny)phenyl)-4-(pyridin-2-yl)-1H-imidazol-1-yl)pyrimidine

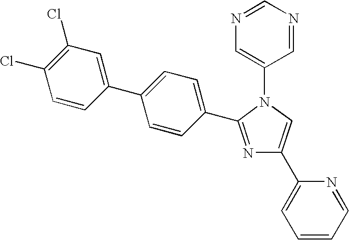

A mixture of 5-(2-(4-bromophenyl)-4-(pyridin-2-yl)-1H-imidazol-1-yl)pyrimidine (100 mg, 0.27 mmol). 3,4-dichlorophenylboronic acid (50 mg, 0.27 mmol), 2M aqueous sodium carbonate (0.26 ml, 0.52 mmol) and tetrakis-(triphenylphosphine)palladium(0) (6 mg; in toluene (1 ml; and ethanol (1 mL) was heated by microwave at 130° C. for 15 min. The mixture was combined with another similarly prepared (0.13 mmol scale) and treated with aqueous 3% hydrogen peroxide (4 mL). The mixture was partitioned between aqueous NaOH (5 ml) and DCM (30 ml) and separated. The organic layer was washed with water, dried, concentrated, and the residue purified by SGC (1% MeOH in DCM giving an off-white solid, Yield 75 mg. ¹H NMR (CDCb) δ 9.24 (s, 1H), 8.73 (s, 2H), 8.58 (ddd, 1H, J=1.2.5 Hz), 8.13 (m, 1H), 7.93 (br, 1H), 7.77 (dt, 1H, J=2, 8 Hz), 7.64 (d, 1H, J=2 Hz). 7.53-7.47 (m, 5H), 7.38 (dd, 1H, J=2, 8.5 Hz), 7.22 (m, 1H). MS (AP+) m/e 444/446 (2.1. MH+). IC₅₀=216 nM.

### Example 70A

### 4-bromo-N′-(pyrimidin-5-yl)benzamidine

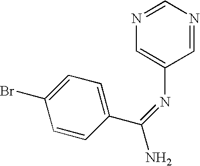

According to General Procedure 1, sodium hydride dispersion (60%, 5.52 g, 138 mmol), 4-bromobenzonitrile (11.4 g, 63.0 mmol), and 5-aminopyrimidine (6.00 g, 63.0 mmol, Philips et al. Can. J. Chem 1999, 77, 216-222) in anhydrous dimethylsulfoxide (120 mL; at 55° C. for 3 h gave a mixture which was poured into ice water (200 ml) and 1:1 EtOAc-hexanes (100 ml). After stirring the precipitate was filtered and washed with water (4×100 ml) and 1:1 EtOAc-hexanes (2×100 ml) and dried. Yield 8.53 g, 50%. ¹H NMR (CDCb) δ 8.92 (s, 1H), 8.43 (s, 2H), 7.74 (d, 2H, J=8.5 Hz), 7.61 (d, 2H, J=8.5 Hz), 4.98 (br, 2H). MS (AP+) m/e 277/279 (1:1, MH+).

### Preparation 70B

### 5-(2-(4-bromophenyl)-4-(pyridin-2-yl)-1H-imidazol-1-yl)pyrimidine

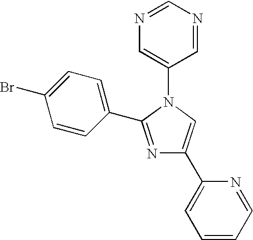

According to General Procedure 2,4-bromo-N′-(pyrimidin-5-yl)benzamidine (2.00 g, 7.25 mmol), LiHMDS in THF (18.1 mL of 1.0 M), and 2-bromo-1-(pyridin-2-yl)ethanone hydrobromide (2.04 g, 7.25 mmol) gave after acetic acid treatment, extraction with DCM-aqueous NaOH and washing with aqueous citric acid a crude solid which was further triturated with diethyl ether. Yield 850 mg. ¹H NMR (CDCl₃) δ 9.24 (s, 1H). 8.70 (s, 2H), 8.56 (m, 1H), 8.08 (dt, 1H, J=1, 8 Hz), 7.88 (s, 1H), 7.76 (dt, 1H, J=2, 8 Hz), 7.48 (m, 2H), 7.28 (m, 2H), 7.20 (ddd, 1H, J=1.5, 8 Hz).

### Example 71

### 5-(2-(4-(4-chlorophenyl)phenyl)-4-(pyridin-2-yl)-1H-imidazol-1-yl)pyrimidine

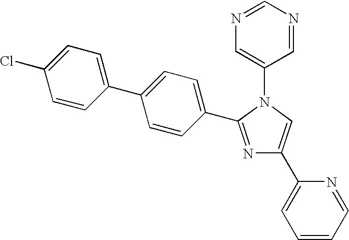

In an analogous manner to the preparation given for 5-(2-(4-(3,4-dichlorophenyl)phenyl)-4-(pyridin-2-yl)-1H-imidazol-1-yl)pyrimidine, except that the reaction mixture was heated by microwave for 60 mm, 4-chlorophenylboronic acid (42 mg, 0.27 mmol) and 5-(2-(4-bromophenyl)-4-(pyridin-2-yl)-1H-imidazol-1-yl)pyrimidine (100 mg, 0.27 mmol) gave an off-white solid. Yield 65 mg. ¹H NMR (CDCb) δ 9.24 is, 1H), 8.74 (s, 2H), 8.57 (m, 1H), 8.14 (d, 1H, J=7.5 Hz), 7.94 (br, 1H), 7.79 (t, 1H), 7.65 (m, 1H), 7.53 (m, 2H), 7.49-7.46 (m, 3H), 7.40 (m, 2H), 7.22 (m, 1H). MS (AP+) m/e 410 (MH+). IC₅₀=67.6 nM.

### Example 72

### 5-(4-pyridin-2-yl)-2-(4-(pyridin-4-yl)phenyl)-1H-imidazol-1-yl)pyrimidine

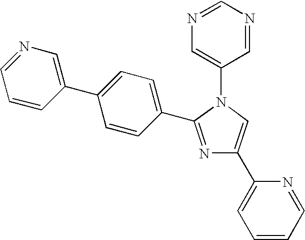

In an analogous manner to the preparation given for 5-(2-(4-(3,4-dichlorophenyl)phenyl)-4-(pyridin-2-yl)-1H-imidazol-1-yl)pyrimidine, except that the reaction mixture was first heated by microwave for 95 min, then with a second equivalent of boronic acid for 80 min, 3-pyridylboronic acid (99 mg, 0.80 mmol in too portions) and 5-(2(4-bromophenyl)-4-(pyridin-2-yl)-1H-imidazol-1-yl)pyrimidine (150 mg, 0.40 mmol) gave a brown solid. Yield 25 mg. ¹H NMR (CDCb) δ 9.24 (s, 1H) 8.82 (d, 1H, J=1.7 Hz), 8.75 (s, 2H), 8.60 (m, 1H), 8.58 (m, 1H), 8.12 (d, 1H, J=8 Hz). 7.92 (s, 1H), 7.86 (ddd, 1H), 7.77 (dt. 1H, J=1.8, 8 Hz), 7.57 (m, 2H). 7.52 (m, 2H), 7.36 (dd, 1H, J=2, 8 Hz), 7.21 (ddd, 1H, J=1.5.7 Hz). MS (AP+) m/e 377 (MH+), IC₅₀=160 nM.

### Example 73

### 5-(4-(pyridin-2-yl)-2-(4-(phenyl)-1H-imidazol-1-yl)pyrimidine

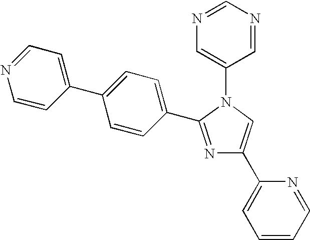

In an analogous manner to the preparation given for 5-(2-(4-(3,4-dichlorophenyl)phenyl)-4-(pyridin-2-yl)-1H-imidazol-1-yl)pyrimidine, except that the reaction mixture was heated at 140° C. by microwave for 30 min, 4-pyridylboronic acid (131 mg, 1.06 mmol) and 5-(2-(4-bromophenyl)-4-(pyridin-2-yl)-1H-imidazol-1-yl)pyrimidine (200 mg, 0.53 mmol) gave an off-white solid. Yield 52 mg. ¹H NMR (CDCb) δ 9.25 (s, 1H), 8.74 (s, 2H), 8.67-8.65 (m, 2H), 8.55 (ddd, 1H, J=1, 2, 5 Hz), 8.13 (d, 1H, J=8 Hz), 7.96 (br, 1H), 7.79 (dt, 1H, J=2, 8 Hz), 7.61 (m, 2H), 7.54 (m, 2H), 7.50-7.46 (m, 2H), 7.22 (m, 1H). MS (AP+) m/e 377 (MH+). IC₅₀=74.3 nM.

### Example 74

### 7-(4-(1-(6-methylpyridin-3-yl)-4-(pyridin-2-yl)-1H-imidazol-2-yl)phenyl)-7H-pyrrolo[2,3-d]pyrimidine

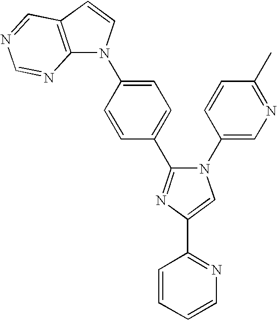

A mixture of 2-(2-(4-iodophenyl)-1-(6-methylpyridin-3-yl)-1H-imidazol-4-yl)pyridine (200 mg, 0.45 mmol), 7H-pyrrolo[2,3-d]pyrimidine (54 mg, 0.54 mmol), CuI (4 mg, 0.022 mmol), K₃PO₄ (218 mg, 1.03 mmol) and N,N-dimethyl-trans-1,2-cyclohexanediamine (6 mg, 0.045 mmol) in p-dioxane (0.3 mL) was heated by microwave at 150° C. for 2 h, and at 180° C. for 1 h, filtered through silica using DCM-MeOH, the filtrate concentrated and the residue purified by SGC (1-2% MeOH in DCM, 0.5% NH₄OH) giving a yellow solid. Yield 51 mg. ¹H NMR (CDCl_{3) δ} 9.04 (s, 1H). 8.93 (s, 1H), 8.59 (m, 1H). 8.54 (d, 1H, J=2.5 Hz), 8.20 (br, 1H), 7.86 (br, 1H), 7.78 (m, 2H). 7.63 (m, 2H), 7.55-7.50 (m, 2H), 7.26-7.24 (m, 3H), 6.73 (d, 1H, J=3.7 Hz), 2.63 (s, 3H). MS (AP+) m/e 430 (MH+). IC₅₀=2.27 nM.

### Example 75

### 7-methyl-5-(1-(6-methylpyridin-3-yl)-4-(pyridin-2-yl)-1H-imidazol-2-yl)phenyl-5H-pyrrolo-2,3-b]pyrazine

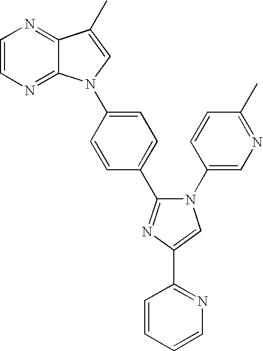

A mixture of 2-(2-(4-iodophenyl)-1-(8-methylpyridin-3-yl)-1H-imidazol-4-yl)pyridine (200 mg, 0.45 mmol). 7-methyl-5H-pyrrolo[2,3-b]pyrazine (76 mg. 0.45 mmol), CuI (4 mg, 0.022 mmol), K₃PO₄ (296 mg, 14 mmol), and N,N-dimethyl-trans-1,2-cyclohexanediamine (6 mg, 0.045 mmol) was heated by microwave at 150° C. for 1 h and at 180° C. for 1 h, filtered through silica using 3:1 DCM-MeOH, concentrated, and the residue purified by SGC in 1-2% MeOH in DCM giving a yellow solid (102 mg). This was further purified by RP-HPLC (basic conditions) giving 50 mg of a yellow solid. ¹H NMR (CDCl₃) δ 8.58 (d, 1H, J=5 Hz), 8.54 (d, 1H, J=2.5 Hz), 8.46 (d, 1H, J=3 Hz), 8.27 (d, 1H, J=2.5 Hz), 8.15 (m, 1H), 7.9 (br, 1H), 7.81-7.77 (m, 2H and br, 1H), 7.64 (s, 1H), 7.60 (m, 2H), 7.51 (dd, 1H, J=2.5, 8 Hz), 7.23-7.19 (m, 2H), 2.63 (s, 3H), 2.45 (s, 3H). MS (AP+) m/e 444 (MH+). IC₅₀=2.41 nM.

### Example 76

### 1-(4-(4-benzo[d]thiazol-2-yl)-1-(pyridin-3-yl)-1H-b]pyridine

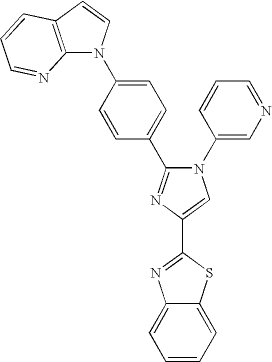

According to General Procedure 2, N′-(pyridin-3-yl)-4-(1H-pyrrolo[2,3-b]pyridin-1-yl)benzamidine (613 mg, 1.96 mmol), LiHMDS (4.3 mL of 1M in THF), and 1-(benzo[d]thiazol-2-yl)-2-bromoethanone (500 mg, 196 mmol) gave a brown solid after SGC and trituration with ether. Yield 131 mg. ¹H NMR (CDCl₃) δ 8.71 (m, 2H), 8.38 (dd, 1H, J=1.7, 4.5 Hz), 8.06 (br, 1H), 8.03 (d, 1H, J=8.3 Hz), 7.98-7.93 (m, 2H), 7.84 (m, 2H), 7.66 (m, 1H), 7.60 (m, 2H), 7.52 (d, 1H, J=3.7 Hz), 7.49 (m, 1H), 7.43 (dd, 1H, J=4.6, 8 Hz), 7.38 (dt, 1H, J=1, 7 Hz), 7.14 (dd, 1H, J=5, 8 Hz), 6.64 (d, 1H, J=3.3 Hz). MS (AP+) m/e 471 (MH+). IC₅₀=544 nM.

### Preparation 76A

### 1-benzo[d]thiazol-2-yl-2-bromoethanone

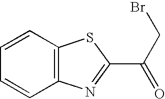

n-Butyllithium in hexanes (6.21 mL of 2.5 M) was added dropwise to a stirred −78° C. solution of benzothiazole (2.00 g, 14.8 mmol) in ether (20 ml). After 15 min, methyl bromoacetate (2.4 g; 15.5 mmol) was added in one portion at −78° C. giving a suspension, which was stirred at −78° C. for 15 min. Acetic acid (1.8 g, 31 mmol) was added at −78° C. and the mixture was warmed to RT. Ether (20 mL) and water were added. The organic layer was separated, dried, and concentrated. The resulting oily solid was dissolved in hot isopropyl ether and the suspension filtered. The filtrate was evaporated and the residue suspended in hexanes. The solid was filtered and dried. Yield 1.02 g. 27%, orange solid. ¹H NMR (CDCl₃, 400 mHz) δ 8.20 (m, 1H), 8.01 (m, 1H), 7.63-7.55 (m, 2H), 4.84 (s, 2H).

### Example 77

### 4-methoxy-6-methyl-8-(4-(4-(pyridin-2-yl)

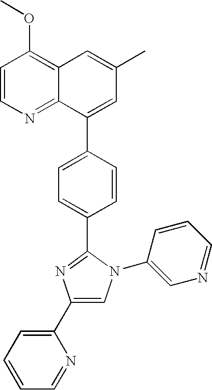

2-(2-(4-(trimethylstannyl)phenyl)-1-(pyridin-3-yl)-1H-imidazol-4-yl)pyridine (153 mg, 0.33 mmol), 8-bromo-4-methoxy-6-methylquinoline (88 mg, 0.35 mmol), tetrakis-(triphenylphosphine)palladium (38 mg, 0.033 mmol), CuI (19 mg, 0.10 mmol) and p-dioxane (3 ml) were heated at 125° C. for 20 h, The mixture was concentrated and purified by SGC (0.5% and 1% MeOH in DCM, 0.5% NH₄OH) giving a yellow solid (95 mg). This was further purified by RP-HPLC (basic conditions) giving a white solid. Yield 69 mg, 44%. ¹H NMR (CDCl₃) δ 9.03 (d, 1H, J=6 Hz), 8.87 (d, 1H, J=4.8 Hz), 8.82 (s, 1H), 8.78 (m, 1H), 8.73 (d, 1H, J=4.5 Hz), 8.61 (d, 1H, J=8.3 Hz), 8.32 (t, 1H, J=8 Hz), 8.19 (br, 1H), 8.02 (d, 1H J=8 Hz), 7.73 (d, 1H, J=17 Hz), 7.66-7.60 (m, 4H), 7.44 (d, 2H, J=8 Hz), 7.13 (d, 1H, J=6 Hz), 4.32 (s, 3H), 2.64 (s, 3H). MS (AP+) m/e 470 (MH+). IC₅₀=156 nM.

### Preparation 77A

### 2-(2-(4-(trimethylstannyl)phenyl)-1-(pyridin-3-yl)-1H-imidazol-4-yl)pyridine

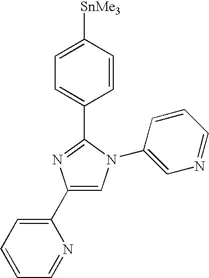

A mixture of 2-(2-(4-iodophenyl)-1-(pyridin-3-yl)-1H-imidazol-4-yl)pyridine (1.24 g, 2.93 mmol), hexamethylditin (1.15 g, 3.51 mmol), tetrakis-(triphenylphosphine)palladium(0) (338 mg, 0.293 mmol) in p-dioxane was heated at 110° C. for 24 h, concentrated, and the residue purified by SGC (4:1 EtOAc-hexanes containing 0.5% triethylamine, giving a yellow solid. Yield 1.05 g, 80%. ¹H NMR (CDCl₃) 5 (partial) 8.65 (dd, 1H, J=1.5, 4.7 Hz), 8.57 (m, 2H), 8.23 (br, 1H), 7.87 (br, 1H), 7.42 (d, 2H), 7.35 (d, 2H), 0.26 (s, 9H)

### Example 78

### 8-(4-(4-pyridin-2-yl)-1-(pyridin-3-yl)-1H-imidazol-2-yl)phenyl)-1,7-naphthyridine

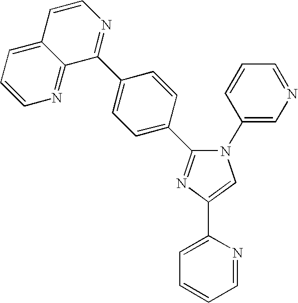

2-(2-(4-(tributylstannyl)phenyl)-1-(pyridin-3-yl)-1H-imidazol-4-yl)pyridine (180 mg, 0.31 mmol), 8-bromo-1,7-naphthyridine (77 mg. 0.37 mmol), tetrakis-(triphenylphosphine)palladium (38 mg, 0.033 mmol), CuI (17 mg, 0.092 mmol) and p-dioxane (4 ml) were heated by microwave at 160° C. for 2 h. The mixture was concentrated and purified by RP-HPLC (basic conditions) giving a white solid. Yield 4 mg. ¹H NMR (CDCl₃) δ 9.03 (dd, 1H, J=1.7, 4 Hz), 8.75 (d, 1H, J=5 Hz), 8.71-8.68 (m, 3H), 8.43-8.40 (m, 2H). 8.20 (dd, 1H, J=2, 8.3 Hz), 8.15 (m, 2H), 8.05 (m, 1H), 7.68 (m, 1H), 7.64 (d, 1H, J=5 Hz), 7.63-7.57 (m, 3H), 7.43-7.39 (m, 2H). HPLCMS 4.65 mm, m/e 427 (MH+)) IC₅₀=3.26 nM.

### Preparation 78A

### 2-(2-(4-(tributylstannyl)phenyl)-1-(pyridin-3-yl)-1H-imidazol-4-yl)pyridine

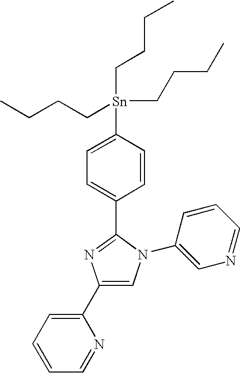

A mixture of 2-(2-(4-iodophenyl)-1-(pyridin-3-yl)-1H-imidazol-4-yl)pyridine (753 mg, 1.78 mmol), hexabutylditin (1.23 g, 2.13 mmol), tetrakis-(triphenylphosphine)palladium(0) (163 mg, 0.14 mmol) in p-dioxane (10 mL) and triethylamine (1 mL) was heated at 150° C. for 21 h, concentrated, and the residue purified by SGC (EtOAc containing 0.5% triethylamine, giving a yellow solid. Yield 500, 48%. ¹H NMR (CDCl₃) δ 8.65 (dd, 1H, J=1.5, 4.8 Hz), 8.61 (d, 1H, J=2 Hz), 8.57 (d, 1H, J=4 Hz), δ 20 (br, 1H), 7.81 (br, 1H), 7.68-7.66 (m, 1H), 7.53 (m, 1H), 7.47-7.42 (m, 1H), 7.40-7.33 (m, 5H), 1.48 (m, 6H), 1.29 (m, 6H), 102 (m, 8H), 0.85 (t, 9H, J=7 Hz).

### Example 79

### 8-(4-(4-(pyridin-2-yl)-1-(pyridin-3-yl)-1H-imidazol-2-yl)phenyl)quinoline

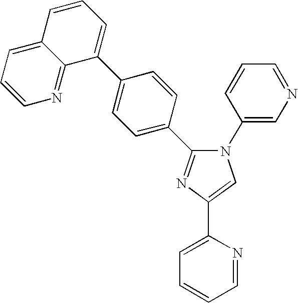

2-(2-(4-(tributylstannyl)phenyl)-1-(pyridin-3-yl)-1H-imidazol-4-yl)pyridine (258 mg, 0.44 mmol), 8-bromoquinoline (100 mg, 0.48 mmol), tetrakis-(triphenylphosphine)palladium (51 mg, 0.044 mmol), Cut (25 mg, 0.13 mmol) and p-dioxane (3 mL) were heated by microwave at 150° C. for 3 h. The mixture was concentrated, filtered through silica, and purified by RP-HPLC (basic conditions) giving a yellow solid. Yield 36 mg, 20%, About 5% of 2-(2-phenyl-1-(pyridin-3-yl)-1H-imidazol-4-yl)pyridine derived from destannylation of starting material was judged to be present by HPLCMS. ¹H NMR (CDCl_{3) δ 9.03} (m, 1H), 8.94 (br, 1H), 8.82 (d, 1H, J=6 Hz), 8.71 (m, 2H), 8.63 (d, 1H, J=8 Hz), 8.38 (d, 1H, J=8 Hz), 8.30 (t, 1H, J=7 Hz), 7.92 (dd, 1H, J=1.5, 8 Hz), 7.86 (d, 1H, J=9 Hz), 7.76 (m, 1H), 7.70-7.50 (m, 8H). MS (AP+) m/e 426 (MH+). IC₅₀=3.94 nM.

### Example 80

### 6-methoxy-8-(4-(4-(pyridin-2-yl)-1-(pyridin-3-yl)-1H-imidazol-2-yl)phenyl)quinoline

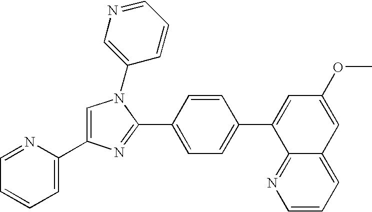

2-(2-(4-(trimethylstannyl)phenyl)-1-(pyridin-3-yl)-1H-imidazol-4-yl)pyridine (188 mg, 0.41 mmol), 8-bromo-6-methoxyquinoline (102 mg, 0.43 mmol), tetrakis-(triphenylphosphine)palladium (50 mg, 0.042 mmol), CuI (24 mg, 0.13 mmol) and p-dioxane (3 mL) were heated at 125° C. for 19 h. The mixture was concentrated, filtered through silica, and purified by RP-HPLC (basic conditions) giving a green solid. Yield 54 mg, 29%. ¹H NMR (CDCl₃) δ (partial) 8.97 (d, 1H, J=5 Hz), 8.89 (d, 1H, J=6 Hz), 8.74 (s, 1H), 8.63-8.59 (m, 2H), 8.32 (t, 1H, J=8 Hz), 7.96 (d, 1H, J=8 Hz), 7.76 (m, 1H), 7.59 (m, 2H), 7.53-7.50 (m, 3H), 7.29 (d, 1H, J=2.5 Hz), 4.02 (s, 3H). HPLCMS 5.96 min, m/e 456 (MH+). IC₅₀=0.794 nM.

### Example 81

### 2-methoxy-3-(4-(1-(6-methylpyridin-3-yl)-4-(thiazol-2-yl)-1H-imidazol-2-yl)phenyl)-3H-imidazo[4,5-b]pyridine

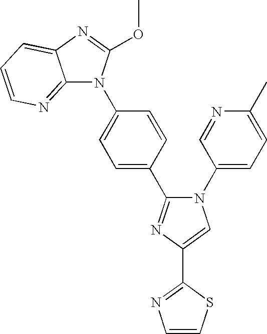

A mixture of N²-(4-(1-(6-methylpyridin-3-yl)-4-(thiazol-2-yl)-1H-imidazol-2-yl)phenyl)pyridine-2,3-diamine (2.1 g, 4.94 mmol), tetramethylorthocarbonate (13 mL), and propionic acid (approximately 120 mg, 0.35 equiv) was heated at 80° C. for 2 h. Another portion of propionic acid (approx. 75 mg) was added and the mixture heated again at 85° C. for 3 h, The mixture was evaporated, dissolved in DCM and washed with aqueous NaHCO₃. The aqueous layer was extracted with 5:1 DCM.2-propanol. The organic layers were combined, dried over Na₂SO₄, and concentrated. Chromatograpy on silica (gradient of 0.5%-2% MeOH in DCM, 0.5% NH₄OH gave 5.0 g of a colorless solid which was dissolved in ether. The solid which formed was filtered, washed with ether and dried (4.28 g). Recrystallization of this material from acetonitrile containing 2% water gave crystalline material, m.p, 180-181° C., On a different occasion, recrystallization from hot acetonitrile gave another form, m.p. 190-192° C. ¹H NMR (CDCl₃) δ 8.56 (d, 1H, J=2.5 Hz), 8.15 (dd, 1H, J=1.5, 5 Hz), 7.82 (d, 1H, J=3 Hz), 7.81 (dd, 1H, J=1.7, 7.9 Hz), 7.77 (s, 1H), 7.61 (s, 4H), 7.50 (dd, 1H, J=2.5, 8.5 Hz), 7.31 (d, 1H, J=3.3 Hz), 7.24 (d, 1H, J=8.3 Hz), 7.16 (dd, 1H, J=5.0, 7.9 Hz), 4.20 (s, 3H), 2.63 (s, 3H). HPLCMS 7.04 min, m/e 466 (MH+). IC₅₀=0.330 nM.

### Preparation 81A

### 5-(2-(4-iodophenyl)-4-(thiazol-2-yl)-1H-imidazol-1-yl)-2-methylpyridine

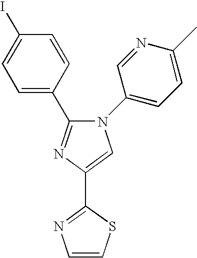

According to General Procedure 2,4-iodo-N′-(8-methylpyridin-3-yl)benzamidine (25.6 g, 76.0 mmol) and 2-bromoacetylthiazole (18.7 g) were condensed using LiHMDS (80 mL of 1M in THF, 80 mmol), and the crude product isolated by EtOAc extraction and treated with hot acetic acid. At this point this mixture was combined with another mixture identically prepared from 8.13 mmol of amidine (now 84.13 mmol total), and after EtOAc-aqueous NaOH extraction and citric acid washing, the crude product was purified by SGC using a gradient of 50% to 100% EtOAc in hexanes giving 10.6 g of the title product. ¹H NMR (CDCl₃) δ 8.45 (d, 1H, J=2.5 Hz), 7.81 (d, 1H, J=3.3 Hz), 7.75 (s, 1H), 7.64 (m, 2H), 7.42 (dd, 1H, J=2.5, 8.3 Hz), 7.30 (d, 1H, J=3.3 Hz), 7.22 (d, 1H, J=8.3 Hz), 7.13 (m, 2H), 2.63 (s, 3H). HPLCMS 9.17 min, m/e 445 (MH+).

### Preparation 81B

### N-(4-(1-(6-methylpyridin-3-yl)-4-(thiazol-2-yl)-1H-imidazol-2-yl)phenyl)-3-nitropyridin-2-amine

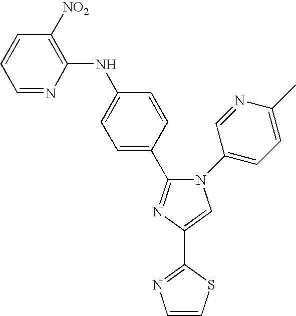

A mixture of 5-(2-(4-iodophenyl)-4-(thiazol-2-yl)-1H-imidazol-1-yl)-2-methylpyridine (2.70 g, 6.1 mmol), 2-amino-3-nitropyridine (1.01 g, 7.3 mmol), tris(dibenzylideneacetone)dipalladium(0) (166 mg, 0.18 mmol), 4,5-bis(diphenylphosphino)-9,9-dimethylxanthene (263 mg, 0.46 mmol), Cs₂CO₃ (2.97 g, 9.12 mmol) and p-dioxane (20 mL) was heated by microwave at 150° C. for 3 h. The mixture was combined with one prepared identical/from 3.75 g (8.44 mmol) of starting iodide, filtered, concentrated, and the residue purified by SGC (gradient of 30% to 100% EtOAc-hexanes) giving 4.6 grams (70%) of a red solid. ¹H NMR (CDCl₃) δ 10.23 (br, 1H)< 8.54-8.48 (m, 2H), 7.81 (d, 1H, J=3.3 Hz). 7.78 (br, 1H), 7.69 (m, 2H), 7.48 (d, 1H, J=2.5 Hz), 7.47 (dd, 1H), 7.44 (m, 2H), 7.31 (d, 1H, J= 3 Hz). 7.22 (d, 1H, J=8.3 Hz), 8.87 (dd, 1H, J=4.6, 8.3 Hz), 2.62 (s, 3H). HPLCMS 8.92 min, m/e 456 (MH+),

### Preparation 81C

### N₂-(4-(1-(6-methylpyridin-3-yl)-4-(thiazol-2-yl)-1H-imidazol-2-yl)phenyl)pyridine-2,3-diamine

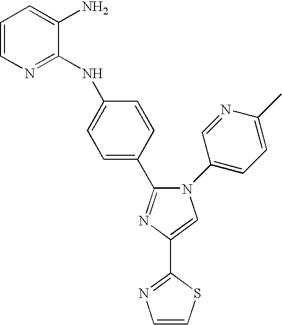

A mixture of N-(4-(1-(6-methylpyridin-3-yl)-4-(thiazol-2-yl)-1H-imidazol-2-yl)phenyl)-3-nitropyridin-2-amine (6.4 g, 14.0 mmol), and 10% palladium on carbon (1 g) was shaken under 45 p.s.i. hydrogen pressure for 4 h, filtered, and concentrated giving 6.0 g of a solid (100%). ¹H NMR (CDCl₃) δ 8.51 (d, 1H), 7.80 (dd, 1H, J=1, 5 Hz). 7.79 (d, 1H, J=3 Hz). 7.71 (s, 1H), 7.42 (dd, 1H, J=8.0, 2.7 Hz), 7.32 (d, 2H), 7.28 (d, 1H, J=3.3 Hz), 7.24-7.17 (m; 3H), 7.01 (dd, 1H, J=1.5, 8 Hz), 6.78 (dd, 1H, J=4.8, 7.7 Hz), 6.46 (br, 1H), 2.60 (s, 3H), 1.65 (br, 2H). HPLCMS 4.10 min, m/e 426 (MH+).

### Example 82

### 2-ethyl-3-(4-(1-(6-methylpyridin-3-yl)-4-(5-methylthiazol-2-yl)-1H-imidazol-2-yl)phenyl)-3H-imidazo[4,5-b]pyridine

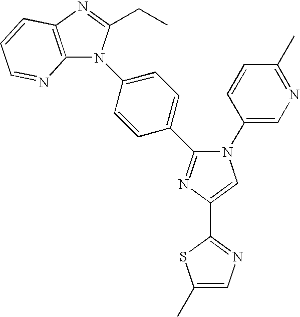

A mixture of 4-(2-ethyl-3H-imidazo[4,5-b]3-pyridin-3-yl)-NH₆-methylpyridin-3-yl)benzamidine (140 mg, 0.39 mmol), 2-bromoacetyl-5-methyl (thiazole (128 mg, 0.59 mmol), NaHCO₃ (131 mg, 1.58 mmol) and 2-propanol (2 ml) was heated in a capped vial at 90° C. for 2 h. The mixture was filtered and concentrated and the residue dissolved in acetic acid and heated at 90° C. for 20 min. The mixture was concentrated and the residue dissolved in dichoromethane. The solution was washed with 10% citric acid (2×5 mL), brine, dried (MgSO₄) and concentrated. The residue was purified by SGC (2% MeOH in DCM, NH₄OH) followed by RP-HPLC (basic system) giving 14 mg of an off-white solid. ¹H NMR (CDCl₃) δ 8.55 (d, 1H, J=2.5 Hz), 8.29 (dd, 1H, J=1.5, 5 Hz), 8.04 (dd, 1H, J=1.5, 8 Hz), 7.78 (br, 1H), 7.67 (m, 2H), 7.53 (dd, 1H, J=2.5, 8.3 Hz), 7.46 (m, 1H), 7.37 (m, 2H), 7.26 (d, 1H, J=8.5 Hz), 7.24 (dd, 1H, J=5, 8 Hz), 2.64 (s, 3H), 2.52 (d, 3H, J=1 Hz). 2.81 (q, 2H, J=7.5 Hz), 1.34 (t, 3H, J=7.5 Hz), HPLCMS 8.93 min, m/e 478 (MH+).

### Preparation 82A

### 4-(2-ethyl-3H-imidazo[4,5-b]pyridin-3-yl)benzonitrile

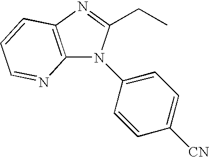

A solution of 4-(3-aminopyridin-2-ylamino)benzonitrile (J, Med. Chem. 1992, vol. 17, p. 3197, 2.40 g, 11.0 mmol) in propionic acid (5 mL) was heated at 150° C. for 8 h and 170° C. for 3.5 h. The mixture was concentrated and the residue purified by SGC (gradient of 1%-10% MeOH in DCM, 0.5% NH₄OH). The product so obtained was dissolved in DCM and the solution washed with aqueous NaHCO₃, dried and concentrated. Yield 1.89 g, 67%. ¹H NMR (CDCl₃) δ 8.29 (dd, 1H, J=1.5.5 Hz), 8.06 (dd, 1H, J=1.5, 8 Hz), 7.89 (m, 2H), 7.59 (m, 2H), 7.27 (dd, 1H, J=5, 8 Hz), 2.87 (q, 2H, J=7.5 Hz), 1.39 (t, 3H, J=7.5 Hz). HPLCMS 6.49 min, m/e 249 (MH+),

### Preparation 82B

### 4-(2-ethyl-3H-imidazo[4,5-b]pyridin-3-yl)-N′-(6-methylpyridin-3-yl)benzamidine

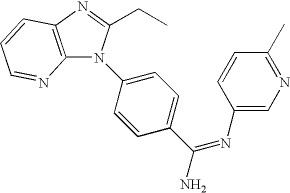

According to General Procedure 1, 4-(2-ethyl-3H-imidazo[4,5-b]pyridin-3-yl)benzonitrile (1.61 g, 6 mmol) and 6-methyl-3-aminopyridine (653 mg, 6 mmol) and sodium hydride dispersion (528 mg, 13.2 mmol) gave a reaction mixture which was poured on ice and the product isolated by filtration and subsequently purified by SGC (gradient of 1%-10% MeOH in DCM, 0.5% NHOH)). Yield 315 mg, brown solid, ¹H NMR (CDCl₃) δ 8.27 (dd, 1H, J= 1.5 Hz), 8.22 (br, 1H). 8.10 (br, 1H), 8.09 (br, 1H), 8.04 (dd, 1H, J=1.5, 8 Hz). 7.52 (d, 2H, J=8.7 Hz), 7.24 (dd, 1H, J=5.8 Hz), 7.25 (m, 1H), 7.15 (d, 1H, J=8 Hz), 2.86 (q, 2H, J=7.5 Hz), 1.36 (t, 3H, J=7.5 Hz).

### Example 83

### 2-ethyl-3-(4-(1-(6-methylpyridin-3-yl)-4-(4-methylthiazol-2-yl)-1H-imidazol-2-yl)phenyl)-3H-imidazo[4,5-b]pyridine

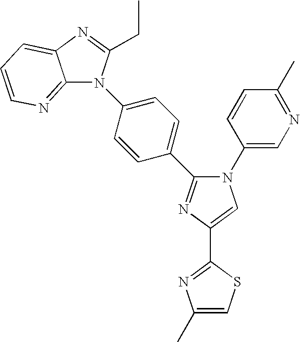

N²-(4-(1-(6-methylpyridin-3-yl)-4-(4-methylthiazol-2-yl)-1H-imidazol-2-yl)phenyl)pyridine-2,3-diamine (65 mg, 0.14 mmol) and propionic acid (0.5 mL) were combined in a screw-cap vial and heated at 155° C. for 3 h. The mixture was concentrated and the residue purified by SGC (2% MeOH-DCM, 0.5% NH₄OH) giving the title substance. ¹H NMR (CDCl₃) δ 8.58 (d, 1H, J=2.5 Hz), 8.28 (dd, 1H, J=1.5.5 Hz), 8.03 (dd, 1H, J=1.2, 8 Hz), 7.82 (br, 1H), 7.68 (m, 2H), 7.53 (dd, 1H, J=2.5, 8 Hz), 7.38 (m, 2H). 7.26 (d, 1H, J=8 Hz). 7.23 (dd, 1H, J=5, 8 Hz), 6.87 (m, 1H). 2.80 (q, 2H, J=75 Hz), 2.64 (s, 3H), 2.50 (s, 3H), 1.34 (t, 3H, J=7.5 Hz), HPLCMS 6.75 min, m/e 478 (MH+).

### Preparation 83A

### 5-(2-(4-iodophenyl)-4-(4-methylthiazol-2-yl)-1H-imidazol-1-yl)-2-methylpyridine

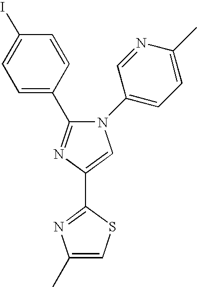

4-iodo-N′-(6-methylpyridin-3-yl)benzamidine (2.56 g, 78 mmol), 2-bromo-1-(4-methylthiazol-2-yl)ethanone (2.5 g, 7.4 mmol), NaHCO₃ (2.48 g, 29.6 mmol) and isopropyl alcohol (20 mL) were heated at 100° C. in a sealed tube for 2 h, filtered, concentrated, and the residue purified by SGC (30%-70% EtOAc in hexanes) giving the title substance (820 mg, 24%), a brown solid, ¹H NMR (CDCl₃) δ 8.45 (d, 1H, J=2.5 Hz), 7.73 (s, 1H). 7.64 (m, 2H). 7.40 (dd, 1H, J=8, 2.5 Hz), 7.21 (d, 1H, J=8 Hz), 7.14 (m, 2H), δ 84 (q, 1H, J=1 Hz), 2.62 (s, 3H), 2.47 (d, 3H, J=1 Hz). MS (AP+) m/e 459 (MH+).

### Preparation 83B

### N-(4-(1-(6-methylpyridin-3-yl)-4-(4-methylthiazol-2-yl)-1H-imidazol-2-yl)phenyl)-3-nitropyridin-2-amine

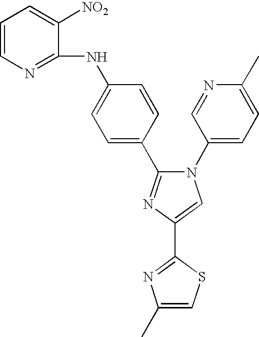

5-(2-(4-iodophenyl)-4-(4-methylthiazol-2-yl)-1H-imidazol-1-yl)-2-methylpyridine (500 mg, 1.29 mmol), 2-amino-3-nitropyridine (167 mg, 1.29 mmol), tris(dibenzylideneacetone)dipalladium(0) (10 mg, 0.011 mmol), 4,5-bis(diphenylphosphino)-9,9-dimethylxanthene (16 mg, 0.027 mmol), Cs₂CO₃ (497 mg, 1.5 mmol) and p-dioxane (2 ml) were combined and heated by microwave at 145° C. for 1 h. The mixture was filtered, evaporated and the residue purified by SGC (3% MeOH in DCM, NH₄OH) giving 409 mg of a red solid (95%). ¹H NMR (CDCl₃) δ 10.23 (br, 1H), 8.53 (dd, 1H, J=1.5, 8 Hz), 8.51 (d, 1H, J=2.5 Hz), 8.49 (dd, 1H, J=1.5, 4.5 Hz), 7.73 (s, 1H), 7.68 (m, 2H), 7.46-7.42 (m, 3H), 7.21 (d, 1H, J=8 Hz), 6.87 (dd, 1H, J=4.5, 8 Hz), 6.84 (q, 1H, J=1 Hz), 2.62 (s, 3H), 2.48 (s, 3H)HPLCMS 9.15 min, m/e 470 (MH+).

### Preparation 83C

### N²-(4-(1-(6-methylpyridin-3-yl)-4-(4-methylthiazol-2-yl)-1H-imidazol-2-yl)phenyl)pyridine-2,3-diamine

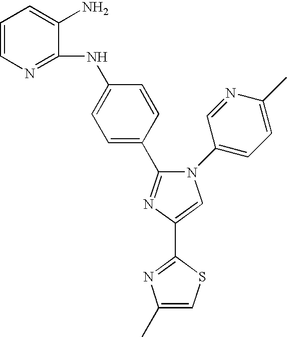

A mixture of N-(4-(1-(6-methylpyridin-3-yl)-4-(4-methylthiazol-2-yl)-1H-imidazol-2-yl)phenyl)-3-nitropyridin-2-amine (358 mg, 0.78 mmol) and 10% Pd/C (150 mg) in MeOH (30 mL) was shaken under 45 p.s.i. hydrogen pressure at RT for 1.5 h, filtered, and concentrated. Yield 301 mg, 90%. ¹H NMR (CDCl₃) δ 8.50 (d, 1H, J=2.5 Hz), 7.82 (dd, 1H, J=1.7, 5 Hz), 7.70 (s, 1H), 7.40 (dd, 1H, J=2.5 Hz), 7.82 (m, 2H), 7.24 (m, 2H), 7.17 (d, 1H, J=8 Hz), 7.02 (dd, 1H, J=1.7, 7.7 Hz), 6.82 (q, 1H, J=1 Hz), 6.78 (dd, 1H, J=5, 7.7 Hz), 6.33 (br, 1H), 3.39 (br, 2H), 2.62 (s, 3H), 2.47 (d, 1H, J=1 Hz). HPLCMS 4.15 min, m/e 440 (MH+).

### Example 84

### 2-(difluoromethyl)-3-(4-(1-(6-methylpyridin-3-yl)-4-imidazol-2-yl)phenyl)-3H-imidazo[4,5-b]pyridine

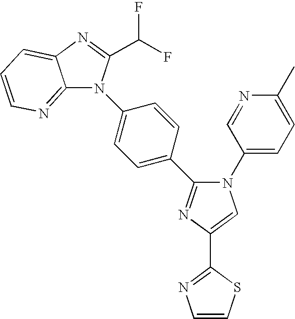

N²-(4-(1-(6-methylpyridin-3-yl)-4(thiazol-2-yl)-1H diamine (42 mg) and difluoroacetic acid (0.5 mL) were combined and heated at 90° C. for 1.5 h. The mixture was dissolved in DCM and the solution extracted with aqueous NaHCO₃. The extracts were dried, concentrated, and the residue purified by SGC (0.5%-2.5% MeOH in DCM, 0.5% NH₄OH, Yield 30 mg of colorless solid ¹H NMR (CDCl₃) δ 8.58 (d, 1H, J=2.5 Hz), 8.47 (dd, 1H, J=1.2, 5 Hz), 8.18 (dd, 1H, J=1.5, 8 Hz), 7.81 (d, 1H, J=3.3 Hz), 7.78 (s, 1H), 7.68 (m, 2H), 7.52 (dd, 1H, J=2.7, 8 Hz). 7.31 (d, 1H, J=3.3 Hz), 7.25 (d, 1H, J=8 Hz), 6.78 (t, 1H, J=52 Hz), 2.63 (s, 3H). HPLCMS 7.4 min, m/e 486 (MH+). IC₅₀=0.730 nM.

### Example 85

### 2-ethyl-3-(4-(1-(6-methylpyridin-3-yl)-4-(thiazol-2-yl)-1H-imidazol-2-yl)phenyl)-3H-imidazo[4,5-b]pyridine

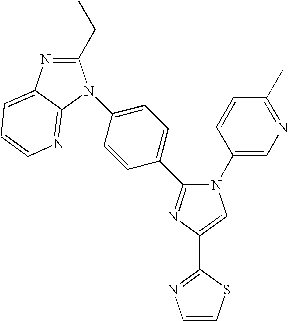

4-(2-ethyl-3H-imidazo[4,5-b]pyridin-3-yl)-N′-(6-methylpyridin-3-yl)benzamidine (140 mg, 0.39 mmol), 2-bromoacetylthiazole (121 mg, 0.59 mmol), NaHCO₃ (131 mg, 1.56 mmol), and 2-propanol (2 ml) were combined in a screw-cap vial and heated at 90° C. for 2 h. The mixture was filtered and evaporated and the residue dissolved in acetic acid (2 ml). The resulting solution was heated at 90° C. 15 min and concentrated. The residue was dissolved in DCM and washed twice with aqueous 10% citric acid and wafer, dried, concentrated and the residue purified by SGC (1%-2% MeOH in DCM, 0.5% NH₄OH). Yield 27 mg of brown solid. ¹H NMR (CDCl₃) δ 8.60 (d, 1H, J=25 Hz), 8.30 (dd, 1H, J=15, 5 Hz), 7.83 (d, 1H J=3 Hz), 7.80 (br, 1H), 7.69 (m, 2H). 7.54 (dd, 1H, J=2.5, 8 Hz). 7.39 (m, 2H), 7.32 (d, 1H, J=3 Hz), 7.27 (d, 1H, J=8 Hz), 7.24 (m, 1H), 8.06 (d, 1H, J=7-8 Hz), 2.82 (q, 2H, J=7.5 Hz), 1.35 (t, 3H, J=7.5 Hz). HPLCMS 6.41 min, m/e 464 (MH+).

### Example 86

### 2-isopropyl-3-(4-(1-(6-methylpyridin-3-yl)-4-(thiazol-2-yl)-1H-imidazol-2-yl)phenyl)-3-H-imidazo[4,5-b]pyridine

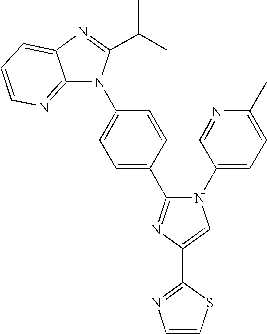

Isobutyric anhydride (26 uL, 0.16 mmol) was added to a mixture of N²-(4-(1-(6-methylpyridin-3-yl)-4-(thiazol-2-yl)-1H-imidazol-2-yl)phenyl)pyridine-2,3-diamine (44 mg, 0.104 mmol) in isobutyric acid (0.7 mL). The mixture was heated at 90° C. for 1.5 h, dissolved in DCM, and the solution extracted with aqueous NaHCO₃, dried and concentrated. The residue was purified by SGC (0.5%-2% MeOH-DCM, 0.5% NH₄OH). Yield 28 mg, colorless solid. ¹H NMR (CDCl₃) δ 8.59 (d, 1H, J=2.5 Hz), 8.27 (dd, 1H, J=1.5, 5 Hz), 8.03 (dd, 1H, J= 1, 7 Hz), 7.82 (d, 1H, J=3 Hz), 7.78 (s, 1H), 7.69 (m, 2H), 7.53 (dd, 1H, J=3, 8 Hz), 7.36 (m, 2H), 7.31 (d, 1H, J=3 Hz), 7.26 (d, 1H, J=8 Hz), 7.21 (dd, 1H, J=5, 8 Hz), 3.08 (septet, 1H, J=6.6 Hz), 2.64 (s, 3H), 1.31 (d, 6H, J=6.6 Hz). HPLCMS 6.94 min, m/e 478 (MH+).

### Example 87

### 2(trifluoromethyl)-3-(4-(1-(6-methylpyridin-3-yl)-4-(thiazol-2-yl)-1H-imidazol-2-yl))phenyl)-3H-imidazo[4,5-b]pyridine

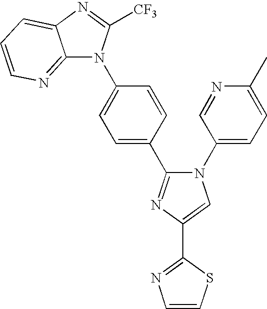

A solution of N²-(4-(1-(6-methylpyridin-3-yl)-4-(thiazol-2-yl)-1H-imidazol-2-yl)phenyl)pyridine-2,3-diamine (4.06 g, 9.54 mmol) in 40 mL TFA was sealed in a screw cap glass pressure vessel (caution), heated in an oil bath at 90-95° C. for 3 h, cooled, and concentrated. The residue was extracted using 3×100 mL DCM and excess 1N NaOH and the organic layers dried, concentrated, and the product purified by SGC (1% and 1.5% MeOH in DCM, 0.5% NH₄OH) giving 3.6 g of off-white solid. Recrystallization from ether gave 3.4 g of a colorless solid. ¹H NMR (CDCl₃) δ 8.59 (d, 1H, J=2.5 Hz), δ 51 (dd, 1H, J=1.5, 5 Hz). 8.24 (dd, 1H, J=1.5, 8 Hz), 7.82 (d, 1H, J=3.3 Hz), 7.79 (s, 1H), 7.69 (m, 2H), 7.50 (dd, 1H, J=3, 8 Hz), 7.42 (m, 2H), 7.41 (dd, 1H, J=4.6, 8 Hz), 7.32 (d, 1H, J=3 Hz), 7.26 (d, 1H, J=8, 3 Hz), 2.64 (s, 3H). HPLCMS 8.16 min, m/e 504 (MH+). IC₅₀=<1000 nM.

### Example 88

### 3-(4-(3-(pyridin-2-yl)-5-(pyridin-3-yl)-1H-1,2,4-triazol-1-yl)phenyl)-1H-imidazo[4,5-b]pyridin-2(3H)-one

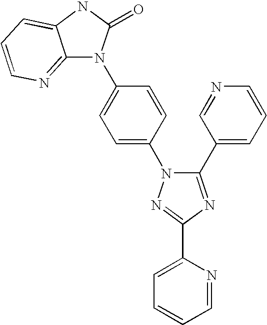

A mixture of N²-(4-(3-(pyridin-2-yl)-5-(pyridin-3-yl)-1H-1,2,4-triazol-1-yl)phenyl)pyridine-2,3-diamine (143 mg, 0.35 mmol), propionic acid (2 uL), and tetramethylorthocarbonate (0.5 mL) was heated at 110° C. for 4 h and concentrated. The residue was chromatographed on silica giving two fractions. The less polar fraction contained a mixture of two isomeric substances with masses of 447. The more polar fraction contained the title substance, a colorless solid (16 mg) ¹H NMR (CDCl₃) δ 10.29 (s, 1H), 8.94 (m, 1H), 8.82 (m, 1H), 8.67 (dd, 1H, J=2, 5 Hz), 8.27 (dt, 1H, J=8.5 Hz), 8.06 (dd, 1H, J=1, 5 Hz), 8.02 (m, 2H), 7.98 (ddd, 1H, J=2, 2, 8 Hz). 7.84 (dt. 1H, J=2, 8 Hz). 7.61 (m, 2H). 7.4-7.3 (m, 4H), 7.06 (dd, 1H, J=5, 7.7 Hz); HPLCMS 5.66 min. m/e 433 (MH+).

### Preparation 88a

### 1-(4-iodophenyl)-2-((pyridin-2-yl))methylene)hydrazine

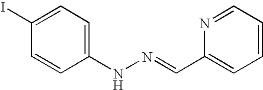

A solution of 4-iodophenylhydrazine (1.04 g, 4.44 mmol), 2-pyridinecarbaldehyde (476 mg, 4.44 mmol) and 1 mL acetic acid in ethanol (20 mL) was heated at reflux for 5 h and concentrated. The residue was triturated with ether giving 780 mg (54%) of a greenish solid. ¹H NMR (DMSO-d₆) δ 10.79 (s, 1H), 8.47 (ddd, 1H, J=1, 2.5 Hz). 7.89 (d, 1H, J=7.9 Hz), 7.84 (s, 1H), 7.76 (td, 1H, J=1.5, 7.8 Hz), 7.50 (m, 2H), 7.25 (ddd, 1H, J=1, 5, 7 Hz), δ 92 (m, 2H). HPLCMS 6.82 min, m/e 324 (MH+).

### Preparation 88b

### 2-(1-(4-iodophenyl)-5-(pyridin-3-yl)-1H-1,2,4-triazol-3-yl)pyridine

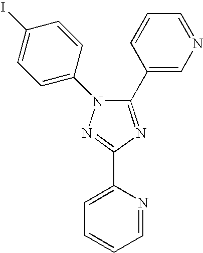

Pyridinium tribromide (745 mg. 2.33 mmol) was added to a solution of 1-(4-iodophenyl)-2-((pyridin-2-yl)methylene)hydrazine (752 mg, 2.33 mmol) in THF (10 mL) at 0° C. and the mixture was stirred at 0° C. for 1.5 h and RT for 2 h and concentrated. The brown solid residue (1.47 g) was dissolved in 2-propanol (15 mL), treated with (pyridin-3-yl)methanamine (500 mg. 4.7 mmol) and triethylamine (1.17 g, 11.6 mmol), stirred at RT 10 h, and 55° C. 1 h, and concentrated. The residue (1.7 g) was dissolved in acetonitrile (10 mL) 2.33 mmol). Silver carbonate (645 mg, 2.33 mmol) was added and the mixture stirred at RT for 18 h and filtered. The filtered solid was washed with EtOAc and the organic layers combined and washed with water and dried giving 1.1 g dark solid. SGC (50%-100% linear gradient of EtOAc-hexanes) gave 100 mg (10%) of the title substance. ¹H NMR (CDCl₃) δ 3.78 (m, 2H), 8.67 (dd, 1H, J=1.7, 4.6 Hz), 8.24 (d, 1H, J=7.9 Hz), 7.96 (ddd, 1H, J=2, 2, 8 Hz), 7.84 (td, 1H, J=1.9, 7.8 Hz), 7.79 (m, 2H), 7.39-7.35 (m, 2H), 7.19 (m, 2H). HPLCMS 7.89 min, m/e 426 (MH+).

### Preparation 88C

### 3-nitro-N-(4-(3-(pyridin-2-yl)-5-(pyridin-3-yl)-1H-1,2,4-triazol-1-yl)phenyl)pyridin-2-amine

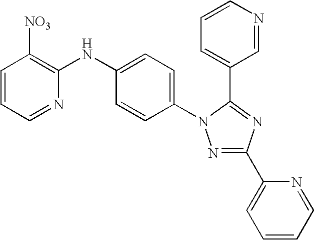

2-(1-(4-iodophenyl)-5-(pyridin-3-yl)-1H-1,2,4-triazol-3-yl)pyridine (217 mg, 0.51 mmol), 2-amino-3-nitropyridine (78 mg, 0.56 mmol), tris(dibenzylideneacetone)dipalladium(0) (5 mg, 0.0051 mmol), 4,5-bis(diphenylphosphino)-9,9-dimethylxanthene (7.4 mg, 0.013 mmol), Cs₂CO₃. (250 mg, 0.77 mmol) and p-dioxane (3 mL) were combined and heated by microwave at 150° C. for 2 h. The mixture was filtered, evaporated and the residue purified by SGC (a gradient of 25% to 100% EtOAc in hexanes giving a reddish solid (140 mg ¹H NMR (CDCl₃) δ 10.33 (s, 1H), 8.87 (m, 1H), 8.79 (m, 1H), 865 (dd, 1H, J=1.7, 5 Hz), 8.56 (dd, 1H, J=1.7, 8 Hz), 8.53 (dd, 1H, J=1.7, 5 Hz), 8.26 (dt, 1H, J=8 Hz), 7.99 (ddd, 1H, J=2.28 Hz), 7.87 (m, 2H), 7.83 (dt, 1H, J=1.7, 8 Hz), 7.45 (m, 2H), 7.37-7.31 (m, 2H), 6.93 (dd, 1H, J=4.7, 8.3 Hz), MS (AP+) m/e 437 (MH+).

### Preparation 88D

### N²-(4-(3-(pyridin-2-yl)-5-(pyridin-3-yl)-1H-1,2,4-triazol-1-yl)phenyl)pyridine-2,3-diamine

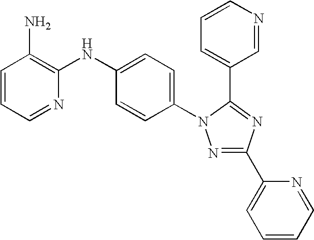

A mixture of 3-nitro-N-(4-(3-(pyridin-2-yl)-5-(pyridin-3-yl)-1H-1,2,4-triazol-1-yl)phenyl)pyridin-2-amine (120 mg, 0.275 mmol) and 10% palladium on carbon (50 mg) in MeOH (10 ml) was shaken under 45 p.s.i. hydrogen pressure for 3 h, filtered, and concentrated giving a red solid (143 mg). MS (AP+) m/e 407 (MH+).

### Example 89

### 2-methyoxy-1-(4-(1-(6-methylpyridin-3-yl)-4-(pyridin-2-yl)-1H-imidazol-2-yl)phenyl)-1-H-imidazo[4,5-c]pyridine

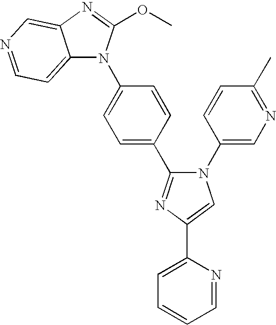

N⁴-(4-(1-(6-methylpyridin-3-yl)-4-(pyridin-2-yl)-1H-imidazol-2-yl)phenyl)pyridine-3,4-diamine (75 mg, 0.18 mmol), tetramethylorthacarbonate (0.5 mL) and propionic acid (1 uL) were heated at reflux temperature for 1 h. Acetic acid (0.5 mL) was added and the mixture stirred at RT for 18 h, concentrated, and the residue purified by SGC (2%-6% MeOH in DCM, 0.5% NH₄OH) giving the title substance. Yield 2 mg. ¹H NMR (CDCl₂) δ 8.87 (s, 1H), 8.58 (m, 1H), 8.52 (d, 1H, J=2.5 Hz), 8.34 (m, 1H), 8.12 (dt, 1H, J=8 Hz), 7.87 (s, 1H), 7.77 (dt, 1H, J=2, 8 Hz), 7.64 (m, 2H), 7.57 (dd, 1H, J=2.5, 8.3 Hz), 7.41 (m, 2H), 7.28 (d, 1H, J=8.3 Hz), 7.19 (dd, 1H, J=5, 8 Hz), 7.11 (dd, 1H, J=1, 5 Hz), 4.21 (s, 3H), 2.64 (s, 3H). HPLCMS 1.5 min, m/e 460 (MH+).

### Preparation 89A

### N-(4-(1-(6-methylpyridin-3-yl)-4-(pyridin-2-yl)-1H-imidazol-2-yl)phenyl)-3-nitropyridin-4-amine

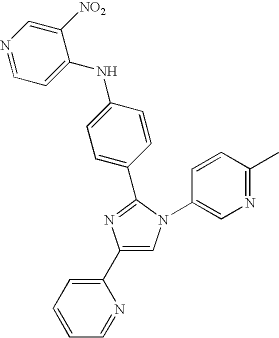

2-(2-(4-iodophenyl)-1-(6-methylpyridin-3-yl)-1H-imidazol-4-yl)pyridine (100 mg, 0.23 mmol), 4-amino-3-nitropyridine (35 mg, 0.25 mmol), tris(dibenzylideneacetone)dipalladium(0) (2 mg), 4,5-bis(diphenylphosphino)-9,9-dimethylxanthene (3 mg.), Cs₂CO₃ (105 mg, 0.32 mmol) and p-dioxane (0.5 mL) were heated by microwave at 155° C. for 2 h, combined with a second reaction mixture prepared identically on the same scale, filtered, concentrated and the residue purified by SGC (gradient of 0-3% MeOH in DCM) giving the title substance (138 mg, 66%). HPLCMS 4.02 min, m/e 450 (MH+).

### Preparation 89B

### N⁴-(4-(1-(6-methylpyridin-3-yl)-4-(pyridin-2-yl)-1H-imidazol-2-yl)phenyl)pyridine-3,4-diamine

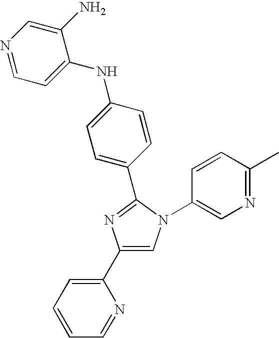

A mixture of N-(4-(1-(6-methylpyridin-3-yl)-4-(pyridin-2-yl)-1H-imidazol-2-yl)phenyl)-3-nitropyridin-4-amine (120 mg, 0.26 mmol), 10% palladium on carbon (80 mg) in MeOH (20 mL) was shaken under 45 p.s.i. hydrogen pressure for 1 h, filtered, and concentrated giving 101 mg of the title substance. HPLCMS 1.52 and 2.03 min, m/e 210 and 420 (MH+).

### Example 90

### 2-methoxy-3-(4-(1-(6-methylpyridin-3-yl)-4-(4-methylthiazol-2-yl)-1H-imidazol-2-yl)phenyl)-3H-imidazol[4,5-b]pyridine

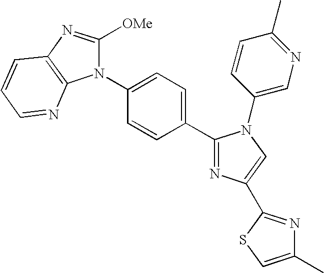

N²-(4-(1-(6-methyl pyridin-3-yl)-4(4-methylthiazol-2-yl)-1H-imidazol-2-yl)phenyl)pyridine-2,3-diamine (150 mg, 0.34 mmol), tetramethylorthocarbonate (0.6 mL) and propionic acid (1 uL) were heated at reflux temperature for 1 h, evaporated, and the residue purified by SGC (1-2% MeOH in DCM, 0.5% NH₄OH) giving a colorless solid (110 mg after trituration with ether and drying). ¹H NMR (CDCl₃) δ 8.55 (d, 1H, J=2 Hz), 8.16 (dd, 1H, J=1, 5 Hz), 7.81 (dd, 1H, J=1.5, 7.7 Hz), 7.76 (s, 1H), 7.60 (s, 4H), 7.49 (dd, 1H, J=3, 8.3 Hz), 7.23 (d, 1H, J=8 Hz), 7.17 (dd, 1H, J=5, 8 Hz), 6.85 (d, 1H, J=1 Hz), 4.20 (s, 3H), 2.83 (s, 1H). 2.49 (d, 3H, J=1 Hz). HPLCMS 7.37 min, m/e 480 (MH+). IC₅₀=0.287 nM.

### Example 91

### 3-(4-(1-(6-methylpyridin-3-yl)-4-(pyridin-2-yl)-1H-imidazol-2-yl)phenyl)-2-propoxy-3H-imidazo[4,5-b]pyridine

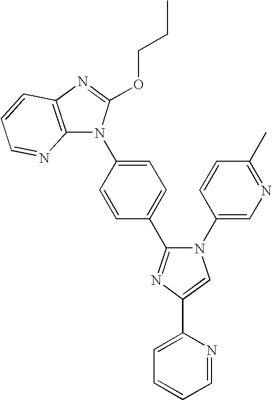

N²-(4-(1-(6-methylpyridin-3-yl)-4-(pyridin-2-yl)-1H-imidazol-2-yl)phenyl)pyridine-2,3-diamine (75 mg, 0.18 mmol), tetramethylorthocarbonate (0.5 mL) and propionic acid (1 uL) were heated in a capped vial at 120° C. for 1 h, concentrated, and purified by SGC (1% and 2% MeOH in DCM, 0.5% NH₄OH) giving the title substance as a pink solid. Yield 54 mg, 62%. ¹H NMR (CDCl₃) δ 8.58 (d, 1H, J=3 Hz), 8.58 (m, 1H). 8.16 (dd, 1H, J=1.7, 5 Hz), 8.12 (dt, 1H, J=1, 8 Hz), 7.85 f>. 1H), 7.79 (dd, 1H, J=1, 8 Hz), 7.76 (dt, 1H, J=1, 8 Hz), 7.63 (s, 4H). 7.50 (dd, 1H, J=2.5, 8.3 Hz), 7.22 (d, 1H, J=8.3 Hz), 7.18 (ddd, 1H, J=1, 5, 8 Hz), 7.15 (dd, 1H, J=5, 8 Hz), 4.53 (t, 2H, J=6.6 Hz), 1.83 (dq, 2H, J=8.6, 7 Hz), 0.99 (t, 3H, J=7 Hz). HPLCMS 5.88 min, m/e 488 (MH+). IC₅₀=0.741 nM.

### Example 92

### 2-(methoxymethyl)-3-(4-(1-(6-methylpyridin-3-yl)-4-(thiazol-2-yl)-1H-imidazol-2-yl)phenyl)-3H-imidazo[4,5-b]pyridine

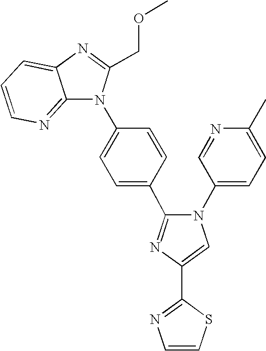

A mixture of N²-(4-(1-(6-methylpyridin-3-yl)-4-(thiazol-2-yl)-1H-imidazol-2-yl)phenyl)pyridine-2,3-diamine (82 mg, 0.146 mmol), methoxyacetic acid (0.7 ml) and methoxyacetyl chloride (20 uL, 0.218 mmol) were heated at 90° C. for 3.5 h and cooled. Methanesulfonic acid (0.5 ml) was added and the mixture was heated at 90° C. for 1 h, cooled, treated with saturated aqueous NaHCO₃, and extracted with DCM giving crude product which was purified by SGC (0.5% to 2% MeOH in DCM, NH₄OH) giving 37 mg of yellow solid. RP-HPLC purification (basic system) gave the title substance (13 mg). ¹H NMR (CDCl₃) δ 8.58 id, 1H, J=2.5 Hz), 8.38 (dd, 1H, J=1.5, 5 Hz), 8.10 (dd, 1H, J=1, 8 Hz), 7.82 (d, 1H, J=3.3 Hz), 7.78 (s, 1H), 7.67 (m, 2H), 7.56-7.51 (m, 3H), 7.32 (d, 1H, J=3.3 Hz), 7.29 (dd, 1H, J=5, 8 Hz), 7.26 (d, 1H, J=8.5 Hz), 4.54 (s, 2H), 3.40 (s, 3H), 2.63 (s, 3H)HPLCMS 6.4 min, m/e 480 (MH+).

### Example 93

### 2-methoxy-3-(4-(1-(6-methylpyridin-3-yl)-4-(thiophen-2-yl)-1H-imidazol-2-yl)phenyl)-3H-imidazo[4,5-b]pyridine

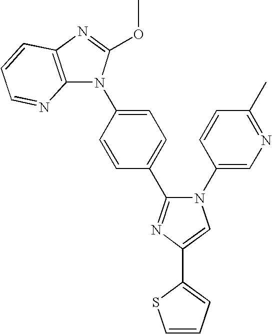

N²(4-(1-(6-methylpyridin-3-yl)-4-(thiophen-2-yl)diamine (128 mg, 0.3 mmol), tetramethylorthocarbonate (0.7 mL) and propionic acid were heated together at reflux temperature for 1 h and concentrated. The residue was purified by SGC (1% MeOH in DCM, 0.5% NHOH) giving the title substance (95 mg), NMR (CDCl₃) δ 8.56 (d, 1H, J=2.5 Hz), 8.18 (dd, 1H, J=1, 5 Hz), 7.81 (dd, 1H, J=1.7, 8 Hz), 7.62-7.57 (m, 4H), 7.48 (dd, 1H, J=2.5, 8 Hz), 7.38 (dd, 1H, J=1, 3.5 Hz), 7.34 (s, 1H), 7.24-7.21 (m, 2H), 7.16 (dd, 1H, J=5, 8 Hz), 7.06 (dd, 1H, J=3.3, 5 Hz), 4.20 (s, 3H), 2.62 (s, 3H). HPLCMS 8.02 min, m/e 465 (MH+). IC₅₀=<0.424 nM.

### Preparation 93A

### 5-(2-(4-iodophenyl)-4-(thiophen-2-yl)-1H-imidazol-1-yl)-2-methylpyridine

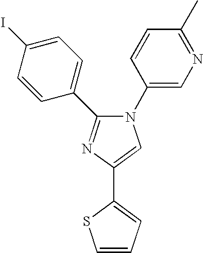

A mixture of 4-iodo-N″-(6-methylpyridin-3-yl)benzamidine (2.50 g, 7.4 mmol), NaHCO₃ (2.48 g, 29.6 mmol), 2-chloroacetylthiazole (1.66 g. 10.4 mmol) in 2-propanol (15 mL) was heated at reflux overnight, cooled, filtered and the filtrate evaporated. Acetic acid (20 mL) was added to the residue and the resulting solution was heated at 70° C. for 20 min and concentrated. The residue was extracted (DCM and aqueous NaHCO₃) and the organic layers dried and concentrated. SGC eluting with 30% EtOAc-hexanes and 50% EtOAc-hexanes, 0.5% NH₄OH) gave a brown solid (1.05 g). 5H NMR (CDCl₃) δ 8.46 (d, 1H, J=2.5 Hz), 7.61 (m, 2H), 740 (dd, 1H, J=2.5, 8.3 Hz), 7.35 (dd, 1H, J=1.3, 3.7 Hz), 7.32 (s, 1H), 7.23 (dd, 1H, J=1.2, 5.0 Hz), 7.20 (d, 1H, J=8.3 Hz), 7.14 (m, 2H), 7.05 (dd, 1H, J=3.3, 5.0 Hz), 2.62 (s, 3H). HPLCMS (method 2) 10.9 min, m/e 444 (MH+).

### Preparation 93B

### N-(4-(1-(6-methylpyridin-3-yl)-4-(thiophen-2-yl) amine

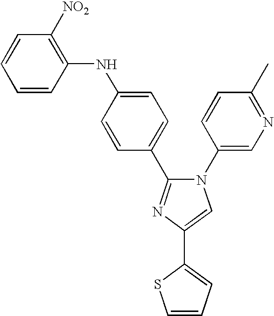

5-(2-(4-iodophenyl)-4-(thiophen-2-yl)-1H-imidazol-1-yl)-2-methylpyridine (500 mg, 1.12 mmol), 2-amino-3-nitropyridine (172 mg, 1.24 mmol), tris(dibenzylideneacetone)dipalladium(0) (10 mg, 0.011 mmol), 4,5-bis(diphenylphosphino)-9,9-dimethylxanthene (16 mg, 0.028 mmol), Cs₂CO₃ (511 mg, 1.58 mmol) and p-dioxane (2 mL) were heated by microwave at 145° C. for 1 h. The mixture was filtered, concentrated, and the residue purified by SGC (1% MeOH in DCM, 0.5% NH₄OH) giving 370 mg of red solid. HPLCMS (method 2) 9.59 min, m/e 455 (MH+).

### Preparation 93C

### N²-(4-(1-(6-methylpyridin-3-yl)-4-(thiophen-2-yl)-1H-imidazol-2-yl)phenyl)pyridine-2,3-diamine

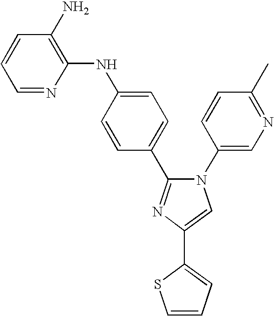

A mixture of N-(4-(1-(6-methylpyridin-3-yl)-4-(thiophen-2-yl)-1H-imidazol-2-yl)phenyl) 3-nitropyridin-2-amine (310 mg, 0.88 mmol) and 10% palladium-on-carbon (150 mg) in 30 mL MeOH was shaken under 45 p.s.i. hydrogen pressure for 1.5 h, filtered, and concentrated giving a solid (240 mg), HPLCMS 4.44 min, m/e 425 (MH+).

### Example 94

### 2-ethoxy-3-(4-(1-(6-methylpyridin-3-yl)-4-(pyridin-2-yl)-1H-imidazol-2-yl)phenyl)-3H-imidazol[4,5-b]pyridine

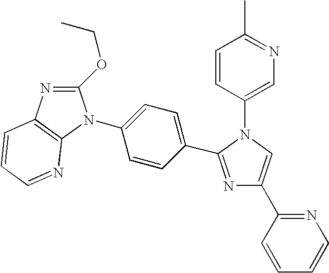

N²-(4-(1-(6-methylpyridin-3-yl)-4-(pyridin-2-yl)-1H-imidazol-2-yl)phenyl)pyridine-2,3-diamine (75 mg, 0.18 mmol), tetraethylorthocarbonate (0.5 mL) and propionic acid (1 uL) were heated at reflux temperature for 1 h, concentrated, and the residue purified by SGC (2% to 8% MeOH in DCM, 0.5% NH₄OH) giving the title substance as an off-white solid, Yield 35 mg. ¹H NMR (CDCl₃) δ 8.59-8.57 (m, 2H), 8.17 (br, 1H), 8.15 (dd, 1H, J=1.2, 5.0 Hz), 8.10-7.90 (br, 1H), 7.82 (br, 1H), 7.80 (dd, 1H, J=1.7, 7.9 Hz), 7.63 (m, 4H), 7.52 (dd, 1H, J=2.5, 8.3 Hz), 7.23 (br, 1H and d, 1H, J=8 Hz), 7.16 (dd, 1H, J=5.0, 7.9 Hz), 4.84 (q, 2H, J=7 Hz), 2.63 (s, 3H), 1.45 (t, 3H, J=7 Hz). HPLCMS 5.55 min, m/e 474 (MH+). IC₅₀=1.02 nM.

### Example 95

### 3-(4-(1-(6-methylpyridin-3-yl)-4-(pyridin-2-yl)-1H-imidazol-2-yl)-1H-imidazo[4,5-b]pyridin-2(3H)-one

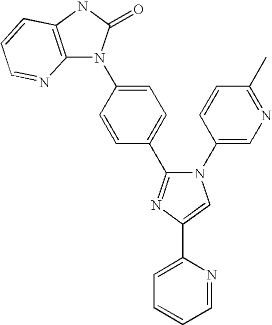

N²-(4-(1-(6-methylpyridin-3-yl)-4-(pyridin-2-yl)-1H-imidazol-2-yl)phenyl)pyridine-2,3-diamine (75 mg, 0.18 mmol) and 1,1′-carbonyldiimidazole (32 mg, 0.2 mmol) were combined in THF at RT for 18 h, concentrated, and purified by SGC (a gradient of 1-4% MeOH in DCM. 0.5% NH₄OH) giving 25 mg (31%) of a light pink solid. ¹H NMR (CDCl₃, partial) δ 2.62 (s, 3H). HPLCMS 4.52 min, m/e 446 (MH+), IC₅₀=3.91 nM.

### Example 96

### 2-methoxy-3-(4-(1-(6-methylpyridin-3-yl)-4-(thiazol-4-yl)-1H-imidazol-2-yl)phenyl)-3H-imidazo[4,5-b]pyridine

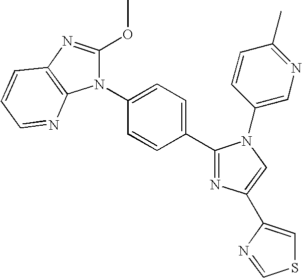

N²-(4-(1-(6-methylpyridin-3-yl)-4-(thiazol-4-yl)-1H-imidazol-2-yl)phenyl)pyridine-2,3-diamine (98 mg, 0.23 mmol), tetramethylorthocarbonate (0.5 mL), and propionic acid (2 uL) were combined, stirred in a sealed vial at 110° C. for 40 min, concentrated, and the residue purified by SGC (2% MeOH in DCM, 0.5% NH₄OH) giving 71 mg of a solid. RP-HPLC purification (basic system) provided the title substance as a colorless solid, yield 28 mg. ¹H NMR (CDCl₃) δ 8.84 (d, 1H, J=2 Hz), 8.56 (d, 1H, J=2 Hz), 8.16 (dd, 1H, J=1.7, 5 Hz). 7.9 (br, 1H), 7.81 (dd, 1H, J=1.7, 8 Hz), 7.68 (s, 1H), 7.63 (m, 4H), 7.50 (dd, 1H, J=2.5, 8 Hz), 7.23 (d, 1H, J=8 Hz), 7.17 (dd, 1H, J=5.8 Hz), 4.21 (s, 3H), 2.63 (s, 3H). HPLCMS, 6.63 min, m/e 466 (MH+). IC₅₀=0.695 nM.

### Preparation 96A

### Thiazole-4-carbonyl chloride

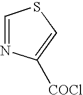

A mixture of thiazole-4-carboxylic acid (30.0 g, 232 mmol) and thionyl chloride (200 mL) was heated at reflux for 2 h. The resulting solution was evaporated and the residue dried giving a yellow solid Yield 34.0 g, 99%. ¹H NMR (CDCl₃) δ 8.91 (d, 1H, J=2 Hz), 8.49 (d, 1H, J=2 Hz).

### Preparation 96B

### N-methyl-N-methylthiazole-4-carboxamide

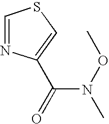

Thiazole-4-carbonyl chloride (43.6 g, 297 mmol) was added in portions at 0-15° C. to a solution of triethylamine (90 g, 890 mmol) and N,O-dimethylhydroxylamine hydrochloride (43.4 g, 445 mmol) in DCM (600 ml). After 20 min the mixture was warmed rapidly to RT. After being stirred 30 min, 2N NaOH (150 mL) was added, and the organic layer was separated and extracted with 150 ml 2N NaOH, The aqueous layers were extracted with 250 mL DCM. The organic layers were separated, dried and concentrated giving a brown oil which was dissolved in EtOAc and the resulting solution washed twice with aqueous 1N NaOH (2×100 mL). The organic layers was dried and concentrated giving an oil (37.4 g, 73%). ¹H NMR (CDCl₃) δ 8.78 (d, 1H, J=2.1 Hz), 8.05 (d, 1H, J=2.1 Hz), 3.73 (s, 3H), 3.40 (s, 3H).

### Preparation 98C

### 1-(thiazol-4-yl)ethanone

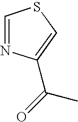

Methylmagnesium iodide (109 mL of 3M in ether, 325 mmol) was added dropwise at 0° C. to a stirred solution of N-methoxy-N-methylthiazole-4-carboxamide (37.4 g, 217 mmol) in ether (500 mL). The mixture was warmed to RT for 40 min and poured onto about 200 g of ice and 2N HCl (250 ml). After being stirred for 10 min the mixture was basified to pH>10 using 2N NaOH (about 200 mL). The layers were separated and the aqueous layer extracted with ether (3×200 mL). The combined organic layers were dried (MgSO₄) and concentrated giving an off-white solid (21.0 g, 77%). ¹H NMR (CDCl₃) δ8.81 (d, 1H, J=2.1 Hz), 8.19 (d, 1H, J=2.1 Hz). 2.68 (s, 3H).

### Preparation 96D

### 2-bromo-1-(thiazol-4-yl)ethanone hydrobromide

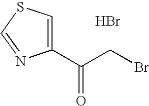

Pyridinium tribromide (42.1 g, 119 mmol of 90%) was added to a stirred solution of 1-(thiazol-4-yl)ethanone (15.1 g, 119 mmol), 33% HBr in acetic acid (320 mL, 178 mmol) and acetic acid (60 ml) at RT. The mixture was warmed to about 40° C. in a warm water bath and stirred at RT overnight. The suspension was filtered and the colorless solid washed with several portions of acetic acid and dried at 100° C. in vacuo. Yield 26 g, 76%. ¹H NMR (CD₃OD) showed a 2:1 mixture of ketone and corresponding trideuterio MeOH hemiketal forms. For the ketone form: δ 9.13 (d, 1H, j=2 Hz). 8.59 (d, 1H, J=2 Hz). 4.71 (s, 2H). For the hemiketal form: δ 9.98 (d, 1H, J=2 Hz), 8.17 (d, 1H, J=2 Hz), 3.82 (A of AB, 1H, J=11 Hz), 3.75 (B of AB, 1H, J=11 Hz). About 10% of a third entity was present: δ 9.83 (d, 1H, J=2 Hz), 8.12 (d, 1H, J=2 Hz), 3.88 (s, 2H). Anal. Calcd for C₅H₅Br₂NOS: C. 20.93; H, 1.76; N, 4.88. Found: C, 21.39; H, 1.79; N. 4.90.

### Preparation 96E

### 5-(2-(4-iodophenyl)-4-(thiazol-4-yl)-1H-imidazol-1-yl)-2-methylpyridine

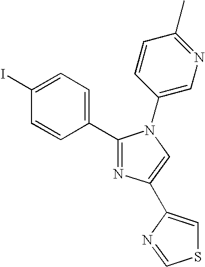

According to General Procedure 2,4-iodo-N′-(6-methylpyridin-3-yl)benzamidine (1.17 g, 3.5 mmol) and 2-bromo-1-(thiazol-4-yl)ethanone hydrobromide (1.00 g, 3.5 mmol) were condensed using 7.7 mL of 1M LiHMDS in THF (10 mL) and DCM as the extraction solvent and SGC as specified therein. Yield 605 mg, 26%, a light brown foam. NMR indicated about 90% purity which could be increased by further chromatography, or recrystallization from hot acetonitrile. ¹H NMR (CDCl₃) δ 8.82 (d, 1H, J=2.1 Hz), 8.45 (d, 1H, J=2.5 Hz), 7.84 (br, 1H), 7.65 (s, 1H), 7.54 (m, 2H), 7.42 (dd, 1H, J=2.5, 8.3 Hz), 721 (d, 1H, J=8.3 Hz), 7.16 (m, 2H), 2.62 (s, 3H). HPLCMS 8.54 min, m/e 445 (MH+).

### Preparation 96F

### N-(4-(1-(6-methylpyridin-3-yl)-4-(thiazol-4-yl)-1H-imidazol-2-yl)phenyl)-3-nitropyridin-2-amine

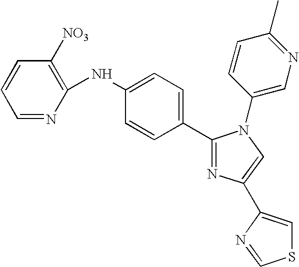

5-(2-(4-iodophenyl)-4-(thiazol-4-yl)-1H-imidazol-1-yl)-2-methylpyridine (487 mg, 1.09 mmol), 2-amino-3-nitropyridine (167 mg, 1.20 mmol), tris(dibenzylideneacetone)dipalladium(0) (40 mg, 0.044 mmol), 4,5-bis(diphenylphosphino)-9,9-dimethylxanthene (63 mg, 0.11 mmol), Cs₂CO₃ (497 mg, 1.53 mmol) and p-dioxane (3 ml) were heated by microwave at 165° C. for 70 min. The mixture was filtered, concentrated, and the residue purified by SGC (2% MeOH in DCM, 0.5% NH₄OH). Yield 290 mg, red solid, 58%. ¹H NMR (CDCl₃) δ 10.23 (s, 1H), 8.83 (d, 1H, J=2 Hz), 8.52 (dd, 1H, J=1.7, 5 Hz), 8.51 (d, 1H, J=2 Hz), 8.50 (dd, 1H, J=1.7, 4.5 Hz), 7.90 (br, 1H), 7.69 (m, 2H). 7.66 (s, 1H), 7.48-7.44 (m, 3H), 7.21 (d, 1H, J=8 Hz), 6.87 (dd, 1H, J=4.5, 8 Hz), 2.62 (s, 3H). HPLCMS 7.92 min, m/e 456 (MH+).

### Preparation 96G

### N²-(4-(1-(6-methylpyridin-3-yl)-4-(thiazol-4-yl)-1H-imidazol-2-yl)phenyl)pyridine-2,3-diamine

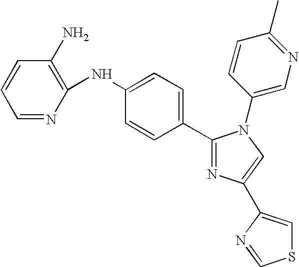

A mixture of N-(4-(1-(6-methylpyridin-3-yl)-4-(thiazol-4-yl)-1H-imidazol-2-yl)phenyl)-3-nitropyridin-2-amine (1.5 g, 33 mmol) and 10% palladium on carbon (1.0 g) in MeOH (25 mL) and DCM (5 mL) was shaken under 45 p.s.i. hydrogen pressure for 3 h, filtered, and concentrated. Yield 1.35 g, 98%. ¹H NMR (CDCl₃ with aq. NaHCO₃ on top) d 8.81 (d, 1H, J=2 Hz), 8.49 (d, 1H, J=3 Hz), 7.83 (m, 1H), 7.79 (br, 1H), 7.62 (s, 1H), 7.42 (dd, 1H, J=2.5, 8 Hz), 7.29 (m, 2H), 7.23 (m, 2H), 7.16 (d, 1H, J=8 Hz), 7.01 (dd, 1H, J=1.5, 8 Hz), 6.77 (dd, 1H, J=5, 8 Hz), 6.70 (br, 2-3H), 2.59 (s, 3H). HPLCMS 3.71 min, m/e 426 (MH+).

### Example 97

### 2-isopropyl-3-(4-(1-(6-methylpyridin-3-yl)-4-(thiazol-5-yl)-1H-imidazol-2yl)-3H-imidazo[4,5-b]pyridine

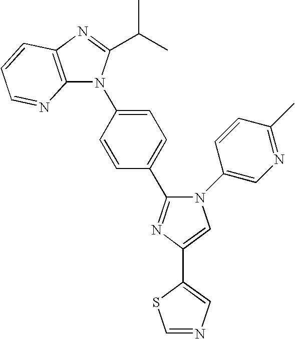

4-(2-isopropyl-3H-imidazo[4,5-b]pyridin-3-yl)-N′-(6-methylpyridin-3-yl)benzamidine (1.00 g, 2.7 mmol), 2-chloro-1-(thiazol-5-yl)ethanone hydrochloride (Helvetica Chim. Acta, 1948, vol 31, pp 26-28, 1.07 g, 5.4 mmol), and NaHCO₃ (910 mg, 10.8 mmol) were combined in 2-propanol (10 ml) and the mixture was heated at 100° C. (bath temperature) in a sealed vessel for 18 h, cooled, filtered, and concentrated. The residue was dissolved in DCM (100 mL) and extracted with 10% aqueous citric acid (2×50 mL), water (50 mL), dried and concentrated. Pure title substance was obtained after two successive SGC purifications (1%-2% MeOH-DCM, 0.5% NH₄OH) followed by RP-HPLC (acidic system). Yield 34 mg. ¹H NMR (CDCl₃) δ 8.75 (s, 1H), 8.60 (d, 1H, J=2.5 Hz), 8.29 id, 1H, J=5 Hz), 8.18 (s, 1H), 8.08 (d, 1H, J=8 Hz), 7.68 (m, 2H), 7.54 (dd, 1H, J=2.5, 8.3 Hz), 7.42 (s, 1H), 7.36 (m, 2H), 7.27 (d, 1H, J=8 Hz), 7.25-7.23 (m, 1H), 3.10 (septet, 1H, J=7 Hz), 2.65 (s, 3H), 1.33 (d, 6H J=7 Hz). The NMR did not change when aq, NaHCO₃ was added to the tube HPLCMS 6.63 min, m/e 478 (MH+). IC₅₀=1.96 nM.

### Example 98

### 2-isopropyl-3-(4-(1-(6-methylpyridin-3-yl)-4-(thiazol-4-yl)-1H-imidazol-2-yl)phenyl)-3H-imidazo[4,5-b]pyridine

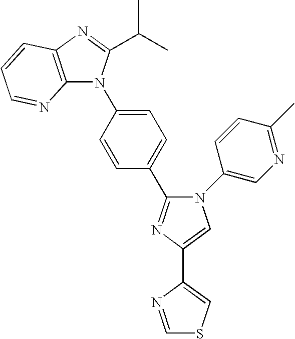

4-(2-isopropyl-3H-imidazo[4,5-b]pyridin-3-yl)-N′-(6-methylpyridin-3-yl)benzamidine (500 mg, 1.35 mmol), 2-bromo-1(thiazol-4-yl)ethanone hydrobromide (776 mg, 2.7 mmol) and NaHCO₃ (680 mg, 8.1 mmol) were mixed in 2-propanol (5 mL) and stirred at 100° C. in a sealed vessel for 18 h, filtered, and concentrated. The residue was dissolved in DCM (50 mL) and the solution washed with aqueous 10% citric acid (2×30 mL), dried, concentrated and the residue purified by SGC (2% MeOH in DCM, 0.5% NH₄OH) giving impure material which was further purified by RP-HPLC (basic system). Yield 72 mg. HPLCMS 6.59 min, m/e 4/8 (MH+), IC₅₀=1.13 nM.

### Preparation 98A

### 4-(2-isopropyl-3H-imidazo[4,5-b]pyridin-3-yl)benzonitrile

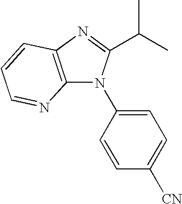

A solution of 4-(3-aminopyridin-2-ylamino)benzonitrile (J, Med. Chem., 1992, vol. 35, p 3127, 5.37 g, 25.6 mmol), and isobutyric anhydride (4.04 g, 25.6 mmol) in isobutyric acid (25 mL) was heated in a seated vessel at 120° C. for 1 h and concentrated. The residue was dissolved in DCM (200 mL) and washed successively with aqueous saturated NaHCO₃ (2×), water, and brine, dried and concentrated giving the title substance (6.11 g). ¹H NMR (CDCl₃) δ 8.28 (dd, 1H, J=1.5, 4.8 Hz), 8.07 (dd, 1H, J=1.5, 8.1 Hz), 7.90 (m, 2H), 7.58 (m, 2H). 7.26 (dd, 1H, J=4.8, 8.1 Hz), 3.14 (septet, 1H, J=88 Hz), 1.37 (d, 6H, J=6.6 Hz), HPLCMS 7.13 min, m/e 263 (MH+).

### Preparation 98B

### 4-(2-isopropyl-3H-imidazo[4,5-b]pyridin-3-yl)-N′-(6-methylpyridin-3-yl)benzamidine

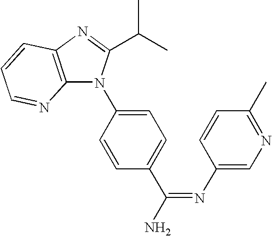

According to General Procedure 1, 4-(2-isopropyl-3H-imidazo[4,5-b]pyridin-3-yl)benzonitrile (6.0 g, 22.9 mmol), 3-amino-8-methylpyridine (2.5 g, 22.9 mmol), and sodium hydride dispersion (60% in oil, 2.0 g, 50.4 mmol) gave a reaction mixture which was poured on ice and brine giving a precipitate which was washed well with water and hexanes and dried in vacuo at 100° C. SGC (3% to 10% MeOH-DCM, 0.5% NH₄OH) gave a fight brown solid. 3.9 g.

### Example 99

### 2-(trifluoromethyl)-3-(4-(1-(6-methylpyridin-3-yl)-4-(thiazol-4-yl)-1H-imidazol-2-yl)phenyl)-3H-imidazo[4,5-b]pyridine

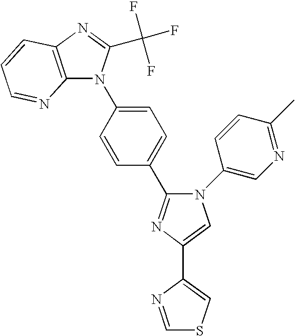

N²-(4-(1-(6-methylpyridin-3-yl)-4-(thiazol-4-yl)-1H-imidazol-2-yl)phenyl)pyridine-2,3-diamine (890 mg, 2.1 mmol) was dissolved in TFA (10 mL) and the resulting solution heated in a sealed glass vessel (caution) at 95° C. (bath) for 5.5 h. The mixture was concentrated and the residue dissolved in 20 mL DCM and the solution washed with sat. aqueous NaHCO₃ (3×10 mL), dried and concentrated. The residue was purified by SGC (a gradient of 0-3% MeOH in DCM, 0.5% NH₄OH) giving 721 mg of an off-white solid. Recrystallization from 98:2 acetonitrile-water gave 240 mg of a crystalline solid (two crops), MP 203° C. This material could also be recrystallized from 2-propanol, m.p. 201-204° C. ¹H NMR (CDCl₃) δ 8.85 (d, 1H, J=2 Hz). 8.59 (d, 1H, J=2.5 Hz), 8.52 (dd, 1H, J=1.5, 5 Hz), 8.24 (dd, 1H, J=1.7, 8.3 Hz), 7.86 (br, 1H), 7.71 (s, 1H), 7.70 (m, 2H), 7.51 (d, 1H, J=2.5, 8.3 Hz). 7.42 (m, 2H), 7.41 (dd, 1H, J=5, 8.3 Hz), 7.25 (d, 1H, J=8 Hz). 2.64 (s, 3H). HPLCMS 7.83 min, m/e 504 (MH+). IC₅₀=1.54 nM.

### Example 100

### 2-ethoxy-3-(4-(1-(6-methylpyridin-3-yl)-4-(thiazol-4-yl)-1H-imidazol-2-yl)phenyl)-3H-imidazo[4,5-b]pyridine

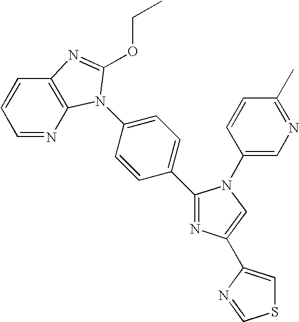

N²-(4-(1-(6-methylpyridin-3-yl)-4-(thiazol-4-yl)-1H-imidazol-2-yl)phenyl)pyridine-2,3-diamine (95 mg), tetraethylorthocarbonate (2 ml), and 5 uL propionic acid were combined in a teflon-capped vial and heated at 150° C. for 4 h. The mixture was concentrated at high vacuum and 130° C. and the residue purified by SGC (1% and 3% MeOH in DCM, 0.5% NH₄OH) giving 42 mg of an off-white solid. ¹H NMR (CDCl_{3) δ} 8.84 (d, 1H, J=2 Hz), 8.57 (d, 1H, J=2.5 Hz), 8.15 (dd, 1H, J=1.7, 4.8 Hz), 7.90 (br, 1H), 7.80 (dd, 1H, J=1, 8 Hz), 7.68 (s, 1H), 7.63 (m, 4H). 7.50 (dd, 1H, J=2.7, 8 Hz), 7.23 (d, 1H, J=8 Hz), 7.16 (dd, 1H, J=5, 8 Hz), 464 (q, 2H, J=7 Hz), 1.44 (t, 3H, J=7 Hz). HRMS 7.11 min, m/e 480 (MH+). IC₅₀=1.31 nM.

### Example 101

### 3-(4-(5-(4-methoxyphenyl)-2-(thiophen-2-yl)-1H-imidazol-4-yl)phenyl)-3H-imidazol[4,5-b]pyridine and 1-(4-(5-(4-methoxyphenyl)-2-(thiophen-2-yl)-1H-imidazo[4,5-b]pyridine

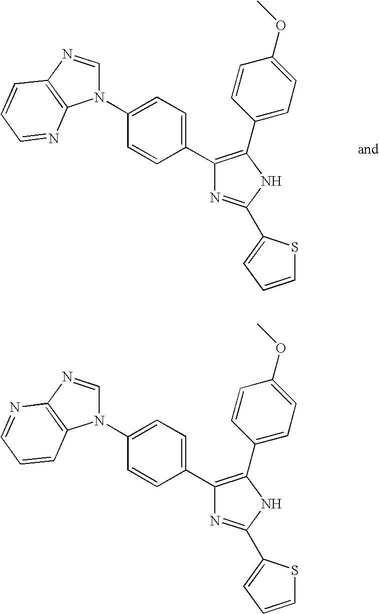

4-(4-bromophenyl)-5-(4-methoxyphenyl)-2-(thiophen-2-yl)-1 W-imidazole (205 mg, 0.50 mmol), 2H-imidazo[4,5-b]pyridine (71.4 mg, 0.6 mmol), K₂CO₃ (138 mg, 1.0 mmol), CuI (4.8 mg, 0.025 mmol), and trans-1,2-diaminocyclohexane (5.7 mg, 0.050 mmol) were combined in 1 mL p-dioxane and the resulting mixture was heated in a sealed vial at 110° C. for 24 h and then 150° C. for 24 h. The mixture was filtered, concentrated and the residue purified by SGC giving 20 mg of the title compound. The ratio of the two title substances was not determined MS (AP+) m/e 450 (MH+). HPLC (Method 3, 50/50) 2.57 min (91%). IC₅₀=0.708 nM.

### Preparation 101a

### 2-(4-bromophenyl)-2-(trimethylsilyloxy)acetonitrile

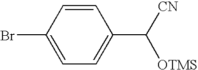

Cyanotrimethylsilane (11.9 ml, 89.0 mmol) was added slowly to a stirred mixture of 4-bromobenzaldehyde (16.5 g) and zinc iodide (241 mg) in DCM (200 mL) at 0° C. After being stirred 15 h at RT, the mixture was concentrated and the residue dissolved in ether and filtered through activated carbon. The filtrate was dried and concentrated giving a light green oil. Yield 25 g, 99%.

### Preparation 101b

### 2-(4-bromophenyl)-2-hydroxy-1-(4-methoxyphenyl)ethanone

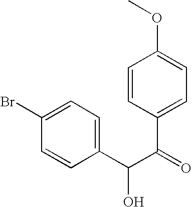

4-methoxyphenylmagnesium bromide (400 mL of 0.5 M in THF) was added dropwise to a solution of 2-(4-bromophenyl)-2-(trimethylsityloxy)acetonitrile (15.2 g, 53.5 mmol) in 600 ml THF at 0° C. and the mixture was stirred at RT for 16 h. 1N HCl (200 mL) was added and the mixture was stirred at RT 4 h. The organic layer was separated and washed with 1N HCl (200 mL), brine, dried and concentrated. The residue was purified by SGC (20% EtOAc-hexanes) giving 4.84 g of a yellow solid (28%).

### Preparation 101c

### 4-(4-bromophenyl)-5-(4-methoxyphenyl)-2-(thiophen-2-yl)-1H-imidazole

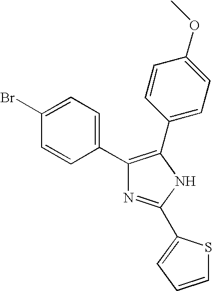

2-(4-bromophenyl)-2-hydroxy-1-(4-methoxyphenyl)ethanone (4.84 g, 15.1 mmol), 2-thiophenecarboxaldehyde (2.03 g, 18.1 mmol), cupric acetate (5.47 g, 30.1 mmol), and ammonium acetate (11.5 g, 150 mmol) were combined in 50 mL acetic acid and the mixture heated at reflux 19 h. The mixture was poured on ice and NH₄OH and extracted with EtOAc (3×50 mL). The organic layers were dried and concentrated and the product purified by SGC (20% and 40% EtOAc-hexanes) giving 2.0 g of an off-white solid.

### Example 102

### 5-methoxy-1-(4-(5-(4-methoxyphenyl)-2-(thiophen-2-yl)-1H-imidazol-4-yl)phenyl)-1H-indole

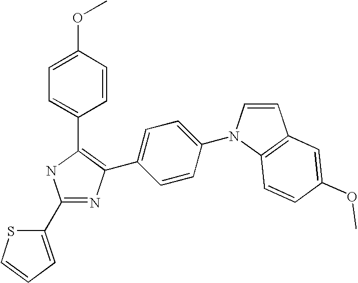

A mixture of 4-(4-bromophenyl)-5-(4-methoxyphenyl)-2-(thiophen-2-yl)-1H-imidazole (113 mg, 0.27 mmol), 5-methoxyindole (61 mg, 0.41 mol), tris(dibenzylideneacetorie)dipalladium(0) (50.3 mg, 0.055 mmol), 2′-(dicyclohexylphosphino)-N,N-dimethyl-[1,1′-biphenyl]-2-amine (32.5 mg, 0.083 mmol) and potassium t-butoxide (62 mg, 0.55 mmol) in 1,2-dimethoxyethane (3 mL) was heated at 100° C. for 18 h. SGC (3% EtOAc in hexanes) gave 18 mg of the title compound as a dark solid. ¹H NMR (CDCl₃) δ 7.64 (br, 1H), 7.62 (br, 1H), 7.45-7.41 (m, 4H), 7.36 (d, 2H, J=9 Hz), 7.30 (d, 1H, J=5 Hz), 7.27 (d, 1H, J=3 Hz), 7.10 (d, 1H, J=2.5 Hz), 7.05 (dd, 1H, J=3.7, 5 Hz), 6.88 (d, 2H, J=9 Hz), 6.84 (dd, 1H, J=2.7, 9 Hz), 6.57 (d, 1H, J=2.5 Hz), 3.85 (s, 3H), 3.81 (s, 3H). MS (AP+) m/e 478 (MH+) IC₅₀=15.8 nM.

### Example 103

### 1(4-(5-(4-methoxyphenyl)-2-(thiophen-2-yl)-1H-imidazol-4-yl)phenyl)-1H-imidazol-4-yl)phenyl)-1H-pyrrolo[2,3-b]pyridine

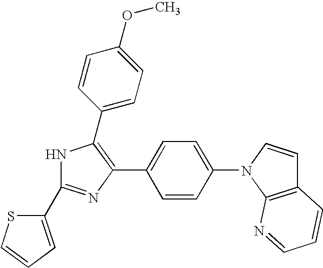

A mixture of 4-(4-bromopheny-1)-5-(4-methoxyphenyl)-2-(thiophen-2-yl)-1H-imidazole (188 mg. 0.46 mmol), 7-azaindole (85 mg. 0.55 mmol), K₂CO₃ (158 mg, 1.14 mmol), and CuI (17.4 mg, 0.091 mmol) in 3 ml DMF was heated by microwave at 235° C. for 1.5 h. The mixture was diluted with 50 mL DCM and 20 ml aqueous saturated NaHCO₃. The aqueous phase was extracted with DCM and the combined organic phases were dried and evaporated. SGC (40% EtOAc-hexanes) gave 15 mg a yellow solid. ¹H NMR (CDCb, partial) δ 8.35 (br, 1H), 7.97 (s, 2H), 7.00 (m, 1H), 6.77 (d, 2H, J=9 Hz), 6.60 (m, 1H), 3.76 (s, 3H). MS (AP+) m/e 449 (MH+). IC₅₀=2.82 nM.

### Example 104

### 1-(4-(1-hydroxy-5-(pyrazin-2-yl)-2-(thiophen-2-yl)-1H-imidazol-4-yl)phenyl)-1H-pyrrolo[2,3-b]pyridine

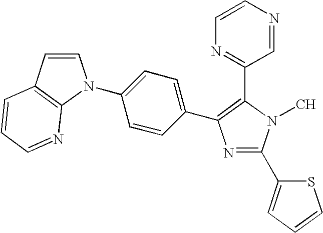

A mixture of 1-(4-(1H-pyrrolo[2,3-b]pyridin-1-yl)phenyl)-2-(hydroxyimino)-2-(pyrazin-2-yl)ethanone (300 mg, 0.88 mmol), thiophene-2-carhaldehyde (0.15 g, 1.3 mmol), and ammonium acetate (0.34 g, 4.4 mmol) in 4 ml HOAc was heated at 100° C. for 20 h. The mixture was poured onto a mixture of ice and cone. NH₄OH, and product isolated by extraction with DCM SGC (1% to 5% MeOH-DCM, 0.5% cone NH₄OH) gave 125 mg of an off-white solid. ¹H NMR (CDCl₃) δ 8.91 (br, 1H), 8.44 (br, 1H), 8.39 (m, 1H), 7.97 (dd, 1H, J=1.5, 7.7 Hz), 7.94 (br, 1H), 7.92 (d, 2H, J=8 Hz), 7.77 (d, 2H, J=8 Hz), 7.55 (d, 1H, J=4 Hz), 7.44 (dd, 1H, J=1, 5 Hz), 7.16-7.13 (m, 3H), 6.65 (d, 1H, J=3.7 Hz) MS (AP+) m/e 437 (MH+), IC₅₀=<2.45 nM.

### Preparation 104A

### Ethyl 4-(1H-pyrrolo[2,3-b]pyridin-1-yl)benzoate

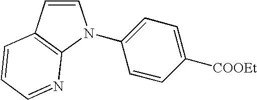

A mixture of ethyl 4-bromobenzoate (3.1 g. 13.4 mmol), 7-azaindole (0.685 g, 5.80 mmol), K2CO₃ (0.8 g, 5.80 mmol), CuSO₄ (46 mg, 0.29 mmol) was heated by microwave at 220° C. for 3.5 h. SGC (0% and 1% EtOAc in hexanes) gave a clear oil (900 mg, 58%)

### Preparation 104B

### 1-(4-(1H-pyrrolo[2,3-b]pyridin-1-yl)phenyl)-2-(pyrazin-2-yl)ethanone

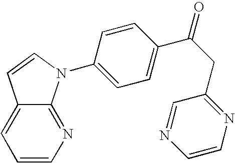

LDA (1.8 M in heptane/THF, 2.0 mL., 3.74 mmol) was added to a solution of 2-methylpyrazine (283 mg, 3.12 mmol) in THF (5 mL) at 0° C. After 5 min a solution of ethyl 4-(1H-pyrrolo[2,3-b]pyridin-1-yl)benzoate (830 mg, 3.12 mmol) in 5 mL THF was added and the mixture was stirred at RT for 17 h. Water (1 mL) and 1:1 EtOAc-hexanes was added, and the resulting yellow precipitate was filtered, washed with 1:1 EtOAc-hexanes and dried. Yield 500 mg.

### Preparation 1048

### 1-(4-(1H-pyrrolo[2,3-b]pyridin-1-yl)phenyl)-2-(hydroxylamino)-2-(pyrazin-2-yl)ethanone

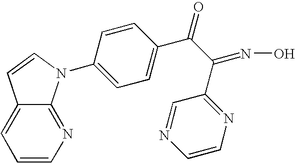

Sodium nitrite (165 mg, 2.39 mmol) was added to 1-(4-(1H-pyrrolo[2,3-b]pyridin-1-yl)phenyl)-2-(pyrazin-2-yl)ethanone (500 mg, 1.59 mmol) in acetic acid (12 ml) and water (2.5 ml) at RT. The mixture was stirred at RT overnight, filtered, and the yellow solid which formed was washed with water and dried. Yield 300 mg, 55%.

### Example 105

### 1-(4-(5-(pyrazin-2-yl)-2-(thiophen-2-yl)-1H-imidazol-4-yl)phenyl)-1H-pyrrolo-2,3-b]pyridine

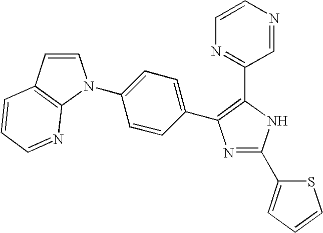

A mixture of 1-(4-(1-hydroxy-5-(pyrazin-2-yl)-2-(thiophen-2-yl)-1H-imidazol-4-yl)phenyl)-1H-pyrrolo[2,3-b]pyridine (110 mg, 0.25 mmol), and P(OEt)₃ (50 mg, 0.30 mmol) in 2 mL DMF was heated at 90° C. for 20 h. Water (10 ml) was added and the mixture extracted with DCM (10 mL×3), dried, and concentrated. SGC (0-4% MeOH in DCM, 0.5% NH₄OH) gave 40 mg of the title compound, ¹H NMR (DMSO-d₆) δ 8.94 (br, 1H), 8.62 (m, 1H), 8.54 (d, 1H, J=2.6 Hz), 8.33 (dd, 1H, J=1.7, 4.6 Hz), 8.10 (dd, 1H, J=1.7, 7.9 Hz), 8.06 (d, 2H, J=5 Hz), 8.05 (s, 1H), 7.93 (br, 1H), 7.84 (m, 2H), 7.72 (d, 1H, J=5 Hz), 7.25-7.21 (m, 2H), 6.76 (d, 1H, J=37 Hz), MS (AP+) m/e 421 (MH+). IC₅₀=9.21 nM.

### Example 106

### 1-(4-(5-(4-methoxyphenyl)-2-(thiophen-2-yl)-1H-imidazol-4-yl)phenyl)-1H-imidazole

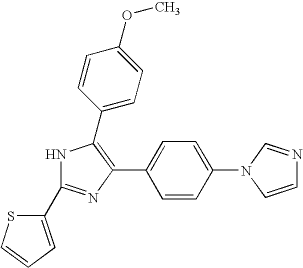

A mixture of 2-(4-(1H-imidazol-1-yl)phenyl)-2-hydroxy-1-(4-methoxyphenyl)ethanone (1.9 g, 6.1 mmol), Cu(OAc)₃ (2.2 g, 12 mmol), NH₄OAc (4.7 g, 61 mol), and thiophene-2-carbaldehyde (0.82 g, 7.3 mmol) in acetic acid (15 ml) was heated at 100° C. for 15 hours and poured onto cone. NH₄OH and ice. The resulting mixture was extracted with 4:1 DCM 2-propanol (50 ml×3), dried over Na₂SO₄ and concentrated SGC (0-2% MeOH in DCM) gave 250 mg of a solid which was dissolved in EtOAc and precipitated with 1 vol, hexanes The yellow solid was filtered and dried. Yield 64 mg. MS (AP+) m/e 399 (MH+). ¹H NMR (CDCl₃, partial) δ 7.80 (br, 1H), 7.57 (s, 2H, J=7.9 Hz), 7.47 (d, 1H, J=3.3 Hz), 7.34 (d, 2H, J=8.3 Hz), 7.27-7.24 (m, 4H), 7.09 (br, 1H), 7.01 (dd, 1H, J=3.7, 5 Hz), 6.84 (d, 2H, J=8.7 Hz), 3.77 (s, 3H). MS (AP+) m/e 399 (MH+). IC₅₀=11.0 nM.

### Preparation 106 A

### 2-(4-(1H-imidazol-1-yl)phenyl)-2-(trimethylsilyloxy)acetonitrile

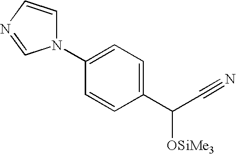

Cyanotrimethylsilane (2.26 g, 22.8 mmol) was added to a solution of 4-(1H-imidazol 1-yl)benzaldehyde (3.93 g, 22.8 mmol) in DMF (25 ml) at 0° C., The suspension was stirred at RT for 18 h, and the resulting solution was concentrated in vacuo giving the title substance as an oil (5.6 g).

### Preparation 106b

### 2-(4-(1H-imidazol-1-yl)phenyl)-2-hydroxy-1-(4-methoxyphenyl)ethanone

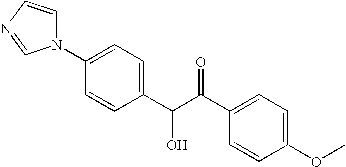

4-methoxyphenylmagnesium bromide (200 mL of 0.5 M in THF) was added dropwise at 0° C. to a solution of 2-(4-(1H-imidazol-1-yl)phenyl)-2-(trimethylsilyloxy)acetonitrile (5.6 g) in 300 mL THF and the mixture was stirred 48 h at RT. 1N HCl (400 mL) was added and after being stirred 4 h at RT, 1N NaOH was added to give a pH between 8 and 9. The organic layer was separated, dried over Na₂SO₄, and evaporated giving a yellow solid which was used without purification.

### Example 107

### 1-(4-(5-(6-(4-methylpiperazin-1-yl)pyridin-3-yl)-2-(thiazol-5-yl)-1H-imidazol-4-yl)-1H-pyrrolo[2,3-b]pyridine

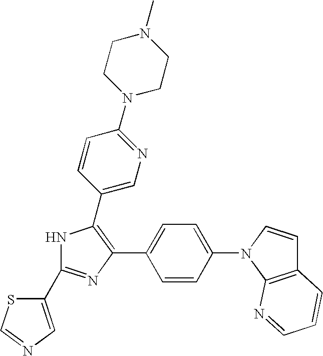

A mixture of 1-(4-(1-hydroxy-5-(8-(4-methylpiperazin-1H-imidazol-4-yl)phenyl)-1H-pyrrolo[2,3-b]pyridine (700 mg, 0.96 mmol) and triethyl phosphite (0.24 g, 1.43 mmol) in 5 mL DMF was heated at 90° C. for 18 h. 5 ml aq, 1M sodium carbonate was added and the aqueous phase was extracted with 4:1 DCM: 2-propanol (20 mL×4). The organic layers were dried and concentrated. SGC (5% and 7% MeOH in DCM, 0.5% NH₄OH) gave 60 mg (12%) of a slightly colored solid. ¹H NMR (CDCl₃, partial) δ 3.50 (m, 4H), 2.46 (m, 4H), 2.30 (s, 3H). MS (AP+) m/e 519 (MH+). HPLC 4.90 min. IC₅₀=2.26 nM.

### Preparation 107A

### Methyl 4-(1H-pyrrolo-2,3-b]pyridin-t-yl)benzoate

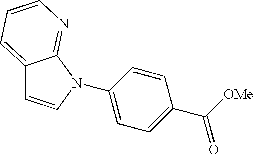

A mixture of methyl 4-iodobenzoate (26.4 g, 0.101 mol), 7-azaindole (11.9 g, 0.101 mol), CuI (964 mg, 5.1 mmol), trans-N,N′-dimethyl-cyclohexane-1,2-diamine (1.15 g, 10.1 mmol), K₃PO₄ (42 g, 0.202 mol) in p-dioxane (200 mL) was heated at reflux for 20 h, cooled, and filtered. The filtrate was concentrated and the residue purified by SGC (15% EtOAc in hexanes) giving a white solid (20 g, 78%).

### Preparation 107B

### 1-(4-(1H-pyrrolo-[2,3-b]pyridin-1-yl)phenyl)-2-(6-bromopyridin-3-yl)ethanone

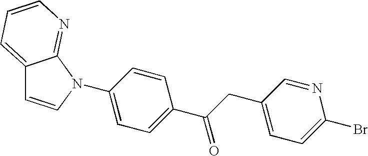

Sodium bis-(trimethylsilyl)amide (51.5 mL of 1M in THF) was added dropwise at 0° C. to a mixture of methyl 4-(1H-pyrrolo[2,3-b]pyridin-1-yl)benzoate (5.91 g, 23.4 mmol) and 2-bromo-5-methylpyridine (4.23 g, 24.6 mmol) in THF (300 mL). The mixture was stirred at RT for 27 h. Water was added and the mixture was extracted with DCM (3×100 ml). The combined organic layers were dried and concentrated. SGC (20% to 50% EtOAc-hexanes) provided 3.5 g of a light yellow solid (38%).

### Preparation 107C

### 1-[4-(1H-pyrrolo[2,3-b]pyridin-1-yl)phenyl)-2-(6-4-methylpiperazin-1-yl)pyridin-3-yl)ethanone

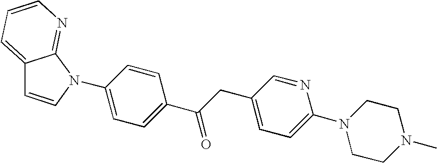

A mixture of 1-(4-(1H-pyrrolo[2,3-b]pyridin-1-yl)phenyl)-2-(6-bromopyridin-3-yl)ethanone (1.3 g. 3.31 mmol). CuI (126 mg, 0.66 mmol), K2CO₃ (913 mg, 6.82 mmol), and 1-methylpiperazine (2.32 g, 23.2 mmol) in p-dioxane (3 ml) was heated at 150° C. in a sealed vessel for 20 h. The mixture was filtered, concentrated, treated with water and DCM (50 ml) and adjusted to pH 1 using 2N HCl. After 48 h, 2N NaOH was added to give pH 10 and the mixture was extracted with 4:1 DCM: 2-propanol (5×30 ml) and the combined organic layers were dried and concentrated. SGC (0% to 2% MeOH in DCM gave a colorless solid (400 mg, 29.5%).

### Preparation 107d

### 1-(4-(1H-pyrrolo[2,3-b]pyridin-1-yl)phenyl)-2-(hydroxyimino)-2-(6-(4-methylpiperazin-1-yl)pyridin-3-yl)ethanone

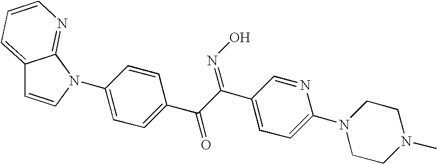

Sodium nitrite (101 mg, 1.46 mmol) was added portionwise to a stirred mixture of 1-(4-(1H-pyrrolo[2,3-b]pyridin-1-yl)phenyl)-2-(6-(4-methylpiperazin-1-yl)pyridin-3-yl)ethanone (400 mg, 0.97 mmol) in acetic acid (7.5 ml) and water (5 ml) at RT. After 20 h the mixture was concentrated, the residue mixed with saturated aqueous NaHCO₃, and this mixture extracted with 4:1 DCM: 2-propanol (4×15 mL). The organic layers were dried and concentrated giving a yellow solid (420 mg) which contained about 20% starting material but was used without further purification.

### Preparation 107E

### 1-(4-(1-hydroxy-5-(6-(4-methylpiperazin-1-yl)pyridin-3-yl)-2-(thiazol-5-yl)-1H-imidazol-4-yl)phenyl)-1H-pyrrolo[2,3-b]pyridine

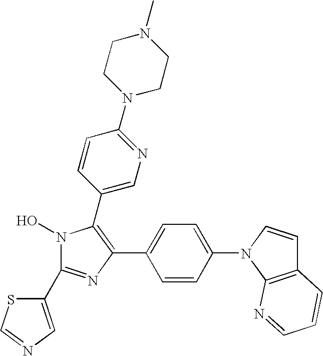

A mixture of crude 1-(4-(1H-pyrrolo[2,3-b]pyridin-1-yl)phenyl)-2-(hydroxyimino)-2-(6-(4-methylpiperazin-1-yl)pyridin-3-yl)ethanone (420 mg, approx. 0.76 mmol), thiazole-5-carbaldehyde (161 mg, 1.43 mmol), and NH4OAc (514 mg, 6.68 mmol) in acetic acid (7 mL) was heated at 100° C. for 24 h and concentrated. SGC (3% and 25% MeOH in DCM, 0.5% NHOH) gave 0.7 g of impure title substance as a brown solid which was used without purification.

### Example 108

### 1-(4-(5-(4-methoxyphenyl-2-(thiophen-2-yl)-1H-imidazol-4-yl)phenyl)-4-phenyl-1H-imidazole

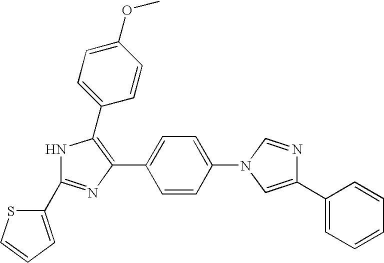

A mixture of 4-(4-bromophenyl)-5-(4-methoxyphenyl)-2-(thiophen-2-yl)-1H-imidazole (250 mg, 0.61 mmol), 4-phenyl-1H-imidazole (175 mg, 1.22 mmol), CuI (11.6 mg, 0.061 mmol), trans-N,N′-dimethyl-cyclohexane-1,2-diamine (13.9 mg, 0.122 mmol), potassium carbonate (168 mg, 1.22 mmol), and N-methyl-2-pyrrolidone (3 mL) was heated in a seated vessel at 180° C. for 24 h. The reaction mixture was treated with 20 mL water and extracted with DCM (20 ml×3). The combined organic layers were washed with 4% aqueous MgSO₄, brine, dried (Na₂SO₄), and concentrated. SGC (50% and 67% EtOAc/hexanes) provided 38 mg (13%) of a light yellow solid. ¹H NMR (CDCl₃, partial) δ 7.88 (s, 1H), 7.82 (m. 1H), 7.80 (m, 1H), 7.70 (d, 2H, J=9 Hz), 7.54 (m, 1H), 7.51 (m, 1H), 7.41-7.33 (m, 6H). 7.25 (m, 1H), 7.08 (dd, 1H, J=3.5, 5 Hz), 8.91 (d, 2H, J=9 Hz), 3.82 (s, 3H). MS (AP+) m/e 475 (MH+). IC₅₀=602 nM.

### Example 109

### 1-(4-(1-hydroxy-5-(pyrazin-2-yl))-2-(thiophen-2-yl)-1H-imidazol-4-yl)-2-methylphenyl-1H-pyrrolo[2,3-b]pyridine

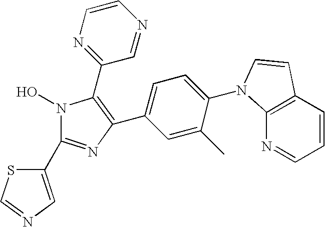

A mixture of 2-(hydroxyimino)-1-(3-methyl-4-(1H-pyrrolo[2,3-b]pyridin-1-yl)phenyl)-2-(pyrazin-2-yl)ethanone (570 mg, 1.60 mmol), thiazole-5-carbaldehyde (273 mg, 2.40 mmol), and ammonium acetate (860 mg, 11.2 mmol), in acetic acid (10 mL) was heated at 100° C. for 20 h, cooled, and poured into a mixture of NH₄OH and ice. The precipitate was filtered, dried, and triturated with Et₂O giving 520 mg (72%) of a light brown solid. ¹H NMR (DMSO-d₆, partial) δ 1.95 (s, 3H). MS (AP+) m/e 445 (MH+). IC₅₀=18.4 nM.

### Preparation 109a

### Methyl 3-methyl-4-(1H-pyrrolo[2,3-b]pyridin-1-yl)benzoate

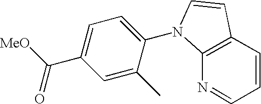

A mixture of methyl 4-bromo-3-methylbenzoate (10 g, 43.7 mmol), 7-azaindole (5.15 g, 43.7 mmol), Cut (167 mg, 0.87 mmol), trans-N,N′-dimethyl-cyclohexane-1,2-diamine (0.49 g), K₃PO₄ (9.26 g, 87.4 mmol), and p-dioxane (30 mL) was heated at reflux for 30 h, cooled, and filtered. Concentration of the filtrate and SGC of the residue (hexanes and 10% EtOAc-hexanes) gave 1.6 g (14%) of a colorless oil.

### Preparation 109b

### 1-(3-methyl-4-(1H-pyrrolo[2,3-b]pyridin-1-yl)phenyl)-2-(pyrazin-2-yl)ethanone

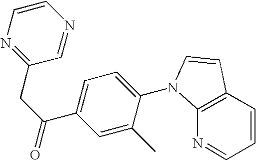

LiHMDS (12.1 mL of 1M in THF) was added at 0° C. to a solution of 2-methylpyrazine (0.57 g, 6.04 mmol) and methyl 3-methyl-4-(1H-pyrrolo[2,3-b]pyridin-1-yl)benzoate (1.61 g, 6.04 mmol) in THF (10 mL) and stirred 0.5 h at 0° C. and 3 h at RT. Water (20 mL) was added and the mixture extracted with DCM (3×20 ml). The combined organic layers were dried, concentrated, and the resulting solid triturated with ether. Yield 1.6 g, brown solid.

### Preparation 109C

### 2-(hydroxyimino)-1-(3-methyl-4-(1H-pyrrolo[2,3-b]pyridin-1-yl)phenyl)-2-(pyrazin-2-yl)ethanone

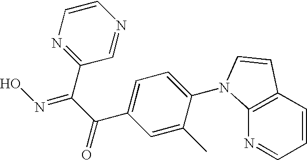

Sodium nitrite (473 mg, 6.9 mmol) was added to a stirred solution of 1-(3-methyl-4-(1H-pyrrolo[2,3-b]pyridin-1-yl)phenyl)-2-(pyrazin-2-yl)ethanone (1.5 g, 4.57 mmol) in acetic acid (15 mL) and water (5 mL) and the mixture was stirred overnight at RT and concentrated. The residue was triturated with ether and dried giving 1.6 g of a dark solid

### Example 110

### 1-(4-(1-hydroxy-5-(pyrazin-2-yl)-2-(thiophen-2-yl)-1H-imidazol-4-yl)-2-methylphenyl)-1H-pyrrolo[2,3-b]pyridine

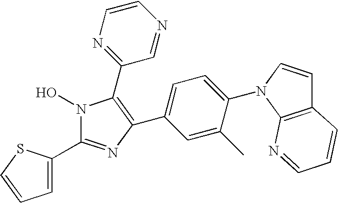

A mixture of 2-(hydroxyimino)-1-(3-methyl-4-(1H-pyrrolo[2,3-b]pyridin-1-yl)phenyl)-2-(pyrazin-2-yl)ethanone (0.65 g, 1.82 mmol), thiophene-2-carbaldehyde (0.306 g. 2.73 mmol), and ammonium acetate (1.12 g, 14.6 mmol) were dissolved in acetic acid (10 mL) was heated at 100° C. for 20 h and then NH₄OH and ice was added. The precipitate was filtered and dried. SGC (EtOAc) provided 150 mg of the title substance, impure starting material isolated from the less polar fractions was resubjected to the above conditions and worked up and purified as above giving 230 mg more product. Total yield 380 mg, 46%. ¹H NMR (CDCl₃, partial) δ 6.62 (d, 1H, J=3.7 Hz), 1.95 (br, 3H). HPLC (50/50, method 3, 4.42 min). MS (AP+) m/e 451 (MH+). IC₅₀=36.6 nM.

### Example 111

### 1-(4-(1-hydroxy-5-(pyrazin-2-yl)-2-(thiophen-2-yl)-1H-imidazol-4-yl)-2-methylpheyl)-1H-pyrrolo[2,3-b]pyridine

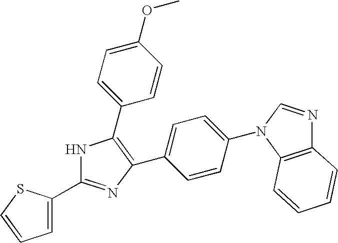

A stirred mixture of 4(4-bromophenyl)-5-(4-methoxyphenyl)-2-(thiophen-2-yl)-1H-imidazole (284 mg, 0.691 mmol), benzimidazole (122 mg, 1.036 mmol), CuI (6.6 mg), trans-N,N′-dimethyl-cyclohexane-1,2-diamine (8 mg, 0.07 mmol), and potassium carbonate (193 mg, 1.4 mmol) in 5 mL p-dioxane and heated in a sealed vessel at 190° C. for 48 h, cooled, filtered, and concentrated SGC (1:1 and 3:1 EtOAc/hexanes) gave a solid which was triturated with Et₂O/hexanes and dried giving an off-white solid. Yield 115 mg (37%). ¹H NMR (CDCl₃) δ 8.07 (s, 1H), 7.83 (m, 1H), 7.78 (d, 2H), 7.52 (m, 2H), 7.45-7.40 (m, 4H), 7.35-7.32 (m, 3H), 7.09 (dd, 1H, J=3.7.5 Hz). 6.92 (d, 2H, J=8.3 Hz), 3.83 (s, 3H). MS (AP+) m/e 449 (MH+). IC₅₀=9.33 nM.

### Example 112

### 1-(2-methyl-4-(5-(pyrazin-2-yl)-2-yl)-2-(thiazol-5-yl)-1H-pyrrolo[2,3-b]pyridine

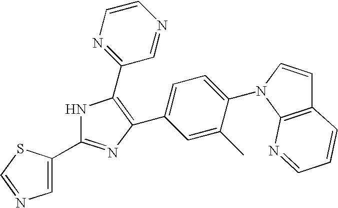

A solution of 1-(2-methyl-4-(5-(pyrazin-2-yl)-2-(thiazol-5-yl)-1-hydroxy-imidazol-4-yl)phenyl)-1H-pyrrolo[2,3-b]pyridine (451 mg, 1.00 mmol) and triethyl phosphite (174 mg. 1.05 mmol) in 5 mL DMF was heated at 100° C. for 20 h and concentrated. SGC (50% and 100% EtOAc-hexanes) provided 150 mg (34%) of product as light yellow solid. ¹H NMR (DMSO-d₆) showed a 2:1 mixture of tautomeric forms: 8 (partial, minor and major tautomers, respectively) 13.60 and 13.34 (s, 1H), 7.33 and 7.38 (d, 1H, J=8 Hz), 6.67 and 6.69 (d, 1H, J=3.7 Hz), 2.02 and 2.04 (s, 3H). MS (AP+) m/e 436 (MH+). IC₅₀=175 nM.

### Example 113

### 1-(2-methyl-4-(5-(pyrazin-2-yl)-2-(thiazol-5-yl)-2-(thiazol-5-yl)-1H-imidazol-4-yl)phenyl)-1H-pyrrolo[2,3-b]pyridine

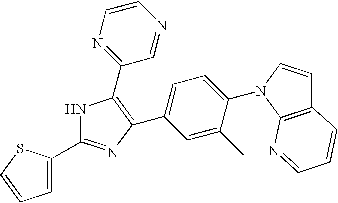

A solution of 1-(4-(1-hydroxy-5-(pyrazin-2-yl)-2-(thiophen-2-yl)-1H-imidazol-4-yl)-2-methylphenyl)-1H-pyrrolo[2,3-b]pyridine (380 mg, 0.842 mmol) and triethyl phosphite (0.154 mL, 0.885 mmol) in 5 mL DMF was heated at 100° C. for 24 h. Water (20 ml) was added and the mixture extracted with methylene chloride (3×20 mL). The combined organic layers were washed with 4% aq. MgSO₄, dried and concentrated. SGC (1:1 EtOAc/hexanes) gave 130 mg (36%) a fight yellow solid. ¹H NMR (CDCl₃, partial) δ 8.96 (s, 1H), 6.62 (d, 1H, J=3.3 Hz), 2.05 (br, 1.5H), 1.80 (br, 1.5H). MS (AP+) m/e 435 (MH+). HPLC (50/50, method 3) 5.68 min (96%). IC₅₀=47.4 nM.

### Example 114

### 1-(4-(2-(pyridin-2-yl)-4-(pyridin-3-yl)-1H-imidazol-5-yl)phenyl)-1H-pyrrolo[2,3-b]pyridine

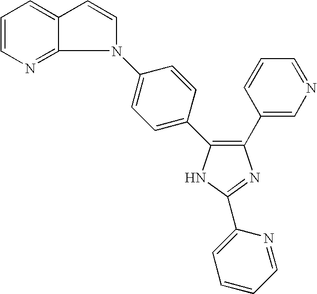

A mixture of 1-(4-(1H-pyrrolo[2,3-b]pyridin-1-yl)phenyl)-2-hydroxyimino-2-<pyridin-3-yl)ethanone (309 mg, 0.903 mmol), 2-pyridinecarboxaldehyde (118 mg, 1.08 mmol), and ammonium acetate (283 mg, 3.61 mmol) in 2 mL acetic acid was heated by microwave at 200° C. for 20 min, cooled and concentrated. Water (10 mL) was added and the mixture extracted with EtOAc (3×10 ml). The organic layers were dried and concentrated. SGC (1%-3% MeOH in DCM, 0.5% NH₄OH) followed by RP-HPLC purification gave 44 mg (12%) of an off-white solid. ¹H NMR (CDCb) δ 9.23 (s, 1H), 8.86 (d, 1H, J=5 Hz), 8.62 (d, 1H, J=4.5 Hz), 8.49 (m, 2H), 8.38 (d, 1H, J=4.6 Hz), 8.08 (t, 1H, J=7.7 Hz), 8.03 (dd, 1H, J=1.7, 7.9 Hz), 7.94 (d, 2H, J=8.3 Hz), 7.68-7.85 (m, 3H), 7.59 (d, 1H, J=3.7 Hz), 7.51 (m, 1H), 7.19 (dd, 1H, J=5.0, 7.9 Hz), 6.70 (d, 1H, J=3.7 Hz). MS (AP+) m/e 415 (MH+). IC₅₀=18.6 nM.

### Preparation 114B

### 1-(4-(1H-pyrrolo[2,3-b]pyridin-1-yl)phenyl)-2-(pyridin-3-yl)ethanone

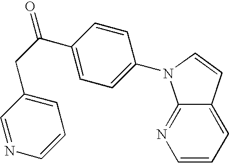

Lithium diisopropylamide (2.0M in heptane-THF-ethylbenzene, Aldrich, 15.0 mL), was added to a stirred solution of 3-methylpyridine (1.40 g, 15.0 mmol) in THF (50 mL) at 0° C. After 30 min, a solution of 4-(1H-pyrrolo[2,3-b]pyridin-1-yl)benzonitrile (3.28 g, 15.0 mmol) in THF (10 ml) was added at 0° C. and the mixture was stirred 1 h at 0° C. Water (40 ml) was added and the mixture was extracted with EtOAc (2×50 ml). The organic layers were dried over Na₂SO₄ and concentrated, SGC (50% and 100% EtOAc-hexanes) gave 1.8 g of a yellow solid (38%).

### Preparation 114b

### 1-(4-(1H-pyrrolo[2,3-b]pyridin-1-yl)phenyl)-2-hydroxyimino-2-(pyridin-3-yl)ethanone

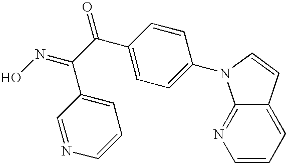

Sodium nitrite (113 mg) was added to a suspension of 1-(4-(1H-pyrrolo[2,3-b]pyridin-1-yl)phenyl)-2-(pyridin-3-yl)ethanone (343 mg, 1.1 mmol) in 2:1 acetic acid:water (5 mL) at RT. After about 30 min, 3 ml more water was added and after being stirred another 5 min, the mixture was filtered and the solid washed with water and hexanes and dried. Yield 399 mg, off-white solid.

### Example 115

### 1-(4-(3-(pyridin-2-yl)-5-(pyridine-3-yl)-1H-1,2,4-triazol-1-yl)phenyl)-1H-pyrrolo[2,3-b]pyridine

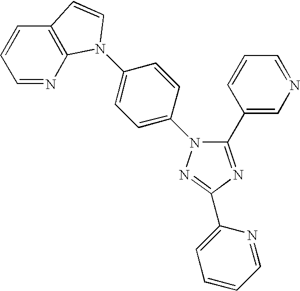

2-(1-(4-iodophenyl)-5-(pyridin-3-yl)-1H-1,2,4-triazol-3-yl)pyridine (150 mg, 0.35 mmol), 7-azaindole (50 mg, 0.42 mmol), CuI (1.5 mg, 0.007 mmol), K₃PO₄ (148 mg, 8.70 mmol), trans-N,N′-dimethyl-cyclohexane-1,2-diamine (5 mg, 0.035 mmol), and p-dioxane (4 mL) were combined and heated in a sealed vial at 115° C. for 18 h. The mixture was diluted with DCM and filtered and the filtrate evaporated giving a brown solid, SGC (linear gradient 0%-5% MeOH in DCM, 0.5% NH₄OH) gave 108 mg of an off-white solid. ¹H NMR (CDCl₃) δ 8.91 (br, 1H), 8.80 (br, 1H), 8.7 (br, 1H), 8.38 (dd, 1H, J=1.7, 4.6 Hz), 8.26 (br, 1H), 8.02-8.00 (m, 3H), 7.98 (dd, 1H, J=1.7, 7.9 Hz), 7.84 (m, 1H), 7.60 (m, 2H), 7.56 (d, 1H, J=3.7 Hz), 7.36 (br, 2H), 7.16 (dd, 1H, J=5.0, 7.9 Hz), 6.67 (d, 1H, J=3.7 Hz). MS (AP+) m/e 416 (MH+). HPLCMS 7.78 min, m/e 416. IC₅₀=17.9 nM.

### Example 116

### 2-methoxy-3-(4-(1-(6-methylpyridin-3-yl)-4-(thiazol-5-yl)-1H-imidazo imidazo[4,5-b]pyridine

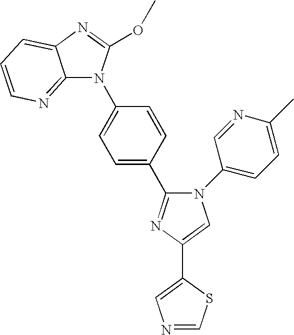

N²-(1-(6-methylpyridin-3-yl)-4-(thiazol-5-yl)-1H-imidazol-2-yl)phenyl)pyridine-2,3-diamine (2.0 g, 4.7 mmol), tetramethylorthocarbonate (10 ml), and propionic acid (40 mg, 0.3 equiv) were heated in a sealed tube immersed in an oil bath at 90° C. for 3 h, at 100° C. for 1 h, and concentrated in vacuo. The reissue was dissolved in 50 mL of 5:1 DCM: 2-propanol and the resulting solution washed with sat. aqueous NaHCO₃, dried and concentrated. SGC (0% and 5% MeOH—CHCl3 with 0.5% NH₄OH) gave 1.65 g of a colorless solid (75%) which was recrystallized from 98:2 MeCN-water giving 1.30 g of a white solid. ¹H NMR (CDCl₃) δ 8.73 (s, 1H), 8.55 (d, 1H, J=3 Hz), δ 8.16 (s, 1H), 8.15 (dd, 1H, J=1, 5 Hz), 7.81 (dd, 1H, J=1.7, 7.9 Hz), 7.62-7.57 (m, 4H), 7.50 (dd, 1H, J=2.5, 8 Hz), 7.4 (s, 1H), 7.24 (d, 1H, J=8 Hz), 7.17 (dd, 1H, J=5, 8 Hz), 4.20 (s, 3H). HPLCMS 6.73 min, m/e 466 (MH+) IC₅₀=0.305 nM.

### Preparation 118a

### 1-(thiazol-5-yl)ethanone

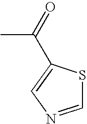

A solution of n-butyllithium in hexanes (77 mL of 2.5M) was added at below −65° C. to a stirred solution of 2-trimethylsilylthiazole (28.9 g, 0.184 mol) in ether (500 mL), and the resulting mixture was stirred 30 min at −78° C. A solution of N-methoxy-N-methylacetamide (20.9 g) in about 70 ml ether was added over 5 min with cooling so the reaction temperature did not exceed −85° C., and the mixture was allowed to warm to about 10° C. over 40 min. 1N HCl (200 mL) was added, followed by 40 mL of 12N HCl to give a pH between 0 and 1, and the mixture was stirred for 45 min at RT. The pH was brought to 7 with solid NaHCO₃, the layers separated, and the aqueous layer extracted with about 700 mL ether in 3 portions. The combined organic layers were dried over giving 21.2 g (91%) of the title substance as a light brown solid, ¹H NMR (CDCl₃) δ 8.98 (s, 1H), 8.40 (s, 1H), 2.61 (s, 3H).

### Preparation 116B

### 2-bromo-1-(thiazol-5-yl)ethanone hydrobromide

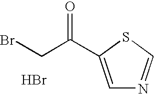

Pyridinium tribromide (50.8 g, 0.144 mol) was added to a solution of 1-(thiazol-5-yl)ethanone (18.2 g, 0.143 mol) in 39 mL 33% HBr-acetic acid and 39 mL acetic acid at RT and the resulting mixture stirred 15 h at RT. The suspension was filtered and the resulting solid washed with acetic acid (2×50 mL) and dried at 70° C. in vacuo. Yield 40 g (98%) of an off-white solid, ¹H NMR (CD₃OD) showed a 1.6:1 mixture of ketone and corresponding trideuterioMeOH hemiketal forms, respectively. For the ketone form: δ 9.66 (s, 1H). 8.72 (s, 1H), 4.63 (s, 3H). For the hemiketal form: δ 10.03 (s, 1H), 8.41 (s, 1H), 3.84 (d, 1H, A of AB. J=11 Hz), 3.76 (d, 1H, B of AB, J=11 Hz). Anal Calcd for C₅H₅Br₂NOS: C, 20.93; H, 1.76; N, 4.88. Found: C, 21.39; H, 1.79; N, 4.90.

### Preparation 116C

### 5-(2-(4-iodophenyl)-4-(thiazol-5-yl)-1H-imidazol-1-yl)-2-methylpyridine

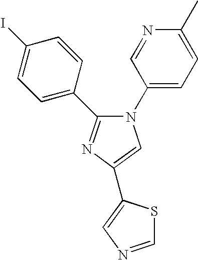

A mixture of 4-iodo-N′-(6-methylpyridin-3-yl)benzamidine (34.8 g, 103 mmol), 2-bromo-1-(thiazol-5-yl)ethanone hydrobromide (31 g, 108 mmol), KHCO3 (41 g. 412 mmol), and 3-t-butyl-4-hydroxy-5-methylphenylsulfide (5 mg) in t-butanol (300 mL) was stirred at 50° C. in the dark for 17 h. The suspension was filtered and the solids washed with 2-propanol. The filtrate was concentrated, the residue dissolved in acetic acid (40 ml) and the resulting solution heated at 90° C. for 20 min and concentrated. The residue was dissolved in 1M NaHCO₃ (300 ml) and the mixture extracted with EtOAc (3×300 ml). The organic layers were washed with 10% aq. citric acid, water, dried (Na₂SO₄) and concentrated. SGC (linear gradient of 3%-100% EtOAc gave 10.7 g of a yellow solid (23%). ¹H NMR (CDCl₃) δ8.72 (s, 1H), 8.45 (d, 1H, J=2.5 Hz), 8.14 (s, 1H), 7.64 (d, 2H, J=8.3 Hz), 7.42 (dd, 1H, J=2.5, 8.3 Hz), 7.38 (s, 1H), 7.22 (d, 1H, J=8.3 Hz), 7.13 (d, 2H, J=8.3 Hz), 2.63 (s, 3H). HPLCMS 8.74 min, m/e 445 (MH+).

### Preparation 116D

### N-(4-(1-(6-methylpyridin-3-yl)-4-(thiazol-5-yl)-1H-imidazol-2-yl)phenyl)-3-nitropyridin-2-amine

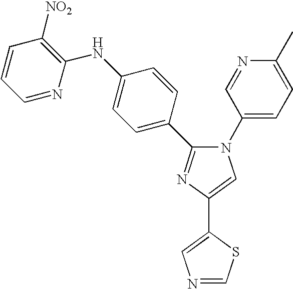

A mixture of 5-(2-(4-iodophenyl)-4-(thiazol-5-yl)-1H-imidazol-1-yl)-2-methylpyridine (2.7 g, 6.08 mmol), 2-amino-3-nitropyridine (928 mg, 6.69 mmol), tris(dibenzylideneacetone)dipalladium(0) (167 mg, 0.182 mmol), 4,5-bis(diphenylphosphino)-9,9-dimethylxanthene (264 mg, 0.456 mmol), Cs₂CO₃ (2.96 g, 9.12 mmol) and p-dioxane (18 ml.) was heated by microwave at 150° C. for 2.5 h. The mixture was filtered, concentrated, and combined with another mixture prepared identically on an 8.49 mmol scale (3.77 g of starting iodide), SGC (35%-100% EtOAc-hexanes, linear gradient) provided 3.45 g of a red solid (52%). ¹H NMR (CDCl₃) δ 10.22 (s, 1H), 8.72 (s, 1H), 8.53-8.50 (m, 2H), 8.48 (dd, 1H, J=1.7, 5 Hz), δ 8.17 (s, 1H), 7.88 (m, 2H). 7.46 (dd, 1H, J=2.5, 8.3 Hz), 7.42 (m, 2H), 7.38 (s, 1H), 7.22 (d, 1H, J=8.3 Hz), 6.87 (dd, 1H, J=4.6, 8.3 Hz), 2.62 (s, 3H). HPLCMS 8.38 min, m/e 456 (MH+).

### Preparation 116E

### N⁴-(1-(6-methylpyridin-3-yl)-4-(thiazol-5-yl)-1H

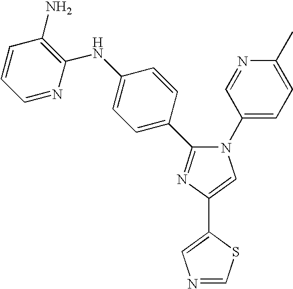

A mixture of N-(4-(t-(6-methylpyridin-3-yl)-4-(thiazol-5-yl)-1H-imidazol-2-yl)phenyl)-3-nitropyridin-2-amine (1.0 g, 2.19 mmol) and palladium-on-carbon (200 mg) in MeOH (20 ml) was shaken under 45 p.s.i. hydrogen pressure for 7 h, filtered, and concentrated. The product was combined with that obtained identically in a separate reaction using 3.4 g (7.45 mmol) of starting nitro compound. Yield 4.1 g, (100%). ¹H NMR (CDCl₃) δ 8.68 (m, 1H), 8.47 (m, 1H), 8.12 (m, 1H), 7.79 (d, 1H), 739 (d, 1H), 7.31 (s, 1H), 7.28 (m, 2H), 7.23-7.14 (m, 3H), δ 99 (m, 1H), 6.75 (m, 1H), 6.35 (br, 1H), 3.41 (br, 2H), 2.58 (s, 3H)HPLCMS 3.92 min, m/e 426 (MH+).

### Example 117

### 2-(trifluoromethyl)-3-(4-(1-(6-methylpyridin-3-yl)-4-(thiazol-5-yl)-1H-imidazol-2-yl)phenyl)-3H-imidazo[4,5-b]pyridine

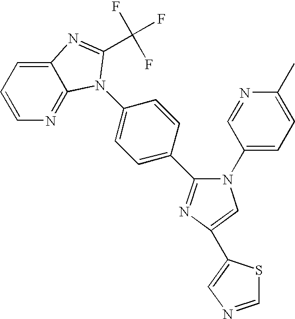

N²-(4-(1-(6-methylpyridin-3-yl)-4-(thiazol-5-yl)-1H-imidazol-2-yl)phenyl)pyridine-2,3-diamine (2.00 g, 4.71 mmol) was dissolved in 10 mL CF3COOH, The solution was heated in a sealed vessel in a 90° C. oil bath for 4 h, cooled, concentrated, and the residue partitioned between 50 mL 4:1 DCM: 2-propanol and 30 mL 1M NaHCO₃. The aqueous layer was separated and extracted with DCM (2×20 ml). The combined organic layers were dried and concentrated SGC (0%-5% MeOH—CHCl3/0.5% NH₄OH, linear gradient), provided 1.85 g of an off-white solid which was triturated with ether-hexanes. Yield 1.30 g, 55%. ¹H NMR (CDCl₃) δ 8.74 (s, 1H), 8.58 (d, 1H, J=2.5 Hz), 8.51 (dd, 1H, J=1.5, 4.8 Hz), 8.24 (dd, 1H, J= 1.2, 8.3 Hz), 8.17 (s, 1H), 7.67 (m, 2H), 7.51 (dd, 1H, J=2.5, 8.3 Hz). 7.43-7.38 (m, 4H), 7.26 (d, 1H, J=8.3 Hz), 2.64 (s, 3H). HPLCMS 7.84 min, m/e 504 (MH+). IC₅₀=0.614 nM.

### Example 118

### 3-(4-(1-(6-methylpyridin-3-yl)-4-(thiazol-2-yl)-1H-imidazol-2-yl)phenyl)-1H-imidazo[4,5-b]pyridin-2(3H)-one

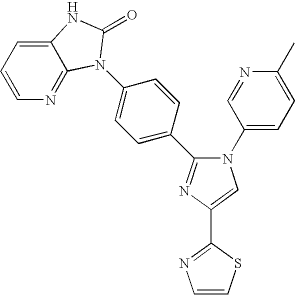

A solution of 2-methoxy-3(4-(1-(6-methylpyridin-3-yl)-4-(thiazol-2-yl)-1H-imidazol-2-yl)phenyl)-3H-imidazo[4,5-b]pyridine (140 mg, 0.30 mmol) was in p-dioxane (20 mL) and 1N HCl (20 mL) was stirred at RT for 72 h and heated at 65° C. for 24 h. 1N NaHCO₃ was added to give pH 8, and the mixture was extracted with EtOAc (3×40 mL). The organic layers were combined, dried and concentrated. SGC (0-5% MeOH in CHCl3, 0.5% NH₄OH) gave 80 mg of a colorless solid (59%). ¹H NMR (CDCl₃) δ 9.30 (s, 1H), 8.56 (d, 1H, J=2.5 Hz), 8.06 (dd, 1H, J=1, 5 Hz), 7.82 (d, 1H, J=3.3 Hz), 7.81-7.78 (m, 3H), 7.61 (m, 2H), 7.48 (dd, 1H, J=3, 8.3 Hz), 7.34 (dd, 1H, J=17, 8 Hz), 7.31 (d, 1H, J=3.3 Hz), 7.23 (d, 1H, J=8 Hz), 7.06 (dd, 1H, J=5, 7.7 Hz), 2.63 (s, 3H). HPLCMS 6.48 min, m/e 452 (MH+). IC₅₀=0.748 nM.

### Example 119

### 1-(4-(2-(pyridin-2-yl)-5-(pyridin-3-yl)-2H-1,2,3-triazol-4-yl)phenyl)-1H-pyrrolo[2,3-b]pyridine

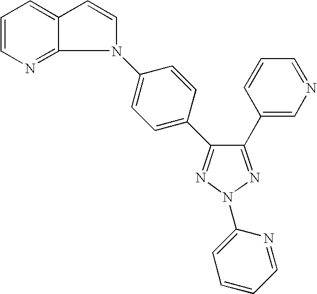

1-(4-(5-(pyridin-3-yl)-2H-1,2,3-triazol-4-yl)phenyl)-1H-pyrrolo[2,3-b]pyridine (200 mg, 0.58 mmol), N-fluoropyridinium triflate (287 mg, 1.17 mmol), Cs2CO₃ (380 mg, 1.17 mmol) and MeOH (5 mL) were stirred at RT for 16 h and concentrated. Aqueous 1N NaOH (20 mL) was added to the residue and the mixture extracted three times with EtOAc (60 mL total). The organic layers were dried and concentrated. SGC (0.5%-1% MeOH in DCM/0.5% NH₄OH) gave 100 mg of a colorless solid. ¹H NMR (CDCl₃) δ 8.98 (dd, 1H, J=1, 2 Hz), 8.86-8.82 (m, 2H), 8.37 (dd, 1H, J=1.7, 4.6 Hz), 8.19 (dt, 1H, J=1, 8 Hz), 8.03 (dt, 1H, J=2, 8 Hz), 7.97 (dd, 1H, J=1.7, 7.9 Hz), 7.95-7.87 (m, 3H), 7.79 (m, 2H), 7.55 (d, 1H, J=3.3 Hz), 7.38 (dd, 1H, J=1, 4.5 Hz), 7.35 (d, 1H, J=4.6 Hz), 7.14 (dd, 1H, J=4.6, 7.9 Hz), 6.65 (d, 1H, J=3.7 Hz). HPLCMS (method 2) 9.58 min, m/e 416 (MH+). The sample appeared to contain isomers of the title substance (6.83 min, 2%, m/e 416, and 8.97 min, 8%, m/e 416). IC₅₀=134 nM.

### Preparation 119A

### 1-(4-ethynylphenyl)-1H-pyrrolo[2,3-b]pyridine

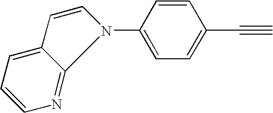

A mixture of 1-bromo-4-ethynylbenzene (3.1 g, 17.1 mmol), 7-azaindole (2.43 g, 20.5 mmol), trans-N,N′-dimethyl-cyclohexane-1,2-diamine (490 mg, 3.42 mmol), CuI (163 mg, 0.88 mmol), and K₃PO₄ (7.3 g, 34.2 mmol) in toluene was heated at reflux for 20 h, cooled, and filtered. The solid was washed with 5:1 DCM; 2-propanol and the filtrates were concentrated. SGC (10% EtOAc-hexanes) gave 1.5 g of a yellow oil which solidified on standing. ¹H NMR (CDCb) δ 8.36 (dd, 1H, J=1.7, 4.6 Hz), 7.95 (dd, 1H, J=1.7, 7.9 Hz), 7.78 (m, 2H), 7.82 (m, 2H), 7.49 (d, 1H, J=3.7 Hz). 7.13 (dd, 1H, J=4.6, 7.9 Hz), 6.62 (d, 1H, J=3.7 Hz), 3.10 (s, 3H). MS (AP+) m/e 219 (MH+).

### Preparation 119B

### 1-(4-(2-(pyridin-3-yl)ethynyl)phenyl)-1H-pyrrolo[2,3-b]pyridine

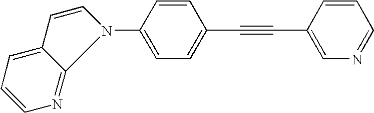

A mixture of 1-(4-ethynylphenyl)-1H-pyrrolo[2,3-b]pyridine (1.5 g, 6.88 mmol), 3-iodopyridine (1.48 g, 7.22 mmol), bis-(triphenylphosphine)palladium (II) dichloride (241 mg, 0.344 mmol) and CuI (65 mg, 0.344 mmol) in triethylamine (5 ml) and DMF (4 ml) was heated at 80° C. for 2.5 h, cooled, and concentrated. SGC (20%-50% EtOAc in hexanes, linear gradient) gave 1.5 g of a yellow solid (74%). ¹H NMR (CDCl₃) δ 8.77 (s, 1H), 8.53 (d, 1H, J=4 Hz), 8.37 (dd, 1H, J=1.7, 4.8 Hz), 7.95 (dd, 1H, J=1.7, 7.9 Hz), 7.83 (m, 2H), 7.80 (m, 1H), 7.67 (m, 2H), 7.51 (d, 1H, J=3.7 Hz), 7.26 (dd, 1H, J=5.0, 7.8 Hz), 7.13 (dd, 1H, J= 5.0, 7.8 Hz). 8.83 (d, 1H, J=3.7 Hz). MS (AP+) m/e 296 (MH+).

### Preparation 119C

### 1-(4-(5-(pyridin-3-yl)-2H-1,2,3-triazol-4-yl)phenyl)-1H-pyrrolo[2,3-b]pyridine

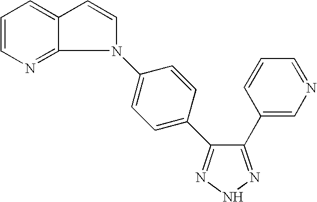

1-(4-(2-(pyridin-3-yl)ethynyl)phenyl)-1H-pyrrolo[2,3-b]pyridine (742 mg, 2.52 mmol) and azidotrimethylsilane (579 mg, 5.0 mmol) were heated together in a sealed vial at 150° C. for about 100 h. SGC (linear gradient of 50% to 100% EtOAc in hexanes) provided a colorless solid. Yield 500 mg. 49%. ¹H NMR (CDCl₃) δ 8-39 (br, 1H), 8.83 (dd, 1H, J=1.7, 4.6 Hz), 8.43 (dd, 1H, J=1.7, 4.6 Hz), 8.03-7.98 (m, 2H), 7.79 (m, 2H), 7.64 (m, 2H), 7.52 (d, 1H, J=3.7 Hz), 7.40 (dd, 1H, J=5.0, 7.9 Hz), 7.18 (dd, 1H, J=4.6, 7.9 Hz), 6.67 (d, 1H, J=3.7 Hz). HPLCMS 7.30 min, m/e 339 (MH+).

### Example 120

### 1-(4-(1-(pyridin-2-yl)-4-(pyridin-3-yl)-1H-pyrazol-3-yl)phenyl)-1H-pyrrolo[2,3-b]pyridine

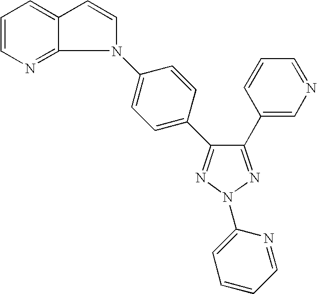

A mixture of 1-(4-(4-pyridin-3-yl)-1H-pyrazol-3-yl)phenyl)-1H-pyrrolo[2,3-b]pyridine (110 mg, 0.326 mmol), 2-iodopyridine (74 mg, 0.36 mmol), trans-N,N′-dimethyl-cyclohexane-1,2-diamine (4.7 mg, 0.033 mmol), CuI (3 mg, 0.016 mmol), K2CO3 (95 mg, 0.68 mmol) in toluene (2 ml) was heated in a sealed vial at 110° C. for 17 h. SGC (1% MeOH in DCM, 0.5% NH₄OH) gave an off-white solid, 75 mg (56%). ¹H NMR (CDCl₃) δ8.75 (s, 1H), 8.74 (br, 1H), 8.56 (br, 1H), 8.43 (ddd, 1H, J=0.8, 1.7, 5.0 Hz), 8.37 (dd, 1H, J=1.5, 4.8 Hz), 8.11 (dt, 1H, J=1, 8.3 Hz), 7.96 (dd, 1H, J=17, 7.9 Hz), 7.86 (m, 1H), 7.81 (m, 2H), 7.73-7.69 (m, 3H), 7.53 (d, 1H, J=3.5 Hz), 7.30 (br, 1H), 7.22 (ddd, 1H), 7.13 (dd, 1H, J=5.0, 8.0 Hz), 6.63 (d, 1H, J=3.7 Hz). HPLCMS 9.28 min, 96%, m/e 415 (MH+). IC₅₀=42.9 nM.

### Preparation 120A

### 1-(4-(1H-pyrrolo[2,3-b]pyridin-1-yl)phenyl)-3-(dimethylamino)-2-(pyridin-3-yl)prop-2-en-1-one

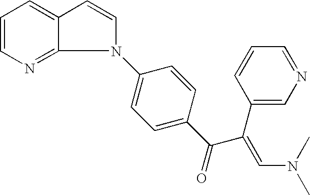

1-(4-(1H-pyrrolo[2,3-b[pyridin-1-yl)phenyl)-2-(pyridin-3-yl)ethanone (257 mg, 0.82 mmol) and diethoxy-N,N-dimethylmethanamme (DMF diethyl acetal, 483 mg, 3.28 mmol) were combined in a sealed vial, heated at 134° C. with stirring for 2 h giving a solution, and concentrated. A yellow-brown solid was thus obtained, 380 mg. ¹H NMR (CDCl₃) δ 8.45 (dd, 1H, J=1, 2 Hz), 8.44 (dd, 1H, J=1.7, 5.0 Hz), 8.36 (dd, 1H, J=1.7, 5.0 Hz), 7.96 (dd, 1H, J= 1.7, 7.9 Hz), 7.79 (m, 2H), 7.62 (m, 2H), 7.54 (dt, 1H, J=2, 7.8 Hz), 7.51 (d, 1H, J=3.7 Hz), 7.45 (s, 1H), 7.22 (ddd, 1H, J=0.8, 4.8, 7.9 Hz), 7.13 (dd, 1H, J=5.0, 7.9 Hz), 8.63 (d, 1H, J=3.7 Hz), 2.77 (br s, 6H). MS (AP+) 369 (MH+).

### Preparation 1208

### 1-(4-(4-(pyridin-3-yl)-1H-pyrazol-3-yl)phenyl)-1H-pyrrolo[2,3-b]pyridine

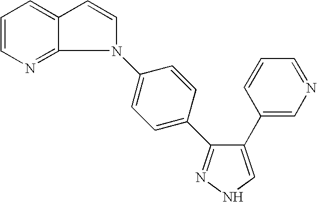

Hydrazine hydrate (32 mg, 164 mmol) was added to a solution of 1-(4-(1H-pyrrolo[2,3-b]pyridin-1-yl)phenyl)-3-(dimethylamino)-2-(pyridin-3-yl)prop-2-en-1-one (380 mg, 0.82 mmol) in 4 ml MeOH, and the resulting solution was heated at reflux for 2 h and concentrated. The residue was dissolved in EtOAc (25 ml) and the solution washed with water, dried, and concentrated giving a yellow solid (280 mg, 100%). ¹H NMR (CDCl₃) δ 8.78 (br, 1H), 8.51 (br, 1H), 8.40 (dd, 1H, J=1.5, 4.8 Hz), 7.98 (dd, 1H, J=1.7, 7.9 Hz), 7.79 (m, 2H), 7.73 (m, 2H), 7.56 (d, 2H, J=8.7 Hz), 7.51 (d, 1H, J=3.7 Hz), 7.32 (dd, 1H, J=5, 7.7 Hz), 7.18 (dd, 1H, J=4.6, 7.9 Hz), 8.65 (d, 1H, J=3.7 Hz). HPLCMS 5.38 min. 97%. MS (AP+) m/e 338 (MH+).

### Example 121

### 1-(4-(3-(pyridin-2-yl)-5-(pyridin-3-yl)-1H-pyrazol-1-yl)phenyl)-1H-pyrrolo)-2,3-b]pyridine bis-TFA salt

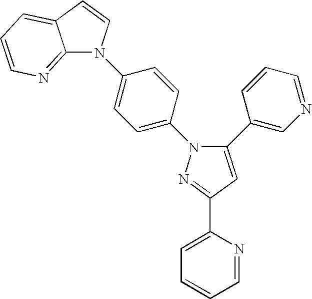

The mixture of 2-(1-(4-iodophenyl)-5-(pyridin-3-yl)-1H-pyrazol-3-yl)pyridine and 3-(1-(4-iodophenyl)-5-(pyridin-2-yl)-1H-pyrazol-3-yl)pyridine described below (880 mg, 2.0 mmol), 7-azaindole (294 mg, 2.5 mmol), CuI (20 mg, 0.104 mmol), K₂PO₄ (880 mg, 4.15 mmol), trans-N,N′-dimethyl-cyclohexane-1,2-diamine (24 mg, 0.21 mmol), and p-dioxane (10 mL) were combined and heated in a sealed vial at 120° C. for 18 h. the mixture was filtered and the filtrate evaporated giving a brown solid. The major spot by TIC was isolated by SGC (MeOH-DCM gradient) and proved to be a 3:1 mixture of two substances by HPLCMS 8.12 min and 7.35 min, respectively, both showing m/e 415 (MH+)). Preparative RP-HPLC (acidic system) provided 156 mg of the major isomer. The structure, including salt stoichiometry, was assigned by X-ray diffraction spectroscopy on a crystal grown from 98:2 MeCN—H₂O. ¹H NMR (CDCl₃) δ 115 (br, 3-4H), 8.98 (d, 1H, J=4.6 Hz), 8.69 (m, 2H), 8.45 (d, 1H, J=8.3 Hz), 8.38 (dd, 1H, J=1.7, 5.0 Hz). 8.25 (dt, 1H, J=17, 79 Hz), 8.08 (dd, 1H, J=17, 7.9 Hz), 8.03 (ddd, 1H, J=2, 2, 8 Hz), 7.87 (m, 2H), 7.76 (s, 1H), 7.69 (m, 1H), 7.63 (dd, 1H, J=5, 8 Hz), 7.58 (d, 1H, J=3.7 Hz), 7.51 (m, 2H), 7.24 (dd, 1H, J=4.6, 7.9 Hz), 6.72 (d, 1H, J=3.7 Hz). MS (AP+) m/e 415 (MH+). HPLC 8.15 min. Anal. Gated for C₂₀H₁₅N₆+2 CF3COOH: C, 58.08; H, 3.14; N, 13.08. Found; C, 55.76; H, 3.04; N, 13.01. IC₅₀=9.29 nM.

### Preparation 121A

### 1-(pyridin-2-yl)-3-(pyridin-3-yl)propane-1,3-dione

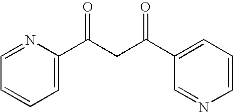

Sodium methoxide (4.35 g, 30.6 mmol) was added at RT to a solution of 2-acetylpyridine (6.13 g, 67.2 mmol) and methyl nicotinate (9.21 g, 67.2 mmol) in THF (200 mL). The mixture was heated at 50° C. for 1.5 h and at RT for 18 h. The resulting suspension was filtered and the orange solid (12 g) dissolved in water. The resulting solution was brought to pH 6-7 with aqueous NaH₂PO₄ and extracted with 5:1 DCM: 2-propanol. The organic layers were dried and concentrated giving an off-white solid (7 g, 67%). MS (AP+) m/e 227 (MH+).

### Preparation 121B

### 2-(1-(4-iodophenyl)-5-(pyridin-3-yl)-1H-pyrazol-3-yl)pyridine and 3-(1-(4-iodophenyl)-5-(pyridin-2-yl)-1H-pyrazol-3-yl)pyridine

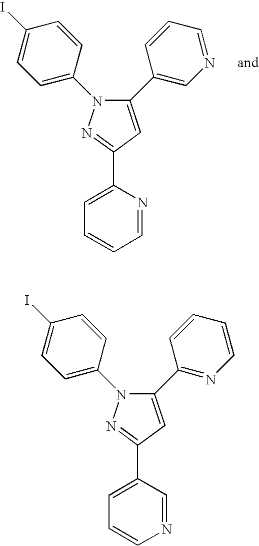

A mixture of 1-(pyridin-2-yl)-3-(pyridin-3-yl)propane-1,3-dione (1.61 g; 7.12 mmol) and p-iodophenylhydrazine (2.5 g, 10.7 mmol) in acetic acid (15 mL) was heated at 70° C. for 90 min and concentrated. The resulting oil was dissolved in 1M NaHCO₃ (40 mL) and extracted with DCM (3×20 ml). The organic layers were dried and concentrated giving 3.7 g of a dark solid. SGC (1% MeOH in DCM, 0.5% NH₄OH) provided 1.75 g (58%) of a yellow solid, HPLCMS 7.75 min (30% of total), m/e 425 (MH+), and 8.59 min (60% of total), m/e 425 (MH+).

### Example 122

### 1-(4-(5-(pyridin-2-yl)-3-(pyridin-3-yl)-1H-pyrazol-1-yl)phenyl)-1H-pyrrolo[2,3-b]pyridine TFA salt

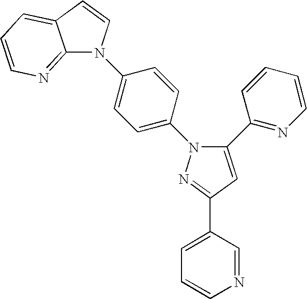

The minor substance from the CuI-catalyzed coupling of 2-(1-(4-iodophenyl)-5-(pyridin-3-yl)-1H-pyrazol-3-yl)pyridine and 3-(1-(4-iodophenyl)-5-(pyridin-2-yl-1H-pyrazol-3-yl)pyridine with 7-azaindole as described in the preceding Example was isolated by preparative RP-HPLC (acidic system) and assigned the title structure. Yield 50 mg. ¹H NMR (CDCl₃) δ 9.30 (br, 1H), 8.73-8.70 (m, 2H), 7.78 (dd, 1H, J=5.4, 7.9 Hz), 8.37 (dd, 1H J=137, 4.6 Hz), 7.98 (dd, 1H, J=4.8, 7.7 Hz), 6.66 (d, 1H, J=3.7 Hz), HPLCMS 7.35 min. m/e 415 (MH+). Anal. Calcd for C₂₈H₁₈N₈+CF₃COOH+H₂O: C, 63.51; H, 3.81; N, 15.87. Found: C. 63.61; H, 3.93, N, 16.08. IC₅₀=21.1 nM.

### Preparation 123

### 1-(4-(5-(pyridin-2-yl)-2-(pyridin-3-yl)-2H-1,2,4-triazol-3-yl)phenyl)-1H-pyrrolo[2,3-b]pyridine

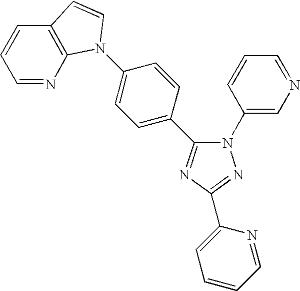

2-(5-(4-iodophenyl)-1-(pyridin-3-yl)-1H-1,2,4-triazol-3-yl)pyridine (350 mg. 0.82 mmol), 7-azaindole (117 mg. 0.99 mmol.) CuI (2.5 mg. 0.012 mmol), K₃PO₄ (349 mg, 1.65 mmol), trans-N,N′-dimethyl-cyclohexane-1,2-diamine (9.4 mg. 0.082 mmol) and p-dioxane (8 mL) were combined in a sealed vial, stirred at 120° C. for 48 h), filtered and the filtrate concentrated. SGC (linear gradient of 0.5%-1.5% MeOH in DCM, 0.5% NH₄OH) gave a light yellow solid, 163 mg (51%). IC₅₀=26.2 nM.

### Preparation 123A

### 3-Pyridylhydrazine

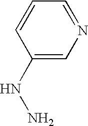

A solution of sodium nitrite (12.2 g, 177 mmol) in water (100 mL) was added portionwise at 0° C. to a solution of 3-aminopyridine (16.7 g. 177 mmol) in 6N HCl (180 mL) and the mixture was stirred at 0° C. for 30 min. A solution of SnCl2.2H2O (100 g. 443 mmol) in 6N HCl (100 mL) was added and the mixture was stirred at 0° C. for 3 h. 40% aqueous KOH was added to give pH 14, and the mixture extracted with six portions of EtOAc. The organic layers were dried and concentrated. The residue was purified by SGC (3% MeOH in DCM, 0.5% NH4OH) giving a yellow oil which solidified on standing (4.9 g. 25%).

### Preparation 123B

### 2-((pyridin-2-yl)methylene)-1-(pyridin-3-yl)hydrazine

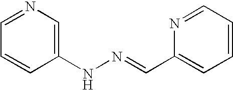

A solution of 3-pyridlhydrazine (3.7 g, 34.0 mmol) and 2-pyridinecarbaidehyde (3.63 g. 34.0 mmol) 80 mL ethanol and 5 mL acetic acid was heated at 78° C. for 3 h and concentrated. The residue was triturated with either giving a yellow solid. Yield 4.3 g. 65%.

### Preparation 123C

### 2-(5-(4-iodophenyl)-1-(pyridin-3-yl)-1H-1,2,4-triazol-3-yl)pyridine

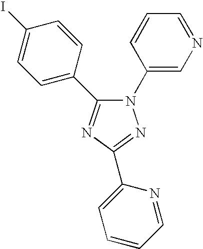

Pyridinium tribromide (6.14 g, 19.2 mmol) was added at 0° C. to a solution of 2-((pyridin-2-yl)methylene)-1-(pyridin-3-yl)hydrazine (3.8 g, 19.2 mmol) in THF (40 mL) and the mixture was stirred at 0° C. for 3 h. 4-lodobenzylamine (4.47 g, 19.2 mmol) and triethylamine (9.6 g, 96 mmol) were added sequentially, and the mixture was stirred at RT for 2 h, and 65° C. for 1 h and concentrated. The brown solid residue (7 g) was suspended in acetonitrile (50 mL), silver carbonate (5.29 g, 19.2 mmol) was added, and the mixture was stirred at RT for 18 h. filtered, and the solid washed with DCM. The filtrates were combined and concentrated and the residue purified by SGC (1% and 2% MeOH in DCM, 0.5% NH₄OH) giving the title substance as a reddish solid (1.0 g, 12% for 3 steps).

The invention described and claimed herein is not to be limited in scope by the specific examples and embodiments herein disclosed, since these examples and embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fail within the scope of the appended claims.

1. A compound of formula I, 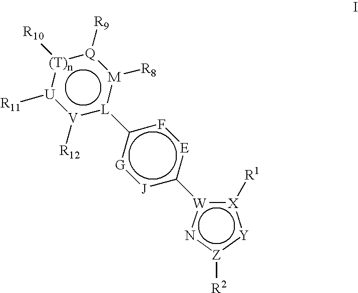
or a pharmaceutically acceptable salt thereof;
wherein N, W, X, Y, and Z together form a 5-membered heteroaromatic ring;
W, X, and Z are independently selected from the group consisting of carbon and nitrogen;
Y is selected from the group consisting of CR²⁰, M, N(O), NR²¹, and O;
with the proviso that at least two of W, X, and Z are carbon or at least one of W, X, and Z is carbon and Y is CR²⁰;
R¹ is selected from the group consisting of phenyl, a 5 to 6-membered heteroaryl, naphthyl, a 5 to 6-membered heteroaryl fused to a 5 to 6-membered heteroaromatic ring, phenyl fused to a 5 to 6-membered heteroaromatic ring, a 5 to 6-membered heteroaryl fused to benzene, a phenyl fused to a 5 to 7-membered cycloalkane, a 5 to 6-membered heteroaryl fused to a 5 to 7-membered cycloalkane, phenyl fused to a 5 to 7-membered heterocycloalkane, and a 5 to 6-membered heteroaryl fused to a 5 to 7-membered heterocycloalkane, wherein said heteroaromatic rings, heteroaryls, and heterocycloalkanes independently contain 1 to 4 heteroatoms independently selected from the group consisting of O, N, and S; and wherein said phenyl and heteroaryl groups of said fused groups are directly bonded to X; and wherein R¹ is optionally substituted with 1 to 3 substituents, independently selected from the group consisting of hydroxy, nitro, oxo, and R³; wherein one of said substituents is optionally further selected from the group consisting of R^{3a};
wherein each R³ is independently selected from the group consisting of halo, cyano, formyl, carbamoyl, carboxy, amino, (C₁-C₆) alkyl, cyclopropyl, (C₃₋C₇)cycloalkyl-(C₁-C₃)alkyl, cyano-(C₁-C₄)alkyl, —OR¹³ hydroxy(C₁-C₆)alkyl, R¹³O—(C₁-C₆)alkyl, R¹³S—(C₁-C₆)alkyl, hydroxy-(C₁-C₆)alkoxy, R¹³O—(C₁-C₆)alkoxy, amino-(C₂-C₆)alkoxy, R¹³R¹⁴(C₂-C₆)alkoxy, hydroxy-(C₂-C₆)alkyl-N(R¹⁴), R¹³O—(C₂-C₆)alkyl-N(R¹⁴), hydroxy-(C₁-C₆)alkyl-S, R¹³O—(C₁-C₅)alkyl-S—, —SR¹³, —S(O)R¹³, —S(O)₂R¹³, —S(O)₂NH₂, —S(O)₂R¹³R¹⁴, —C(═O)R¹³, —OC(═O)H, OC(═O)R¹³, —OC(═O)OR¹³, —C(═O)OR¹³, carboxy-(C₁-C₄)alkyl, R¹³OC(═O)—(C₁-C₄)alkyl, carbamoyl-(C₁-C₄)alkyl, R¹³R¹⁴NC(═O)—(C₁₋C₄)alkyl, carboxy-(C₁-C₄)alkoxy, R¹³OC(═O)—(C₁-C₄)alkoxy, carbamoyl-(C₁₋C₄)alkoxy, R¹³R¹⁴NC(═O)—(C₁-C₄)alkoxy, amino-(C₁-C₆)alkyl, R¹³R¹⁴N—(C₁-C₆)alkyl, R¹³R¹⁴N—(C₂-C₆)alkoxy, —C(═O)NR¹³R¹⁴, —OC(═O)NH₂, —OC(═O)NR¹⁸R¹⁴, —N(R¹⁴)C(═O)H, —N(R¹⁴)C(═O)R¹³, phenyl-A-, 5 to 6-membered heteroaryl-A-, phenyl-(A)ₘ-(C₁-C₄alkyl), and 5 to 6-membered heteroaryl-(A)ₘ, —(C₁-C₄)alkyl); wherein said phenyls and heteroaryls are optionally substituted with 1 to 3 substituents independently selected from halo, trifluoromethyl, hydroxy, cyano, cyano-(C₁-C₄)alkyl, R¹³, —OR¹³ hydroxy-(C₁-C₅)alkyl, and R¹³O—(C₁-C₆)alkyl; and wherein said alkyl, cycloalkyl, cycloalkyl-alkyl, and alkoxy groups are optionally independently substituted with 1 to 5 fluorine atoms; wherein A is independently O or S; and wherein m is independently 0 or 1;
wherein each R^{3a} is independently (C₄-C₇)cycloalkyl, (C₂-C₅)alkenyl, (C₂-C₆)alkynyl, —NR¹³R¹⁴, phenyl, 5 to 6-membered heteroaryl, or 4 to 6-membered heterocyclyl containing 1 to 3 heteroatoms selected from N, O, and S; wherein said cycloalkyl, alkenyl, and alkynyl groups are optionally independently substituted with 1 to 3 fluorine atoms; and wherein said phenyl, heteroaryl, and heterocyclic groups are optionally substituted with 1 to 3 substituents independently selected from halo, trifluoromethyl, hydroxy, cyano, cyano-(C₁-C₄)alkyl, R¹³, —OR¹³, hydroxy-(C₁-C₆)alkyl, and R¹³O—(C₁-C₆)alkyl;
wherein each R¹³ is independently selected from the group consisting of (C₁-C₆)alkyl, (C₃-C₇)cycloalkyl, and (C₃-C₇)cycloalkyl-(C₁-C₃)alkyl; wherein said alkyl, cycloalkyl, and cycloalkyl-alkyl-groups are optionally independently substituted with 1 to 5 fluorine atoms;
wherein each R¹⁴ is independently selected from the group consisting of H, (C₁-C₅)alkyl, (C₁-C₅)alkoxy, (C₃-C₅)cycloalkyl, and (C₃-C₅)cycloalkyl-(C₁-C₃)alkyl; wherein said alkyl, alkoxy, and cycloalkyl groups are optionally independently substituted with 1 to 3 fluorine atoms;
or optionally R¹³ and R¹⁴ together with the nitrogen to which they are attached form a 4 to 6-membered heterocyclic ring containing 1 to 3 heteroatoms selected from N, O, and S; wherein said heterocyclic ring may be optionally substituted with 1 to 4 substituents independently selected from fluoro, (C₁-C₄)alkyl, and (C₁-C₄)alkoxy; and wherein 1 to 2 of said substituents may be further selected from hydroxy, oxo, and trifluoromethyl;
R² is selected from the group consisting of phenyl, a 5 to 6-membered heteroaryl, naphthyl, a 6 to 6-membered heteroaryl fused to a 5 to 6-membered heteroaromatic ring, phenyl fused to a 5 to 6-membered heteroaromatic ring, and a 5 to 6-membered heteroaryl fused to benzene; wherein said heteroaryls and heteroaromatic rings each independently contain 1 to 3 heteroatoms independently selected from the group consisting of O, N, and S; and wherein said phenyl and heteroaryl groups of said fused groups are directly bonded to Z;
and wherein R² is optionally substituted with 1 to 3 substituents, wherein one substituent may be selected from the group consisting of halo, OH, CN, amino, R¹⁵, hydroxy-(C₁-C₄)alkyl, R¹⁵O—(C₁₋C₂)alkyl(cyano(C₁-C₄)alkyl, —OR¹⁵, —SR¹⁵, —SO₂R¹⁵, and —NR¹⁵R¹⁶; and wherein 1 to 2 substituents may be independently selected from halo, methyl, ethyl, n-propyl, methoxy, ethoxy, difluoromethyl, and trifluoromethyl;
wherein each R¹⁵ is independently selected from the group consisting of (C₁-C₄)alkyl, (C₂-C₄)alkenyl, cyclopropyl, and cyclopropylmethyl, optionally independently substituted with 1 to 3 fluorine atoms;
R¹⁶ is H, (C₁-C₃)alkyl, or (C₁-C₃)alkoxy;
R²⁰ is selected from the group consisting of H, NHR¹⁸, (C₂-C₆)alkynyl, and R³;
R²¹ is selected from the group consisting of H, (C₁-C₈)alkyl, (C₃-C₅)cycloalkyl-(C₁-C₃)alkyl, (C₂-C₅)alkenyl, (C₂-C₆)alkynyl, cyano(C₁-C₄)alkyl, hydroxy, —OR¹³, hydroxy-(C₁-C₅)alkyl, R¹³O—(C₁-C₆)alkyl, R¹³S—(C₁-C₆)alkyl, hydroxy-(C₁-C₆)alkoxy, R¹³O(C₁₋C₆)alkoxy, amino-(C₂-C₆)alkoxy, R¹³R¹⁴N—(C₂-C₆)alkoxy, —S(O)₂R¹³, —S(O)₂NR¹³R¹⁴, —S(O)₂NH₂, carboxy-(C₁-C₄)alkyl, R¹³OC(═O)—(C₁-C₄)alkyl, R¹³R¹⁴N-(═O)—(C₁-C₄)alkyl, carbamoyl-(C₁-C₄)alkyl, carboxy-(C₁-C₄)alkoxy, R¹³OC(═O)—(C₂-C₆)alkoxy, carbamoyl-(C₁-C₄)alkoxy, R¹³R¹⁴NC(═O)—(C₁₋C₄)alkoxy, amino-(C₂-C₆)alkyl, R¹³R¹⁴N—(C₂-C₆)alkyl, amino-(C₂-C₆)alkoxy, R¹³R¹⁴N—(C₂-C₆)alkoxy, —OC(═O)NR¹³R¹⁴, phenyl-A-, 5 to 6-membered heteroaryl-A-, phenyl-(A)ₘ-(C₁-C₄ alkyl), and heteroaryl-(A)ₘ-(C₁-C₄ alkyl); wherein said phenyl or heteroaryl is optionally substituted with 1 to 3 substituents independently selected from halo, cyano, cyano-(C₁-C₄)alkyl, R¹³, OR¹³, and R¹³O—(C₁-C₆)alkyl; and wherein said alkenyl, alkynyl, alkyl, or alkoxy group is optionally substituted with 1 to 3 fluorine atoms;
E, F, G, J, and the two carbons to which they are attached, together form a 6-membered aromatic or heteroaromatic ring;
wherein E is selected from N, N(O), and CR⁴; wherein R⁴ is selected from the group consisting of H, halogen, methyl, —OH, and —NH₂,
F is selected from N, N(O), and CR⁵;
G is selected from N, N(O), and CR⁵;
J is selected from N, N(O), and CR⁷;
wherein R⁵, R⁶, and R⁷ are independently selected from the group consisting of H, halogen, cyano, hydroxy, amino, (C₁-C₄)alkyl, cyclopropyl, cyclopropylmethyl, hydroxy(C₁-C₃)alkyl, (C₁-C₃)alkoxy, (C₁-C₃)alkylamino, and di(C₁-C₃)alkylamino; wherein said alkyl and alkoxy groups are independently optionally substituted with 1 to 3 fluorine atoms;
L, M, Q, T, U, and V together form an aromatic or a heteroaromatic ring;
L is carbon or nitrogen;
n is zero or 1;
wherein when n is zero, then M, Q, U, and V are independently selected from the group consisting of C, N, O, and S; and
when n is 1, then M, Q, T, U, and V are independently selected from the group consisting of carbon and nitrogen;
R⁸, R⁹, R¹¹, and R¹², when present, are independently selected from the group consisting of H, hydroxy, nitro, R³, and R^{3a};
R¹⁰, when present, is selected from the group consisting of H, hydroxy, nitro, NHR¹³, and R³;
or optionally R⁵-M-Q-R⁹ are taken together to form a ring, or R⁸-M-Q-R⁹ are taken together to form a ring and R¹¹-U—V—R¹² are taken together to form another ring;
or optionally when n is zero, R⁹-Q-U—R¹¹ are taken together to form a ring;
or optionally when n is 1, R⁹-Q-T-R¹⁰ are taken together to form a ring;
or R⁸-M-Q-R⁹ are taken together to form a ring and R¹⁰-T-U—R¹¹ are taken together to form another ring,
wherein said rings formed from R⁸-M-Q-R⁹, R¹¹-U—V—R¹², R⁹-Q-U—R¹¹, R⁹-Q-T-R¹⁰, and/or R¹⁰-T-U—R¹¹ are 5 to 7 membered carbocyclic or heterocyclic rings, wherein said heterocyclic rings independently contain 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁₋C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally independently substituted with 1 to 5 fluorine atoms:
and wherein when the ring formed by W, X, Y, Z, and the nitrogen to which W and Z are attached, is selected from the group consisting of b, c, f, and i; 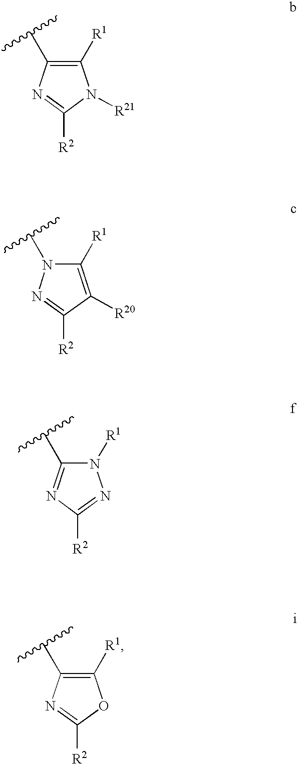
then 2 or more of the group consisting of R¹; R²; and the ring formed by L, M, Q, (T)ₙU, and V; must be heteroaryls.

2. A compound of claim 1, wherein the ring comprising of W, X, Y, and Z is selected from the group consisting of a, c, d, e, f, and g; 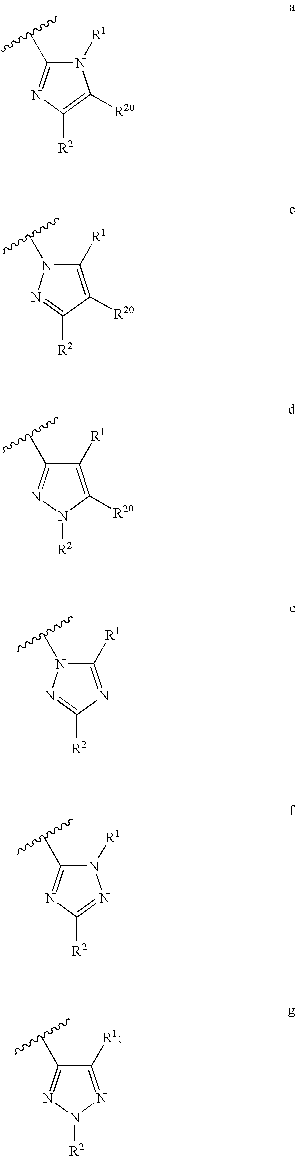
or a pharmaceutically acceptable salt thereof.

3. A compound of claim 1, wherein W, X, and Z are carbon and Y is NR²¹, or a pharmaceutically acceptable salt thereof.

4. A compound of claim 1, wherein W and Z are carbon, X is nitrogen, and Y is CR²⁶; or a pharmaceutically acceptable salt thereof.

5. A compound of claim 1, wherein R⁸-M-Q-R⁹ are taken together to form a ring; R¹¹ and R¹², when present, are independently selected from the group consisting of H, hydroxy, nitro, R³, and R^{3a}; and wherein R¹⁰, when present, is selected from the group consisting of H, hydroxy, nitro, NHR¹³, and R³;
or optionally when n is zero, R⁹-Q-U—R¹¹ are taken together to form a ring; and R⁶ and R¹², when present, are independently selected from the group consisting of H, hydroxy, nitro, R³, and R^{3a};
or optionally when n is 1, R⁹-Q-T-R¹⁵ are taken together to form a ring; R⁸, R¹¹, and R¹², when present, are independently selected from the group consisting of H, hydroxy, nitro, R³, and R^{3a};
wherein said rings are 5 to 7 membered carbocyclic or heterocyclic rings; wherein said heterocyclic rings contain 1 to 4 heteroatoms selected independently from the group consisting of N, O, and S; and wherein said rings are optionally substituted with 1 to 3 substituents independently selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms;
or a pharmaceutically acceptable salt thereof.

6. A compound of claim 1, wherein R⁸-M-Q-R⁹ are taken together to form a 6-membered aromatic or heteroaromatic ring; wherein said heteroaromatic ring contains 1 to 4 heteroatoms selected independently from the group consisting of N, O, and S; and wherein said ring is optionally substituted with 1 to 3 substituents selected independently from halo, oxo, cyano, formyl, amino, hydroxy, (C₁-C₃)alkyl, cyclopropyl, cyclopropylmethyl, (C₁-C₃)alkoxy, (C₁-C₃)alkylthio(hydroxy-(C₁-C₃)alkyl, (C₁-C₃)alkylthio-(C₁-C₂)alkyl), and (C₁-C₃)alkylthio(C₁-C₂)alkyl); wherein said alkyl and alkoxy groups are optionally independently substituted with 1 to 5 fluorine atoms;
R¹¹ and R¹², when present, are independently selected from the group consisting of H, hydroxy, nitro, R³, and R^{3a};
R¹⁰, when present, is selected from the group consisting of H, hydroxy, nitro, NHR¹³, and R³;
or a pharmaceutically acceptable salt thereof.

7. A compound of claim 1, wherein R² is a 5 to 6-membered heteroaryl containing 1 to 3 heteroatoms independently selected from the group consisting of O, N, and S; wherein R² is optionally substituted with 1 to 3 substituents; wherein one substituent may be selected from the group consisting of halo, OH, ON, amino, R¹⁵, hydroxy-(C₁-C₄alkyl, R¹⁵O—(C₁-C₂)alkyl, cyano-(C₁-C₄)alkyl, OR¹², SR¹⁵, SO₂R¹⁵(and NR¹⁵R¹⁶; and wherein 1 to 2 substituents may be independently selected from halo, methyl, ethyl, n-propyl, methoxy, ethoxy, difluoromethyl, and trifluoromethyl:
or a pharmaceutically acceptable salt thereof.

8. A compound of claim 1, wherein R² is selected from the group consisting of pyridyl and a 5-membered heteroaryl containing 1 to 2 heteroatoms independently selected from N, O, and S; and wherein said group is optionally substituted with 1 to 2 substituents independently selected form chloro, fluoro, or methyl:
or a pharmaceutically acceptable salt thereof.

9. A compound of claim 1, wherein R² is selected from the group consisting of thienyl, thiazoyl, oxazolyl, 2-pyridyl, and 3-pyridyl; wherein said group is optionally substituted with 1 to 2 substituents independently selected from chloro, fluoro, or methyl;
or a pharmaceutically acceptable salt thereof.

10. A compound of claim 2, wherein R⁸-M-Q-R⁹ are taken together to form a ring; R¹¹ and R¹², when present, are independently selected from the group consisting of H, hydroxy, nitro, R³, and R^{3a}; and wherein R¹⁰, when present, is selected from the group consisting of H, hydroxy, nitro, NHR¹³, and R³;
or optionally when n is zero, R⁹-Q-U—R¹¹ are taken together to form a ring; and R⁸ and R¹², when present, are independently selected from the group consisting of H, hydroxy, nitro, R³, and R^{3a};
or optionally when n is 1, R⁹-Q-T-R¹⁰ are taken together to form a ring; R⁸, R¹¹, and R¹², when present, are independently selected from the group consisting of H, hydroxy, nitro, R³, and R^{3a};
wherein said rings are carbocyclic or heterocyclic; wherein said heterocyclic ring contains 1 to 4 heteroatoms selected independently from the group consisting of N, O, and S; and wherein said rings are optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃))alkyl, (C₁-C₃)alkylthio-(C₁-C₂)alkyl), and (C₁-C₃)alkylthio(C₁-C₂)alkyl); wherein said alkyl and alkoxy groups are optionally independently substituted with 1 to 5 fluorine atoms;
or a pharmaceutically acceptable salt thereof.

11. A compound of claim 6, wherein W and Z are carbon; X is nitrogen; Y is CR²⁰; and R² is selected from the group consisting of thienyl, thiazoyl, oxazolyl, 2-pyridyl, and 3-pyridyl; wherein said group is optionally substituted with 1 to 2 substituents independently selected from chloro, fluoro, or methyl;
or a pharmaceutically acceptable salt thereof.

12. A compound of claim 1, wherein E, F, G, and J are carbon; wherein E, F, G, and J are optionally independently substituted with fluorine, chlorine, or methyl;
W and Z are carbon;
X is nitrogen:
Y is CR²⁰; wherein R²⁰ is hydrogen or halo;
R² is selected from the group consisting of thienyl, thiazoyl, oxazolyl, 2-pyridyl, and 3-pyridyl; wherein R² is optionally substituted with 1 to 2 substituents selected from fluorine, chlorine, and methyl;
R⁸M-Q-R⁹ are taken together to form a 6-membered aromatic or heteroaromatic ring; wherein said heteroaromatic ring contains 1 to 4 heteroatoms selected independently from the group consisting of N, O, and S; wherein said ring is optionally substituted with 1 to 3 substituents selected from halo, oxo, cyano, formyl, amino, hydroxy, (C₁-C₃)alkyl, cyclopropyl, cyclopropylmethyl, (C₁-C₃)alkoxy, (C₁-C₃)alkylthio, hydroxy-(C₁-C₃)alkyl, (C₁₋C₃)alkylthio-(C₁-C₂)alkyl), and (C₁-C₃)alkylthio(C₁-C₂)alkyl); wherein said alkyl and alkoxy groups are optionally substituted with 1 to 5 fluorine atoms;
R¹¹ and R¹², when present, are independently selected from the group consisting of H, hydroxy, nitro, R³, and R^{3a};
R¹⁰, when present, is selected from the group consisting of H, hydroxy, nitro, NHR¹³, and R³;
or a pharmaceutically acceptable salt thereof.

13. A compound of claim 12, wherein n is zero; or a pharmaceutically acceptable salt thereof.

14. A compound of claim 13, wherein R¹ is selected from the group consisting of pyridyl, pyrimidinyl, and phenyl; wherein R¹ is optionally substituted with 1 to 3 substituents independently selected from the group consisting of halo, (C₁-C₃)alkyl, and (C₁-C₃)alkoxy;
or a pharmaceutically acceptable salt thereof.

15. A compound of claim 1, wherein R¹ is pyridyl optionally substituted with one or two substituents independently selected from (C₁-C₅)alkyl and halo;
R² is thiazolyl, oxazolyl, or thienyl optionally substituted 1 or 2 substituents independently selected from methyl, chloro, and fluoro;
E, F, G, and J are carbon;
R⁴ R⁵, R⁶, and R⁷ are independently selected from the group consisting of hydrogen, halo, and methyl;
L is nitrogen;
n is zero;
V is carbon;
U is carbon or nitrogen;
R⁸-M-Q-R⁹ are taken together to form a 6-membered aromatic or heteroaromatic ring; optionally substituted with one or two substituents independently selected from the group consisting of halo, cyano, (C₁-C₄)alkyl, and (C₁-C₃)alkoxy; and wherein said heteroaromatic ring contains one nitrogen atom;
R¹¹ when present, is selected from hydrogen, halo, (C₁-C₃)alkyl, CF₂H, CF₃, CF₂CF₃, cyano, and (C₁-C₅)alkoxy;
R¹² is selected from the group consisting of hydrogen, halo, (C₁-C₅)alkyl, CF₂H, CF₃, (C₁-C₃), cyano, (C₁-C₅)alkoxy, (C₃-C₇)cycloalkyl, (C₃-C₇)cycloalkyl-(C₁-C₃)alkyl, (C₁-C₃)alkoxy-(C₁-C₃)alkyl, phenyl, pyridyl, phenoxy, pyridyloxy, benzyl, and pyridylmethyl; wherein said phenyl, pyridyl, phenoxy, pyridyloxy, benzyl, and pyridylmethyl are optionally substituted with 1 or 2 substituents independently selected from halo and methyl;
or a pharmaceutically acceptable salt thereof.

16. A compound of claim 1, selected from the group consisting of
1 (4-(1-(4-methoxyphenyl)-4-(thiophen-2-yl)-1H-imidazol-2-yl)phenyl)-1H-pyrrolo[2,3-b]pyridine,
1-(4-(1-(4-methoxyphenyl)-4-(thiazol-2-yl)-1H-imidazol-2-yl)phenyl)-1H-pyrrolo[2,3-b]pyridine,
1-(4-(1,4-di(thiazol-2-yl)-1H-imidazol-2-yl)phenyl)-1H-pyrrolo[2,3-b]pyridine,
1-(4-(4-(pyridin-2-yl)-1-(pyrimidin-5-yl)-1H-imidazol-2-yl)phenyl)-1H-pyrrolo[2,3-b]pyridine,
1-(4-(1-(2-methylpyridin-4-yl)-4-(pyridin-2-yl)-1H-imidazol-2-yl)phenyl)-1H-pyrrolo[2,3-b]pyridine,
1-(4-(1-(6-methylpyridin-3-yl)-4-(pyridin-2-yl)-1H-imidazol-2-yl)phenyl)-1H-pyrrolo[2,3-b]pyridine,
1-(4-(4-(pyridin-2-yl)-1-(pyridin-3-yl)-1H-imidazol-2-yl)phenyl)-1H-pyrrolo[2,3-b]pyridine,
1-(4-(1-(6-(1H-imidazol-1-yl)pyridin-3-yl)-4-(pyridin-2-yl)-1H-imidazol-2-yl)phenyl)-1H-pyrrolo[2,3-b]pyridine,
1-(4-(1-(6-methoxy pyridin-3-yl)-4-(pyridin-2-yl)-1H-imidazol-2-yl)phenyl)-1H-pyrrolo[2,3-b]pyridine,
N,N-dimethyl-2-(1-(4-(4-(pyridin-2-yl)-1-(pyridin-3-yl)-1H-imidazol-2-yl)phenyl)-1H-pyrrolo[2,3-b]pyridin-3-yl)ethanamine,
1-(3-fluoro-4-(4-(pyridin-2-yl)-1-(pyridin-3-yl)-1H-imidazol-2-yl)phenyl)-1H-pyrrolo[2,3-b]pyridine,
1-(2-methyl-4-(1-(6-methylpyridin-3-yl)-4-(pyridin-2-yl)-1H-imidazol-2-yl)phenyl)-1H-pyrrolo[2,3-b]pyridine,
1-(3-methyl-4-(1-(6-methylpyridin-3-yl)-4-(pyridin-2-yl)-1H-imidazol-2-yl)phenyl)-1H-pyrrolo[2,3-b]pyridine,
1-(4-(4-(pyridin-2-yl)-1-(1-oxido-pyridin-3-yl)-1H-imidazol-2-yl)phenyl)-1H-pyrrolo[2,3-b]pyridine,
1-(4-(1-(1-oxido-6-methylpyridin-3-yl)-4-(1-oxido-pyridin-2-yl)-1H-imidazol-2-yl)phenyl)-1H-indole,
1-(4-(1-(6-methylpyridin-3-yl)-4-(1-oxido-pyridin-2-yl)-1H-imidazol-2-yl)phenyl)-1H-pyrrolo[2,3-b]pyridine,
9-[4-(4-pyridin-2-yl-1-pyridin-3-yl-1H-imidazol-2-yl)phenyl]5,7,8,9-tetrahydrothiopyrano[3′,4′,4,5]pyrrolo[2,3-b]pyridine,
N,N-dimethyl(1-(4-(4-pyridin-2-yl)-1-(pyridin-3-yl)-1H-imidazol-2-yl)phenyl)-1H-pyrrolo[2,3-b]pyridin-3-yl)methanamine,
9-(4-(4-(pyridin-2-yl)-1-(pyridin-3-yl)-1H-imidazol-2-yl)phenyl)-9H-pyrido[2,3-b]indole,
5-chloro-1-(4-(4-(pyridin-2-yl)-1-(6-methylpyridin-3-yl)-1H-imidazol-2-yl)phenyl)-1H-pyrrolo[2,3-b]pyridine,
5-fluoro-1-(4-(1-(6-methylpyridin-3-yl)-4-(pyridin-2-yl)-1H-imidazol-2-yl)phenyl)-1H-pyrrolo[2,3-b]pyridine,
5-methyl-1-(4-(1-(6-methylpyridin-3-yl)-4-(pyridin-2-yl)-1H-imidazol-2-yl)phenyl)-1H-pyrrolo[2,3-b]pyridine,
1-(4-(1-(pyridin-3-yl)-4-(thiazol-2-yl)-1H-imidazol-2-yl)phenyl)-1H-pyrrolo[2,3-b]pyridine,
1-(4-(1-(pyridin-2-yl)-4-(thiazol-2-yl)-1H-imidazo-2-yl)phenyl)-1H-pyrrolo[2,3-b]pyridine,
1-(4-(1-(pyridin-4-yl)-4-(thiazol-2-yl)-1H-imidazol-2-yl)phenyl)-1H-pyrrolo[2,3-b]pyridine,
1-(4-(1-(pyrimidin-5-yl)-4-(thiazol-2-yl)-1H-imidazol-2-yl)phenyl)-1H-pyrrolo[2,3-b]pyridine,
1-(4-(1-(6-methylpyridin-3-yl)-4-(thiazol-2-yl)-1H-imidazol-2-yl)phenyl)-1H-pyrrolo[2,3-b]pyridine,
1-(4-(1-(2-methylpyridin-3-yl)-4-(thiazol-2-yl)-1H-imidazol-2-yl)phenyl)-1H-pyrrolo[2,3-b]pyridine,
1-(4-(1-(6-methoxypyridin-3-yl)-4-(thiazol-2-yl)-1H-imidazol-2-yl)phenyl)-1H-pyrrolo[2,3-b]pyridine,
5-(2-(4-(1H-pyrrolo[2,3-b]pyridin-1-yl)phenyl)-4-(thiazol-2-yl)-1H-imidazol-1-yl-N,N-dimethylpyridin-2-amine,
2-(4-(2-(4-(1H-pyrrolo[2,3-b]pyridin-1-yl)phenyl)-4-(thiazol-2-yl)-1H-imidazol-1-yl)phenyl)-N-methylethanamine,
1-(4-(1-(6-(trifluoromethyl)pyridin-3-yl)-4-(thiazol-2-yl)-1H-imidazol-2-yl)phenyl-pyrrolo[2,3-b]pyridine,
(4-(2-(4-(1H-pyrrolo[2,3-b]pyridin-1-yl)phenyl)-4-(thiazol-2-yl)-1H-imidazol-1-yl)phenyl)-N-methylmethanamine Hydrochloride,
1-(4-(1-(6-morpholinopyridin-3-yl)-4-(thiazol-2-yl)-1H-imidazol-2-yl)phenyl)-1H-pyrrolo[2,3-b]pyridine,
1-(4-(4-(pyridin-2-yl)-1-(pyridin-3-yl)-1H-imidazol-2-yl)phenyl)-1H-indazole,
1-(4-(4-(pyridin-2-yl)-1-(pyridin-3-yl)-1H-imidazol-2-yl)phenyl)-1H-indole,
7-fluoro-1-(4-(4-(pyridin-2-yl)-1-(pyridin-3-yl)-1H-imidazol-2-yl)phenyl)-1H-indole,
4,5,6,7-tetrafluoro-1-(4-(4-(pyridin-2-yl)-1-(pyridin-3-yl)-1H-imidazol-2-yl)phenyl)-1H-indole,
4-chloro-1-(4-(4-(pyridin-2-yl)-1-(pyridin-3-yl)-1H-imidazol-2-yl)phenyl)-1H-indole,
1-(4-(4-(pyridin-2-yl)-1-(pyridin-3-yl)-1H-imidazol-2-yl)phenyl)-1H-indole-4-carbonitrile,
3-(2-(4-(4-methyl-1H-imidazol-1-yl)phenyl)-4-(pyridin-2-yl)-1H-imidazol-1-yl)pyridine,
1-(4-(4-(pyridin-2-yl)-1-(pyridin-3-yl)-1H-imidazol-2-yl)phenyl)-1H-benzo[d][1,2,3]triazole,
2-(pyridin-2-yl)-1-(4-(4-(pyridin-2-yl)-1-(pyridin-3-yl)-1H-imidazol-2-yl)phenyl)-1H-benzo[d]imidazole,
3-(2-(4-(1H-imidazol-1-yl)phenyl)-4-(pyridin-2-yl)-1H-imidazol-1-yl)pyridine,
1-(4-(4-(pyridin-2-yl)-1-(pyridin-3-yl)-1H-imidazol-2-yl)phenyl)-1H-benzo[d]imidazole,
1-(4-(4-(pyridin-2-yl)-1-(pyridin-3-yl)-1H-imidazol-2-yl)phenyl)-1H-imidazo[4,5-b]pyridine,
3-(4-(4-(pyridin-2-yl)-1-(pyridin-3-yl)-1H-imidazol-2-yl)phenyl)-3H-imidazo[4,5-b]pyridine,
1-(4-(1-(6-methylpyridin-3-yl)-4-(pyridin-2-yl)-1H-imidazol-2-yl)phenyl)-1H imidazo[4,5-b]pyridine,
3-(4-(1-(6-methylpyridin-3-yl)-4-(pyridin-2-yl)-1H-imidazo imidazo[4,5-b]pyridine,
5-(4-(1-(6-methylpyridin-3-yl)-4-(pyridin-2-yl)-1H-imidazol-2-yl)phenyl)-5H-pyrrolo[3,2-b]pyrazine,
3-(4-(4-pyridin-2-yl)-1-(pyridin-3-yl)-1H-imidazol-2-yl)phenyl)-3H-[1,2,3]triazole[4,5-b]pyridine,
1-(4-(1-(6-methylpyridin-3-yl)-4-(pyridin-2-yl)-1H-imidazol-2-yl)phenyl)-1H-pyrrolo[3,2-b]pyridine,
1-(4-(1-(6-methylpyridin-3-yl)-4-(pyridin-2-yl)-1H-imidazol-2-yl)phenyl)-1H-pyrrolo[3,2-b]pyridine,
1-(4-(1-(6-methylpyridin-3-yl)-4-(pyridin-2-yl)-1H-imidazol-2-yl)phenyl)-1H-pyrrolo[3,2-c]pyridine,
9-(4-(1-(6-methylpyridin-3-yl)-4-(pyridin-2-yl)-1H-imidazol-2-yl)phenyl)-9H-purine,
7-(4-(1-(6-methylpyridin-3-yl)-4-(pyridin-2-yl)-1H-imidazol-2-yl)phenyl)-7H-purine,
1-(4-(1-(6-methylpyridin-3-yl)-4-(pyridin-2-yl)-1H-imidazol-2-yl)phenyl)-1H-pyrazolo[3,4-c]pyridine,
2-methyl-3-(4-(1-(6-methylpyridin-3-yl)-4-(pyridin-2-yl)-1H-imidazol-2-yl)phenyl)-3H-imidazo[4,5-b]pyridine,
2-(trifluoromethyl)-3-(4-(1-(6-methylpyridin-3-yl)-1-(pyridin-2-yl)-1H-imidazol-2-yl)phenyl)-3H-imidazo[4,5-b]pyridine,
2-isopropyl-3-(4-(H6-methylpyridin-3-yl)-4-(pyridin-2-yl)-1H-imidazol-2-yl)phenyl)-3H-imidazo[4,5-b]pyridine,
2-methoxy-3-(4-(1-(6-methylpyridin-3-yl)-4-(pyridin-2-yl)-1H-imidazol-2-yl)phenyl)-3H-imidazo[4,5-b]pyridine,
1-(4-(1-(6-methylpyridin-3-yl)-4-(5-methylthiazol-2-yl)-1H-imidazol-2-yl)phenyl)-1H pyrrolo[2,3-b]pyridine,
1-(4-(4-(5-chlorothiophen-2-yl)-1-(pyrimidin-5-yl)-1H-imidazol-2-yl)phenyl)-1H-pyrrolo[2,3-b]pyridine,
1-(4-(4-(4-methylthiazol-2-yl)-1-(pyrimidin-5-yl)-1H-imidazol-2-yl)phenyl)-1H-pyrrolo[2,3-b]pyridine,
1-(4-(4-(5-fluorothiophen-2-yl)-1-(6-methylpyridin-3-yl)-1H-imidazol-2-yl)phenyl)-1H-pyrrolo[2,3-b]pyridine,
1-(4-(4-(4,5-dimethylthiazol-2-yl)-1-(pyrimidin-5-yl)-1H-imidazol-2-yl)phenyl)-1H-pyrrolo[2,3-b]pyridine,
1-(4-(4-(1-methyl-1H-imidazol-2-yl)-1-(2-methylpyridin-4-yl)-1H-imidazol-2-yl)phenyl)-1H-pyrrolo[2,3-b]pyridine,
1-(4-(4-(1-methyl-1H-imidazol-2-yl)-1-(pyrimidin-5-yl)-yl)-1H-imidazol-2-yl)phenyl)-1H-pyrrolo[2,3-b]pyridine,
1-(4-(1-(2-methylpyridin-4-yl)-4-(pyridin-3-yl)-1H-imidazol-2-yl)phenyl)-1H-pyrrolo[2,3-b]pyridine,
1-(4-(1-(2-methylpyridin-4-yl)-4-(pyridin-4-yl)-imidazol-2-yl)-1H-pyrrolo[2,3-b]pyridine,
5-(2-(4-(3,4-dichlorophenyl)phenyl)-4-(pyridin-2-yl)-1H-imidazol-1-yl)pyrimidine,
5-(2-(4-(4-chlorophenyl)phenyl)-4-(pyridin-2-yl)-1H-imidazol-1-yl)pyrimidine,
5-(4-(pyridin-2-yl)-2-(4-(pyridin-3-yl)phenyl)-1H-imidazol-1-yl)pyrimidine,
5-(4-(pyridin-2-yl)-2-(4-(pyridin-4-yl)phenyl)-1H-imidazol-1-yl)pyrimidine,
7-(4-(1-(6-methylpyridin-3-yl)-4-(pyridin-2-yl)-1H-imidazol-2-yl)phenyl)-7H-pyrrolo[2,3-d]pyrimidine,
7-methyl-5-(4-(1-(6-methylpyridin-3-yl)-4-(pyridin-2-yl)-1H-imidazol-2-yl)phenyl)-5H-pyrrolo[2,3-b]pyrazine,
1-(4-(4-(benzo[d]thiazol-2-yl)-1-(pyridin-3-yl)-1H-imidazol-2-yl)phenyl)-1H-pyrrolo[2,3-b]pyridine,
4-methoxy-6-methyl-8-(4-(4-(pyridin-2-yl)-1-(pyridin-3-yl)-1H-imidazol-2-yl)phenyl)quinoline,
8-(4-(4-(pyridin-2-yl)-1-(pyridin-3-yl)-1H-imidazol-2-yl)phenyl)-1,7-naphthyridine,
8-(4-(4-(pyridin-2-yl)-1-(pyridin-3-yl)-1-(imidazol-2-yl)phenyl)quinoline,
6-methoxy-8-(4-(4-(pyridin-2-yl)-1-(pyridin-3-yl)-1H-imidazol-2-yl)phenyl)quinoline
2-methoxy-3-(4-(1-(6-methylpyridin-3-yl)-4-(thiazol-2-yl)-1H-imidazol-2-yl)phenyl)-3H-imidazo[4,5-b]pyridine,
2-ethyl-3-(4-(1-(6-methylpyridin-3-yl)-4-(5-methylthiazol-2-yl)-1H-imidazol-2-yl)phenyl)-3H-imidazo[4,5-b]pyridine,
2-ethyl-3-(4-(1-(6-methylpyridin-3-yl)-4-(4-methylthiazol-2-yl)-1H-imidazol-2-yl)phenyl)-3H-imidazo[4,5-b]pyridine,
2-(difluoromethyl)-3-(4-(1-(6-methylpyridin-3-yl-4-(thiazol-2-yl)-1H-imidazol-2-yl)phenyl)-3H-imidazo[4,5-b]pyridine,
2-ethyl-3-(4-(1-(6-methylpyridin-3-yl)-4-(thiazol-2-yl)-1H-imidazol-2-yl)phenyl)-3H-imidazo[4,5-b]pyridine,
2-isopropyl-3-(4-(1-(6-methylpyridin-3-yl)-4-(thiazol-2-yl)-1H-imidazol-2-yl)phenyl)-3H-imidazo[4,5-b]pyridine,
2-(trifluoromethyl)-3-(4-(1-(6-methylpyridin-3-yl)-4-(thiazol-2-yl)-1H-imidazol-2-yl)phenyl)-3H-imidazo[4,5-b]pyridine,
3-(4-(3-(pyridin-2-yl)-5-(pyridin-3-yl)-1H-1,2,4-triazol-1-yl)phenyl)-1H-imidazo[4,5-b]pyridin-2(3H)-one,
2-methoxy-t-(4-(t-(6-methylpyridin-3-yl)-4-(pyridin-2-yl)-1H-imidazol-2-yl)phenyl)-1H-imidazo[4,5-c]pyridine,
2-methoxy-3-(4-(1-(6-methylpyridin-3-yl)-4-(4-methylthiazol-2-yl)-1H-imidazol-2-yl)phenyl)-3H-imidazo[4,5-b]pyridine,
3-(4-(1-(6-methylpyridin-3-yl)-4-(pyridin-2-yl)-1H-imidazol-2-yl)phenyl)-2-propoxy-3H-imidazo[4,5-b]pyridine,
2-(methoxymethyl)-3-(4-(1-(8-methylpyridin-3-yl)-4-(thiazol-2-yl)-1H-imidazol-2-yl)phenyl)-3H-imidazo[4,5-b]pyridine,
2-methoxymethyl)-3-(4-(1-(6-methylpyridin-3-yl)-4-3H-imidazo)-4,5-b]pyridine,
2-ethoxy-3-(4-(1-(6-methylpyridin-3-yl)-4-(pyridin-2-yl)-1H-imidazol-2-yl)phenyl)-3H-imidazo[4,5-b]pyridine,
3-(4-(1-(6-methylpyridin-3-yl)-4-(pyridin-2-yl)-1H-imidazol-2-yl)phenyl)-1H-imidazo[4,5-b]pyridin-2(3H)-one,
2-methoxy-3-(4-(1-(6-methylpyridin-3-yl)-4-(thiazol-4-yl)-1H-imidazol-2-yl)phenyl)-3-imidazo[4,5-b]pyridine,
2-isopropyl-3-(4-(1-(6-methylpyridin-3-yl)-4-(thiazol-4-yl)-1H-imidazol-2-yl)phenyl)-3H-imidazo[4,5-b]pyridine,
2-isopropyl-3-(4-(1-(6-methylpyridin-3-yl)-4-(thiazol-4-yl)-1H-imidazol-2-imidazo[4,5-b]pyridine,
2-(trifluoromethyl)-3-(4-(1-(6-methylpyridin-3-yl)-1H-(thiazol-4-yl)-1H-imidazol-2-yl)phenyl)-3H-imidazo[4,5-b]pyridine,
2-ethoxy-3-(4-(t-(6-methylpyridin-3-yl)-4-(thiazol-4-yl)-1H-imidazol-2-yl)phenyl)-3H-imidazo[4,5-b]pyridine,
3-(4-(5-(4-methoxyphenyl)-2-(thiophen-2-yl)-1H-imidazol-4-yl)phenyl)-3H-imidazo[4,5-b]pyridine,
1-(4-(5-(4-methoxyphenyl)-2-(thiophen-2-yl)-1H-imidazol-4-yl)phenyl)-1H-imidazol-[4,5-b]pyridine,
5-methoxy-1-(4-(5-(4-methoxyphenyl)-2-(thiophen-2-yl)-1H-imidazol-4-yl)phenyl)-1H-indole,
1-(4-(5-(4-methoxyphenyl)-2-(thiophen-2-yl)-1H-imidazol-4-yl)phenyl)-1H-pyrrolo[2,3-b]pyridine,
1-(4-(1-hydroxy-5-(pyrazin-2-yl)-2-(thiophen-2-yl)-1H-imidazol-4-yl)phenyl)-1H-pyrrolo[2,3-b]pyridine,
1-(4-(5-(pyrazin-2-yl)-2-(thiophen-2-yl)-1H-imidazol-4-yl)phenyl)-1H-pyrrolo[2,3-b]pyridine,
1-(4-(S-(4-methoxyphenyl)-2-(thiophen-2-yl)-1H-imidazol-4-yl)phenyl)-1H-imidazole,
1-(4-(5-(6-(4-methylpiperazin-1-yl)pyridin-3-yl)-2-(thiazol-5-yl)-1H-imidazol-4-yl)phenyl)-1H-pyrrolo-[2,3-b]pyridine,
1-(4-(5-(4-methoxyphenyl)-2-(thiophen-2-yl)-1H-imidazol-4-yl)phenyl)-4-phenyl-1-H-imidazole,
1-(4-(1-hydroxy-5-(pyrazin-2-yl)-2-(thiophen-2-yl)-1H-imidazol-4-yl)-2-methylphenyl)-1H-pyrrolo[2,3-b]pyridine,
1-(4-(1-hydroxy-5-(pyrazin-2-yl)-2-(thiophen-2-yl)-1H-(imidazol-4-yl)-2-methylphenyl)-1H-pyrrolo[2,3-b]pyridine,
1-(4-(1-hydroxy-5-(pyrazin-2-yl)-2-(thiophen-2-yl)(1H-imidazol-4-yl)phenyl)-1H-pyrrolo[2,3-b]pyridine,
1-(2-methyl-4-(5-(pyrazin-2-yl)-2-(thiazol-5-yl)-1H-imidazol-4-yl)phenyl)-1-H-pyrrolo[2,3-b]pyridine,
1-(2-methyl-4-(5-(pyrazin-2-yl)-2-(thiazol-5-yl-1H-imidazol-4-yl)phenyl)-1H-pyrrolo[2,3-b]pyridine,
1-(4-(2-(pyridin-2-yl)-4-(pyridin-3-yl)-1H-imidazol-5-yl)phenyl)-1H-pyrrolo[2,3-b]pyridine,
1-(4-(3-(pyridin-2-yl)-5-(pyridin-3-yl)-1H-1,2,4-triazol-1-yl)phenyl)-1H-pyrrolo[2,3-b]pyridine,
2-methoxy-3-(4-(1-(6-methylpyridin-3-yl)-4-(thiazol-5-yl)-1H-imidazol-2-yl)phenyl)-3H-imidazo[4,5-b]pyridine,
2-(difluoromethyl)-3-(4-(1-(6-methylpyridin-3-yl)-4-(thiazol-5-yl)-1H-imidazol-2-yl)phenyl)-3H-imidazo[4,5-b]pyridine,
3-(4-(1-(6-methylpyridin-3-yl)-4-(thiazol-2-yl)-1H-imidazol-2-yl)phenyl)-1H-imidazo[4,5-b]pyridin-2(3H)-one,
1-(4-(2-(pyridin-2-yl)-5-(pyridin-3-yl)-2H-1,2,3-triazol-4-yl)-phenyl)-1H-pyrrolo[2,3-b]pyridine,
1-(4-(1-(pyridin-2-yl)-4-(pyridin-3-yl)-1H-pyrazol-3-yl)phenyl)-1H-pyrrolo-2,3-b]pyridine,
1-(4-(3-(pyridin-2-yl)-5-(pyridin-3-yl)-1H-pyrazol-1-yl)phenyl)-1H-pyrrolo-2,3-b]pyridine,
1-(4-(5-(pyridin-2-yl)-3-(pyridin-3-yl)-1H-pyrazol-1-yl)phenyl)-1H-pyrrolo[2,3-b]pyridine, and
1-(4-(5-(pyridin-2-yl)-2-(pyridin-3-yl)-2H-1,2,4-triazol-3-yl)phenyl)-1H-pyrrolo[2,3-b]pyridine,
and pharmaceutical acceptable salts thereof.

17. A pharmaceutical composition for treating a disorder or condition selected from psychotic disorders, delusional disorders and drug induced psychosis; anxiety disorders, movement disorders, mood disorders, neurodegenerative disorders, obesity, and drug addiction, comprising an amount of a compound according to claim 1, or pharmaceutically acceptable salt thereof, effective in treating said disorder or condition.

18. A method of treating a disorder or condition selected from psychotic disorders, delusional disorders and drug induced psychosis; anxiety disorders, movement disorders, mood disorders, obesity, and neurodegenerative disorders, which method comprises administering an amount of a compound of claim 1, or pharmaceutically acceptable salt thereof, effective in treating said disorder or condition.
